(19) United States
(12) Patent Application Publication
(10) Pub. No.: US 2010/0143341 A1
(43) Pub. Date: Jun. 10, 2010

(54) THIENOPYRIMIDINES FOR PHARMACEUTICAL COMPOSITIONS

(75) Inventors: Steven Taylor, Didcot (GB); Stephen Murfin, Didcot (GB); Thomas Stephen Coulter, Wantage (GB); Stefan Jakel, Darmstadt (DE); Babette Aicher, Frankfurt am Main (DE); Arndt-Rene Kelter, Alfter (DE); Joachim Kraemer, Ellerbek (DE); Christian Kirchhoff, Tornesch (DE); Andreas Scheel, Halstenbek (DE); Julian Woelcke, Freiburg im Breisgau (DE)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: DEVELOGEN AKTIENGESELLSCHAFT, Gottingen (DE)

(21) Appl. No.: 11/993,433

(22) PCT Filed: Jun. 21, 2006
(86) PCT No.: PCT/EP06/05980

§ 371 Date: Aug. 20, 2008

(30) Foreign Application Priority Data
Jun. 22, 2005 (EP) EP05013500.3

(51) Int. Cl. A61K 31/519; C07D 495/04; A61K 31/5377; A61K 38/28; A61K 31/60; A61K 39/395; A61K 31/704; A61P 3/00

(52) U.S. Cl. 424/133.1; 544/278; 544/117; 514/260.1; 514/234.2; 514/4; 514/161; 514/34

(57) ABSTRACT

The present invention relates to novel pharmaceutical compositions of general formula (I) comprising thienopyrimidine compounds. Moreover, the present invention relates to the use of the thienopyrimidine compounds of the invention for the production of pharmaceutical compositions for the prophylaxis and/or treatment of diseases which can be influenced by the inhibition of the kinase activity of Mnk1 and/or Mnk2 (Mnk2a or Mnk2b) and/or variants thereof.

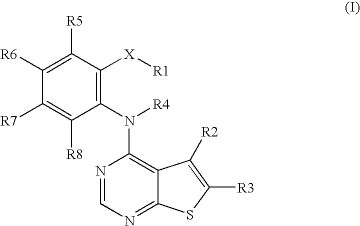

The present invention relates to thienopyrimidine compounds and to novel pharmaceutical compositions comprising thienopyrimidine compounds.

Moreover, the present invention relates to the use of the thienopyrimidine compounds of the invention for the production of pharmaceutical compositions for the prophylaxis and/or treatment of diseases which can be influenced by the inhibition of the kinase activity of Mnk1 (Mnk1a or MnK1b) and/or Mnk2 (Mnk2a or Mnk2b) or further variants thereof. Particularly, the present invention relates to the use of the thienopyrimidine compounds of the invention for the production of pharmaceutical compositions for the prophylaxis and/or therapy of metabolic diseases, such as diabetes, hyperlipidemia and obesity, hematopoietic disorders and cancer and their consecutive complications and disorders associated therewith.

Metabolic diseases are diseases caused by an abnormal metabolic process and may either be congenital due to an inherited enzyme abnormality or acquired due to a disease of an endocrine organ or failure of a metabolically important organ such as the liver or the pancreas.

The present invention is more particularly directed to the treatment and/or prophylaxis of in particular metabolic diseases of the lipid and carbohydrate metabolism and the consecutive complications and disorders associated therewith.

Lipid disorders cover a group of conditions which cause abnormalities in the level and metabolism of plasma lipids and lipoproteins. Thus, hyperlipidemias are of particular clinical relevance since they constitute an important risk factor for the development of atherosclerosis and subsequent vascular diseases such as coronary heart disease.

Diabetes mellitus is defined as a chronic hyperglycemia associated with resulting damages to organs and dysfunctions of metabolic processes. Depending on its etiology, one differentiates between several forms of diabetes, which are either due to an absolute (lacking or decreased insulin secretion) or to a relative lack of insulin. Diabetes mellitus Type I (IDDM, insulin-dependent diabetes mellitus) generally occurs in adolescents under 20 years of age. It is assumed to be of auto-immune etiology, leading to an insulitis with the subsequent destruction of the beta cells of the islets of Langerhans which are responsible for the insulin synthesis. In addition, in latent autoimmune diabetes in adults (LADA; Diabetes Care. 8: 1460-1467, 2001) beta cells are being destroyed due to autoimmune attack. The amount of insulin produced by the remaining pancreatic islet cells is too low, resulting in elevated blood glucose levels (hyperglycemia). Diabetes mellitus Type II generally occurs at an older age. It is above all associated with a resistance to insulin in the liver and the skeletal muscles, but also with a defect of the islets of Langerhans. High blood glucose levels (and also high blood lipid levels) in turn lead to an impairment of beta cell function and to an increase in beta cell apoptosis.

Diabetes is a very disabling disease, because today's common anti-diabetic drugs do not control blood sugar levels well enough to completely prevent the occurrence of high and low blood sugar levels. Out of range blood sugar levels are toxic and cause long-term complications for example retinopathy, renopathy, neuropathy and peripheral vascular disease. There is also a host of related conditions, such as obesity, hypertension, heart disease and hyperlipidemia, for which persons with diabetes are substantially at risk.

Obesity is associated with an increased risk of follow-up diseases such as cardiovascular diseases, hypertension, diabetes, hyperlipidemia and an increased mortality. Diabetes (insulin resistance) and obesity are part of the “metabolic syndrome” which is defined as the linkage between several diseases (also referred to as syndrome X, insulin-resistance syndrome, or deadly quartet). These often occur in the same patients and are major risk factors for development of diabetes type II and cardiovascular disease. It has been suggested that the control of lipid levels and glucose levels is required to treat diabetes type II, heart disease, and other occurrences of metabolic syndrome (see e.g., Diabetes 48: 1836-1841, 1999; JAMA 288: 2209-2716, 2002).

In one embodiment of the present invention the compounds and compositions of the present invention are useful for the treatment and/or prophylaxis of metabolic diseases of the carbohydrate metabolism and their consecutive complications and disorders such as impaired glucose tolerance, diabetes (preferably diabetes type II), diabetic complications such as diabetic gangrene, diabetic arthropathy, diabetic osteopenia, diabetic glomerosclerosis, diabetic nephropathy, diabetic dermopathy, diabetic neuropathy, diabetic cataract and diabetic retinopathy, diabetic maculopathy, diabetic feet syndrome, diabetic coma with or without ketoacidosis, diabetic hyperosmolar coma, hypoglycemic coma, hyperglycemic coma, diabetic acidosis, diabetic ketoacidosis, intracapillary glomerulonephrosis, Kimmelstiel-Wilson syndrome, diabetic amyotrophy, diabetic autonomic neuropathy, diabetic mononeuropathy, diabetic polyneuropathy, diabetic angiopathies, diabetic peripheral angiopathy, diabetic ulcer, diabetic arthropathy, or obesity in diabetes.

In a further embodiment the compounds and compositions of the present invention are useful for the treatment and/or prophylaxis of metabolic diseases of the lipid metabolism (i.e. lipid disorders) and their consecutive complications and disorders such as hypercholesterolemia, familial hypercholesterolemia, Fredrickson's hyperlipoproteinemia, hyperbetalipoproteinemia, hyperlipidemia, low-density-lipoprotein-type [LDL] hyperlipoproteinemia, pure hyperglyceridemia, endogenous hyperglyceridemia, isolated hypercholesterolemia, isolated hypertroglyceridemia, cardiovascular diseases such as hypertension, ischemia, varicose veins, retinal vein occlusion, atherosclerosis, angina pectoris, myocardial infarction, stenocardia, pulmonary hypertension, congestive heart failure, glomerulopaty, tubulointestitial disorders, renal failure, angiostenosis, or cerebrovascular disorders, such as cerebral apoplexy.

In a further embodiment of the present invention the compounds and compositions of the present invention are useful for the treatment and/or prophylaxis of hematopoetic disorders and their consecutive complications and disorders such as acute myeloid leukemia (AML), Morbus Hodgkin, Non-Hodgkin's lymphoma; hematopoetic disease, acute non-lymphocytic leukemia (ANLL), myeloproliferative disease acute promyelocytic leukemia (APL), acute myelomonocytic leukemia (AMMoL), polycythemia vera, lymphoma, acute lymphocytic leukemia (ALL), chronic lymphocytic leukemia (CCL), Wilm's tumor, or Ewing's Sarcoma.

In a further embodiment of the present invention the compounds and compositions of the present invention are useful for the treatment and/or prophylaxis of cancer and consecutive complications and disorders such as cancer of the upper gastrointestinal tract, pancreatic carcinoma, breast cancer, colon cancer, ovarian carcinoma, cervix carcinoma, corpus carcinoma, brain tumor, testicular cancer, laryngeal carcinoma, osteocarcinoma, prostatic cancer, retinoblastoma, liver carcinoma, lung cancer, neuroblastoma, renal carcinoma, thyroid carcinoma, espohageal cancer, soft tissue sarcoma, cachexia, or pain.

Protein kinases are important enzymes involved in the regulation of many cellular functions. The LK6-serine/threonine-kinase gene of Drosophila melanogaster was described as a short-lived kinase which can associate with microtubules (J. Cell Sci. 1997, 110(2): 209-219). Genetic analysis in the development of the compound eye of Drosophila suggested a role in the modulation of the RAS signal pathway (Genetics 2000 156(3): 1219-1230). The closest human homologues of Drosophila LK6-kinase are the MAP-kinase interacting kinase 2 (Mnk2, e.g. the variants Mnk2a and Mnk2b) and MAP-kinase interacting kinase 1 (Mnk1) and variants thereof. These kinases are mostly localized in the cytoplasm. Mnks are phosphorylated by the p42 MAP kinases Erk1 and Erk2 and the p38-MAP kinases. This phosphorylation is triggered in a response to growth factors, phorbol esters and oncogenes such as Ras and Mos, and by stress signaling molecules and cytokines. The phosphorylation of Mnk proteins stimulates their kinase activity towards eukaryotic initiation factor 4E (eIF4E) (EMBO J. 16: 1909-1920, 1997; Mol Cell Biol 19, 1871-1880, 1990; Mol Cell Biol 21, 743-754, 2001). Simultaneous disruption of both, the Mnk1 and Mnk2 gene in mice diminishes basal and stimulated eIF4E phosphorylation (Mol Cell Biol 24, 6539-6549, 2004). Phosphorylation of eIF4E results in a regulation of the protein translation (Mol Cell Biol 22: 5500-5511, 2001).

There are different hypotheses describing the mode of the stimulation of the protein translation by Mnk proteins. Most publications describe a positive stimulatory effect on the cap-dependent protein translation upon activation of MAP kinase-interacting kinases. Thus, the activation of Mnk proteins can lead to an indirect stimulation or regulation of the protein translation, e.g. by the effect on the cytosolic phospholipase 2 alpha (BBA 1488:124-138, 2000).

WO 03/037362 discloses a link between human Mnk genes, particularly the variants of the human Mnk2 genes, and diseases which are associated with the regulation of body weight or thermogenesis. It is postulated that human Mnk genes, particularly the Mnk2 variants are involved in diseases such as e.g. metabolic diseases including obesity, eating disorders, cachexia, diabetes mellitus, hypertension, coronary heart disease, hypercholesterolemia, dyslipidemia, osteoarthritis, biliary stones, cancer of the genitals and sleep apnea, and in diseases connected with the ROS defense, such as e.g. diabetes mellitus and cancer. WO 03/03762 moreover discloses the use of nucleic acid sequences of the MAP kinase-interacting kinase (Mnk) gene family and amino acid sequences encoding these and the use of these sequences or of effectors of Mnk nucleic acids or polypeptides, particularly Mnk inhibitors and activators in the diagnosis, prophylaxis or therapy of diseases associated with the regulation of body weight or thermogenesis.

WO 02/103361 describes the use of kinases 2a and 2b (Mnk2a and Mnk2b) interacting with the human MAP kinase in assays for the identification of pharmacologically active ingredients, particularly useful for the treatment of diabetes mellitus type 2. Moreover, WO 02/103361 discloses also the prophylaxis and/or therapy of diseases associated with insulin resistance, by modulation of the expression or the activity of Mnk2a or Mnk2b. Apart from peptides, peptidomimetics, amino acids, amino acid analogues, polynucleotides, polynucleotide analogues, nucleotides and nucleotide analogues, 4-hydroxybenzoic acid methyl ester are described as a substance which binds the human Mnk2 protein.

Inhibitors of Mnk (referred to as CGP57380 and CGP052088) have been described (cf. Mol. Cell. Biol. 21, 5500, 2001; Mol Cell Biol Res Comm 3, 205, 2000; Genomics 69, 63, 2000). CGP052088 is a staurosporine derivative having an IC₅₀ of 70 nM for inhibition of in vitro kinase activity of Mnk1. CGP57380 is a low molecular weight selective, non-cytotoxic inhibitor of Mnk2 (Mnk2a or Mnk2b) or of Mnk1: The addition of CGP57380 to cell culture cells, transfected with Mnk2 (Mnk2a or Mnk2b) or Mnk1 showed a strong reduction of phosphorylated eIF4E.

The problem underlying the present invention is to provide potent and selective Mnk1 and/or Mnk2 inhibitors which may effectively and safely be used for the treatment of metabolic diseases and their consecutive complication and disorders.

It has now been surprisingly found that certain thienopyrimidine compounds are potent inhibitors of the kinase enzymes Mnk1 and/or Mnk2 and/or variants thereof and as such may be useful in the prophylaxis and/or therapy of diseases which can be influenced by the inhibition of the kinase activity of Mnk1 and/or Mnk2 (Mnk2a or Mnk2b) and/or variants thereof.

Thienopyrimidine compounds of the present invention are compounds of the general formula (1):

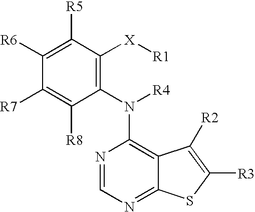

wherein X is O, S, SO₂, CH₂, CHR₁ₐ, CR₁ₐR_{1b}, CH(halogen), C(halogen)₂, C═O, C(O)NR₁ₐ, NH or NR₁ₐ, wherein R₁ₐ and R_{1b} are C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, wherein R₁ₐ and R_{1b} are optionally substituted with one or more R₉;

R₁ is hydrogen, C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, C₆₋₁₀ aryl, C₁₋₆ alkyl C₆₋₁₀ aryl, C₅₋₁₀ heteroaryl comprising at least one heteroatom selected from N, S and O, C₁₋₆ alkyl C₅₋₁₀ heteroaryl comprising at least one heteroatom selected from N, S and O, wherein R₁ is optionally substituted with one or more R₉;

or if X is NR₁ₐ, CHR₁ₐ, C(O)NR₁ₐ or CR₁ₐR_{1b}, R₁ may form a carbocyclic or heterocyclic ring with R₁ₐ and the N or C atom to which they are attached, which may contain one or more additional heteroatoms selected from N, S and O, which may be substituted with one or more R₉;

R₂ and R₃ are the same or different and are independently selected from hydrogen, C₁₋₆ alkyl, C₁₋₆ alkyl C₃₋₁₀ cycloalkyl, C₃₋₁₀ cycloalkyl, C₆₋₁₀ aryl, C₁₋₆ alkyl C₆₋₁₀ aryl, C₅₋₁₀ heteroaryl comprising at least one heteroatom selected from N, S and O, C₁₋₆ alkyl C₅₋₁₀ heteroaryl comprising at least one heteroatom selected from N, S and O, C₁₋₆ alkyl 3 to 10 membered heterocycloalkyl comprising at least one heteroatom selected from N, S and O, 3 to 10 membered heterocycloalkyl comprising at least one heteroatom selected from N, S and O, or together with the C atoms that they are attached to form a C₃₋₇ cycloalkyl or a 3 to 10 membered heterocycloalkyl group, wherein R₂ and R₃ are optionally substituted with one or more R₉, R₂ may also be R₉ and R₃ may also be R₁₀;

R₄ is hydrogen, C₁₋₄ alkyl, urea, thiourea or acetyl optionally substituted with one or more R₉;

or R₄ may form a 5 or 6 membered heterocyclic ring with R₁;

R₅, R₆, R₇ and R₈ are the same or different and are independently selected from H or R₉;

R₉ is independently halogen; CN; COOR₁₁; OR₁₁; C(O)N(R₁₁R₁₁ₐ); S(O)₂N(R₁₁R₁₁ₐ); S(O)N(R₁₁R₁₁ₐ); S(O)₂R₁₁; N(R₁₁)S(O)₂N(R₁₁ₐR_{11b}); SR₁₁; N(R₁₁R₁₁ₐ); OC(O)R₁₁; N(R₁₁)C(O)R₁₁ₐ; N(R₁₁)S(O)₂R₁₁ₐ; N(R₁₁)S(O)R₁₁ₐ; N(R₁₁)C(O)N(R₁₁ₐR_{11b}); N(R₁₁)C(O)OR₁₁ₐ; OC(O)N(R₁₁R₁₁ₐ); oxo (═O), where the ring is at least partially saturated; C(O)R₁₁; C₁₋₆ alkyl; phenyl; C₃₋₇ cycloalkyl; or heterocyclyl, wherein C₁₋₆ alkyl; phenyl; C₃₋₇ cycloalkyl; and heterocyclyl are optionally substituted with one or more R₁₀;

R₁₀ is independently halogen; CN; OR₁₁; S(O)₂N(R₁₁R₁₁ₐ); S(O)N(R₁₁R₁₁ₐ); S(O)₂R₁₁; N(R₁₁)S(O)₂N(R₁₁ₐR_{11b}); SR₁₁; N(R₁₁R₁₁ₐ); OC(O)R₁₁; N(R₁₁)C(O)R₁₁ₐ; N(R₁₁)S(O)₂R₁₁ₐ; N(R₁₁)S(O)R₁₁ₐ; N(R₁₁)C(O)N(R₁₁ₐR_{11b}); N(R₁₁)C(O)OR₁₁ₐ; OC(O)N(R₁₁R₁₁ₐ); oxo (═O), where the ring is at least partially saturated; C(O)R₁₁; C₁₋₆ alkyl; phenyl; C₃₋₇ cycloalkyl; or heterocyclyl, wherein C₁₋₆ alkyl; phenyl; C₃₋₇ cycloalkyl; and heterocyclyl are optionally substituted with one or more R₉;

R₁₁, R₁₁ₐ, R_{11b} are independently selected from the group consisting of hydrogen, C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, C₆₋₁₀ aryl, 5 to 10 membered heteroaryl comprising at least one heteroatom selected from N, S and O, wherein R₁₁, R₁₁ₐ, R_{11b} are optionally substituted with one or more R₉;

or a metabolite, prodrug or a pharmaceutically acceptable salt thereof.

Compounds in which X is O, S, SO₂, CH₂, CHR₁ₐ, CR₁ₐR_{1b}, CH(halogen), C(halogen)₂, C═O, C(O)NR₁ₐ, NH or NR₁ₐ, wherein R₁ₐ and R_{1b} are C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, wherein R₁ₐ and R_{1b} are optionally substituted with one or more R₉;

R₁ is hydrogen, C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, C₆₋₁₀ aryl, C₁₋₆ alkyl C₆₋₁₀ aryl, C₅₋₁₀ heteroaryl comprising at least one heteroatom selected from N, S and O, C₁₋₆ alkyl C₅₋₁₀ heteroaryl comprising at least one heteroatom selected from N, S and O, wherein R₁ is optionally substituted with one or more R₉;

or if X is NR₁ₐ, CHR₁ₐ, C(O)NR₁ₐ or CR₁ₐR_{1b}, R₁ may form a carbocyclic or heterocyclic ring with R₁ₐ and the N or C atom to which they are attached, which may contain one or more additional heteroatoms selected from N, S and O, which may be substituted with one or more R₉;

R₂ and R₃ are the same or different and are independently selected from hydrogen, methyl, phenyl, ethyl, propyl, perfluoromethyl, or form together with the C atoms to which they are attached a 5-membered carbocyclic ring;

R₄ is hydrogen or C₁₋₄ alkyl;

R₅, R₆, R₇ and R₈ are the same or different and are independently selected from hydrogen, CONH₂, CO₂H, CO₂CH₃, Cl and F;

R₉ is as defined above;

or a metabolite, prodrug or pharmaceutically acceptable salt thereof are preferred.

Also preferred are compounds in which X is O, S, SO₂, CH₂, CHR₁ₐ, CR₁ₐR_{1b}, CH(halogen), C(halogen)₂, C═O, C(O)NR₁ₐ, NH or NR₁ₐ, wherein R₁ₐ and R_{1b} are C₁₋₆ alkyl;

R₁ is hydrogen, methyl, ethyl, propyl, butyl, difluoromethyl, bromoethyl, 1,1,2,2-tetrafluoroethyl, 1,1,1-trifluoropropyl, perfluoromethyl, cyclopropylmethyl, cyclopentyl, cyclohexyl, adamantyl, norbonanyl, tetrahydrofuranyl, tetrahydropyranyl, phenyl or pyrrolidin-3-yl substituted at the nitrogen with R₉;

or if X is NR₁ₐ, R₁ forms a morpholino group, a pyrrolidino group or a piperidino group together with R₁ₐ and the N atom to which they are attached, which may be substituted with —CH₃ or —C(O)OC₄H₉;

R₂ and R₃ are the same or different and are independently selected from hydrogen, methyl, phenyl, ethyl, propyl, perfluoromethyl, or form together with the C atoms to which they are attached a 5-membered carbocyclic ring;

R₄ is hydrogen or C₁₋₄ alkyl;

R₅, R₆, R₇ and R₈ are the same or different and are independently selected from hydrogen, CONH₂, CO₂H, CO₂CH₃, Cl and F;

R₉ is as defined above;

or a metabolite, prodrug or pharmaceutically acceptable salt thereof.

Compounds wherein R₂ and R₃ are the same or different and are selected from methyl, hydrogen and perfluoromethyl are more preferred.

The present invention also relates to compounds in which X is O, S, SO₂, CH₂, CHR₁ₐ, CR₁ₐR_{1b}, CH(halogen), C(halogen)₂, C═O, C(O)NR₁ₐ, NH or NR₁ₐ, wherein R₁ₐ and R_{1b} are C₁₋₆ alkyl;

R₁ is hydrogen, C₁₋₆ alkyl, C₁₋₆ alkyl C₃₋₁₀ cycloalkyl, C₃₋₁₀ cycloalkyl, 5 to 10 membered heterocyclyl comprising at least one heteroatom selected from N, S and O, C₆₋₁₀ aryl, C₁₋₆ alkyl C₆₋₁₀ aryl, C₅₋₁₀ heteroaryl comprising at least one heteroatom selected from N, S and O, C₁₋₆ alkyl C₅₋₁₀ heteroaryl comprising at least one heteroatom selected from N, S and O, wherein R₁ is optionally substituted with one or more R₉;

or if X is NR₁ₐ, R₁ may form a heterocyclic ring together with R₁ₐ and the N atom to which they are attached, which may contain an additional heteroatom selected from N, S and O, which may be substituted with one or more R₉;

R₂ and R₃ are the same or different and are independently selected from hydrogen, C₁₋₄ alkyl which may optionally be substituted with one or more halogen atoms, an acetyl group, a urea, a hydroxyl, a phenyl group and an amino group or form together with the C atoms to which they are attached a C₃₋₆ cycloalkyl group;

R₄ is hydrogen or C₁₋₄ alkyl;

R₅, R₆, R₇ and R₈ are the same or different and are independently selected from hydrogen, CO₂H, CO₂R_{1c}, CONH₂, CONHR_{1d} and halogen, whereby R_{1c} and R_{1d} are C₁₋₆ alkyl;

R₉ is as defined above;

with the proviso that if R₃ is H or C₁₋₄ alkyl, R₂ cannot be hydrogen;

or a metabolite, prodrug or pharmaceutically acceptable salt thereof.

Compounds in which R₄ is hydrogen are preferred as well as compounds in which X represents O and/or compounds in which the cycloalkyl group is adamantyl or norbonanyl, cyclohexyl or cyclopentyl.

The compounds of the present invention may contain a halogen atom preferable selected from Cl, Br and F.

In one aspect, the present invention relates to compounds in which R₅, R₆, R₇ and R₈ are hydrogen and, in another aspect, to compounds in which at least one of R₅, R₆, R₇ and R₈ represents F, CONH₂ or CO₂CH₃.

In a preferred embodiment, the compounds of the present invention contain a R₁ group which is selected from hydrogen, methyl, ethyl, propyl, butyl, difluoromethyl, bromoethyl, 1,1,2,2-tertrafluoroethyl, 1,1,1-tritluoropropyl, perfluoromethyl, cyclopropylmethyl, cyclopentyl, cyclohexyl, adamantyl, norbonanyl, tetrahydrofuranyl, tetrahydropyranyl, phenyl or pyrrolidin-3-yl substituted at the nitrogen with R₉, wherein R₉ is as defined above.

Particularly preferred compounds are selected from:

(5,6-Dimethyl-thieno[2,3-d]pyrimidin-4-yl)-[2-(tetrahydro-furan-3-yloxy)-phenyl]-amine,

(5,6-Dimethyl-thieno[2,3-d]pyrimidin-4-yl)-[2-((R)-tetrahydro-furan-3-yloxy)-phenyl]-amine,

(5,6-Dimethyl-thieno[2,3-d]pyrimidin-4-yl)-[2-((S)-tetrahydro-furan-3-yloxy)-phenyl]-amine,

(5-Methyl-thieno[2,3-d]pyrimidin-4-yl)-[2-(tetrahydro-furan-3-yloxy)-phenyl]-amine,

(2-Cyclopentyloxy-phenyl)-(5-methyl-thieno[2,3-d]pyrimidin-4-yl)-amine,

(5,6-Dimethyl-thieno[2,3-d]pyrimidin-4-yl)-[2-(tetrahydro-pyran-4-yloxy)-phenyl]-amine,

(2-sec-Butoxy-phenyl)-(5,6-dimethyl-thieno[2,3-d]pyrimidin-4-yl)-amine,

(2-Isopropoxy-phenyl)-(5-methyl-thieno[2,3-d]pyrimidin-4-yl)-amine,

(5,6-Dimethyl-thieno[2,3-d]pyrimidin-4-yl)-(2-methoxy-phenyl)-amine,

(5-Methyl-thieno[2,3-d]pyrimidin-4-yl)-[2-((R)-tetrahydro-furan-3-yloxy)-phenyl]-amine,

(5-Methyl-thieno[2,3-d]pyrimidin-4-yl)-[2-(tetrahydro-pyran-4-yloxy)-phenyl]-amine,

(2-sec-Butoxy-phenyl)-(5-methyl-thieno[2,3-d]pyrimidin-4-yl)-amine,

4-(5,6-Dimethyl-thieno[2,3-d]pyrimidin-4-ylamino)-3-methoxy-benzamide,

(2-Cyclopropylmethoxy-phenyl)-(5-methyl-thieno[2,3-d]pyrimidin-4-yl)-amine,

(5,6-Dimethyl-thieno[2,3-d]pyrimidin-4-yl)-(2-methoxy-phenyl)-amine,

(5,6-Dimethyl-thieno[2,3-d]pyrimidin-4-yl)-(2-isopropoxy-phenyl)-amine,

(2-Ethoxy-phenyl)-(5-methyl-thieno[2,3-d]pyrimidin-4-yl)-amine,

3-Methoxy-4-(5-methyl-thieno[2,3-d]pyrimidin-4-ylamino)-benzamide,

(5-Methyl-thieno[2,3-d]pyrimidin-4-yl)-[2(S)-tetrahydro-furan-3-yloxy)-phenyl]-amine,

(2-Cyclohexyloxy-phenyl)-(5-methyl-thieno[2,3-d]pyrimidin-4-yl)-amine,

(2-tert-Butoxy-phenyl)-(5,6-dimethyl-thieno[2,3-d]pyrimidin-4-yl)-amine,

(5,6-Dimethyl-thieno[2,3-d]pyrimidin-4-yl)-(2-ethoxy-phenyl)-amine,

(2-Cyclohexyloxy-phenyl)-(5,6-dimethyl-thieno[2,3-d]pyrimidin-4-yl)-amine,

(5-Methyl-thieno[2,3-d]pyrimidin-4-yl)-(2-propoxy-phenyl)-amine,

(2,4-Dimethoxy-phenyl)-(6-phenyl-thieno[2,3-d]pyrimidin-4-yl)-amine,

(2-Methoxy-phenyl)-(5,6,7,8-tetrahydro-benzo[4,5]thieno[2,3-d]pyrimidin-4-yl)-amine,

(2-Cyclopentyloxy-phenyl)-(5,6-dimethyl-thieno[2,3-d]pyrimidin-4-yl)-amine,

(5,6-Dimethyl-thieno[2,3-d]pyrimidin-4-yl)-[2-(1-ethyl-pyrrolidin-3-yloxy)-phenyl]-amine,

(2-tert-Butoxy-phenyl)-(5-methyl-thieno[2,3-d]pyrimidin-4-yl)-amine,

(5,6-Dimethyl-thieno[2,3-d]pyrimidin-4-yl)-(2-methylsulfanyl-phenyl)-amine,

(2-Methylsulfanyl-phenyl)-(5-methyl-thieno[2,3-d]pyrimidin-4-yl)-amine,

(3-Chloro-2-methoxy-phenyl)-(5-methyl-thieno[2,3-d]pyrimidin-4-yl)-amine,

(2-Difluoromethoxy-phenyl)-(5-methyl-thieno[2,3-d]pyrimidin-4-yl)-amine,

[2-(1-Ethyl-pyrrolidin-3-yloxy)-phenyl]-(5-methyl-thieno[2,3-d]pyrimidin-4-yl)-amine,

(5-Methyl-thieno[2,3-d]pyrimidin-4-yl)-[2-(1,1,2,2-tetrafluoro-ethoxy)-phenyl]-amine,

(2-sec-Butoxy-phenyl)-thieno[2,3-d]pyrimidin-4-yl-amine,

(2-Ethoxy-phenyl)-(6-methyl-thieno[2,3-d]pyrimidin-4-yl)-amine,

(2-Cyclopentyloxy-phenyl)thieno[2,3-d]pyrimidin-4-yl-amine,

(5,6-Dimethyl-thieno[2,3-d]pyrimidin-4-yl)-(2-isobutoxy-phenyl)-amine,

(2-Isopropoxy-phenyl)-thieno[2,3-d]pyrimidin-4-yl-amine,

(2-Difluoromethoxy-phenyl)-(5,6-dimethyl-thieno[2,3-d]pyrimidin-4-yl)-amine,

(2-Cyclohexyloxy-phenyl)thieno[2,3-d]pyrimidin-4-yl-amine,

(2-Isobutoxy-phenyl)-(5-methyl-thieno[2,3-d]pyrimidin-4-yl)-amine,

(5,6-Dimethyl-thieno[2,3-d]pyrimidin-4-yl)-[2-(1,1,2,2-tetrafluoro-ethoxy)-phenyl]-amine,

3-Methoxy-4-(thieno[2,3-d]pyrimidin-4-ylamino)-benzamide,

(6-Methyl-thieno[2,3-d]pyrimidin-4-yl)-[2-(tetrahydro-furan-3-yloxy)-phenyl]-amine,

[2-(Tetrahydro-furan-3-yloxy)-phenyl]-thieno[2,3-d]pyrimidin-4-yl-amine,

[2-(Adamantan-2-yloxy)-phenyl]-(5-methyl-thieno[2,3-d]pyrimidin-4-yl)-amine,

[2-((S)-Tetrahydro-furan-3-yloxy)-phenyl]-thieno[2,3-d]pyrimidin-4-yl-amine,

[2-(Adamantan-2-yloxy)-phenyl]-(5,6-dimethyl-thieno[2,3-d]pyrimidin-4-yl)-amine,

(5-Chloro-2-methoxy-phenyl)-thieno[2,3-d]pyrimidin-4-yl-amine,

(2-tert-Butoxy-phenyl)-thieno[2,3-d]pyrimidin-4-yl-amine,

(2-Morpholin-4-yl-phenyl)thieno[2,3-d]pyrimidin-4-yl-amine,

[2-(Tetrahydro-pyran-4-yloxy)-phenyl]thieno[2,3-d]pyrimidin-4-yl-amine,

(5-Methyl-thieno[2,3-d]pyrimidin-4-yl)-(2-phenoxy-phenyl)-amine,

(5,6-Dimethyl-thieno[2,3-d]pyrimidin-4-yl)-(2-isobutylsulfanyl-phenyl)-amine,

(5-Methyl-thieno[2,3-d]pyrimidin-4-yl)-(2-trifluoromethoxy-phenyl)-amine,

(2-Ethoxy-phenyl)-thieno[2,3-d]pyrimidin-4-yl-amine,

(2-Methylsulfanyl-phenyl)thieno[2,3-d]pyrimidin-4-yl-amine,

(5,6-Dimethyl-thieno[2,3-d]pyrimidin-4-yl)-(2-propyl-phenyl)-amine,

(5,6-Dimethyl-thieno[2,3-d]pyrimidin-4-yl)-(2-isopropyl-phenyl)-amine,

(2-Methoxy-phenyl)-(5-methyl-thieno[2,3-d]pyrimidin-4-yl)-amine,

(5,6-Dimethyl-thieno[2,3-d]pyrimidin-4-yl)-(2-ethyl-phenyl)-amine,

[2-(Bicyclo[2.2.1]hept-2-yloxy)-phenyl]-thieno[2,3-d]pyrimidin-4-yl-amine,

[2-(Adamantan-2-yloxy)-phenyl]-thieno[2,3-d]pyrimidin-4-yl-amine,

(2-Methoxy-phenyl)-thieno[2,3-d]pyrimidin-4-yl-amine,

(2-Isobutoxy-phenyl)thieno[2,3-d]pyrimidin-4-yl-amine,

(2-Methoxy-phenyl)-(6-methyl-thieno[2,3-d]pyrimidin-4-yl)-amine,

(2-sec-Butyl-phenyl)-(5,6-dimethyl-thieno[2,3-d]pyrimidin-4-yl)-amine,

(2-Piperidin-1-yl-phenyl)-thieno[2,3-d]pyrimidin-4-yl-amine,

[2-(Adamantan-1-yloxy)-phenyl]-(5-methyl-thieno[2,3-d]pyrimidin-4-yl)-amine,

(2-Isobutylsulfanyl-phenyl)-thieno[2,3-d]pyrimidin-4-yl-amine,

2-(5-Methyl-thieno[2,3-d]pyrimidin-4-ylamino)-phenol,

(3-Chloro-2-methoxy-phenyl)thieno[2,3-d]pyrimidin-4-yl-amine,

(2-sec-Butyl-phenyl)-(5-methyl-thieno[2,3-d]pyrimidin-4-yl)-amine,

(2-sec-Butyl-phenyl)-(5,6-dimethyl-thieno[2,3-d]pyrimidin-4-yl)-amine,

(6-Ethyl-thieno[2,3-d]pyrimidin-4-yl)-[2-(tetrahydro-furan-3-yloxy)-phenyl]-amine,

(2-Bromo-phenyl)-(5-methyl-thieno[2,3-d]pyrimidin-4-yl)-amine,

(2-Cyclohexylsulfanyl-phenyl)-thieno[2,3-d]pyrimidin-4-yl-amine,

(2-Phenoxy-phenyl)thieno[2,3-d]pyrimidin-4-yl-amine,

2-(Thieno[2,3-d]pyrimidin-4-ylamino)-phenol,

(2-Isobutylsulfanyl-phenyl)-(5-methyl-thieno[2,3-d]pyrimidin-4-yl)-amine,

(2-Isopropoxy-phenyl)-(6-methyl-5-propyl-thieno[2,3-d]pyrimidin-4-yl)-amine,

[2-(Tetrahydro-furan-3-yloxy)-phenyl]-(5-trifluoromethyl-thieno[2,3-d]pyrimidin-4-yl)-amine,

(6-Isopropyl-thieno[2,3-d]pyrimidin-4-yl)-[2-(tetrahydro-furan-3-yloxy)-phenyl]-amine,

(6-Isopropyl-thieno[2,3-d]pyrimidin-4-yl)-(2-methoxy-phenyl)-amine,

(6-Ethyl-thieno[2,3-d]pyrimidin-4-yl)-(2-isopropoxy-phenyl)-amine,

(2-Methanesulfonyl-phenyl)-(5-methyl-thieno[2,3-d]pyrimidin-4-yl)-amine,

(5,6-Dimethyl-thieno[2,3-d]pyrimidin-4-yl)-(2-phenoxy-phenyl)-amine,

(5-Methyl-thieno[2,3-d]pyrimidin-4-yl)-(2-piperidin-1-yl-phenyl)-amine,

(6-Isopropyl-thieno[2,3-d]pyrimidin-4-yl)-(2-methoxy-phenyl)-amine,

(2-sec-Butoxy-phenyl)-(6-isopropyl-thieno[2,3-d]pyrimidin-4-yl)-amine,

(2-Cyclopentylsulfanyl-phenyl)-thieno[2,3-d]pyrimidin-4-yl-amine,

[2-(endo-Bicyclo[2.2.1]hept-2-yloxy)-phenyl]-(5,6-dimethyl-thieno[2,3-d]pyrimidin-4-yl)-amine,

[2-(endo-Bicyclo[2.2.1]hept-2-yloxy)-phenyl]-thieno[2,3-d]pyrimidin-4-yl-amine,

[2-(endo-Bicyclo[2.2.1]hept-2-yloxy)-phenyl]-(5-methyl-thieno[2,3-d]pyrimidin-4-yl)-amine,

(5,6-Dimethyl-thieno[2,3-d]pyrimidin-4-yl)-[2-(tetrahydro-furan-3-ylmethoxy)-phenyl]-amine,

[2-(Tetrahydro-furan-3-ylmethoxy)-phenyl]-thieno[2,3-d]pyrimidin-4-yl-amine,

(5-Methyl-thieno[2,3-d]pyrimidin-4-yl)-[2-(tetrahydro-furan-3-ylmethoxy)-phenyl]-amine,

(2-Isopropoxy-phenyl)-(6-isopropyl-thieno[2,3-d]pyrimidin-4-yl)-amine,

[2-(1,2-Dimethyl-propoxy)-phenyl]-thieno[2,3-d]pyrimidin-4-yl-amine,

(2-Cyclopentyloxy-phenyl)-(6-isopropyl-thieno[2,3-d]pyrimidin-4-yl)-amine,

[2-(1,2-Dimethyl-propoxy)-phenyl]-(5-methyl-thieno[2,3-d]pyrimidin-4-yl)-amine,

[2-(1,2-Dimethyl-propoxy)-phenyl]-(5,6-dimethyl-thieno[2,3-d]pyrimidin-4-yl)-amine,

2,6-Dimethyl-4-[2-(thieno[2,3-d]pyrimidin-4-ylamino)-phenyl]-piperazine-1-carboxylic acid tert-butyl ester,

[5-Fluoro-2-(tetrahydro-furan-3-yloxy)-phenyl]-(5-methyl-thieno[2,3-d]pyrimidin-4-yl)-amine,

(5,6-Dimethyl-thieno[2,3-d]pyrimidin-4-yl)-[5-fluoro-2-(tetrahydro-furan-3-yloxy)-phenyl]-amine,

[5-Fluoro-2-(tetrahydro-furan-3-yloxy)-phenyl]-thieno[2,3-d]pyrimidin-4-yl-amine,

(2-Cyclopentyloxy-phenyl)-(6-ethyl-thieno[2,3-d]pyrimidin-4-yl)-amine,

(2-Methoxy-phenyl)-(6-methyl-5-propyl-thieno[2,3-d]pyrimidin-4-yl)-amine,

(2-Ethoxy-phenyl)-(6-methyl-5-propyl-thieno[2,3-d]pyrimidin-4-yl)-amine,

(2-Isopropoxy-phenyl)-(6-methyl-5-propyl-thieno[2,3-d]pyrimidin-4-yl)-amine,

(2-sec-Butoxy-phenyl)-(6-methyl-5-propyl-thieno[2,3-d]pyrimidin-4-yl)-amine,

3-(Tetrahydro-furan-3-yloxy)-4-(thieno[2,3-d]pyrimidin-4-ylamino)-benzoic acid methyl ester,

4-(5-Methyl-thieno[2,3-d]pyrimidin-4-ylamino)-3-(tetrahydro-furan-3-yloxy)-benzoic acid methyl ester,

4-(5,6-Dimethyl-thieno[2,3-d]pyrimidin-4-ylamino)-3-(tetrahydro-furan-3-yloxy)-benzoic acid methyl ester,

3-(Tetrahydro-furan-3-yloxy)-4-(thieno[2,3-d]pyrimidin-4-ylamino)-benzamide,

N-Isopropyl-N′-thieno[2,3-d]pyrimidin-4-yl-benzene-1,2-diamine,

(5,6-Dimethyl-thieno[2,3-d]pyrimidin-4-yl)-(2-methanesulfonyl-phenyl)-amine,

[2-(Tetrahydro-furan-3-yloxy)-phenyl]-(5-trifluoromethyl-thieno[2,3-d]pyrimidin-4-yl)-amine,

(2-Cyclopentyloxy-phenyl)-(5-trifluoromethyl-thieno[2,3-d]pyrimidin-4-yl)-amine,

2,6-Dimethyl-4-[2-(5-methyl-thieno[2,3-d]pyrimidin-4-ylamino)-phenyl]-piperazine-1-carboxylic acid tert-butyl ester,

(2-Ethoxy-5-fluoro-phenyl)-thieno[2,3-d]pyrimidin-4-yl-amine,

(2-sec-Butoxy-phenyl)-(5-trifluoromethyl-thieno[2,3-d]pyrimidin-4-yl)-amine,

(5,6-Dimethyl-thieno[2,3-d]pyrimidin-4-yl)-[2-(1-ethyl-2-methyl-propoxy)-phenyl]-amine,

3-[2-(5-Methyl-thieno[2,3-d]pyrimidin-4-ylamino)-phenoxy]-pyrrolidine-1-carboxylic acid tert-butyl ester,

3-[2-(5,6-Dimethyl-thieno[2,3-d]pyrimidin-4-ylamino)-phenoxy]-pyrrolidine-1-carboxylic acid tert-butyl ester,

[2-(3,5-Dimethyl-piperazin-1-yl)-phenyl]-(5-methyl-thieno[2,3-d]pyrimidin-4-yl)-amine,

(2-Pyrrolidin-1-yl-phenyl)thieno[2,3-d]pyrimidin-4-yl-amine,

(5-Methyl-thieno[2,3-d]pyrimidin-4-yl)-(2-pyrrolidin-1-yl-phenyl)-amine,

(5,6-Dimethyl-thieno[2,3-d]pyrimidin-4-yl)-(2-pyrrolidin-1-yl-phenyl)-amine,

(2-Cyclopentyloxy-phenyl)-(6-methyl-5-propyl-thieno[2,3-d]pyrimidin-4-yl)-amine,

N-Isopropyl-N′-(5-methyl-thieno[2,3-d]pyrimidin-4-yl)-benzene-1,2-diamine,

N-Cyclopentyl-N′-(5-methyl-thieno[2,3-d]pyrimidin-4-yl)-benzene-1,2-diamine,

N-sec-Butyl-N′-(5-methyl-thieno[2,3-d]pyrimidin-4-yl)-benzene-1,2-diamine,

(6-Ethyl-thieno[2,3-d]pyrimidin-4-yl)-(2-methoxy-phenyl)-amine,

(2-Ethoxy-phenyl)-(6-ethyl-thieno[2,3-d]pyrimidin-4-yl)-amine,

(2-sec-Butoxy-phenyl)-(6-ethyl-thieno[2,3-d]pyrimidin-4-yl)-amine,

(2-Ethoxy-5-fluoro-phenyl)-(5-methyl-thieno[2,3-d]pyrimidin-4-yl)-amine,

(2-Ethoxy-phenyl)-(6-isopropyl-thieno[2,3-d]pyrimidin-4-yl)-amine,

[2-(1-Ethyl-2-methyl-propoxy)-phenyl]-(5-methyl-thieno[2,3-d]pyrimidin-4-yl)-amine,

(6-Methyl-5-propyl-thieno[2,3-d]pyrimidin-4-yl)-[2-(tetrahydro-furan-3-yloxy)-phenyl]-amine,

(2-Isopropoxy-phenyl)-(5-trifluoromethyl-thieno[2,3-d]pyrimidin-4-yl)-amine,

[2-(1-Ethyl-2-methyl-propoxy)-phenyl]-thieno[2,3-d]pyrimidin-4-yl-amine,

Thieno[2,3-d]pyrimidin-4-yl)-[2-(3,3,3-trifluoro-propoxy)-phenyl]-amine,

(5-Methyl-thieno[2,3-d]pyrimidin-4-yl)-[2-(3,3,3-trifluoro-propoxy)-phenyl]-amine,

(5,6-Dimethyl-thieno[2,3-d]pyrimidin-4-yl)-[2-(3,3,3-trifluoro-propoxy)-phenyl]-amine,

(5-Ethyl-thieno[2,3-d]pyrimidin-4-yl)-(2-isopropoxy-phenyl)-amine,

(2-sec-Butoxy-phenyl)-(5-ethyl-thieno[2,3-d]pyrimidin-4-yl)-amine,

(2-Cyclopentyloxy-phenyl)-(5-ethyl-thieno[2,3-d]pyrimidin-4-yl)-amine,

[2-(3-Ethoxy-propoxy)-phenyl]-thieno[2,3-d]pyrimidin-4-yl-amine,

(5,6-Dimethyl-thieno[2,3-d]pyrimidin-4-yl)-[2-(3-ethoxy-propoxy)-phenyl]-amine,

(5-Ethyl-thieno[2,3-d]pyrimidin-4-yl)-[2-(tetrahydro-furan-3-yloxy)-phenyl]-amine,

3-Ethoxy-4-(5-methyl-thieno[2,3-d]pyrimidin-4-ylamino)-benzamide,

3-Isopropoxy-4-(5-methyl-thieno[2,3-d]pyrimidin-4-ylamino)-benzamide,

3-sec-Butoxy-4-(5-methyl-thieno[2,3-d]pyrimidin-4-ylamino)-benzamide,

3-Cyclopentyloxy-4-(5-methyl-thieno[2,3-d]pyrimidin-4-ylamino)-benzamide,

4-(5-Methyl-thieno[2,3-d]pyrimidin-4-ylamino)-3-(tetrahydro-furan-3-yloxy)-benzamide,

(5,6-Dimethyl-thieno[2,3-d]pyrimidin-4-yl)-[3-fluoro-2-(tetrahydro-furan-3-yloxy)-phenyl]-amine,

[2-(3-Ethoxy-propoxy)-phenyl]-(5-methyl-thieno[2,3-d]pyrimidin-4-yl)-amine,

[2-(2-Ethoxy-ethoxy)-phenyl]-(5-methyl-thieno[2,3-d]pyrimidin-4-yl)-amine,

[2-(2-Ethoxy-ethoxy)-phenyl]-thieno[2,3-d]pyrimidin-4-yl-amine,

(5,6-Dimethyl-thieno[2,3-d]pyrimidin-4-yl)-[2-(2-ethoxy-ethoxy)-phenyl]-amine,

3-Ethoxy-4-(thieno[2,3-d]pyrimidin-4-ylamino)-benzamide,

3-Isopropoxy-4-(thieno[2,3-d]pyrimidin-4-ylamino)-benzamide,

3-sec-Butoxy-4-(thieno[2,3-d]pyrimidin-4-ylamino)-benzamide,

3-Cyclopentyloxy-4-(thieno[2,3-d]pyrimidin-4-ylamino)-benzamide,

3-(Tetrahydro-furan-3-yloxy)-4-(5-trifluoromethyl-thieno[2,3-d]pyrimidin-4-ylamino)-benzamide,

(2,3-Dihydro-1H-8-thia-5,7-diaza-cyclopenta[a]inden-4-yl)-(2-methoxy-phenyl)-amine,

[2-(exo-Bicyclo[2.2.1]hept-2-yloxy)-phenyl]-(5,6-dimethyl-thieno[2,3-d]pyrimidin-4-yl)-amine,

(5-Methyl-thieno[2,3-d]pyrimidin-4-yl)-[2-((R)-tetrahydro-furan-3-yloxy)-phenyl]-amine,

(5,6-Dimethyl-thieno[2,3-d]pyrimidin-4-yl)-(2-morpholin-4-yl-phenyl)-amine,

(5-Methyl-thieno[2,3-d]pyrimidin-4-yl)-(2-phenoxy-phenyl)-amine,

(2-Ethyl-phenyl)-(5-methyl-thieno[2,3-d]pyrimidin-4-yl)-amine,

(2-Isopropyl-phenyl)-(5-methyl-thieno[2,3-d]pyrimidin-4-yl)-amine,

[2-(2-Bromo-ethoxy)-phenyl]-(5-methyl-thieno[2,3-d]pyrimidin-4-yl)-amine, and (5-Methyl-thieno[2,3-d]pyrimidin-4-yl)-(2-propyl-phenyl)-amine,

4-(5,6-Dimethyl-thieno[2,3-d]pyrimidin-4-yl)-3,4-dihydro-2H-benzo[1,4]oxazine,

[2-(Bicyclo[2.2.1]hept-2-yloxy)-phenyl]-(5-methyl-thieno[2,3-d]pyrimidin-4-yl)-amine,

2,6-Dimethyl-4-[2-(thieno[2,3-d]pyrimidin-4-ylamino)-phenyl]-piperazine-1-carboxylic acid tert-butyl ester,

N-Isopropyl-N′-thieno[2,3-d]pyrimidin-4-yl-benzene-1,2-diamine,

2,6-Dimethyl-4-[2-(5-methyl-thieno[2,3-d]pyrimidin-4-ylamino)-phenyl]-piperazine-1-carboxylic acid tert-butyl ester,

[2-(3,5-Dimethyl-piperazin-1-yl)-phenyl]-thieno[2,3-d]pyrimidin-4-yl-amine,

N-Cyclopentyl-N′-thieno[2,3-d]pyrimidin-4-yl-benzene-1,2-diamine,

N-Cyclohexyl-N′-thieno[2,3-d]pyrimidin-4-yl-benzene-1,2-diamine,

N-sec-Butyl-N′-thieno[2,3-d]pyrimidin-4-yl-benzene-1,2-diamine,

N-Isopropyl-N′-(5-methyl-thieno[2,3-d]pyrimidin-4-yl)-benzene-1,2-diamine,

[2-(3-Ethoxy-propoxy)-phenyl]-thieno[2,3-d]pyrimidin-4-yl-amine,

(5-Ethyl-thieno[2,3-d]pyrimidin-4-yl)-[2-(tetrahydro-furan-3-yloxy)-phenyl]-amine,

[2-(3-Ethoxy-propoxy)-phenyl]-(5-methyl-thieno[2,3-d]pyrimidin-4-yl)-amine,

[2-(2-Ethoxy-ethoxy)-phenyl]-thieno[2,3-d]pyrimidin-4-yl-amine,

[2-(2-Ethoxy-ethoxy)phenyl]-(5-methyl-thieno[2,3-d]pyrimidin-4-yl)-amine,

3-Ethoxy-4-(thieno[2,3-d]pyrimidin-4-ylamino)-benzamide,

(2-Cyclopentyloxy-4-fluoro-phenyl)-(5,6-dimethyl-thieno[2,3-d]pyrimidin-4-yl)-amine,

(2,6-Dimethoxy-phenyl)-thieno[2,3-d]pyrimidin-4-yl-amine,

(2,6-Dimethoxy-phenyl)-(5-methyl-thieno[2,3-d]pyrimidin-4-yl)-amine,

(2,6-Dimethoxy-phenyl)-(5,6-dimethyl-thieno[2,3-d]pyrimidin-4-yl)-amine,

1-{3-[2-(Thieno[2,3-d]pyrimidin-4-ylamino)-phenoxy]-pyrrolidin-1-yl}-ethanone,

3-[2-(Thieno[2,3-d]pyrimidin-4-ylamino)-phenoxy]-pyrrolidine-1-carboxylic acid dimethylamide,

2-Methyl-1-{3-[2-(thieno[2,3-d]pyrimidin-4-ylamino)-phenoxy]-pyrrolidin-1-yl}-propan-1-one,

3-Methoxy-N-methyl-4-(thieno[2,3-d]pyrimidin-4-ylamino)-benzamide,

3-Methoxy-N-methyl-4-(5-methyl-thieno[2,3-d]pyrimidin-4-ylamino)-benzamide,

4-(5,6-Dimethyl-thieno[2,3-d]pyrimidin-4-ylamino)-3-methoxy-N-methyl-benzamide,

3-Methoxy-N,N-dimethyl-4-(thieno[2,3-d]pyrimidin-4-ylamino)-benzamide,

Pyridin-3-yl-{3-[2-(thieno[2,3-d]pyrimidin-4-ylamino)-phenoxy]-pyrrolidin-1-yl}-methanone,

Pyridin-4-yl-{3-[2-(thieno[2,3-d]pyrimidin-4-ylamino)-phenoxy]-pyrrolidin-1-yl}-methanone,

3-Methoxy-N,N-dimethyl-4-(5-methyl-thieno[2,3-d]pyrimidin-4-ylamino)-benzamide,

N-Methyl-3-(tetrahydro-furan-3-yloxy)-4-(thieno[2,3-d]pyrimidin-4-ylamino)-benzamide,

Cyclopropyl-{3-[2-(thieno[2,3-d]pyrimidin-4-ylamino)-phenoxy]-pyrrolidin-1-yl}-methanone,

(2-Cyclopentyloxy-4-fluoro-phenyl)-thieno[2,3-d]pyrimidin-4-yl-amine,

(5-Methyl-thieno[2,3-d]pyrimidin-4-yl)-[2-(pyrrolidin-3-yloxy)-phenyl]-amine,

2-Fluoro-5-(tetrahydro-furan-3-yloxy)-4-(thieno[2,3-d]pyrimidin-4-ylamino)-benzamide,

2-Ethoxy-4-[1,2,4]oxadiazol-5-yl-phenyl)-(5-methyl-thieno[2,3-d]pyrimidin-4-yl)-amine,

[2-(Bicyclo[2.2.1]hept-2-yloxy)-phenyl]-(5,6-dimethyl-thieno[2,3-d]pyrimidin-4-yl)-amine,

(6-Ethyl-thieno[2,3-d]pyrimidin-4-yl)-(2-methoxy-phenyl)-amine,

(2-Ethoxy-phenyl)-(6-isopropyl-thieno[2,3-d]pyrimidin-4-yl)-amine,

(6-Isopropyl-thieno[2,3-d]pyrimidin-4-yl)-(2-methoxy-phenyl)-amine,

(2-sec-Butoxy-phenyl)-(6-isopropyl-thieno[2,3-d]pyrimidin-4-yl)-amine,

(2-Cyclopentylsulfanyl-phenyl)-thieno[2,3-d]pyrimidin-4-yl-amine,

(2-Cyclohexylsulfanyl-phenyl)thieno[2,3-d]pyrimidin-4-yl-amine

[2-(Bicyclo[2.2.1]hept-2-yloxy)-phenyl]-thieno[2,3-d]pyrimidin-4-yl-amine,

[2-(Tetrahydro-furan-3-ylmethoxy)-phenyl]-thieno[2,3-d]pyrimidin-4-yl-amine,

[5-Fluoro-2-(tetrahydro-furan-3-yloxy)-phenyl]-thieno[2,3-d]pyrimidin-4-yl-amine,

(2-sec-Butoxy-phenyl)-(6-methyl-5-propyl-thieno[2,3-d]pyrimidin-4-yl)-amine,

4-(5,6-Dimethyl-thieno[2,3-d]pyrimidin-4-ylamino)-3-(tetrahydro-furan-3-yloxy)-benzoic acid methyl ester,

(5,6-Dimethyl-thieno[2,3-d]pyrimidin-4-yl)-(2-methanesulfonyl-phenyl)-amine,

(2-Ethoxy-5-fluoro-phenyl)-thieno[2,3-d]pyrimidin-4-yl-amine,

(2-Ethoxy-5-fluoro-phenyl)-(5-methyl-thieno[2,3-d]pyrimidin-4-yl)-amine

[2-(3,5-Dimethyl-piperazin-1-yl)-phenyl]-(5-methyl-thieno[2,3-d]pyrimidin-4-yl)-amine,

(2-Cyclopentyloxy-phenyl)-(6-methyl-5-propyl-thieno[2,3-d]pyrimidin-4-yl)-amine,

(2-Pyrrolidin-1-yl-phenyl)thieno[2,3-d]pyrimidin-4-yl-amine,

3-[2-(5-Methyl-thieno[2,3-d]pyrimidin-4-ylamino)-phenoxy]-pyrrolidine-1-carboxylic acid tert-butyl ester,

N-sec-Butyl-N′-(5-methyl-thieno[2,3-d]pyrimidin-4-yl)-benzene-1,2-diamine,

N-Cyclopentyl-N′-(5-methyl-thieno[2,3-d]pyrimidin-4-yl)-benzene-1,2-diamine,

[3-Fluoro-2-(tetrahydro-furan-3-yloxy)-phenyl]-thieno[2,3-d]pyrimidin-4-yl-amine,

(4-Fluoro-2-methoxy-phenyl)-thieno[2,3-d]pyrimidin-4-yl-amine,

3-[2-(Thieno[2,3-d]pyrimidin-4-ylamino)-phenoxy]-pyrrolidine-1-carboxylic acid tert-butyl ester,

[2-(Pyrrolidin-3-yloxy)-phenyl]-thieno[2,3-d]pyrimidin-4-yl-amine,

[2-(1-Methanesulfonyl-pyrrolidin-3-yloxy)-phenyl]-thieno[2,3-d]pyrimidin-4-yl-amine,

{2-[1-(Propane-2-sulfonyl)-pyrrolidin-3-yloxy]-phenyl}-thieno[2,3-d]pyrimidin-4-yl-amine,

3-[2-(Thieno[2,3-d]pyrimidin-4-ylamino)-phenoxy]-pyrrolidine-1-sulfonic acid dimethylamide,

[2-(1-Cyclopropanesulfonyl-pyrrolidin-3-yloxy)-phenyl]-thieno[2,3-d]pyrimidin-4-yl-amine,

3-[2-(Thieno[2,3-d]pyrimidin-4-ylamino)-phenoxy]-pyrrolidine-1-carboxylic acid 4-methoxy-benzylamide,

{3-[2-(5-Methyl-thieno[2,3-d]pyrimidin-4-ylamino)-phenoxy]-pyrrolidin-1-yl}-pyridin-3-yl-methanone,

[2-Ethoxy-4-(4H-[1,2,4]triazol-3-yl)-phenyl]-(5-methyl-thieno[2,3-d]pyrimidin-4-yl)-amine,

[2-(Bicyclo[2.2.1]hept-2-yloxy)-phenyl]-thieno[2,3-d]pyrimidin-4-yl-amine,

(2-Ethoxy-phenyl)-(6-ethyl-thieno[2,3-d]pyrimidin-4-yl)-amine,

(2-sec-Butoxy-phenyl)-(6-ethyl-thieno[2,3-d]pyrimidin-4-yl)-amine,

(6-Ethyl-thieno[2,3-d]pyrimidin-4-yl)-[2-(tetrahydro-furan-3-yloxy)-phenyl]-amine,

[2-(Bicyclo[2.2.1]hept-2-yloxy)-phenyl]-(5-methyl-thieno[2,3-d]pyrimidin-4-yl)-amine,

[2-(Bicyclo[2.2.1]hept-2-yloxy)-phenyl]-(5,6-dimethyl-thieno[2,3-d]pyrimidin-4-yl)-amine,

(5-Methyl-thieno[2,3-d]pyrimidin-4-yl)-[2-(tetrahydro-furan-3-ylmethoxy)-phenyl]-amine,

(5,6-Dimethyl-thieno[2,3-d]pyrimidin-4-yl)-[2-(tetrahydro-furan-3-ylmethoxy)-phenyl]-amine,

(2-Isopropoxy-phenyl)-(6-isopropyl-thieno[2,3-d]pyrimidin-4-yl)-amine,

(2-Cyclopentyloxy-phenyl)-(6-isopropyl-thieno[2,3-d]pyrimidin-4-yl)-amine,

(6-Isopropyl-thieno[2,3-d]pyrimidin-4-yl)-[2-(tetrahydro-furan-3-yloxy)-phenyl]-amine,

[2-(1,2-Dimethyl-propoxy)-phenyl]-thieno[2,3-d]pyrimidin-4-yl-amine,

[2-(1,2-Dimethyl-propoxy)-phenyl]-(5-methyl-thieno[2,3-d]pyrimidin-4-yl)-amine,

[2-(1,2-Dimethyl-propoxy)-phenyl]-(5,6-dimethyl-thieno[2,3-d]pyrimidin-4-yl)-amine,

[5-Fluoro-2-(tetrahydro-furan-3-yloxy)-phenyl]-(5-methyl-thieno[2,3-d]pyrimidin-4-yl)-amine,

(5,6-Dimethyl-thieno[2,3-d]pyrimidin-4-yl)-[5-fluoro-2-(tetrahydro-furan-3-yloxy)-phenyl]-amine,

(6-Ethyl-thieno[2,3-d]pyrimidin-4-yl)-(2-isopropoxy-phenyl)-amine,

(2-Cyclopentyloxy-phenyl)-(6-ethyl-thieno[2,3-d]pyrimidin-4-yl)-amine,

(2-Methoxy-phenyl)-(6-methyl-5-propyl-thieno[2,3-d]pyrimidin-4-yl)-amine,

(2-Ethoxy-phenyl)-(6-methyl-5-propyl-thieno[2,3-d]pyrimidin-4-yl)-amine,

(2-Isopropoxy-phenyl)-(6-methyl-5-propyl-thieno[2,3-d]pyrimidin-4-yl)-amine,

3-(Tetrahydro-furan-3-yloxy)-4-(thieno[2,3-d]pyrimidin-4-ylamino)-benzoic acid methyl ester,

4-(5-Methyl-thieno[2,3-d]pyrimidin-4-ylamino)-3-(tetrahydro-furan-3-yloxy)-benzoic acid methyl ester,

3-(Tetrahydro-furan-3-yloxy)-4-(thieno[2,3-d]pyrimidin-4-ylamino)-benzamide,

[2-(Tetrahydro-furan-3-yloxy)-phenyl]-(5-trifluoromethyl-thieno[2,3-d]pyrimidin-4-yl)-amine,

(2-Cyclopentyloxy-phenyl)-(5-trifluoromethyl-thieno[2,3-d]pyrimidin-4-yl)-amine,

[2-(1-Ethyl-2-methyl-propoxy)-phenyl]-thieno[2,3-d]pyrimidin-4-yl-amine,

[2-(1-Ethyl-2-methyl-propoxy)-phenyl]-(5-methyl-thieno[2,3-d]pyrimidin-4-yl)-amine,

(6-Methyl-5-propyl-thieno[2,3-d]pyrimidin-4-yl)-[2-(tetrahydro-furan-3-yloxy)-phenyl]-amine,

(5,6-Dimethyl-thieno[2,3-d]pyrimidin-4-yl)-[2-(1-ethyl-2-methyl-propoxy)-phenyl]-amine,

(2-Isopropoxy-phenyl)-(5-trifluoromethyl-thieno[2,3-d]pyrimidin-4-yl)-amine,

(2-sec-Butoxy-phenyl)-(5-trifluoromethyl-thieno[2,3-d]pyrimidin-4-yl)-amine,

(5-Methyl-thieno[2,3-d]pyrimidin-4-yl)-(2-pyrrolidin-1-yl-phenyl)-amine,

(5,6-Dimethyl-thieno[2,3-d]pyrimidin-4-yl)-(2-pyrrolidin-1-yl-phenyl)-amine,

3-[2-(5,6-Dimethyl-thieno[2,3-d]pyrimidin-4-ylamino)-phenoxy]-pyrrolidine-1-carboxylic acid tert-butyl ester,

Thieno[2,3-d]pyrimidin-4-yl-[2-(3,3,3-trifluoro-propoxy)-phenyl]-amine,

(5-Methyl-thieno[2,3-d]pyrimidin-4-yl)-[2-(3,3,3-trifluoro-propoxy)-phenyl]-amine

(5,6-Dimethyl-thieno[2,3-d]pyrimidin-4-yl)-[2-(3,3,3-trifluoro-propoxy)-phenyl]-amine,

(5-Ethyl-thieno[2,3-d]pyrimidin-4-yl)-(2-isopropoxy-phenyl)-amine,

(2-sec-Butoxy-phenyl)-(5-ethyl-thieno[2,3-d]pyrimidin-4-yl)-amine,

(2-Cyclopentyloxy-phenyl)-(5-ethyl-thieno[2,3-d]pyrimidin-4-yl)-amine,

(5,6-Dimethyl-thieno[2,3-d]pyrimidin-4-yl)-[2-(3-ethoxy-propoxy)-phenyl]-amine,

[3-Fluoro-2-(tetrahydro-furan-3-yloxy)-phenyl]-(5-methyl-thieno[2,3-d]pyrimidin-4-yl)-amine,

(5,6-Dimethyl-thieno[2,3-d]pyrimidin-4-yl)-[3-fluoro-2-(tetrahydro-furan-3-yloxy)-phenyl]-amine,

(5,6-Dimethyl-thieno[2,3-d]pyrimidin-4-yl)-[2-(2-ethoxy-ethoxy)-phenyl]-amine,

3-Isopropoxy-4-(thieno[2,3-d]pyrimidin-4-ylamino)-benzamide,

3-sec-Butoxy-4-(thieno[2,3-d]pyrimidin-4-ylamino)-benzamide,

3-Cyclopentyloxy-4-(thieno[2,3-d]pyrimidin-4-ylamino)-benzamide,

3-Ethoxy-4-(5-methyl-thieno[2,3-d]pyrimidin-4-ylamino)-benzamide,

3-Isopropoxy-4-(5-methyl-thieno[2,3-d]pyrimidin-4-ylamino)-benzamide,

3-sec-Butoxy-4-(5-methyl-thieno[2,3-d]pyrimidin-4-ylamino)-benzamide,

3-Cyclopentyloxy-4-(5-methyl-thieno[2,3-d]pyrimidin-4-ylamino)-benzamide,

4-(5-Methyl-thieno[2,3-d]pyrimidin-4-ylamino)-3-(tetrahydro-furan-3-yloxy)-benzamide,

3-(Tetrahydro-furan-3-yloxy)-4-(5-trifluoromethyl-thieno[2,3-d]pyrimidin-4-ylamino)-benzamide,

4-(Thieno[2,3-d]pyrimidin-4-ylamino)-3-(3,3,3-trifluoro-propoxy)-benzamide,

4-(5-Methyl-thieno[2,3-d]pyrimidin-4-ylamino)-3-(3,3,3-trifluoro-propoxy)-benzamide,

4-(5,6-Dimethyl-thieno[2,3-d]pyrimidin-4-ylamino)-3-(3,3,3-trifluoro-propoxy)-benzamide,

3-Pyrrolidin-1-yl-4-(thieno[2,3-d]pyrimidin-4-ylamino)-benzamide,

4-(5-Methyl-thieno[2,3-d]pyrimidin-4-ylamino)-3-pyrrolidin-1-yl-benzamide,

4-(5,6-Dimethyl-thieno[2,3-d]pyrimidin-4-ylamino)-3-pyrrolidin-1-yl-benzamide,

4-(5,6-Dimethyl-thieno[2,3-d]pyrimidin-4-ylamino)-3-ethoxy-benzamide,

4-(5,6-Dimethyl-thieno[2,3-d]pyrimidin-4-ylamino)-3-isopropoxy-benzamide,

3-Cyclopentyloxy-4-(5,6-dimethyl-thieno[2,3-d]pyrimidin-4-ylamino)-benzamide,

4-(5,6-Dimethyl-thieno[2,3-d]pyrimidin-4-ylamino)-3-(tetrahydro-furan-3-yloxy)-benzamide,

(2-Ethoxy-4-fluoro-phenyl)-thieno[2,3-d]pyrimidin-4-yl-amine,

[4-Fluoro-2-(tetrahydro-furan-3-yloxy)-phenyl]-thieno[2,3-d]pyrimidin-4-yl-amine,

(4-Fluoro-2-methoxy-phenyl)-(5-methyl-thieno[2,3-d]pyrimidin-4-yl)-amine,

(2-Ethoxy-4-fluoro-phenyl)-(5-methyl-thieno[2,3-d]pyrimidin-4-yl)-amine,

(2-Cyclopentyloxy-4-fluoro-phenyl)-(5-methyl-thieno[2,3-d]pyrimidin-4-yl)-amine,

[4-Fluoro-2-(tetrahydro-furan-3-yloxy)-phenyl]-(5-methyl-thieno[2,3-d]pyrimidin-4-yl)-amine,

(5,6-Dimethyl-thieno[2,3-d]pyrimidin-4-yl)-(4-fluoro-2-methoxy-phenyl)-amine,

(5,6-Dimethyl-thieno[2,3-d]pyrimidin-4-yl)-(2-ethoxy-4-fluoro-phenyl)-amine,

(5,6-Dimethyl-thieno[2,3-d]pyrimidin-4-yl)-(4-fluoro-2-isopropoxy-phenyl)-amine,

(5,6-Dimethyl-thieno[2,3-d]pyrimidin-4-yl)-[4-fluoro-2-(tetrahydro-furan-3-yloxy)-phenyl]-amine,

3-Methoxy-4-(5-trifluoromethyl-thieno[2,3-d]pyrimidin-4-ylamino)-benzamide,

3-Ethoxy-4-(5-trifluoromethyl-thieno[2,3-d]pyrimidin-4-ylamino)-benzamide,

3-Isopropoxy-4-(5-trifluoromethyl-thieno[2,3-d]pyrimidin-4-ylamino)-benzamide,

(4-Fluoro-2-isopropoxy-phenyl)-thieno[2,3-d]pyrimidin-4-yl-amine,

(2-sec-Butoxy-4-fluoro-phenyl)-thieno[2,3-d]pyrimidin-4-yl-amine,

(4-Fluoro-2-isopropoxy-phenyl)-(5-methyl-thieno[2,3-d]pyrimidin-4-yl)-amine,

(2-sec-Butoxy-4-fluoro-phenyl)-(5-methyl-thieno[2,3-d]pyrimidin-4-yl)-amine,

(2-sec-Butoxy-4-fluoro-phenyl)-(5,6-dimethyl-thieno[2,3-d]pyrimidin-4-yl)-amine,

(4-Fluoro-2-methoxy-phenyl)-(5-trifluoromethyl-thieno[2,3-d]pyrimidin-4-yl)-amine,

(4-Fluoro-2-isopropoxy-phenyl)-(5-trifluoromethyl-thieno[2,3-d]pyrimidin-4-yl)-amine,

[4-Fluoro-2-(tetrahydro-furan-3-yloxy)-phenyl]-(5-trifluoromethyl-thieno[2,3-d]pyrimidin-4-yl)-amine,

[4-Fluoro-2-(3,3,3-trifluoro-propoxy)-phenyl]-thieno[2,3-d]pyrimidin-4-yl-amine,

[4-Fluoro-2-(3,3,3-trifluoro-propoxy)-phenyl]-(5-methyl-thieno[2,3-d]pyrimidin-4-yl)-amine,

(5,6-Dimethyl-thieno[2,3-d]pyrimidin-4-yl)-[4-fluoro-2-(3,3,3-trifluoro-propoxy)-phenyl]-amine,

4-(5,6-Dimethyl-thieno[2,3-d]pyrimidin-4-ylamino)-N-methyl-3-(tetrahydro-furan-3-yloxy)-benzamide,

2-Fluoro-5-methoxy-4-(thieno[2,3-d]pyrimidin-4-ylamino)-benzamide,

2-Fluoro-5-isopropoxy-4-(thieno[2,3-d]pyrimidin-4-ylamino)-benzamide,

2-Fluoro-5-methoxy-4-(5-methyl-thieno[2,3-d]pyrimidin-4-ylamino)-benzamide,

2-Fluoro-4-(5-methyl-thieno[2,3-d]pyrimidin-4-ylamino)-5-(tetrahydro-furan-3-yloxy)-benzamide,

4-(5,6-Dimethyl-thieno[2,3-d]pyrimidin-4-ylamino)-2-fluoro-5-methoxy-benzamide,

4-(5,6-Dimethyl-thieno[2,3-d]pyrimidin-4-ylamino)-2-fluoro-5-isopropoxy-benzamide,

4-(5,6-Dimethyl-thieno[2,3-d]pyrimidin-4-ylamino)-2-fluoro-5-(tetrahydro-furan-3-yloxy)-benzamide,

[2-(1-Methanesulfonyl-pyrrolidin-3-yloxy)-phenyl]-(5-methyl-thieno[2,3-d]pyrimidin-4-yl)-amine,

1-{3-[2-(5-Methyl-thieno[2,3-d]pyrimidin-4-ylamino)-phenoxy]-pyrrolidin-1-yl}-ethanone,

3-[2-(5-Methyl-thieno[2,3-d]pyrimidin-4-ylamino)-phenoxy]-pyrrolidine-1-carboxylic acid dimethylamide,

2-Methyl-1-{3-[2-(5-methyl-thieno[2,3-d]pyrimidin-4-ylamino)-phenoxy]-pyrrolidin-1-yl}-propan-1-one,

Cyclopropyl-{3-[2-(5-methyl-thieno[2,3-d]pyrimidin-4-ylamino)-phenoxy]-pyrrolidin-1-yl}-methanone,

Cyclopentyl-{3-[2-(5-methyl-thieno[2,3-d]pyrimidin-4-ylamino)-phenoxy]-pyrrolidin-1-yl}-methanone,

3-[2-(5-Methyl-thieno[2,3-d]pyrimidin-4-ylamino)-phenoxy]-pyrrolidine-1-sulfonic acid dimethylamide,

(5-Methyl-thieno[2,3-d]pyrimidin-4-yl)-{2-[1-(propane-2-sulfonyl)-pyrrolidin-3-yloxy]-phenyl}-amine,

{3-[2-(5-Methyl-thieno[2,3-d]pyrimidin-4-ylamino)-phenoxy]-pyrrolidin-1-yl}-pyridin-4-yl-methanone,

3-sec-Butoxy-4-(5,6-dimethyl-thieno[2,3-d]pyrimidin-4-ylamino)-benzamide,

3-Ethoxy-4-(5-methyl-thieno[2,3-d]pyrimidin-4-ylamino)-benzonitrile,

3-Isopropoxy-4-(5-methyl-thieno[2,3-d]pyrimidin-4-ylamino)-benzonitrile,

3-sec-Butoxy-4-(5,6-dimethyl-thieno[2,3-d]pyrimidin-4-ylamino)-benzonitrile,

(5,6-Dimethyl-thieno[2,3-d]pyrimidin-4-yl)-[2-(1-methanesulfonyl-pyrrolidin-3-yloxy)-phenyl]-amine,

3-(1-Methanesulfonyl-pyrrolidin-3-yloxy)-4-(5-methyl-thieno[2,3-d]pyrimidin-4-ylamino)-benzamide,

4-(5,6-Dimethyl-thieno[2,3-d]pyrimidin-4-ylamino)-3-(1-methanesulfonyl-pyrrolidin-3-yloxy)-benzamide,

More preferred are the following compounds:

[2-(Bicyclo[2.2.1]hept-2-yloxy)-phenyl]-(5,6-dimethyl-thieno[2,3-d]pyrimidin-4-yl)-amine,

(6-Ethyl-thieno[2,3-d]pyrimidin-4-yl)-(2-methoxy-phenyl)-amine,

(2-Ethoxy-phenyl)-(6-isopropyl-thieno[2,3-d]pyrimidin-4-yl)-amine,

(6-Isopropyl-thieno[2,3-d]pyrimidin-4-yl)-(2-methoxy-phenyl)-amine,

(2-sec-Butoxy-phenyl)-(6-isopropyl-thieno[2,3-d]pyrimidin-4-yl)-amine,

(2-Cyclopentylsulfanyl-phenyl)-thieno[2,3-d]pyrimidin-4-yl-amine,

(2-Cyclohexylsulfanyl-phenyl)-thieno[2,3-d]pyrimidin-4-yl-amine

[2-(Bicyclo[2.2.1]hept-2-yloxy)-phenyl]-thieno[2,3-d]pyrimidin-4-yl-amine,

[2-(Tetrahydro-furan-3-ylmethoxy)-phenyl]-thieno[2,3-d]pyrimidin-4-yl-amine,

[5-Fluoro-2-(tetrahydro-furan-3-yloxy)-phenyl]-thieno[2,3-d]pyrimidin-4-yl-amine,

(2-sec-Butoxy-phenyl)-(6-methyl-5-propyl-thieno[2,3-d]pyrimidin-4-yl)-amine,

4-(5,6-Dimethyl-thieno[2,3-d]pyrimidin-4-ylamino)-3-(tetrahydro-furan-3-yloxy)-benzoic acid methyl ester,

(5,6-Dimethyl-thieno[2,3-d]pyrimidin-4-yl)-(2-methanesulfonyl-phenyl)-amine,

(2-Ethoxy-5-fluoro-phenyl)-thieno[2,3-d]pyrimidin-4-yl-amine,

(2-Ethoxy-5-fluoro-phenyl)-(5-methyl-thieno[2,3-d]pyrimidin-4-yl)-amine

[2-(3,5-Dimethyl-piperazin-1-yl)-phenyl]-(5-methyl-thieno[2,3-d]pyrimidin-4-yl)-amine,

(2-Cyclopentyloxy-phenyl)-(6-methyl-5-propyl-thieno[2,3-d]pyrimidin-4-yl)-amine,

(2-Pyrrolidin-1-yl-phenyl)thieno[2,3-d]pyrimidin-4-yl-amine,

3-[2-(5-Methyl-thieno[2,3-d]pyrimidin-4-ylamino)-phenoxy]-pyrrolidine-1-carboxylic acid tert-butyl ester,

N-sec-Butyl-N′-(5-methyl-thieno[2,3-d]pyrimidin-4-yl)-benzene-1,2-diamine,

N-Cyclopentyl-N′-(5-methyl-thieno[2,3-d]pyrimidin-4-yl)-benzene-1,2-diamine,

[3-Fluoro-2-(tetrahydro-furan-3-yloxy)-phenyl]-thieno[2,3-d]pyrimidin-4-yl-amine,

(4-Fluoro-2-methoxy-phenyl)-thieno[2,3-d]pyrimidin-4-yl-amine,

3-[2-(Thieno[2,3-d]pyrimidin-4-ylamino)-phenoxy]-pyrrolidine-1-carboxylic acid tert-butyl ester,

[2-(Pyrrolidin-3-yloxy)-phenyl]-thieno[2,3-d]pyrimidin-4-yl-amine,

[2-(1-Methanesulfonyl-pyrrolidin-3-yloxy)-phenyl]-thieno[2,3-d]pyrimidin-4-yl-amine,

{2-[1-(Propane-2-sulfonyl)-pyrrolidin-3-yloxy]-phenyl}-thieno[2,3-d]pyrimidin-4-yl-amine,

3-[2-(Thieno[2,3-d]pyrimidin-4-ylamino)-phenoxy]-pyrrolidine-1-sulfonic acid dimethylamide,

[2-(1-Cyclopropanesulfonyl-pyrrolidin-3-yloxy)-phenyl]-thieno[2,3-d]pyrimidin-4-yl-amine,

3-[2-(Thieno[2,3-d]pyrimidin-4-ylamino)-phenoxy]-pyrrolidine-1-carboxylic acid 4-methoxy-benzylamide,

{3-[2-(5-Methyl-thieno[2,3-d]pyrimidin-4-ylamino)-phenoxy]-pyrrolidin-1-yl}-pyridin-3-yl-methanone,

[2-Ethoxy-4-(4H-[1,2,4]triazol-3-yl)-phenyl]-(5-methyl-thieno[2,3-d]pyrimidin-4yl)-amine,

[2-(Bicyclo[2.2.1]hept-2-yloxy)-phenyl]-thieno[2,3-d]pyrimidin-4-yl-amine,

(2-Ethoxy-phenyl)-(6-ethyl-thieno[2,3-d]pyrimidin-4-yl)-amine,

(2-sec-Butoxy-phenyl)-(6-ethyl-thieno[2,3-d]pyrimidin-4-yl)-amine,

(6-Ethyl-thieno[2,3-d]pyrimidin-4-yl)-[2-(tetrahydro-furan-3-yloxy)-phenyl]-amine,

[2-(Bicyclo[2.2.1]hept-2-yloxy)-phenyl]-(5-methyl-thieno[2,3-d]pyrimidin-4-yl)-amine,

[2-(Bicyclo[2.2.1]hept-2-yloxy)-phenyl]-(5,6-dimethyl-thieno[2,3-d]pyrimidin-4-yl)-amine,

(5-Methyl-thieno[2,3-d]pyrimidin-4-yl)-[2-(tetrahydro-furan-3-ylmethoxy)-phenyl]-amine,

(5,6-Dimethyl-thieno[2,3-d]pyrimidin-4-yl)-[2-(tetrahydro-furan-3-ylmethoxy)-phenyl]-amine,

(2-Isopropoxy-phenyl)-(6-isopropyl-thieno[2,3-d]pyrimidin-4-yl)-amine,

(2-Cyclopentyloxy-phenyl)-(6-isopropyl-thieno[2,3-d]pyrimidin-4-yl)-amine,

(6-Isopropyl-thieno[2,3-d]pyrimidin-4-yl)-[2-(tetrahydro-furan-3-yloxy)-phenyl]-amine,

[2-(1,2-Dimethyl-propoxy)-phenyl]-thieno[2,3-d]pyrimidin-4-yl-amine,

[2-(1,2-Dimethyl-propoxy)-phenyl]-(5-methyl-thieno[2,3-d]pyrimidin-4-yl)-amine,

[2-(1,2-Dimethyl-propoxy)-phenyl]-(5,6-dimethyl-thieno[2,3-d]pyrimidin-4-yl)-amine,

[5-Fluoro-2-(tetrahydro-furan-3-yloxy)-phenyl]-(5-methyl-thieno[2,3-d]pyrimidin-4-yl)-amine,

(5,6-Dimethyl-thieno[2,3-d]pyrimidin-4-yl)-[5-fluoro-2-(tetrahydro-furan-3-yloxy)-phenyl]-amine,

(6-Ethyl-thieno[2,3-d]pyrimidin-4-yl)-(2-isopropoxy-phenyl)-amine,

(2-Cyclopentyloxy-phenyl)-(6-ethyl-thieno[2,3-d]pyrimidin-4-yl)-amine,

(2-Methoxy-phenyl)-(6-methyl-5-propyl-thieno[2,3-d]pyrimidin-4-yl)-amine,

(2-Ethoxy-phenyl)-(6-methyl-5-propyl-thieno[2,3-d]pyrimidin-4-yl)-amine,

(2-Isopropoxy-phenyl)-(6-methyl-5-propyl-thieno[2,3-d]pyrimidin-4-yl)-amine,

3-(Tetrahydro-furan-3-yloxy)-4-(thieno[2,3-d]pyrimidin-4-ylamino)-benzoic acid methyl ester,

4-(5-Methyl-thieno[2,3-d]pyrimidin-4-ylamino)-3-(tetrahydro-furan-3-yloxy)-benzoic acid methyl ester,

3-(Tetrahydro-furan-3-yloxy)-4-(thieno[2,3-d]pyrimidin-4-ylamino)-benzamide,

[2-(Tetrahydro-furan-3-yloxy)-phenyl]-(5-trifluoromethyl-thieno[2,3-d]pyrimidin-4-yl)-amine,

(2-Cyclopentyloxy-phenyl)-(5-trifluoromethyl-thieno[2,3-d]pyrimidin-4-yl)-amine,

[2-(1-Ethyl-2-methyl-propoxy)-phenyl]-thieno[2,3-d]pyrimidin-4-yl-amine,

[2-(1-Ethyl-2-methyl-propoxy)-phenyl]-(5-methyl-thieno[2,3-d]pyrimidin-4-yl)-amine,

(6-Methyl-5-propyl-thieno[2,3-d]pyrimidin-4-yl)-[2-(tetrahydro-furan-3-yloxy)-phenyl]-amine,

(5,6-Dimethyl-thieno[2,3-d]pyrimidin-4-yl)-[2-(1-ethyl-2-methyl-propoxy)-phenyl]-amine,

(2-Isopropoxy-phenyl)-(5-trifluoromethyl-thieno[2,3-d]pyrimidin-4-yl)-amine,

(2-sec-Butoxy-phenyl)-(5-trifluoromethyl-thieno[2,3-d]pyrimidin-4-yl)-amine,

(5-Methyl-thieno[2,3-d]pyrimidin-4-yl)-(2-pyrrolidin-1-yl-phenyl)-amine,

(5,6-Dimethyl-thieno[2,3-d]pyrimidin-4-yl)-(2-pyrrolidin-1-yl-phenyl)-amine,

3-[2-(5,6-Dimethyl-thieno[2,3-d]pyrimidin-4-ylamino)-phenoxy]-pyrrolidine-1-carboxylic acid tert-butyl ester,

Thieno[2,3-d]pyrimidin-4-yl-[2-(3,3,3-trifluoro-propoxy)-phenyl]-amine,

(5-Methyl-thieno[2,3-d]pyrimidin-4-yl)-[2-(3,3,3-trifluoro-propoxy)-phenyl]-amine

(5,6-Dimethyl-thieno[2,3-d]pyrimidin-4-yl)-[2-(3,3,3-trifluoro-propoxy)-phenyl]-amine,

(5-Ethyl-thieno[2,3-d]pyrimidin-4-yl)-(2-isopropoxy-phenyl)-amine,

(2-sec-Butoxy-phenyl)-(5-ethyl-thieno[2,3-d]pyrimidin-4-yl)-amine,

(2-Cyclopentyloxy-phenyl)-(5-ethyl-thieno[2,3-d]pyrimidin-4-yl)-amine,

(5,6-Dimethyl-thieno[2,3-d]pyrimidin-4-yl)-[2-(3-ethoxy-propoxy)-phenyl]-amine,

[3-Fluoro-2-(tetrahydro-furan-3-yloxy)-phenyl]-(5-methyl-thieno[2,3-d]pyrimidin-4-yl)-amine,

(5,6-Dimethyl-thieno[2,3-d]pyrimidin-4-yl)-[3-fluoro-2-(tetrahydro-furan-3-yloxy)-phenyl]amine,

(5,6-Dimethyl-thieno[2,3-d]pyrimidin-4-yl)-[2-(2-ethoxy-ethoxy)-phenyl]-amine,

3-Isopropoxy-4-(thieno[2,3-d]pyrimidin-4-ylamino)-benzamide,

3-sec-Butoxy-4-(thieno[2,3-d]pyrimidin-4-ylamino)-benzamide,

3-Cyclopentyloxy-4-(thieno[2,3-d]pyrimidin-4-ylamino)-benzamide,

3-Ethoxy-4-(5-methyl-thieno[2,3-d]pyrimidin-4-ylamino)-benzamide,

3-Isopropoxy-4-(5-methyl-thieno[2,3-d]pyrimidin-4-ylamino)-benzamide,

3-sec-Butoxy-4-(5-methyl-thieno[2,3-d]pyrimidin-4-ylamino)-benzamide,

3-Cyclopentyloxy-4-(5-methyl-thieno[2,3-d]pyrimidin-4-ylamino)-benzamide,

4-(5-Methyl-thieno[2,3-d]pyrimidin-4-ylamino)-3-(tetrahydro-furan-3-yloxy)-benzamide,

3-(Tetrahydro-furan-3-yloxy)-4-(5-trifluoromethyl-thieno[2,3-d]pyrimidin-4-ylamino)-benzamide,

4-(Thieno[2,3-d]pyrimidin-4-ylamino)-3-(3,3,3-trifluoro-propoxy)-benzamide,

4-(5-Methyl-thieno[2,3-d]pyrimidin-4-ylamino)-3-(3,3,3-trifluoro-propoxy)-benzamide,

4-(5,6-Dimethyl-thieno[2,3-d]pyrimidin-4-ylamino)-3-(3,3,3-trifluoro-propoxy)-benzamide,

3-Pyrrolidin-1-yl-4-(thieno[2,3-d]pyrimidin-4-ylamino)-benzamide,

4-(5-Methyl-thieno[2,3-d]pyrimidin-4-ylamino)-3-pyrrolidin-1-yl-benzamide,

4-(5,6-Dimethyl-thieno[2,3-d]pyrimidin-4-ylamino)-3-pyrrolidin-1-yl-benzamide,

4-(5,6-Dimethyl-thieno[2,3-d]pyrimidin-4-ylamino)-3-ethoxy-benzamide,

4-(5,6-Dimethyl-thieno[2,3-d]pyrimidin-4-ylamino)-3-isopropoxy-benzamide,

3-Cyclopentyloxy-4-(5,6-dimethyl-thieno[2,3-d]pyrimidin-4-ylamino)-benzamide,

4-(5,6-Dimethyl-thieno[2,3-d]pyrimidin-4-ylamino)-3-(tetrahydro-furan-3-yloxy)-benzamide,

(2-Ethoxy-4-fluoro-phenyl)thieno[2,3-d]pyrimidin-4-yl-amine,

[4-Fluoro-2-(tetrahydro-furan-3-yloxy)-phenyl]-thieno[2,3-d]pyrimidin-4-yl-amine,

(4-Fluoro-2-methoxy-phenyl)-(5-methyl-thieno[2,3-d]pyrimidin-4-yl)-amine,

(2-Ethoxy-4-fluoro-phenyl)-(5-methyl-thieno[2,3-d]pyrimidin-4-yl)-amine,

(2-Cyclopentyloxy-4-fluoro-phenyl)-(5-methyl-thieno[2,3-d]pyrimidin-4-yl)-amine,

[4-Fluoro-2-(tetrahydro-furan-3-yloxy)-phenyl]-(5-methyl-thieno[2,3-d]pyrimidin-4-yl)-amine,

(5,6-Dimethyl-thieno[2,3-d]pyrimidin-4-yl)-(4-fluoro-2-methoxy-phenyl)-amine,

(5,6-Dimethyl-thieno[2,3-d]pyrimidin-4-yl)-(2-ethoxy-4-fluoro-phenyl)-amine,

(5,6-Dimethyl-thieno[2,3-d]pyrimidin-4-yl)-(4-fluoro-2-isopropoxy-phenyl)-amine,

(5,6-Dimethyl-thieno[2,3-d]pyrimidin-4-yl)-[4-fluoro-2-(tetrahydro-furan-3-yloxy)-phenyl]-amine,

3-Methoxy-4-(5-trifluoromethyl-thieno[2,3-d]pyrimidin-4-ylamino)-benzamide,

3-Ethoxy-4-(5-trifluoromethyl-thieno[2,3-d]pyrimidin-4-ylamino)-benzamide,

3-Isopropoxy-4-(5-trifluoromethyl-thieno[2,3-d]pyrimidin-4-ylamino)-benzamide,

(4-Fluoro-2-isopropoxy-phenyl)-thieno[2,3-d]pyrimidin-4-yl-amine,

(2-sec-Butoxy-4-fluoro-phenyl)-thieno[2,3-d]pyrimidin-4-yl-amine,

(4-Fluoro-2-isopropoxy-phenyl)-(5-methyl-thieno[2,3-d]pyrimidin-4-yl)-amine,

(2-sec-Butoxy-4-fluoro-phenyl)-(5-methyl-thieno[2,3-d]pyrimidin-4-yl)-amine,

(2-sec-Butoxy-4-fluoro-phenyl)-(5,6-dimethyl-thieno[2,3-d]pyrimidin-4-yl)-amine,

(4-Fluoro-2-methoxy-phenyl)-(5-trifluoromethyl-thieno[2,3-d]pyrimidin-4-yl)-amine,

(4-Fluoro-2-isopropoxy-phenyl)-(5-trifluoromethyl-thieno[2,3-d]pyrimidin-4-yl)-amine,

[4-Fluoro-2-(tetrahydro-furan-3-yloxy)-phenyl]-(5-trifluoromethyl-thieno[2,3-d]pyrimidin-4-yl)-amine,

[4-Fluoro-2-(3,3,3-trifluoro-propoxy)-phenyl]-thieno[2,3-d]pyrimidin-4-yl-amine,

[4-Fluoro-2-(3,3,3-trifluoro-propoxy)-phenyl]-(5-methyl-thieno[2,3-d]pyrimidin-4-yl)-amine,

(5,6-Dimethyl-thieno[2,3-d]pyrimidin-4-yl)-[4-fluoro-2-(3,3,3-trifluoro-propoxy)-phenyl]-amine,

4-(5,6-Dimethyl-thieno[2,3-d]pyrimidin-4-ylamino)-N-methyl-3-(tetrahydro-furan-3-yloxy)-benzamide,

2-Fluoro-5-methoxy-4-(thieno[2,3-d]pyrimidin-4-ylamino)-benzamide,

2-Fluoro-5-isopropoxy-4-(thieno[2,3-d]pyrimidin-4-ylamino)-benzamide,

2-Fluoro-5-methoxy-4-(5-methyl-thieno[2,3-d]pyrimidin-4-ylamino)-benzamide,

2-Fluoro-4-(5-methyl-thieno[2,3-d]pyrimidin-4-ylamino)-5-(tetrahydro-furan-3-yloxy)-benzamide,

4-(5,6-Dimethyl-thieno[2,3-d]pyrimidin-4-ylamino)-2-fluoro-5-methoxy-benzamide,

4-(5,6-Dimethyl-thieno[2,3-d]pyrimidin-4-ylamino)-2-fluoro-5-isopropoxy-benzamide,

4-(5,6-Dimethyl-thieno[2,3-d]pyrimidin-4-ylamino)-2-fluoro-5-(tetrahydro-furan-3-yloxy)-benzamide,

[2-(1-Methanesulfonyl-pyrrolidin-3-yloxy)-phenyl]-(5-methyl-thieno[2,3-d]pyrimidin-4-yl)-amine,

1-{3-[2-(5-Methyl-thieno[2,3-d]pyrimidin-4-ylamino)-phenoxy]-pyrrolidin-1-yl}-ethanone,

3-[2-(5-Methyl-thieno[2,3-d]pyrimidin-4-ylamino)-phenoxy]-pyrrolidine-1-carboxylic acid dimethylamide,

2-Methyl-1-{3-[2-(5-methyl-thieno[2,3-d]pyrimidin-4-ylamino)-phenoxy]-pyrrolidin-1-yl}-propan-1-one,

Cyclopropyl-{3-[2-(5-methyl-thieno[2,3-d]pyrimidin-4-ylamino)-phenoxy]-pyrrolidin-1-yl}-methanone,

Cyclopentyl-{3-[2-(5-methyl-thieno[2,3-d]pyrimidin-4-ylamino)-phenoxy]-pyrrolidin-1-yl}-methanone,

3-[2-(5-Methyl-thieno[2,3-d]pyrimidin-4-ylamino)-phenoxy]-pyrrolidine-1-sulfonic acid dimethylamide,

(5-Methyl-thieno[2,3-d]pyrimidin-4-yl)-{2-[1-(propane-2-sulfonyl)-pyrrolidin-3-yloxy]-phenyl}-amine,

{3-[2-(5-Methyl-thieno[2,3-d]pyrimidin-4-ylamino)-phenoxy]-pyrrolidin-1-yl}-pyridin-4-yl-methanone,

3-sec-Butoxy-4-(5,6-dimethyl-thieno[2,3-d]pyrimidin-4-ylamino)-benzamide,

3-Ethoxy-4-(5-methyl-thieno[2,3-d]pyrimidin-4-ylamino)-benzonitrile,

3-Isopropoxy-4-(5-methyl-thieno[2,3-d]pyrimidin-4-ylamino)-benzonitrile,

3-sec-Butoxy-4-(5,6-dimethyl-thieno[2,3-d]pyrimidin-4-ylamino)-benzonitrile,

(5,6-Dimethyl-thieno[2,3-d]pyrimidin-4-yl)-[2-(1-methanesulfonyl-pyrrolidin-3-yloxy)-phenyl]-amine,

3-(1-Methanesulfonyl-pyrrolidin-3-yloxy)-4-(5-methyl-thieno[2,3-d]pyrimidin-4-ylamino)-benzamide,

4-(5,6-Dimethyl-thieno[2,3-d]pyrimidin-4-ylamino)-3-(1-methanesulfonyl-pyrrolidin-3-yloxy)-benzamide,

[2-(Bicyclo[2.2.1]hept-2-yloxy)-phenyl]-(5-methyl-thieno[2,3-d]pyrimidin-4-yl)-amine,

(5-Ethyl-thieno[2,3-d]pyrimidin-4-yl)-[2-(tetrahydro-furan-3-yloxy)-phenyl]-amine,

[2-(3-Ethoxy-propoxy)-phenyl]-(5-methyl-thieno[2,3-d]pyrimidin-4-yl)-amine,

[2-(2-Ethoxy-ethoxy)-phenyl]-(5-methyl-thieno[2,3-d]pyrimidin-4-yl)-amine,

3-Ethoxy-4-(thieno[2,3-d]pyrimidin-4-ylamino)-benzamide,

(2-Cyclopentyloxy-4-fluoro-phenyl)-thieno[2,3-d]pyrimidin-4-yl-amine,

2-Fluoro-5-(tetrahydro-furan-3-yloxy)-4-(thieno[2,3-d]pyrimidin-4-ylamino)benzamide.

Most preferred are the following compounds:

[2-(Bicyclo[2.2.1]hept-2-yloxy)-phenyl]-thieno[2,3-d]pyrimidin-4-yl-amine,

(2-Ethoxy-phenyl)-(6-ethyl-thieno[2,3-d]pyrimidin-4-yl)-amine,

(2-sec-Butoxy-phenyl)-(6-ethyl-thieno[2,3-d]pyrimidin-4-yl)-amine,

(6-Ethyl-thieno[2,3-d]pyrimidin-4-yl)-[2-(tetrahydro-furan-3-yloxy)-phenyl]-amine,

[2-(Bicyclo[2.2.1]hept-2-yloxy)-phenyl]-(5-methyl-thieno[2,3-d]pyrimidin-4-yl)-amine,

[2-(Bicyclo[2.2.1]hept-2-yloxy)-phenyl]-(5,6-dimethyl-thieno[2,3-d]pyrimidin-4-yl)-amine,

(5-Methyl-thieno[2,3-d]pyrimidin-4-yl)-[2-(tetrahydro-furan-3-ylmethoxy)-phenyl]-amine,

(5,6-Dimethyl-thieno[2,3-d]pyrimidin-4-yl)-[2-(tetrahydro-furan-3-ylmethoxy)-phenyl]-amine,

(2-Isopropoxy-phenyl)-(6-isopropyl-thieno[2,3-d]pyrimidin-4-yl)-amine,

(2-Cyclopentyloxy-phenyl)-(6-isopropyl-thieno[2,3-d]pyrimidin-4-yl)-amine,

(6-Isopropyl-thieno[2,3-d]pyrimidin-4-yl)-[2-(tetrahydro-furan-3-yloxy)-phenyl]-amine,

[2-(1,2-Dimethyl-propoxy)-phenyl]-thieno[2,3-d]pyrimidin-4-yl-amine,

[2-(1,2-Dimethyl-propoxy)-phenyl]-(5-methyl-thieno[2,3-d]pyrimidin-4-yl)-amine,

[2-(1,2-Dimethyl-propoxy)-phenyl]-(5,6-dimethyl-thieno[2,3-d]pyrimidin-4-yl)-amine,

[5-Fluoro-2-(tetrahydro-furan-3-yloxy)-phenyl]-(5-methyl-thieno[2,3-d]pyrimidin-4-yl)-amine,

(5,6-Dimethyl-thieno[2,3-d]pyrimidin-4-yl)-[5-fluoro-2-(tetrahydro-furan-3-yloxy)-phenyl]-amine,

(6-Ethyl-thieno[2,3-d]pyrimidin-4-yl)-(2-isopropoxy-phenyl)-amine,

(2-Cyclopentyloxy-phenyl)-(6-ethyl-thieno[2,3-d]pyrimidin-4-yl)-amine,

(2-Methoxy-phenyl)-(6-methyl-5-propyl-thieno[2,3-d]pyrimidin-4-yl)-amine,

(2-Ethoxy-phenyl)-(6-methyl-5-propyl-thieno[2,3-d]pyrimidin-4-yl)-amine,

(2-Isopropoxy-phenyl)-(6-methyl-5-propyl-thieno[2,3-d]pyrimidin-4-yl)-amine,

3-(Tetrahydro-furan-3-yloxy)-4-(thieno[2,3-d]pyrimidin-4-ylamino)-benzoic acid methyl ester,

4-(5-Methyl-thieno[2,3-d]pyrimidin-4-ylamino)-3-(tetrahydro-furan-3-yloxy)-benzoic acid methyl ester,

3-(Tetrahydro-furan-3-yloxy)-4-(thieno[2,3-d]pyrimidin-4-ylamino)-benzamide,

[2-(Tetrahydro-furan-3-yloxy)-phenyl]-(5-trifluoromethyl-thieno[2,3-d]pyrimidin-4-yl)-amine,

(2-Cyclopentyloxy-phenyl)-(5-trifluoromethyl-thieno[2,3-d]pyrimidin-4-yl)-amine,

[2-(1-Ethyl-2-methyl-propoxy)-phenyl]-thieno[2,3-d]pyrimidin-4-yl-amine,

[2-(1-Ethyl-2-methyl-propoxy)-phenyl]-(5-methyl-thieno[2,3-d]pyrimidin-4-yl)-amine,

(6-Methyl-5-propyl-thieno[2,3-d]pyrimidin-4-yl)-[2-(tetrahydro-furan-3-yloxy)-phenyl]-amine,

(5,6-Dimethyl-thieno[2,3-d]pyrimidin-4-yl)-[2-(1-ethyl-2-methyl-propoxy)-phenyl]-amine,

(2-Isopropoxy-phenyl)-(5-trifluoromethyl-thieno[2,3-d]pyrimidin-4-yl)-amine,

(2-sec-Butoxy-phenyl)-(5-trifluoromethyl-thieno[2,3-d]pyrimidin-4-yl)-amine,

(5-Methyl-thieno[2,3-d]pyrimidin-4-yl)-(2-pyrrolidin-1-yl-phenyl)-amine,

(5,6-Dimethyl-thieno[2,3-d]pyrimidin-4-yl)-(2-pyrrolidin-1-yl-phenyl)-amine,

3-[2-(5,6-Dimethyl-thieno[2,3-d]pyrimidin-4-ylamino)-phenoxy]-pyrrolidine-1-carboxylic acid tert-butyl ester,

Thieno[2,3-d]pyrimidin-4-yl-[2-(3,3,3-trifluoro-propoxy)-phenyl]-amine,

(5-Methyl-thieno[2,3-d]pyrimidin-4-yl)-[2-(3,3,3-trifluoro-propoxy)-phenyl]-amine

(5,6-Dimethyl-thieno[2,3-d]pyrimidin-4-yl)-[2-(3,3,3-trifluoro-propoxy)-phenyl]-amine,

(5-Ethyl-thieno[2,3-d]pyrimidin-4-yl)-(2-isopropoxy-phenyl)-amine,

(2-sec-Butoxy-phenyl)-(5-ethyl-thieno[2,3-d]pyrimidin-4-yl)-amine,

(2-Cyclopentyloxy-phenyl)-(5-ethyl-thieno[2,3-d]pyrimidin-4-yl)-amine,

(5,6-Dimethyl-thieno[2,3-d]pyrimidin-4-yl)-[2-(3-ethoxy-propoxy)-phenyl]-amine,

[3-Fluoro-2-(tetrahydro-furan-3-yloxy)-phenyl]-(5-methyl-thieno[2,3-d]pyrimidin-4-yl)-amine,

(5,6-Dimethyl-thieno[2,3-d]pyrimidin-4-yl)-[3-fluoro-2-(tetrahydro-furan-3-yloxy)-phenyl]-amine,

(5,6-Dimethyl-thieno[2,3-d]pyrimidin-4-yl)-[2-(2-ethoxy-ethoxy)-phenyl]-amine,

3-Isopropoxy-4-(thieno[2,3-d]pyrimidin-4-ylamino)-benzamide,

3-sec-Butoxy-4-(thieno[2,3-d]pyrimidin-4-ylamino)-benzamide,

3-Cyclopentyloxy-4-(thieno[2,3-d]pyrimidin-4-ylamino)-benzamide,

3-Ethoxy-4-(5-methyl-thieno[2,3-d]pyrimidin-4-ylamino)-benzamide,

3-Isopropoxy-4-(5-methyl-thieno[2,3-d]pyrimidin-4-ylamino)-benzamide,

3-sec-Butoxy-4-(5-methyl-thieno[2,3-d]pyrimidin-4-ylamino)-benzamide,

3-Cyclopentyloxy-4-(5-methyl-thieno[2,3-d]pyrimidin-4-ylamino)-benzamide,

4-(5-Methyl-thieno[2,3-d]pyrimidin-4-ylamino)-3-(tetrahydro-furan-3-yloxy)-benzamide,

3-(Tetrahydro-furan-3-yloxy)-4-(5-trifluoromethyl-thieno[2,3-d]pyrimidin-4-ylamino)-benzamide,

4-(Thieno[2,3-d]pyrimidin-4-ylamino)-3-(3,3,3-trifluoro-propoxy)-benzamide,

4-(5-Methyl-thieno[2,3-d]pyrimidin-4-ylamino)-3-(3,3,3-trifluoro-propoxy)-benzamide,

4-(5,6-Dimethyl-thieno[2,3-d]pyrimidin-4-ylamino)-3-(3,3,3-trifluoro-propoxy)-benzamide,

3-Pyrrolidin-1-yl-4-(thieno[2,3-d]pyrimidin-4-ylamino)-benzamide,

4-(5-Methyl-thieno[2,3-d]pyrimidin-4-ylamino)-3-pyrrolidin-1-yl-benzamide,

4-(5,6-Dimethyl-thieno[2,3-d]pyrimidin-4-ylamino)-3-pyrrolidin-1-yl-benzamide,

4-(5,6-Dimethyl-thieno[2,3-d]pyrimidin-4-ylamino)-3-ethoxy-benzamide,

4-(5,6-Dimethyl-thieno[2,3-d]pyrimidin-4-ylamino)-3-isopropoxy-benzamide,

3-Cyclopentyloxy-4-(5,6-dimethyl-thieno[2,3-d]pyrimidin-4-ylamino)-benzamide,

4-(5,6-Dimethyl-thieno[2,3-d]pyrimidin-4-ylamino)-3-(tetrahydro-furan-3-yloxy)-benzamide,

(2-Ethoxy-4-fluoro-phenyl)thieno[2,3-d]pyrimidin-4-yl-amine,

[4-Fluoro-2-(tetrahydro-furan-3-yloxy)-phenyl]-thieno[2,3-d]pyrimidin-4-yl-amine,

(4-Fluoro-2-methoxy-phenyl)-(5-methyl-thieno[2,3-d]pyrimidin-4-yl)-amine,

(2-Ethoxy-4-fluoro-phenyl)-(5-methyl-thieno[2,3-d]pyrimidin-4-yl)-amine,

(2-Cyclopentyloxy-4-fluoro-phenyl)-(5-methyl-thieno[2,3-d]pyrimidin-4-yl)-amine,

[4-Fluoro-2-(tetrahydro-furan-3-yloxy)-phenyl]-(5-methyl-thieno[2,3-d]pyrimidin-4-yl)-amine,

(5,6-Dimethyl-thieno[2,3-d]pyrimidin-4-yl)-(4-fluoro-2-methoxy-phenyl)-amine,

(5,6-Dimethyl-thieno[2,3-d]pyrimidin-4-yl)-(2-ethoxy-4-fluoro-phenyl)-amine,

(5,6-Dimethyl-thieno[2,3-d]pyrimidin-4-yl)-(4-fluoro-2-isopropoxy-phenyl)-amine,

(5,6-Dimethyl-thieno[2,3-d]pyrimidin-4-yl)-[4-fluoro-2-(tetrahydro-furan-3-yloxy)-phenyl]-amine,

3-Methoxy-4-(5-trifluoromethyl-thieno[2,3-d]pyrimidin-4-ylamino)-benzamide,

3-Ethoxy-4-(5-trifluoromethyl-thieno[2,3-d]pyrimidin-4-ylamino)-benzamide,

3-Isopropoxy-4-(5-trifluoromethyl-thieno[2,3-d]pyrimidin-4-ylamino)-benzamide,

(4-Fluoro-2-isopropoxy-phenyl)-thieno[2,3-d]pyrimidin-4-yl-amine,

(2-sec-Butoxy-4-fluoro-phenyl)-thieno[2,3-d]pyrimidin-4-yl-amine,

(4-Fluoro-2-isopropoxy-phenyl)-(5-methyl-thieno[2,3-d]pyrimidin-4-yl)-amine,

(2-sec-Butoxy-4-fluoro-phenyl)-(5-methyl-thieno[2,3-d]pyrimidin-4-yl)-amine,

(2-sec-Butoxy-4-fluoro-phenyl)-(5,6-dimethyl-thieno[2,3-d]pyrimidin-4-yl)-amine,

(4-Fluoro-2-methoxy-phenyl)-(5-trifluoromethyl-thieno[2,3-d]pyrimidin-4-yl)-amine,

(4-Fluoro-2-isopropoxy-phenyl)-(5-trifluoromethyl-thieno[2,3-d]pyrimidin-4-yl)-amine,

[4-Fluoro-2-(tetrahydro-furan-3-yloxy)-phenyl]-(5-trifluoromethyl-thieno[2,3-d]pyrimidin-4-yl)-amine,

[4-Fluoro-2-(3,3,3-trifluoro-propoxy)-phenyl]-thieno[2,3-d]pyrimidin-4-yl-amine,

[4-Fluoro-2-(3,3,3-trifluoro-propoxy)-phenyl]-(5-methyl-thieno[2,3-d]pyrimidin-4-yl)-amine,

(5,6-Dimethyl-thieno[2,3-d]pyrimidin-4-yl)-[4-fluoro-2-(3,3,3-trifluoro-propoxy)-phenyl]-amine,

4-(5,6-Dimethyl-thieno[2,3-d]pyrimidin-4-ylamino)-N-methyl-3-(tetrahydro-furan-3-yloxy)-benzamide,

2-Fluoro-5-methoxy-4-(thieno[2,3-d]pyrimidin-4-ylamino)-benzamide,

2-Fluoro-5-isopropoxy-4-(thieno[2,3-d]pyrimidin-4-ylamino)-benzamide,

2-Fluoro-5-methoxy-4-(5-methyl-thieno[2,3-d]pyrimidin-4-ylamino)-benzamide,

2-Fluoro-4-(5-methyl-thieno[2,3-d]pyrimidin-4-ylamino)-5-(tetrahydro-furan-3-yloxy)-benzamide,

4-(5,6-Dimethyl-thieno[2,3-d]pyrimidin-4-ylamino)-2-fluoro-5-methoxy-benzamide,

4-(5,6-Dimethyl-thieno[2,3-d]pyrimidin-4-ylamino)-2-fluoro-5-isopropoxy-benzamide,

4-(5,6-Dimethyl-thieno[2,3-d]pyrimidin-4-ylamino)-2-fluoro-5-(tetrahydro-furan-3-yloxy)-benzamide,

[2-(1-Methanesulfonyl-pyrrolidin-3-yloxy)-phenyl]-(5-methyl-thieno[2,3-d]pyrimidin-4-yl)-amine,

1-{3-[2-(5-Methyl-thieno[2,3-d]pyrimidin-4-ylamino)-phenoxy]-pyrrolidin-1-yl}-ethanone,

3-[2-(5-Methyl-thieno[2,3-d]pyrimidin-4-ylamino)-phenoxy]-pyrrolidine-1-carboxylic acid dimethylamide,

2-Methyl-1-{3-[2-(5-methyl-thieno[2,3-d]pyrimidin-4-ylamino)-phenoxy]-pyrrolidin-1-yl}-propan-1-one,

Cyclopropyl-{3-[2-(5-methyl-thieno[2,3-d]pyrimidin-4-ylamino)-phenoxy]-pyrrolidin-1-yl}-methanone,

Cyclopentyl-{3-[2-(5-methyl-thieno[2,3-d]pyrimidin-4-ylamino)-phenoxy]-pyrrolidin-1-yl}-methanone,

3-[2-(5-Methyl-thieno[2,3-d]pyrimidin-4-ylamino)-phenoxy]-pyrrolidine-1-sulfonic acid dimethylamide,

(5-Methyl-thieno[2,3-d]pyrimidin-4-yl)-{2-[1-(propane-2-sulfonyl)-pyrrolidin-3-yloxy]-phenyl}-amine,

{3-[2-(5-Methyl-thieno[2,3-d]pyrimidin-4-ylamino)-phenoxy]-pyrrolidin-1-yl}-pyridin-4-yl-methanone,

3-sec-Butoxy-4-(5,6-dimethyl-thieno[2,3-d]pyrimidin-4-ylamino)-benzamide,

3-Ethoxy-4-(5-methyl-thieno[2,3-d]pyrimidin-4-ylamino)-benzonitrile,

3-Isopropoxy-4-(5-methyl-thieno[2,3-d]pyrimidin-4-ylamino)-benzonitrile,

3-sec-Butoxy-4-(5,6-dimethyl-thieno[2,3-d]pyrimidin-4-ylamino)-benzonitrile,

(5,6-Dimethyl-thieno[2,3-d]pyrimidin-4-yl)-[2-(1-methanesulfonyl-pyrrolidin-3-yloxy)-phenyl]-amine,

3-(1-Methanesulfonyl-pyrrolidin-3-yloxy)-4-(5-methyl-thieno[2,3-d]pyrimidin-4-ylamino)-benzamide,

4-(5,6-Dimethyl-thieno[2,3-d]pyrimidin-4-ylamino)-3-(1-methanesulfonyl-pyrrolidin-3-yloxy)-benzamide,

[2-(Bicyclo[2.2.1]hept-2-yloxy)-phenyl]-(5-methyl-thieno[2,3-d]pyrimidin-4-yl)-amine,

(5-Ethyl-thieno[2,3-d]pyrimidin-4-yl)-[2-(tetrahydro-furan-3-yloxy)-phenyl]-amine,

[2-(3-Ethoxy-propoxy)-phenyl]-(5-methyl-thieno[2,3-d]pyrimidin-4-yl)-amine,

(2-Cyclopentyloxy-4-fluoro-phenyl)-thieno[2,3-d]pyrimidin-4-yl-amine.

Typical methods of preparing the compounds of the invention are described below in the experimental section.

The potent inhibitory effect of the compounds of the invention may be determined by in vitro enzyme assays as described in the Examples in more detail.

Pharmaceutically acceptable salts of the compounds of the invention of formula (1) can be formed with numerous organic and inorganic acids and bases. Exemplary acid addition salts including acetate, adipate, alginate, ascorbate, aspartate, benzoate, benzenesulfonate, bisulfate, borate, butyrate, citrate, camphorate, camphersulfonate, cyclopentanepropionate, digluconate, dodecyl sulfate, ethane sulfonate, fumarate, glucoheptanoate, glycerophosphate, hemisulfate, heptanoate, hexanoate, hydrochloride, hydrobromide, hydroiodide, 2-hydroxyethane sulfonate, lactate, maleate, methane sulfonate, 2-naphthalene sulfonate, nicotinate, nitrate, oxalate, pamoate, pectinate, persulfate, 3-phenyl sulfonate, 3-phenylpropionate, phosphate, picrate, pivalate, propionate, salicylate, succinate, sulfate, sulfonate, tartrate, thiocyanate, toluene sulfonate such as tosylate, undecanoate, or the like.

Basic nitrogen-containing moieties can be quaternized with such agents as lower alkyl halides, such as methyl, ethyl, propyl, and butyl chloride, bromide and iodide; dialkyl sulfates like dimethyl, diethyl, dibutyl, and diamyl sulfates, long-chain alkyl halides such as decyl, lauryl, myristyl and stearyl chloride, bromide and iodide, or aralkyl halides like benzyl and phenethyl bromides, or others. Water soluble or dispersible products are thereby obtained.

Pharmaceutically acceptable basic addition salts include but are not limited to cations based on the alkaline and alkaline earth metals such as sodium, lithium, potassium, calcium, magnesium, aluminum salts and the like, as well as non toxic ammonium quaternary ammonium, and amine cations, including but not limited to ammonium, tetramethylammonium, tetraethylammonium, methylamine, dimethylamine, trimethylamine, triethylamine, ethylamine and the like. Other representative amines useful for the formation of base addition salts include benzazethine, dicyclohexyl amine, hydrabine, N-methyl-D-glucamine, N-methyl-D-glucamide, t-butyl amine, diethylamine, ethylendiamine, ethanolamine, diethanolamine, piperazine and the like and salts with amino acids such as arginine, lysine, or the like.

Compounds of the formula (1) can be present as tautomers. The present invention comprises all tautomeric forms. Furthermore, the present invention also comprises all stereoisomers of the compounds according to the invention, including its enantiomers and diastereomers. Individual stereoisomers of the compounds according to the invention can be substantially present pure of other isomers, in admixture thereof or as racemates or as selected stereoisomers.

As used herein the term “metabolite” refers to (i) a product of metabolism, including intermediate and products, (ii) any substance involved in metabolism (either as a product of metabolism or as necessary for metabolism), or (iii) any substance produced or used during metabolism. In particular it refers to the end product that remains after metabolism.

As used herein the term “prodrug” refers to (i) an inactive form of a drug that exerts its effects after metabolic processes within the body convert it to a usable or active form, or (ii) a substance that gives rise to a pharmacologically active metabolite, although not itself active (i.e. an inactive precursor).

As used herein the term “C₃₋₁₀ cycloalkyl” refers to mono- or polycyclic carbocyclic alkyl substituent or group having 3 to 10 ring atoms, such as cyclopropyl, cyclobutyl, cyclopentyl, cyclohexyl, cycloheptyl, cyclobutenyl, cyclopentenyl, cyclopentadienyl, cyclohexenyl, cyclohexadienyl, cycloheptenyl, cycloheptadienyl, cycloheptatrienyl perhydrated naphthalene or indene, adamantyl or norbonanyl and the like.

The term “C₁₋₆ alkyl” as used herein alone or in combination with other terms such as in alkoxy refers to a C₁₋₆, preferably C₁₋₄ straight or branched alkyl/alkoxy group such as methyl, ethyl, propyl (iso-, n-), butyl (iso-, n-, sec-, tert-), pentyl, hexyl, methoxy, ethoxy, propoxy (iso-, n-), butoxy (iso-, n-, sec-, tert-), pentoxy, hexoxy; moreover, the term “C₁₋₆ alkyl” also includes an alkyl group which may contain oxygen in the chain and may be substituted with halogen to form an ether or halogenated ether group.

The term “halogen” refers to a halogen atom selected from fluorine, chlorine, bromine, iodine, preferably fluorine and chlorine, more preferably fluorine.

The term “aryl” refers to a mono- or bicyclic aromatic group having 6 to 10 backbone carbon atoms, wherein optionally one of the rings of the bicyclic structure is aromatic and the other is a carbocyclic group, such as phenyl, 1-naphthyl, 2-naphthyl, indenyl, indanyl, azulenyl, fluorenyl, 1,2,3,4-tetrahydronaphthyl.

The term “heterocyclyl” refers to monocyclic saturated or unsaturated heterocyclyl groups with 1 to 4 hetero atoms selected from N, S and O, with the remainder of the ring atoms being carbon atoms and having preferably a total number of ring atoms of 3 to 10, such as morpholino, piperazinyl, piperidinyl, pyridyl, pyrimidinyl, thiazolyl, indolyl, imidazolyl, oxadiazolyl, tetrazolyl, pyrazinyl, triazolyl, thiophenyl or furanyl.

The term “heteroaryl” refers to a mono- or bicyclic aromatic group with 1 to 4 hetero atoms selected from N, S and O, with the remainder of the ring atoms being carbon atoms and having preferably a total number of ring atoms of 5 to 10. Examples without limitation of heteroaryl groups are such as benzofuranyl, furyl, thienyl, benzothienyl, thiazolyl, imidazolyl, oxazolyl, oxadiazolyl, thiadiazolyl, benzothiazolyl, triazolyl, tetrazolyl, isoxazolyl, isothiazolyl, pyrrolyl, pyranyl, tetrahydropyranyl, pyrazolyl, pyridyl, quinolinyl, isoquinolinyl, purinyl, carbazolyl, benzoxazolyl, benzamidazolyl, indolyl, isoindolyl, pyrazinyl, diazinyl, pyrazine, triazinyltriazine, tetrazinyl, tetrazolyl, benzothiophenyl, benzopyridyl and benzimidazolyl.

In a further aspect the present invention provides pharmaceutical compositions comprising a thienopyrimidine compound of the present invention and optionally a pharmaceutically acceptable carrier.

The pharmaceutical composition according to the present invention may further comprise an additional therapeutic agent. Particularly preferred are compositions, wherein the additional therapeutic agent is selected from antidiabetics like insulin, long and short acting insulin analogues, sulfonylureas and other antidiabetics derived from thiazolidindiones, lipid lowering agents such as statines, fibrates, ion exchange resins, nicotinic acid derivatives, or HMG-CoA reductase inhibitors, cardiovascular therapeutics such as nitrates, antihypertensiva such as β-blockers, ACE inhibitors, Ca-channel blockers, angiotensin II receptor antagonists, diuretics, thrombocyte aggregation inhibitors, or antineoplastic agents such as alkaloids, alkylating agents, antibiotics, or antimetabolites, or anti-obesity agents.

More particularly preferred are compounds such as human NPH insulin, human lente or ultralente insulin, insulin Lispro, insulin Asptart, or insulin Glargine, atenolol, bisoprolol, metoprolol, esmolol, celiprolol, talinolol, oxprenolol, pindolol, propanolol, bupropanolol, penbutolol, mepindolol, sotalol, certeolol, nadolol, carvedilol, nifedipin, nitrendipin, amlodipin, nicardipin, nisoldipin, diltiazem, enalapril, verapamil, gallopamil, quinapril, captopril, lisinopril, benazepril, ramipril, peridopril, fosinopril, trandolapril, irbesatan, losartan, valsartan, telmisartan, eprosartan, olmesartan, hydrochlorothiazide, piretanid, chlorotalidone, mefruside, furosemide, bendroflumethiazid, triamterene, dehydralazine, acetylsalicylic acid, tirofiban-HCl, dipyramidol, triclopidin, iloprost-trometanol, eptifibatide, clopidogrel, piratecam, abciximab, trapidil, simvastatine, bezafibrate, fenofibrate, gemfibrozil, etofyllin, clofibrate, etofibrate, fluvastatine, lovastatine, pravastatin, colestyramide, colestipol-HCl, xantinol nicotinat, inositol nicotinat, acipimox, nebivolol, glycerolnitrate, isosorbide mononitrate, isosorbide dinitrate, pentaerythrityl tetranitrate, indapamide, cilazepril, urapidil, eprosartan, nilvadipin, metoprolol, doxazosin, molsidormin, moxaverin, acebutolol, prazosine, trapidil, clonidine, vinca alkaloids and analogues such as vinbiastin, vincristin, vindesin, vinorelbin, podophyllotoxine derivatives, etoposid, teniposid, alkylating agents, nitroso ureas, N-lost analogues, cycloplonphamid, estamustin, melphalan, ifosfamid, mitoxantron, idarubicin, doxorubicin, bleomycin, mitomycin, dactinomycin, daptomycin, antimetabolites such as cytarabin, fluorouracil, fluoroarabin, gemcitabin, tioguanin, capecitabin, combinations such as adriamydin/daunorubicin, cytosine arabinosid/cytarabine, 4-HC, or other phosphamides.

It will be appreciated by the person of ordinary skill in the art that the compounds of the invention and the additional therapeutic agent may be formulated in one single dosage form, or may be present in separate dosage forms and may be either administered concomitantly (i.e. at the same time) or sequentially.

The pharmaceutical compositions of the present invention may be in any form suitable for the intended method of administration.

The compounds of the present invention may be administered orally, parenterally, such as bronchopulmonary, subcutaneously, intravenously, intramuscularly, intraperitoneally, intrathecally, transdermally, transmucosally, subdurally, locally or topically via iontopheresis, sublingually, by inhalation spray, aerosol or rectally and the like in dosage unit formulations optionally comprising conventional pharmaceutically acceptable excipients.

Excipients that may be used in the formulation of the pharmaceutical compositions of the present invention comprise carriers, vehicles, diluents, solvents such as monohydric alcohols such as ethanol, isopropanol and polyhydric alcohols such as glycols and edible oils such as soybean oil, coconut oil, olive oil, safflower oil cottonseed oil, oily esters such as ethyl oleate, isopropyl myristate; binders, adjuvants, solubilizers, thickening agents, stabilizers, disintergrants, glidants, lubricating agents, buffering agents, emulsifiers, wetting agents, suspending agents, sweetening agents, colorants, flavors, coating agents, preservatives, antioxidants, processing agents, drug delivery modifiers and enhancers such as calcium phosphate, magnesium state, talc, monosaccharides, disaccharides, starch, gelatine, cellulose, methylcellulose, sodium carboxymethyl cellulose, dextrose, hydroxypropyl-β-cyclodextrin, polyvinylpyrrolidone, low melting waxes, ion exchange resins.

Other suitable pharmaceutically acceptable excipients are described in Remington's Pharmaceutical Sciences, 15^{th} Ed., Mack Publishing Co., New Jersey (1991).

Dosage forms for oral administration include tablets, capsules, lozenges, pills, wafers, granules, oral liquids such as syrups, suspensions, solutions, emulsions, powder for reconstitution.

Dosage forms for parenteral administration include aqueous or olageous solutions or emulsions for infusion, aqueous or olageous solutions, suspensions or emulsions for injection pre-filled syringes, and/or powders for reconstitution.

Dosage forms for local/topical administration comprise insufflations, aerosols, metered aerosols, transdermal therapeutic systems, medicated patches, rectal suppositories, and/or ovula.

The amount of the compound of the present invention that may be combined with the excipients to formulate a single dosage form will vary upon the host treated and the particular mode of administration.

The pharmaceutical compositions of the invention can be produced in a manner known per se to the skilled person as described, for example, in Remington's Pharmaceutical Sciences, 15^{th} Ed., Mack Publishing Co., New Jersey (1991).

In a further aspect of the invention the use of a thienopyrimidine compound of the present invention for the production of a pharmaceutical composition for inhibiting the activity of the kinase activity of Mnk1 or Mnk2 (Mnk2a, Mnk2b) or further variants thereof is provided, in particular for the prophylaxis or therapy of metabolic diseases, hematopoietic disorders, cancer and their consecutive complications and disorders. Whereby the prophylaxis and therapy of metabolic diseases and hematopoietic disorders is preferred.

Diseases of the invention that are influenced by the inhibition of the kinase activity of Mnk1 and/or Mnk2 (Mnk2a or Mnk2b) and/or further variants thereof include diseases related to the regulation of metabolic diseases, such as obesity, eating disorders, cachexia, diabetes mellitus, metabolic syndrome, hypertension, coronary heart diseases, hypercholesterolemia, dyslipidemia, osteoarthritis, biliary stones and/or sleep apnea and diseases related to reactive oxygen compounds (ROS defense) such as diabetes mellitus, neurodegenerative diseases and cancer.

The pharmaceutical compositions of the invention are particularly useful for prophylaxis and treatment of obesity, diabetes mellitus and other metabolic diseases of the carbohydrate and lipid metabolism as stated above, in particular diabetes mellitus and obesity.

Thus in a more preferred embodiment of this invention the use of a thienopyrimidine compound for the production of a pharmaceutical composition for the prophylaxis or therapy of metabolic diseases is provided.

For the purpose of the present invention, a therapeutically effective dosage will generally be from about 1 to 500 mg/day, preferably from about 10 to about 200 mg/day, and most preferably from about 10 to about 100 mg/day, which may be administered in one or multiple doses.

It will be appreciated, however, that specific dose level of the compounds of the invention for any particular patient will depend on a variety of factors such as age, sex, body weight, general health condition, diet, individual response of the patient to be treated time of administration, severity of the disease to be treated, the activity of particular compound applied, dosage form, mode of application and concomitant medication. The therapeutically effective amount for a given situation will readily be determined by routine experimentation and is within the skills and judgment of the ordinary clinician or physician.

### EXAMPLES

General

LCMS analyses of purity and m/z were performed using a Waters Micromass LCT mass spectrometer linked to a ThermoHypersil-Keystone BDS 5μ, 2.1×500 mm column eluting with a gradient of 100% water to 95% acetonitrile in 5% water (0.1% TFA buffer) over 2.1 minutes at a flow rate of 1 ml/min with detection by UV at 215 nm and ELS. Proton nuclear magnetic resonance (NMR) spectra were recorded on a Bruker AVANCE 400 MHz or on a Bruker DPX 250 MHz spectrometer with reference to the deuterated solvent peak.

Starting materials containing the thienopyrimidine ring core are commercially available from suppliers such as Fluorochem Ltd. and Maybridge. Access to thienopyrimidines with structurally diverse R₂ and R₃ groups is achieved from the appropriately substituted 2-amino-thiophene-3-carboxylic ester. This intermediate is prepared via the “Gewald thiophene synthesis” (Chem. Ber. 1966, 99, 94-100) (1. Method, shown below) or an alternative synthetic route described in Pharmazie 1996, 51(11), 833-836 where the R₂ group can be selectively introduced (2. Method, shown below):

1. Method

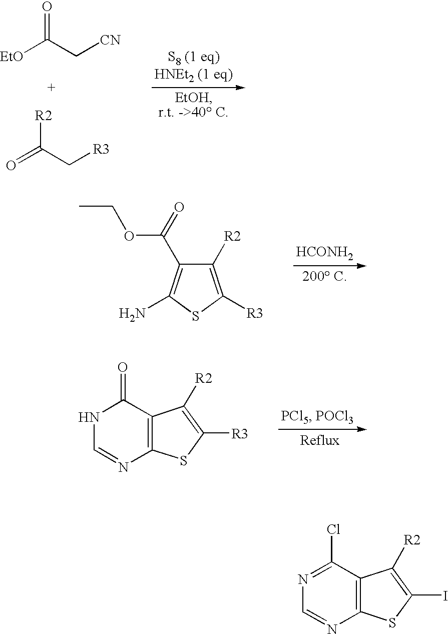

2. Method

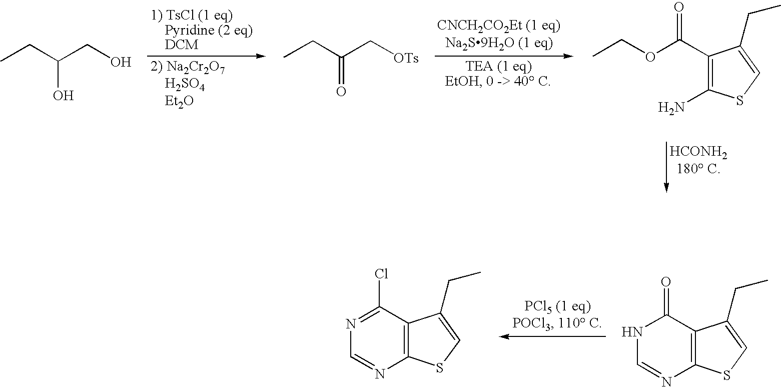

The 2-amino-thiophene-3-carboxylic ester products are cyclized with formamide to yield the corresponding 4-oxo-thienopyrimidine which is readily converted into the activated 4-chloro-thienopyrimidine with a mixture of PCl₅ and POCl₃ or neat POCl₃. The 4-chloro-thienopyrimidines are then reacted with aniline derivatives as described in synthetic routes 1 to 25 described below to afford the compound of the invention.

### Example 1

### Examples of Preparation of the Compounds of the Invention

The compounds of the invention can be produced in a manner known per se and by the synthetic routes 1-5 described below.

### Example 1a

### Synthesis Route 1

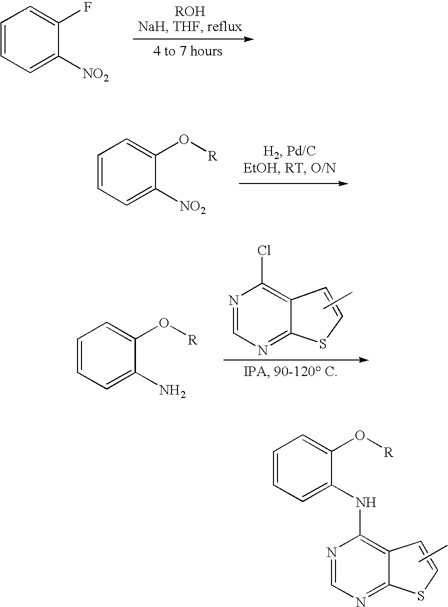

### Compound 1a: 3-(2-Nitro-phenoxy)-tetrahydro-furan

Anhydrous tetrahydrofuran (10 ml) was added to sodium hydride as a 60% dispersion in mineral oil (312 mg, 7.8 mmol, 1.1 eq) in a flask fitted with a condenser, a nitrogen inlet and a bubbler. While stirring, 3-hydroxytetrahydrofuran (624 mg, 7.09 mmol, 1 eq) was added slowly and the mixture was left to stir at room temperature for 10-15 minutes. To the solution of sodium alkoxide in THF was added 2-fluoronitrobenzene (1 g, 7.09 mmol, 1 eq). The reaction mixture was heated to reflux with stirring for 4.5 hours. The reaction was then allowed to cool down to room temperature, then water (20 ml) was added to the reaction mixture. The resulting mixture was extracted three times with ethyl acetate (20 ml), the organics dried over sodium sulphate, filtered and the filtrate evaporated to dryness in vacuo to give the title compound as orange oil (1.48 g, 7.07 mmol, 100%). ¹H NMR indicates desired compound in ca. 90% purity.

### Compound 1b: 2-(Tetrahydro-furan-3-yloxy)-phenylamine

In a flask purged and fitted with a 3 way tap under nitrogen was added 10% w/w palladium on charcoal (150 mg, 10% w/w) followed by ethanol (20 ml). The flask was purged again and placed under nitrogen and 3-(2-Nitro-phenoxy)-tetrahydrofuran (1.48 g, 7.07 mmol, 1 eq) in solution in ethanol (20 ml) was added. The flask was purged and placed under an atmosphere of hydrogen and the reaction mixture was stirred overnight at room temperature. The palladium residues were filtered on glass fibre paper and the filtrate was evaporated to dryness in vacuo to yield the title compound (1.14 g, 6.36 mmol, 90%). ¹H NMR indicates desired compound in ca. 95% purity.

### Compound 1c: (5,6-Dimethyl-thieno[2,3-d]pyrimidin-4-yl)-[2-(tetrahydro-furan-3-yloxy)-phenyl]-amine

2-(Tetrahydro-furan-3-yloxy)-phenylamine (100 mg, 0.58 mmol, 1 eq) was placed in an Ace pressure tube to which was added 4-chloro-5,6-dimethyl-thieno[2,3-d]pyrimidine (111 mg, 0.58 mmol, 1 eq). 2-propanol (4 ml) was added and the reaction mixture was stirred at 90° C. for 7 hours. The title compound precipitated as the hydrochloride salt and was filtered off. It was taken in 4 ml of sodium hydroxide 5N and extracted twice with dichloromethane (3 ml). The organics were filtered through a PS-syringe fitted with a sodium sulphate drying cartridge and the filtrate was evaporated to dryness in vacuo. The crude compound was purified by column chromatography on silica using hexane followed by a hexane/ethyl acetate (9:1) mixture as eluent to yield the title compound (24.5 mg, 0.07 mmol, 13%). LCMS; [M+H]⁺=342, Rt=1.92 min, 100% purity

The compounds listed below were prepared using route 1;

### Compound 2a: 3(S) -(2-Nitro-phenoxy)-tetrahydro-furan

Yield; 1.71 g, 8.17 mmol, 100%

¹H NMR indicates desired compound in ca. 90% purity

### Compound 2b: 2-(Tetrahydro-furan-3-(S)-yloxy)-phenylamine

Yield; 1.03 g, 5.75 mmol, 81%

¹H NMR indicates desired compound in ca. 95% purity

### Compound 2c: (5,6-Dimethyl-thieno[2,3-d]pyrimidin-4-yl)-[2-(tetrahydro-furan-3-yloxy)-phenyl]-amine

Yield; 135.9 mg, 0.398 mmol, 72%

LCMS; [M+H]⁺=342, Rt=1.92 min, 98% purity

### Compound 3a: 3(R)-(2-Nitro-phenoxy)-tetrahydro-furan

Yield; 1.58 g, 7.56 mmol, 100%

¹H NMR indicates desired compound in ca. 90% purity

### Compound 3b: 2-(Tetrahydro-furan-3-(R)-yloxy)-phenylamine

Yield; 985.7 mg, 5.50 mmol, 72%

¹H NMR indicates desired compound in ca. 95% purity

### Compound 3c: (5,6-Dimethyl-thieno[2,3-d]pyrimidin-4-yl)-[2-(tetrahydro-furan-3-(R)-yloxy)-phenyl]-amine

Yield; 125.4 mg, 0.367 mmol, 66%

LCMS; [M+H]⁺=342, Rt=1.92 min, 100% purity

### Compound 4c: (5-Methyl-thieno[2,3-d]pyrimidin-4-yl)-[2-(tetrahydro-furan-3-yloxy)-phenyl]-amine

Yield; 63.9 mg, 0.195 mmol, 35%

LCMS; [M+H]⁺=328, Rt=1.88 min, 100% purity

### Compound 5a: 1-Cyclopentyloxy-2-nitro-benzene

Yield; 664.1 mg, 3.21 mmol, 45%

¹H NMR indicates desired compound in ca. 90% purity

### Compound 5b: 2-Cyclopentyloxy-phenylamine

Yield; 325.4 mg, 1.83 mmol, 58%

¹H NMR indicates desired compound in ca. 95% purity

### Compound 5c: (2-Cyclopentyloxy-phenyl)-(5-methyl-thieno[2,3-d]pyrimidin-4-yl)-amine

Yield; 23 mg, 0.071 mmol, 12.5%

LCMS; [M+H]⁺=326, Rt=2.26 min, 100% purity

### Compound 6c: (5-Methyl-thieno[2,3-d]pyrimidin-4-yl)-[2-(tetrahydro-furan-3-(S)-yloxy)-phenyl]-amine

Yield; 82 mg, 0.448 mmol, 45%

LCMS; [M+H]⁺=328, Rt=1.88 min, 100% purity

### Compound 7a: 4-(2-Nitro-phenoxy)-tetrahydro-pyran

Yield; 1.59 g, 7.25 mmol, 100%

¹H NMR indicates desired compound in ca. 90% purity

### Compound 7b: 2-(Tetrahydro-pyran-4-yloxy)-phenylamine

Yield; 1.24 g, 6.42 mmol, 91%

¹H NMR indicates desired compound in ca. 88% purity (12% w/w EtOH remaining)

### Compound 7c: (5,6-Dimethyl-thieno[2,3-d]pyrimidin-4-yl)-[2-(tetrahydro-pyran-4-yloxy)-phenyl]-amine

Yield; 132.6 mg, 0.373 mmol, 72%

LCMS; [M+H]⁺=356, Rt=1.96 min, 100% purity

### Compound 8a: 1-sec-Butoxy-2-nitro-benzene

Yield; 1.33 g, 6.86 mmol, 97%

LCMS; [M+H]⁺=NI, Rt=1.53 min, 90% purity

¹H NMR indicates desired compound in ca. 95% purity

### Compound 8b: 2-sec-Butoxy-phenylamine

Yield; 902.6 mg, 5.5 mmol, 80%

¹H NMR indicates desired compound in ca. 98% purity

### Compound 8c: (2-sec-Butoxy-phenyl)-(5,6-dimethyl-thieno[2,3-d]pyrimidin-4-yl)-amine

Yield; 17.8 mg, 0.054 mmol, 9%

LCMS; [M+H]⁺=328, Rt=1.69 min, 100% purity

### Compound 9a: 1-Isopropoxy-2-nitro-benzene

Yield; 1.18 g, 6.52 mmol, 92%

LCMS; [M+H]⁺=NI, Rt=1.41 min, 95% purity

### Compound 9b: 2-Isopropoxy-phenylamine

Yield; 0.9 g, 5.96 mmol, 91%

LCMS; [M+H]⁺=152, Rt=0.72 min, 100% purity

### Compound 9c: (2-Isopropoxy-phenyl)-(5-methyl-thieno[2,3-d]pyrimidin-4-yl)-amine

Yield; 35 mg, 0.117 mmol, 22%

LCMS; [M+H]⁺=300, Rt=1.57 min, 100% purity

### Compound 10c: (5,6-Dimethyl-thieno[2,3-d]pyrimidin-4-yl)-[2-(tetrahydro-furan-3(R)-yloxy)-phenyl]-amine

Yield; 138.8 mg, 0.424 mmol, 76%

LCMS; [M+H]⁺=328, Rt=1.88 min, 100% purity

### Compound 11c: (2-sec-Butoxy-phenyl)-(5-methyl-thieno[2,3-d]pyrimidin-4-yl)-amine

Yield; 20.2 mg, 0.064 mmol, 11%

LCMS; [M+H]⁺=314, Rt=1.77 min, 94% purity

### Compound 12c: (5-Methyl-thieno[2,3-d]pyrimidin-4-yl)-[2-(tetrahydro-pyran-4-yloxy)-phenyl]-amine

Yield; 150.2 mg, 0.439 mmol, 85%

LCMS; [M+H]⁺=342, Rt=1.93 min, 100% purity

### Compound 16c: (5,6-Dimethyl-thieno[2,3-d]pyrimidin-4-yl)-(2-isopropoxy-phenyl)-amine

Yield; 66 mg, 0.211 mmol, 39%

LCMS; [M+H]⁺=314, Rt=1.61 min, 89% purity

### Compound 19a: 1-Cyclohexyloxy-2-nitro-benzene

Yield; 1.79 g, 8.09 mmol, 100%

¹H NMR indicates desired compound in ca. 90% purity

### Compound 19b: 2-Cyclohexyloxy-phenylamine

Yield; 1.49 g, 7.78 mmol, 96%

¹H NMR indicates desired compound in ca. 95% purity

### Compound 19c: (2-Cyclohexyloxy-phenyl)-(5-methyl-thieno[2,3-d]pyrimidin-4-yl)-amine

Yield; 47.2 mg, 0.139 mmol, 27%

LCMS; [M+H]⁺=340, Rt=2.33 min, 100% purity

### Compound 20a: 1-Cyclopropylmethoxy-2-nitro-benzene

Yield; 1.22 g, 6.32 mmol, 89%

¹H NMR indicates desired compound in ca. 90% purity

### Compound 20b: 2-Cyclopropylmethoxy-phenylamine

Yield; 954.9 mg, 5.85 mmol, 93%

LCMS; [M+H]⁺=164, Rt=0.84 min, 100% purity

¹H NMR indicates desired compound in ca. 95% purity

### Compound 20c: (2-Cyclopropylmethoxy-phenyl)-(5-methyl-thieno[2,3-d]pyrimidin-4-yl)-amine

Yield; 74.3 mg, 0.239 mmol, 39%

LCMS; [M+H]⁺=312, Rt=1.68 min, 100% purity

### Compound 22c: (2-Cyclohexyloxy-phenyl)-(5,6-dimethyl-thieno[2,3-d]pyrimidin-4-yl)-amine

Yield; 102.9 mg, 0.291 mmol, 56%

LCMS; [M+H]⁺=354, Rt=2.36 min, 97% purity

### Compound 23a: 1-tert-Butoxy-2-nitro-benzene

Yield; 1.08 g, 6.32 mmol, 78%

¹H NMR indicates desired compound in ca. 95% purity

### Compound 23b: 2-tert-Butoxy-phenylamine

Yield; 719.8 mg, 4.36 mmol, 79%

LCMS; [M+H]⁺=166, Rt=0.89 min, 100% purity

¹H NMR indicates desired compound in ca. 95% purity

### Compound 23c: (2-tert-Butoxy-phenyl)-(5,6-dimethyl-thieno[2,3-d]pyrimidin-4-yl)-amine

Yield; 25.3 mg, 0.077 mmol, 13%

LCMS; [M+H]⁺=328, Rt=1.67 min, 94% purity

### Compound 25a: 1-Nitro-2-propoxy-benzene

LCMS; [M+H]⁺=NI, Rt=1.44 min, 100% purity

### Compound 25b: 2-Propoxy-phenylamine

The desired compound was used without purification in the subsequent reaction.

### Compound 25c: (5-Methyl-thieno[2,3-d]pyrimidin-4-yl)-(2-propoxy-phenyl)-amine

Yield; 10 mg, 0.033 mmol, 13%

LCMS; [M+H]⁺=300, Rt=1.54 min, 100% purity

### Compound 26c: (2-Cyclopentyloxy-phenyl)-(5,6-dimethyl-thieno[2,3-d]pyrimidin-4-yl)-amine

Yield; 8.8 mg, 0.026 mmol, 5%

LCMS; [M+H]⁺=340, Rt=2.29 min, 92% purity

### Compound 27a: 1-Ethyl-3-(2-nitro-phenoxy)-pyrrolidine

Yield; 1.70 g, 7.2 mmol, 100%

¹H NMR indicates desired compound in ca. 95% purity

### Compound 27b: 2-(1-Ethyl-pyrrolidin-3-yloxy)-phenylamine

Yield; 1.47 g, 7.13 mmol, 99%

¹H NMR indicates desired compound in ca. 95% purity

### Compound 27c: (5,6-Dimethyl-thieno[2,3-d]pyrimidin-4-yl)-[2-(1-ethyl-pyrrolidin-3-yloxy)-phenyl]-amine

Yield; 8.0 mg, 0.022 mmol, 4.5%

LCMS; [M+H]⁺=369, Rt=1.61 min, 92% purity

### Compound 28c: (2-tert-Butoxy-phenyl)-(5-methyl-thieno[2,3-d]pyrimidin-4-yl)-amine

Yield; 32 mg, 0.102 mmol, 17%

LCMS; [M+H]⁺=314, Rt=2.10 min, 93% purity

### Compound 32c: (2-Cyclopropylmethoxy-phenyl)-(5,6-dimethyl-thieno[2,3-d]pyrimidin-4-yl)-amine

Yield; 88.2 mg, 0.271 mmol, 44%

LCMS; [M+H]⁺=326, Rt=2.20 min, 100% purity

### Compound 34a: 1-Isobutoxy-2-nitro-benzene

Yield; 1.22 g, 6.25 mmol, 88%

¹H NMR indicates desired compound in ca. 95% purity

### Compound 34b: 2-Isobutoxy-phenylamine

Yield; 1.18 g, 7.14 mmol, 100%

LCMS; [M+H]⁺=166, Rt=1.52 min, <98% purity

¹H NMR indicates desired compound in ca. 95% purity

### Compound 34c: (5,6-Dimethyl-thieno[2,3-d]pyrimidin-4-yl)-(2-isobutoxy-phenyl)-amine

Yield; 95.3 mg, 0.291 mmol, 48%

LCMS; [M+H]⁺=328, Rt=2.25 min, 100% purity

### Compound 37c: (5[2-(1-Ethyl-pyrrolidin-3-yloxy)-phenyl]-(5-methyl-thieno[2,3-d]pyrimidin-4-yl)-amine

Yield; 2.7 mg, 0.008 mmol, 1.5%

LCMS; [M+H]⁺=328, Rt=2.25 min, 100% purity

### Compound 38c: (2-Isopropoxy-phenyl)-thieno[2,3-d]pyrimidin-4-yl-amine

Yield; 71 mg, 0.248 mmol, 75%

LCMS; [M+H]⁺=286, Rt=1.18 min, 94% purity

### Compound 39c: (2-sec-Butoxy-phenyl)-thieno[2,3-d]pyrimidin-4-yl-amine

Yield; 5.9 mg, 0.020 mmol, 3.2%

LCMS; [M+H]⁺=300, Rt=1.33 min, 100% purity

### Compound 40c: (2-Isobutoxy-phenyl)-(5-methyl-thieno[2,3-d]pyrimidin-4-yl)-amine

Yield; 132.2 mg, 0.422 mmol, 70%

LCMS; [M+H]⁺=314, Rt=2.23 min, 100% purity

### Compound 43a: 2-(2-Nitro-phenoxy)-adamantane

Yield; 1.7 g, 6.22 mmol, 88%

¹H NMR indicates desired compound in ca. 95% purity

### Compound 43b: 2-(Adamantan-2-yloxy)-phenylamine

Yield; 1.75 g, 7.2 mmol, 100%

LCMS; [M+H]⁺=244, Rt=1.86 min, 89% purity

### Compound 43c: [2-(Adamantan-2-yloxy)-phenyl]-(5-methyl-thieno[2,3-d]pyrimidin-4-yl)-amine

Yield; 22.6 mg, 0.058 mmol, 14%

LCMS; [M+H]⁺=392, Rt=2.42 min, 100% purity

### Compound 45c: [2-(Adamantan-2-yloxy)-phenyl]-(5,6-dimethyl-thieno[2,3-d]pyrimidin-4-yl)-amine

Yield; 27.8 mg, 0.069 mmol, 17%

LCMS; [M+H]⁺=406, Rt=2.44 min, 100% purity

### Compound 50a: 1-Isobutylsulfanyl-2-nitro-benzene

Yield; 1.63 g, 7.72 mmol, 100%

¹H NMR indicates desired compound in ca. 95% purity

### Compound 50b: 2-Isobutylsulfanyl-phenylamine

Yield; 1.23 g, 6.8 mmol, 90%

¹H NMR indicates desired compound in ca. 95% purity

### Compound 50c: (5,6-Dimethyl-thieno[2,3-d]pyrimidin-4-yl)-(2-isobutylsulfanyl-phenyl)-amine

Yield; 22.9 mg, 0.066 mmol, 12%

LCMS; [M+H]⁺=344, Rt=2.34 min, 90% purity

### Compound 55a: 1-(2-Nitro-phenoxy)-adamantane

Yield; 1.91 g, 6.99 mmol, 98%

¹H NMR indicates desired compound in ca. 90% purity

### Compound 55b: 2-(Adamantan-1-yloxy)-phenylamine

Yield; 1.47 g, 6.04 mmol, 87%

LCMS; [M+H]⁺=244, Rt=1.86 min, 98% purity

### Compound 55c: [2-(Adamantan-1-yloxy)-phenyl]-(5-methyl-thieno[2,3-d]pyrimidin-4-yl)-amine

Yield; 35.7 mg, 0.091 mmol, 22%

LCMS; [M+H]⁺=392, Rt=2.46 min, 95% purity

### Compound 58b: 4-Methoxy-pyridin-3-ylamine

Yield; 300 mg, 2.4 mmol, >100%

LCMS: [M+H]⁺=125, Rt=0.51 min, 100% purity

### Compound 58c: (4-Methoxy-pyridin-3-yl)-(5-methyl-thieno[2,3-d]pyrimidin-4-yl)-amine

Yield; 40 mg, 0.15 mmol, 27%

LCMS; [M+H]⁺=273, Rt=0.91 min, 94% purity

### Compound 65c: (2-Isobutylsulfanyl-phenyl)-(5-methyl-thieno[2,3-d]pyrimidin-4-yl)-amine

Yield; 9.8 mg, 0.03 mmol, 5%

LCMS; [M+H]⁺=330, Rt=2.30 min, 96% purity

### Compound 68a: [2-(Bicyclo[2.2.1]hept-2-yloxy)-phenyl]-thieno[2,3-d]pyrimidin-4-yl-amine

Yield; 138.7 mg, 0.41 mmol, 84%

LCMS; [M+H]⁺=338, Rt=1.50 min, 100% purity

### Compound 69a: [2-(Bicyclo[2.2.1]hept-2-yloxy)-phenyl]-(5-methyl-thieno[2,3-d]pyrimidin-4-yl)-amine

Yield; 137 mg, 0.39 mmol, 80%

LCMS; [M+H]⁺=352, Rt=1.88 min, 100% purity

### Compound 70a: [[2-(Bicyclo[2.2.1]hept-2-yloxy)-phenyl]-(5,6-dimethyl-thieno[2,3-d]pyrimidin-4-yl)-amine

Yield; 81.8 mg, 0.22 mmol, 46%

LCMS; [M+H]⁺=366, Rt=2.45 min, 95% purity

### Compound 71a: (2-Ethoxy-phenyl)-(6-isopropyl-thieno[2,3-d]pyrimidin-4-yl)-amine

Yield; 51 mg, 0.16 mmol, 69%

LCMS; [M+H]⁺=314, Rt=1.96 min, 98% purity

### Compound 72a: (6-Isopropyl-thieno[2,3-d]pyrimidin-4-yl)-(2-methoxy-phenyl)-amine

Yield; 66 mg, 0.22 mmol, 94%

LCMS; [M+H]⁺=300, Rt=1.88 min, 96% purity

### Compound 73a: (2-sec-Butoxy-phenyl)-(6-isopropyl-thieno[2,3-d]pyrimidin-4-yl)-amine

Yield; 43 mg, 0.13 mmol, 54%

LCMS; [M+H]⁺=342, Rt=2.12 min, 100% purity

### Compound 74a: (2-Cyclopentylsulfanyl-phenyl)-thieno[2,3-d]pyrimidin-4-yl-amine

Yield; 4.1 mg, 0.01 mmol, 2.4%

LCMS; [M+H]⁺=328, Rt=2.09 min, 92% purity

### Compound 75a: (2-Cyclohexylsulfanyl-phenyl)-thieno[2,3-d]pyrimidin-4-yl-amine

Yield; 9.4 mg, 0.03 mmol, 5.7%

LCMS; [M+H]⁺=342, Rt=1.71 min, 100% purity

### Compound 76a: [2-(Bicyclo[2.2.1]hept-2-yloxy)-phenyl]-thieno[2,3-d]pyrimidin-4-yl-amine

Yield; 70.4 mg, 0.21 mmol, 43%

LCMS; [M+H]⁺=338, Rt=2.02 min, 98% purity

### Compound 77a: [2-(Bicyclo[2.2.1]hept-2-yloxy)-phenyl]-(5-methyl-thieno[2,3-d]pyrimidin-4-yl)-amine

Yield; 64.4 mg, 0.181 mmol, 37%

LCMS; [M+H]⁺=352, Rt=2.36 min, 96% purity

### Compound 78a: [2-(Bicyclo[2.2.1]hept-2-yloxy)-phenyl]-(5,6-dimethyl-thieno[2,3-d]pyrimidin-4-yl)-amine

Yield; 123.4 mg, 0.34 mmol, 69%

LCMS; [M+H]⁺=366, Rt=2.38 min, 98% purity

### Compound 79a: [2-(Tetrahydro-furan-3-ylmethoxy)-phenyl]-thieno[2,3-d]pyrimidin-4-yl-amine

Yield; 36.6 mg, 0.11 mmol, 22%

LCMS; [M+H]⁺=327, Rt=1.64 min, 96% purity

### Compound 80a: (5-Methyl-thieno[2,3-d]pyrimidin-4-yl)-[2-(tetrahydro-furan-3-ylmethoxy)-phenyl]-amine

Yield; 124.5 mg, 0.36 mmol, 70%

LCMS; [M+H]⁺=342, Rt=1.92 min, 100% purity

### Compound 81a: (5,6-Dimethyl-thieno[2,3-d]pyrimidin-4-yl)-[2-(tetrahydro-furan-3-ylmethoxy)-phenyl]-amine

Yield; 78.0 mg, 0.22 mmol, 42%

LCMS; [M+H]⁺=356, Rt=1.96 min, 100% purity

### Compound 82a: (2-Isopropoxy-phenyl)-(6-isopropyl-thieno[2,3-d]pyrimidin-4-yl)-amine

Yield; 67.7 mg, 0.21 mmol, 88%

LCMS; [M+H]⁺=328, Rt=2.05 min, 100% purity

### Compound 83a: (2-Cyclopentyloxy-phenyl)-(6-isopropyl-thieno[2,3-d]pyrimidin-4-yl)-amine

Yield; 50.7 mg, 0.14 mmol, 61%

LCMS; [M+H]⁺=353, Rt=1.56 min, 100% purity

### Compound 84a: (6-Isopropyl-thieno[2,3-d]pyrimidin-4-yl)-[2-(tetrahydro-furan-3-yloxy)-phenyl]-amine

Yield; 7.3 mg, 0.02 mmol, 8.7%

LCMS; [M+H]⁺=356, Rt=1.85 min, 100% purity

### Compound 85a: [2-(1,2-Dimethyl-propoxy)-phenyl]-thieno[2,3-d]pyrimidin-4-yl-amine

Yield; 109.8 mg, 0.35 mmol, 63%

LCMS; [M+H]⁺=314, Rt=1.96 min, 100% purity

### Compound 86a: [2-(1,2-Dimethyl-propoxy)-phenyl]-(5-methyl-thieno[2,3-d]pyrimidin-4-yl)-amine

Yield; 63.0 mg, 0.17 mmol, 30%

LCMS; [M+H]⁺=328, Rt=2.29 min, 96% purity

### Compound 87a: [2-(1,2-Dimethyl-propoxy)-phenyl]-(5,6-dimethyl-thieno[2,3-d]pyrimidin-4-yl)-amine Yield; 26.0 mg, 0.08 mmol, 14%

LCMS; [M+H]⁺=342, Rt=2.31 min, 93% purity

### Compound 88a: [5-Fluoro-2-(tetrahydro-furan-3-yloxy)-phenyl]-thieno[2,3-d]pyrimidin-4-yl-amine

Yield; 94.1 mg, 0.28 mmol, 65%

LCMS; [M+H]⁺=332, Rt=1.28 min, 100% purity

### Compound 89a: [5-Fluoro-2-(tetrahydro-furan-3-yloxy)-phenyl]-(5-methyl-thieno[2,3-d]pyrimidin-4-yl)-amine

Yield; 89.4 mg, 0.26 mmol, 59%

LCMS; [M+H]⁺=346, Rt=1.53 min, 97% purity

### Compound 90a: (5,6-Dimethyl-thieno[2,3-d]pyrimidin-4-yl)-[5-fluoro-2-(tetrahydro-furan-3-yloxy)-phenyl]-amine

Yield; 107.7 mg, 0.30 mmol, 68%

LCMS; [M+H]⁺=360, Rt=1.58 min, 97% purity

### Compound 92a: (2-Ethoxy-5-fluoro-phenyl)-(5-methyl-thieno[2,3-d]pyrimidin-4-yl)-amine

Yield; 134.4 mg, 0.44 mmol, 68%

LCMS; [M+H]⁺=304, Rt=2.24 min, 100% purity

### Compound 93a: [2-(1-Ethyl-2-methyl-propoxy)-phenyl]-thieno[2,3-d]pyrimidin-4-yl-amine

Yield; 77.2 mg, 0.24 mmol, 46%

LCMS; [M+H]⁺=328, Rt=1.49 min, 100% purity

### Compound 94a: [2-(1-Ethyl-2-methyl-propoxy)-phenyl]-(5-methyl-thieno[2,3-d]pyrimidin-4-yl)-amine

Yield; 80.5 mg, 0.24 mmol, 46%

LCMS; [M+H]⁺=342, Rt=1.86 min, 96% purity

### Compound 95a: (5,6-Dimethyl-thieno[2,3-d]pyrimidin-4-yl)-[2-(1-ethyl-2-methyl-propoxy)-phenyl]-amine

Yield; 58.4 mg, 0.16 mmol, 32%

LCMS; [M+H]⁺=356, Rt=2.37 min, 100% purity

### Compound 96a: Thieno[2,3-d]pyrimidin-4-yl)-[2-(3,3,3-trifluoro-propoxy)-phenyl]-amine

Yield; 96.1 mg, 0.28 mmol, 58%

LCMS; [M+H]⁺=340, Rt=1.80 min, 100% purity

### Compound 97a: (5-Methyl-thieno[2,3-d]pyrimidin-4-yl)-[2-(3,3,3-trifluoro-propoxy)-phenyl]-amine

Yield; 100.2 mg, 0.28 mmol, 58%

LCMS; [M+H]⁺=354, Rt=2.06 min, 100% purity

### Compound 98a: (5,6-Dimethyl-thieno[2,3-d]pyrimidin-4-yl)-[2-(3,3,3-trifluoro-propoxy)-phenyl]-amine Yield; 101.0 mg, 0.27 mmol, 56%

LCMS; [M+H]⁺=354, Rt=2.06 min, 97% purity

### Compound 99a: [2-(3-Ethoxy-propoxy)-phenyl]-thieno[2,3-d]pyrimidin-4-yl-amine

Yield; 27.2 mg, 0.08 mmol, 12%

LCMS; [M+H]⁺=330, Rt=1.15 min, 96% purity

### Compound 100a: (5,6-Dimethyl-thieno[2,3-d]pyrimidin-4-yl)-[2-(3-ethoxy-propoxy)-phenyl]-amine

Yield; 31.2 mg, 0.09 mmol, 14%

LCMS; [M+H]⁺=358, Rt=2.10 min, 100% purity

### Compound 101a: [2-(3-Ethoxy-propoxy)-phenyl]-(5-methyl-thieno[2,3-d]pyrimidin-4-yl)-amine

Yield; 17.5 mg, 0.05 mmol, 8%

LCMS; [M+H]⁺=344, Rt=2.07 min, 98% purity

### Compound 102a: [2-(2-Ethoxy-ethoxy)-phenyl]-thieno[2,3-d]pyrimidin-4-yl-amine

Yield; 34.8 mg, 0.11 mmol, 19%

LCMS; [M+H]⁺=316, Rt=1.67 min, 100% purity

### Compound 103a: [2-(2-Ethoxy-ethoxy)-phenyl]-(5-methyl-thieno[2,3-d]pyrimidin-4-yl)-amine

Yield; 27.8 mg, 0.08 mmol, 14%

LCMS; [M+H]⁺=330, Rt=2.00 min, 100% purity

### Compound 104a: (5,6-Dimethyl-thieno[2,3-d]pyrimidin-4-yl)-[2-(2-ethoxy-ethoxy)-phenyl]-amine

Yield; 19.7 mg, 0.06 mmol, 11%

LCMS; [M+H]⁺=344, Rt=2.03 min, 100% purity

### Compound 156a: (2-Ethoxy-5-fluoro-phenyl)-thieno[2,3-d]pyrimidin-4-yl-amine

Yield; 158.3 mg, 0.55 mmol, 85%

LCMS; [M+H]⁺=290, Rt=1.92 min, 100% purity

### Example 1b

### Synthesis Route 2

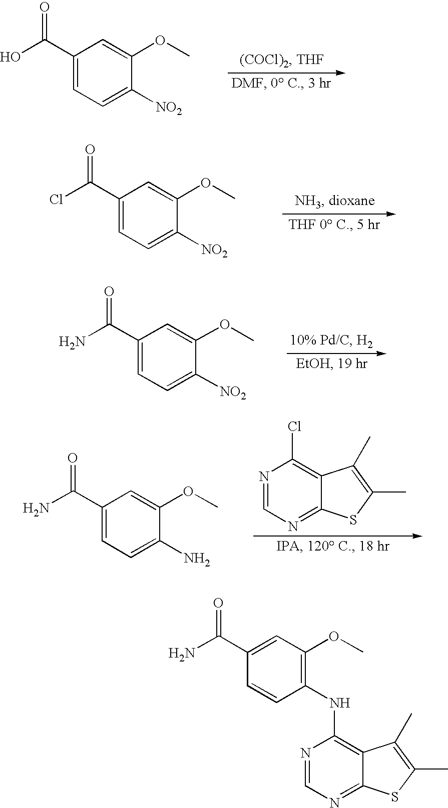

### Compound 14a. 3-Methoxy-4-nitro-benzoyl chloride

To a stirred solution of 3-Methoxy-4-nitro-benzoic acid (1.0 g, 5.1 mmol) in tetrahydrofuran (14 ml) at 0° C. was added a 2 M solution of oxalyl chloride in dichloromethane (2.8 ml, 5.6 mmol) followed by 5 drops of dimethylformamide. The reaction was stirred under a nitrogen atmosphere for 3 hours allowing the temperature to slowly rise to room temperature. The reaction the solvent was removed in vacuo give the title compound as a yellow solid (1.2 g, 5.6 mmol, >100%). LCMS in methanol, trapping Me-ester: [M+H]⁺=212, Rt=1.30 min, 71% purity.

### Compound 14b. 3-Methoxy-4-nitro-benzamide

To a solution of 0.5 M ammonia in dioxane (110 ml, 55 mmol) at 0° C. was added 3-methoxy-4-nitro-benzoyl chloride (1.1 g, 5.1 mmol) in tetrahydrofuran (10 ml). The reaction was stirred at room temperature under a nitrogen atmosphere for 5 hours and then diluted with ethyl acetate (100 ml). The solution was washed with water (2×200 ml), dried (MgSO₄), filtered and the solvent removed in vacuo to give the title compound as a pale yellow solid (813 mg, 4.14 mmol, 81%). LCMS: [M+H]⁺=197, Rt=0.92 min, 100% purity.

### Compound 14c. 4-Amino-3-methoxy-benzamide

3-Methoxy-4-nitro-benzamide (500 mg, 2.55 mmol), 10% palladium on carbon (100 mg), and ethanol (50 ml) were stirred at room temperature under a hydrogen atmosphere for 19 hours. The reaction was then filtered through celite and the solvent removed in vacuo to give the title compound as a beige solid. (450 mg, 2.7 mmol, 100% corrected). LCMS: [M+H]⁺=167, Rt=0.54 min, 70% purity.

### Compound 14d. 4-(5,6-Dimethyl-thieno[2,3-d]pyrimidin-4-ylamino)-3-methoxy-benzamide

4-Chloro-5,6-dimethyl-thieno[2,3-d]pyrimidine (100 mg, 0.50 mmol) and 4-amino-3-methoxy-benzamide (84 mg, 0.50 mmol) were heated at 120° C. in isopropanol (3 ml) for 18 hours in an Ace pressure tube. The reaction was cooled to room temperature, diluted with water (3 ml) and basified to pH 10 with ammonium hydroxide solution. The resulting precipitate was filtered, washed with water (20 ml), and dried in vacuo. The title compound was obtained as a cream solid (132 mg, 0.40 mmol, 80%). LCMS: [M+H]⁺=329, Rt=1.74 min, 82% purity. The compounds listed below were prepared using route 2;

### Compound 15d: 4-(5-Methyl-thieno[2,3-d]pyrimidin-4-ylamino)-3-methoxy-benzamide

Yield; 59 mg, 0.19 mmol, 35%

LCMS; [M+H]⁺=315.24, Rt=1.69 min, 100% purity

### Example 1c

### Synthesis Route 3

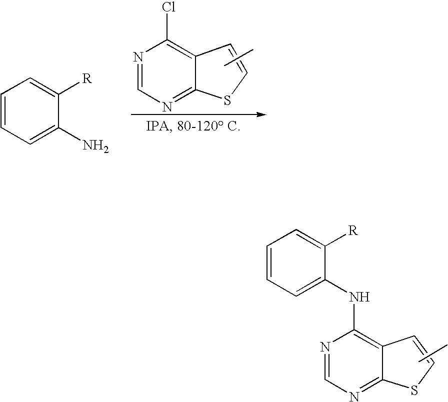

### Compound 29a. (5,6-Dimethyl-thieno[2,3-d]pyrimidin-4-yl)-(2-methylsulfanyl-phenyl)-amine

2-Methylsulfanyl-phenylamine (100 mg, 0.72 mmol, 1 eq) was placed in an Ace pressure and 4-chloro-5,6-dimethyl-thieno[2,3-d]pyrimidine (143 mg, 0.72 mmol, 1 eq) added. 2-propanol (4 ml) was added and the reaction mixture was stirred at 120° C. for 18 hours. The reaction mixture was allowed to cool to room temperature. Ammonium hydroxide (1 ml) was added followed by water (5-6 ml). The product precipitated and was filtered off, washed with 1 ml of water and dried to yield the title compound as a yellow solid (157.2 mg, 0.521 mmol, 72%). LCMS; [M+H]⁺=302, Rt=1.99 min, 100% purity

The compounds listed below were prepared using route 3;

### Compound 13a: (5,6-Dimethyl-thieno[2,3-d]pyrimidin-4-yl)-(2-methoxy-phenyl)-amine

Yield; 1.01 g, 3.54 mmol, 33%

LCMS; [M+H]⁺=286, Rt=1.80 min, 100% purity

### Compound 17a: (2-Ethoxy-phenyl)-(5-methyl-thieno[2,3-d]pyrimidin-4-yl)-amine

Yield; 20.3 mg, 0.071 mmol, 17.%

LCMS; [M+H]⁺=286, Rt=1.48 min, 100% purity

### Compound 21a: (5,6-Dimethyl-thieno[2,3-d]pyrimidin-4-yl)-(2-ethoxy-phenyl)-amine

Yield; 56.8 mg, 0.190 mmol, 38.%

LCMS; [M+H]⁺=300, Rt=1.58 min, 100% purity

### Compound 24a: 3-(5-Methyl-thieno[2,3-d]pyrimidin-4-ylamino)-pyridin-2-o1

Yield; 15 mg, 0.06 mmol, 10%

LCMS; [M+H]⁺=259, Rt=0.97 min, 95% purity

### Compound 30a: (2-Methoxy-pyridin-3-yl)-(5-methyl-thieno[2,3-d]pyrimidin-4-yl)-amine

Yield; 16.5 mg, 0.06 mmol, 11%

LCMS; [M+H]⁺=273, Rt=1.39 min, 100% purity

### Compound 31a: (2-Methylsulfanyl-phenyl)-(5-methyl-thieno[2,3-d]pyrimidin-4-yl)-amine

Yield; 154.6 mg, 0.538 mmol, 75%

LCMS; [M+H]⁺=288, Rt=1.95 min, 100% purity

### Compound 35a: (3-Chloro-2-methoxy-phenyl)-(5-methyl-thieno[2,3-d]pyrimidin-4-yl)-amine

Yield; 63 mg, 0.21 mmol, 38%

LCMS; [M+H]⁺=306, Rt=1.57 min, 100% purity

### Compound 36a: (2-Difluoromethoxy-phenyl)-(5-methyl-thieno[2,3-d]pyrimidin-4-yl)-amine

Yield; 42.4 mg, 0.140 mmol, 44%

LCMS; [M+H]+=308, Rt=1.42 min@95% purity

### Compound 41a: (2-Difluoromethoxy-phenyl)-(5,6-dimethyl-thieno[2,3-d]pyrimidin-4-yl)-amine

Yield; 26.0 mg, 0.081 mmol, 26%

LCMS; [M+H]+=322, Rt=1.50 min@100% purity

### Compound 42a: (5,6-Dimethyl-thieno[2,3-d]pyrimidin-4-yl)-[2-(1,1,2,2-tetrafluoro-ethoxy)phenyl]-amine

Yield; 15 mg, 0.042 mmol, 14%

LCMS; [M+H]⁺=372, Rt=1.49 min, 100% purity

### Compound 44a: (5-Methyl-thieno[2,3-d]pyrimidin-4-yl)-[2-(1,1,2,2-tetrafluoro-ethoxy)-phenyl]-amine

Yield; 16 mg, 0.044 mmol, 15%

LCMS; [M+H]⁺=358, Rt=1.45 min, 93% purity

### Compound 46a: (5-Methyl-thieno[2,3-d]pyrimidin-4-yl)-(2-phenoxy-phenyl)-amine

Yield; 2.9 mg, 0.009 mmol, 1.6%

LCMS; [M+H]⁺=334, Rt=1.71 min, 98% purity

### Compound 47a: (5-Methyl-thieno[2,3-d]pyrimidin-4-yl)-(2-trifluoromethoxy-phenyl)-amine

Yield; 4.5 mg, 0.014 mmol, 5%

LCMS; [M+H]⁺=326, Rt=1.54min@100% purity

### Compound 48a: (5,6-Dimethyl-thieno[2,3-d]pyrimidin-4-yl)-(2-ethyl-phenyl)-amine

Yield; 21 mg, 0.074 mmol, 9%

LCMS; [M+H]⁺=284, Rt=1.82 min, 97% purity

### Compound 49a: (2-Methoxy-phenyl)-(5-methyl-thieno[2,3-d]pyrimidin-4-yl)-amine

Yield; 8 mg, 0.029 mmol, 7%

LCMS; [M+H]⁺=272, Rt=1.30 min, 100% purity

### Compound 51a: (5,6-Dimethyl-thieno[2,3-d]pyrimidin-4-yl)-(2-morpholin-4-yl-phenyl)-amine

Yield; 130 mg, 0.382 mmol, 68%

LCMS; [M+H]⁺=341, Rt=1.96 min, 100% purity

### Compound 52a: (5,6-Dimethyl-thieno[2,3-d]pyrimidin-4-yl)-(2-propyl-phenyl)-amine

Yield; 31.8 mg, 0.107 mmol, 14%

LCMS; [M+H]⁺=298, Rt=1.92 min, 98% purity

### Compound 53a: (5,6-Dimethyl-thieno[2,3-d]pyrimidin-4-yl)-(2-isopropyl-phenyl)-amine

Yield; 28.3 mg, 0.095 mmol, 13%

LCMS; [M+H]⁺=298, Rt=1.91 min, 100% purity

### Compound 54a: (2-Ethyl-phenyl)-(5-methyl-thieno[2,3-d]pyrimidin-4-yl)-amine

Yield; 64.2 mg, 0.238 mmol, 29%

LCMS; [M+H]⁺=270, Rt=1.77 min, 100% purity

### Compound 56a: (2-sec-Butyl-phenyl)-(5,6-dimethyl-thieno[2,3-d]pyrimidin-4-yl)-amine

Yield; 47.2 mg, 0.152 mmol, 23%

LCMS; [M+H]⁺=312, Rt=1.98 min, 97% purity

### Compound 57a: (5,6-Dimethyl-thieno[2,3-d]pyrimidin-4-yl)-(2-isopropyl-phenyl)-amine

Yield; 48 mg, 0.169 mmol, 23%

LCMS; [M+H]⁺=284, Rt=1.86 min, 100% purity

### Compound 59a: (2-sec-Butyl-phenyl)-(5-methyl-thieno[2,3-d]pyrimidin-4-yl)-amine

Yield; 38.7 mg, 0.130 mmol, 19%

LCMS; [M+H]⁺=298, Rt=1.93 min, 100% purity

### Compound 60a: (2-sec-Butyl-phenyl)-(5-methyl-thieno[2,3-d]pyrimidin-4-yl)-amine

Yield; 41.1 mg, 0.145 mmol, 20%

LCMS; [M+H]⁺=284, Rt=1.88 min, 97% purity

### Compound 61a: (5,6-Dimethyl-thieno[2,3-d]pyrimidin-4-yl)-(2-phenoxy-phenyl)-amine

Yield; 155.2 mg, 0.447 mmol, 83%

LCMS; [M+H]⁺=348, Rt=2.22 min, 100% purity

### Compound 63a: (2-Bromo-phenyl)-(5-methyl-thieno[2,3-d]pyrimidin-4-yl)-amine

Yield: 7 mg, 0.21 mmol, 0.4%

LCMS: [M+H]⁺=320, Rt=1.55 min, 100% purity.

### Compound 66a: (5-Methyl-thieno[2,3-d]pyrimidin-4-yl)-(2-piperidin-1-yl-phenyl)-amine

Yield; 10.9 mg, 0.033 mmol, 17%

LCMS; [M+H]⁺=311 Rt=1.12 min@100% purity

### Compound 67a: 2-(5-Methyl-thieno[2,3-d]pyrimidin-4-ylamino)-phenol

Yield; 45 mg, 0.175 mmol, 40%

LCMS; [M+H]⁺=258, Rt=1.18 min, 100% purity

### Compound 105a: (2,6-Dimethoxy-phenyl)-thieno[2,3-d]pyrimidin-4-yl-amine

Yield; 55 mg, 0.19 mmol, 39%

LCMS; [M+H]⁺=288, Rt=1.37 min, 100% purity

### Compound 106a: (2,6-Dimethoxy-phenyl)-(5-methyl-thieno[2,3-d]pyrimidin-4-yl)-amine

Yield; 68 mg, 0.23 mmol, 46%

LCMS; [M+H]⁺=302, Rt=1.81 min, 100% purity

### Compound 107a: (2,6-Dimethoxy-phenyl)-(5,6-dimethyl-thieno[2,3-d]pyrimidin-4-yl)-amine

Yield; 62 mg, 0.20 mmol, 40%

LCMS; [M+H]⁺=316, Rt=1.88 min, 97% purity

### Example 1d

### Synthesis Route 4

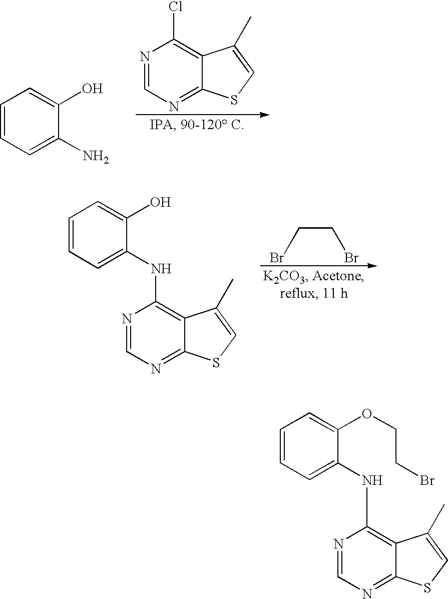

### Compound 62a. 2-(5-Methyl-thieno[2,3-d]pyrimidin-4-ylamino)-phenol

2-Hydroxyaniline (200 mg, 1.83 mmol, 1 eq) was placed in an Ace pressure tube to which was added 4-Chloro-5-methyl-thieno[2,3-d]pyrimidine (338 mg, 1.83 mmol, 1 eq). 2-Propanol (4 ml) was added and the reaction mixture was stirred at 105° C. for 2 hours. The reaction mixture was allowed to cool down to room temperature. The title compound precipitated as the hydrochloride salt and was filtered off. It was then taken up in sodium hydroxide 5N (4 ml) and precipitated in aqueous as the free base. It was filtered off and dried to yield the title compound (230 mg, 0.894 mmol, 49%). LCMS; [M+H]⁺=258, Rt=1.03 min, 83% purity

### Compound 62b. [2-(2-Bromo-ethoxy)-phenyl]-(5-methyl-thieno[2,3-d]pyrimidin-4-yl)-amine

2-(5-Methyl-thieno[2,3-d]pyrimidin-4-ylamino)-phenol, (50 mg, 0.194 mmol, 1 eq) was stirred in solution in acetone (3 ml) and potassium carbonate (54 mg, 0.39 mmol, 2 eq). Dibromoethane (92 mg, 0.49 mmol, 2.5 eq) was added to the mixture and the reaction was heated at reflux for 12 h, after which there was no further evolution. The mixture was allowed to cool to room temperature and water (10 ml) was added. The mixture was extracted twice with ethyl acetate (10 ml), the organics combined, dried over sodium sulphate, filtered and the solvent removed in vacuo. The mixture was purified by column chromatography on silica using dichloromethane as eluent to yield the title compound (6.7 mg, 0.018 mmol, 9%). LCMS; [M+H]⁺=366, Rt=1.52 min, 90% purity.

### Example 1e

### Synthesis Route 5

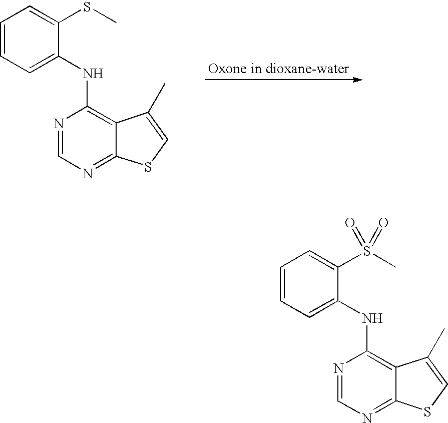

### Compound 64: (2-Methanesulfonyl-phenyl)-(5-methyl-thieno[2,3-d]pyrimidin-4-yl)-amine

(2-Methylsulfanyl-phenyl)-(5-methyl-thieno[2,3-d]pyrimidin-4-yl)-amine (20 mg, 1 eq., 0.069 mmol) and oxone (172 mg, 4 eq., 0.278 mol) were stirred in dioxane-water (4:1, 1 ml) for 1 hours at room temperature. Then to the reaction a saturated aqueous solution of NaHCO₃ (2 ml) was added. The mixture was extracted with ethyl acetate (2×4 ml), the organics combined, dried over sodium sulphate and solvent removed in vacuo to give the title compound (20 mg, 0.062 mmol, 89%). LCMS: [M+H]⁺=320, Rt=1.88 min, 94% purity. The compounds listed below were prepared using route 5;

### Compound 91a: (5,6-Dimethyl-thieno[2,3-d]pyrimidin-4-yl)-(2-methanesulfonyl-phenyl)-amine

Yield; 10 mg, 0.03 mmol, 50%

LCMS; [M+H]⁺=334, Rt=1.40 min, 98% purity

### Example 1f

### Synthesis Route 6

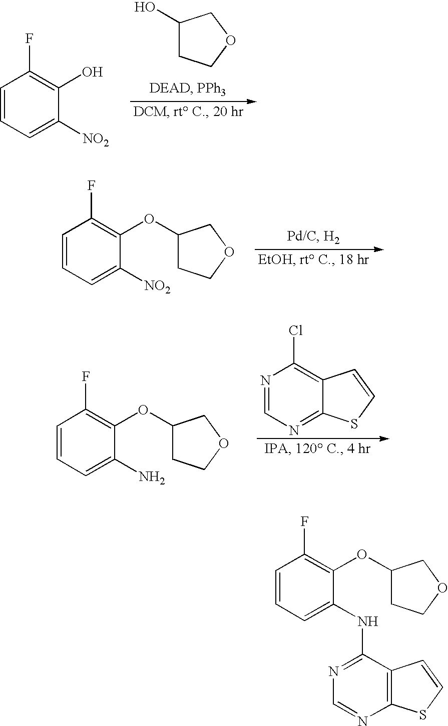

### Compound 108: 3-(2-Fluoro-6-nitro-phenoxy)-tetrahydro-furan

2-Fluoro-6-nitro-phenol (1.0 g, 6.37 mmol, 1.0 eq) was dissolved in DCM (10 ml) and 3-hydroxy-tetrahydrofuran (0.56 g, 6.37 mmol, 1.0 eq), triphenylphosphine (2.0 g, 7.64 mmol, 1.2 eq), and diazodiethyldicarboxylate (1.22 g, 7.01 mmol, 1.2 eq) were added sequentially. The reaction was stirred at room temperature for 20 hours. The reaction mixture was filtered and the solvent removed in vacuo from the filtrate. The resultant residue was purified by column chromatography using 1% DCM/MeOH as eluent to give the title compound (0.99 g, 4.35 mmol, 68%). ¹H NMR indicates desired compound in ca. 95% purity.

### Compound 108b: 3-Fluoro-2-(tetrahydro-furan-3-yloxy)-phenylamine

3-(2-Fluoro-6-nitro-phenoxy)-tetrahydro-furan (0.99 mg, 4.35 mmol), 10% palladium on carbon (0.1 g, 10% w/w), and ethanol (15 ml) were stirred at room temperature under a hydrogen atmosphere for 18 hours. The reaction was filtered through celite and the solvent removed in vacuo to give the title compound as yellow oil (0.81 g, 4.11 mmol, 94%). LCMS: [M+H]⁺=198, Rt=0.90 min, 100% purity.

### Compound 108c: [3-Fluoro-2-(tetrahydro-furan-3-yloxy)-phenyl]-thieno[2,3-d]pyrimidin-4-yl-amine

3-Fluoro-2-(tetrahydro-furan-3-yloxy)-phenylamine (75 mg, 0.38 mmol, 1.0 eq) and 4-chloro-thieno[2,3-d]pyrimidine (65 mg, 0.38 mmol, 1.0 eq) were added to an ACE pressure tube 2-Propanol (2.5 ml) added and the reaction mixture stirred at 120° C. for 18 hours. The reaction mixture was allowed to cool to room temperature then ammonium hydroxide solution (1 ml) and water (4 ml) were added sequentially. The resultant precipitate was isolated by filtration, washed with cyclohexane (2×2 ml) and diethyl ether (2×2 ml) and dried in vacuo. This gave the title compound as an off-white solid (48 mg, 0.15 mmol, 38%). LCMS; [M+H]⁺=332, Rt=1.78 min, 100% purity

The compounds listed below were prepared using route 6;

### Compound 109a: [3-Fluoro-2-(tetrahydro-furan-3-yloxy)-phenyl]-(5-methyl-thieno[2,3-d]pyrimidin-4-yl)-amine

Yield; 40 mg, 0.12 mmol, 30%

LCMS; [M+H]⁺=346, Rt=2.01 min, 100% purity

### Compound 110a: [3-Fluoro-2-(tetrahydro-furan-3-yloxy)-phenyl]-(5,6-dimethyl-thieno[2,3-d]pyrimidin-4-yl)-amine

Yield; 13 mg, 0.04 mmol, 10%

LCMS; [M+H]⁺=360, Rt=2.08 min, 100% purity

### Compound 111a: (4-Fluoro-2-methoxy-phenyl)-thieno[2,3-d]pyrimidin-4-yl-amine

Yield; 65.9 mg, 0.24 mmol, 48%

LCMS; [M+H]⁺=276, Rt=1.93 min, 100% purity

### Compound 112a: (2-Ethoxy-4-fluoro-phenyl)-thieno[2,3-d]pyrimidin-4-yl-amine

Yield; 31.7 mg, 0.11 mmol, 24%

LCMS; [M+H]⁺=290, Rt=2.09 min, 100% purity

### Compound 113a: [4-Fluoro-2-(tetrahydro-furan-3-yloxy)-phenyl]-thieno[2,3-d]pyrimidin-4-yl-amine

Yield; 31.4 mg, 0.09 mmol, 25%

LCMS; [M+H]⁺=332, Rt=1.89 min, 100% purity

### Compound 114a: (4-Fluoro-2-methoxy-phenyl)-(5-methyl-thieno[2,3-d]pyrimidin-4-yl)-amine

Yield; 61.3 mg, 0.21 mmol, 43%

LCMS; [M+H]⁺=290, Rt=2.36 min, 100% purity

### Compound 115a: (2-Ethoxy-4-fluoro-phenyl)-(5-methyl-thieno[2,3-d]pyrimidin-4-yl)-amine

Yield; 47.5 mg, 0.16 mmol, 36%

LCMS; [M+H]⁺=304, Rt=2.53 min, 100% purity

### Compound 116a: (2-Cyclopentyloxy-4-fluoro-phenyl)-(5-methyl-thieno[2,3-d]pyrimidin-4-yl)-amine

Yield; 72.8 mg, 0.21 mmol, 59%

LCMS; [M+H]⁺=344, Rt=2.76 min, 100% purity

### Compound 117a: [4-Fluoro-2-(tetrahydro-furan-3-yloxy)-phenyl]-(5-methyl-thieno[2,3-d]pyrimidin-4-yl)-amine

Yield; 84.3 mg, 0.24 mmol, 64%

LCMS; [M+H]⁺=346, Rt=2.31 min, 100% purity

### Compound 118a: (5,6-Dimethyl-thieno[2,3-d]pyrimidin-4-yl)-(4-fluoro-2-methoxy-phenyl)-amine

Yield; 90.6 mg, 0.30 mmol, 60%

LCMS; [M+H]⁺=304, Rt=2.47 min, 100% purity

### Compound 119a: (5,6-Dimethyl-thieno[2,3-d]pyrimidin-4-yl)-(2-ethoxy-4-fluoro-phenyl)-amine

Yield; 80.6 mg, 0.25 mmol, 56%

LCMS; [M+H]⁺=318, Rt=2.64 min, 100% purity

### Compound 120a: (5,6-Dimethyl-thieno[2,3-d]pyrimidin-4-yl)-(4-fluoro-2-isopropoxy-phenyl)-amine

Yield; 98.5 mg, 0.30 mmol, 72%

LCMS; [M+H]⁺=332, Rt=2.72 min, 100% purity

### Compound 121a: (2-Cyclopentyloxy-4-fluoro-phenyl)-(5,6-dimethyl-thieno[2,3-d]pyrimidin-4-yl)-amine

Yield; 76.9 mg, 0.22 mmol, 60%

LCMS; [M+H]⁺=358, Rt=2.87 min, 100% purity

### Compound 122a: (5,6-Dimethyl-thieno[2,3-d]pyrimidin-4-yl)-[4-fluoro-2-(tetrahydro-furan-3-yloxy)-phenyl]-amine

Yield; 86.3 mg, 0.24 mmol, 63%

LCMS; [M+H]⁺=360, Rt=2.42 min, 100% purity

### Compound 123a: (4-Fluoro-2-isopropoxy-phenyl)-thieno[2,3-d]pyrimidin-4-yl-amine

Yield; 86.6 mg, 0.29 mmol, 69%

LCMS; [M+H]⁺=304, Rt=1.64 min, 100% purity

### Compound 124a: (2-sec-Butoxy-4-fluoro-phenyl)-thieno[2,3-d]pyrimidin-4-yl-amine

Yield; 48.8 mg, 0.15 mmol, 40%

LCMS; [M+H]⁺=318, Rt=1.75 min, 90% purity

### Compound 125a: (4-Fluoro-2-isopropoxy-phenyl)-(5-methyl-thieno[2,3-d]pyrimidin-4-yl)-amine

Yield; 67.2 mg, 0.21 mmol, 51%

LCMS; [M+H]⁺=318, Rt=1.64 min, 90% purity

### Compound 126a: (2-sec-Butoxy-4-fluoro-phenyl)-(5-methyl-thieno[2,3-d]pyrimidin-4-yl)-amine

Yield; 52.4 mg, 0.16 mmol, 41%

LCMS; [M+H]⁺=332, Rt=2.70 min, 90% purity

### Compound 127a: (2-sec-Butoxy-4-fluoro-phenyl)-(5,6-dimethyl-thieno[2,3-d]pyrimidin-4-yl)-amine

Yield; 50.6 mg, 0.15 mmol, 38%

LCMS; [M+H]⁺=346, Rt=2.81 min, 92% purity

### Compound 128a: [4-Fluoro-2-(3,3,3-trifluoro-propoxy)-phenyl]-thieno[2,3-d]pyrimidin-4-yl-amine

Yield; 101.8 mg, 0.28 mmol, 80%

LCMS; [M+H]⁺=358, Rt=2.22 min, 100% purity

### Compound 129a: [4-Fluoro-2-(3,3,3-trifluoro-propoxy)-phenyl]-(5-methyl-thieno[2,3-d]pyrimidin-4-yl)-amine

Yield; 96.5 mg, 0.27 mmol, 73%

LCMS; [M+H]⁺=372, Rt=2.50 min, 100% purity

### Compound 130a: (5,6-Dimethyl-thieno[2,3-d]pyrimidin-4-yl)-[4-fluoro-2-(3,3,3-trifluoro-propoxy)-phenyl]-amine

Yield; 110.9 mg, 0.29 mmol, 80%

LCMS; [M+H]⁺=386, Rt=2.59 min, 100% purity

### Compound 178a: (2-Cyclopentyloxy-4-fluoro-phenyl)-thieno[2,3-d]pyrimidin-4-yl-amine

Yield; 67.5 mg, 0.20 mmol, 57%

LCMS; [M+H]⁺=330, Rt=1.81 min, 94% purity

### Example 1g

### Synthesis Route 7

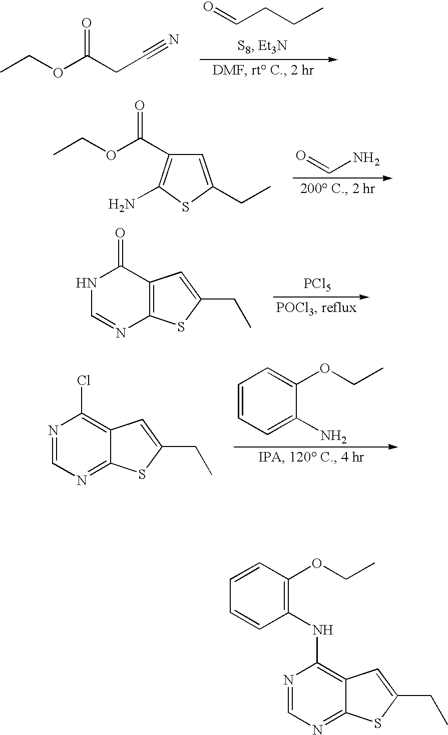

### Compound 131a. 2-Amino-5-ethyl-thiophene-3-carboxylic acid ethyl ester

Ethyl cyanoacetate (5.0 g, 44.0 mmol, 1.0 eq), sulphur (1.42 g, 44.0 mmol, 1.0 eq), and triethylamine (2.24 g, 22.0 mmol, 0.5 eq) were dissolved in DMF (20 ml) and the reaction stirred at room temperature for 10 minutes. Butyraldehyde (3.19 g, 44.0 mmol, 1.0 eq) was added drop-wise to the reaction mixture, keeping the temperature under 50° C. The reaction was then stirred at room temperature for 2 hours then poured into water (80 ml). The resultant solid was isolated by filtration, washed with water (400 ml), dried on the sinter, washed with cyclohexane (200 ml) and dried in vacuo to give the title compound as an orange solid (4.29 g, 21.53, 49%). ¹H NMR shows product in >95% purity

### Compound 131b. 6-Ethyl-3H-thieno[2,3-d]pyrimidin-4-one

A solution of 2-amino-5-ethyl-thiophene-3-carboxylic acid ethyl ester (2.0 g, 10.06 mmol, 1.0 eq) in formamide (4 ml) was heated at 200° C. for 2 hours. The reaction was allowed to cool to room temperature and the resultant precipitate was isolated by filtration, washed with cyclohexane, dried on the sinter, washed with water, then dried in vacuo to give the title compound as an off-white solid (1.37 g, 7.7 mmol, 76%). ¹H NMR shows product in >95% purity.

### Compound 131c. 4-Chloro-6-ethyl-thieno[2,3-d]pyrimidine

6-Ethyl-3H-thieno[2,3-d]pyrimidin-4-one (1.0 g, 5.59 mmol, 1.0 eq) was added to a solution of phosphorous pentachloride (1.16 g, 5.59 mmol, 1.0 eq) in phosphorous oxychloride (4 ml) and the reaction heated at 130° C. for 1 hour. The reaction mixture was allowed to cool to room temperature and the solvent removed in vacuo. This gave the title compound (1.11 g, 5.59 mmol, 100%). ¹H NMR shows product in >95% purity.

### Compound 131d. (2-Ethoxy-phenyl)-(6-ethyl-thieno[2,3-d]pyrimidin-4-yl)-amine

o-Phenetidine (47 mg, 0.343 mmol, 1.0 eq) and 4-Chloro-6-ethyl-thieno[2,3-d]pyrimidine (68 mg, 0.343 mmol, 1.0 eq) were charged to an ACE pressure tube and dissolved in IPA (3 ml). The reaction the heated at 120° C. for 2.5 hours and allowed to cool to room temperature. Then ammonium hydroxide solution (1 ml) and water (4 ml) were added sequentially to the reaction mixture, this was extracted with ethyl acetate (2×5 ml), the organics combined, and the solvent removed in vacuo. The resultant solid was purified by column chromatography using 0.5% MeOH/DCM as eluent to give the title compound as an off-white solid (62 mg, 0.20 mmol, 60%). LCMS; [M+H]⁺=300, Rt=1.88 min, 100% purity

The compounds listed below were prepared via route 7, utilising anilines prepared as per routes 1 & 6;

### Compound 132a: (2-sec-Butoxy-phenyl)-(6-ethyl-thieno[2,3-d]pyrimidin-4-yl)-amine

Yield; 98.0 mg, 0.26 mmol, 76%

LCMS; [M+H]⁺=328, Rt=2.03 min, 100% purity

### Compound 133a: (6-Ethyl-thieno[2,3-d]pyrimidin-4-yl)-[2-(tetrahydro-furan-3-yloxy)-phenyl]-amine

Yield; 41 mg, 0.12 mmol, 35%

LCMS; [M+H]⁺=342, Rt=1.75 min, 100% purity

### Compound 134a: (6-Ethyl-thieno[2,3-d]pyrimidin-4-yl)-(2-methoxy-phenyl)-amine

Yield; 61 mg, 0.21 mmol, 62%

LCMS; [M+H]⁺=286, Rt=1.79 min, 97% purity

### Compound 135a: (6-Ethyl-thieno[2,3-d]pyrimidin-4-yl)-(2-isopropoxy-phenyl)-amine

Yield; 56.4 mg, 0.18 mmol, 36%

LCMS; [M+H]⁺=314, Rt=1.38 min, 100% purity

### Compound 136a: (2-Cyclopentyloxy-phenyl)-(6-ethyl-thieno[2,3-d]pyrimidin-4-yl)-amine

Yield; 95.5 mg, 0.28 mmol, 56%

LCMS; [M+H]⁺=340, Rt=1.48 min, 96% purity

### Compound 137a: (2-Methoxy-phenyl)-(6-methyl-5-propyl-thieno[2,3-d]pyrimidin-4-yl)-amine

Yield; 33 mg, 0.11 mmol, 30%

LCMS; [M+H]⁺=314, Rt=1.64 min, 100% purity

### Compound 138a: (2-Ethoxy-phenyl)-(6-methyl-5-propyl-thieno[2,3-d]pyrimidin-4-yl)-amine

Yield; 48.1 mg, 0.15 mmol, 42%

LCMS; [M+H]⁺=328, Rt=1.69 min, 100% purity

### Compound 139a: (2-Isopropoxy-phenyl)-(6-methyl-5-propyl-thieno[2,3-d]pyrimidin-4-yl)-amine

Yield; 20.7 mg, 0.06 mmol, 17%

LCMS; [M+H]⁺=342, Rt=1.72 min, 94% purity

### Compound 140a: (2-sec-Butoxy-phenyl)-(6-methyl-5-propyl-thieno[2,3-d]pyrimidin-4-yl)-amine

Yield; 27.6 mg, 0.08 mmol, 22%

LCMS; [M+H]⁺=356, Rt=1.82 min, 100% purity

### Compound 141a: (6-Methyl-5-propyl-thieno[2,3-d]pyrimidin-4-yl)-[2-(tetrahydro-furan-3-yloxy)-phenyl]-amine

Yield; 22.3 mg, 0.06 mmol, 17%

LCMS; [M+H]⁺=370, Rt=1.51 min, 97% purity

### Compound 149a: (2-Cyclopentyloxy-phenyl)-(6-methyl-5-propyl-thieno[2,3-d]pyrimidin-4-yl-amine

Yield; 39.2 mg, 0.11 mmol, 30%

LCMS; [M+H]⁺=368, Rt=2.36 min, 96% purity

### Example 1h

### Synthesis Route 8

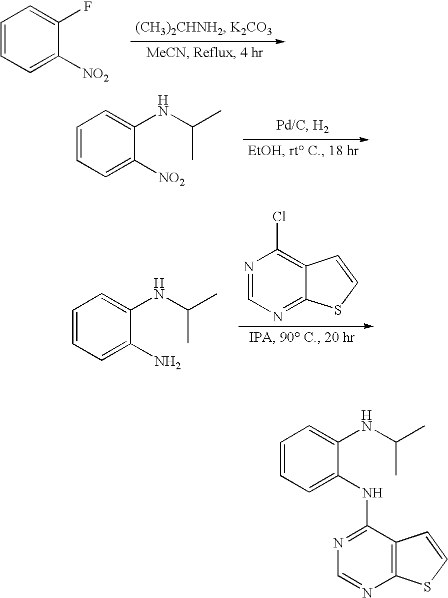

### Compound 142a. Isopropyl-(2-nitro-phenyl)-amine

2-Fluoro-nitrobenzene (0.75 ml, 7.08 mmol, 1.0 eq), isopropylamine (4.19 g, 70.8 mmol, 10 eq), and potassium carbonate (0.68 g, 4.9 mmol, 0.7 eq) were suspended in acetonitrile (8 ml). The reaction was heated at reflux for 4 hours, allowed to cool, the solids removed by filtration, and the solvent removed in vacuo. The resultant residue was partioned between water and ethyl actetate, the organic layer removed, dried over sodium sulphate, and the solvent removed in vacuo. The resultant residue was purified by column chromatography using 20% EtOAc/cyclohexane as eluent to give the title compound (1.22 g, 6.78 mmol, 95%): LCMS; [M+H]⁺=181, Rt=1.54 min, 97% purity

### Compound 142b. N-Isopropyl-benzene-1,2-diamine

Isopropyl-(2-nitro-phenyl)-amine (1.22 g, 6.78 mmol), 10% palladium on carbon (0.12 g, 10% w/w), and ethanol (12 ml) were stirred at room temperature under a hydrogen atmosphere for 18 hours. The reaction was filtered through celite and the filtrate evaporated under reduced pressure to give the title compound as brown oil (0.98 g, 6.53 mmol, 96%). LCMS: [M+H]⁺=151, Rt=0.75 min, 100% purity.

### Compound 142c. N-Isopropyl-N′-thieno[2,3-d]pyrimidin-4-yl-benzene-1,2-diamine

N-Isopropyl-benzene-1,2-diamine (88.2 mg, 0.588 mmol, 1.0 eq) and 4-chloro-thieno[2,3-d]pyrimidine (100 mg, 0.588 mmol, 1.0 eq) were suspended in IPA (2 ml), the reaction then heated at 90° C. for 18 hours. The reaction was allowed to cool to room temperature and the solvent was removed in vacuo. The resultant residue was purified by semi-preparative HPLC to give the title compound (34 mg, 0.12 mmol, 20%). LCMS: [M+H]⁺=285, Rt=0.97 min, 100% purity. The compounds listed below were prepared using route 6;

### Compound 143a: N-Cyclopentyl-N′-thieno[2,3-d]pyrimidin-4-yl-benzene-1,2-diamine

Yield; 7.0 mg, 0.04 mmol, 5%

LCMS; [M+H]⁺=311, Rt=1.22 min, 96% purity

### Compound 144a: N-Cyclohexyl-N′-thieno[2,3-d]pyrimidin-4-yl-benzene-1,2-diamine

Yield; 10.1 mg, 0.03 mmol, 4%

LCMS; [M+H]⁺=325, Rt=1.24 min, 94% purity

### Compound 145a: N-sec-Butyl-N′-thieno[2,3-d]pyrimidin-4-yl-benzene-1,2-diamine

Yield; 22.4 mg, 0.08 mmol, 9%

LCMS; [M+H]⁺=299, Rt=1.18 min, 98% purity

### Compound 146a: N-Isopropyl-N′-(5-methyl-thieno[2,3-d]pyrimidin-4-yl)-benzene-1,2-diamine

Yield; 4.0 mg, 0.01 mmol, 2%

LCMS; [M+H]⁺=299, Rt=1.60 min, 98% purity

### Compound 147a: N-sec-Butyl-N′-(5-methyl-thieno[2,3-d]pyrimidin-4-yl)-benzene-1,2-diamine

Yield; 4.0 mg, 0.01 mmol, 2%

LCMS; [M+H]⁺=313, Rt=1.73 min, 97% purity

### Compound 148a: N-Cyclopentyl-N′-(5-methyl-thieno[2,3-d]pyrimidin-4-yl)-benzene-1,2-diamine

Yield; 7.0 mg, 0.02 mmol, 3%

LCMS; [M+H]⁺=325, Rt=1.81 min, 100% purity

### Example 1i

### Synthesis Route 9

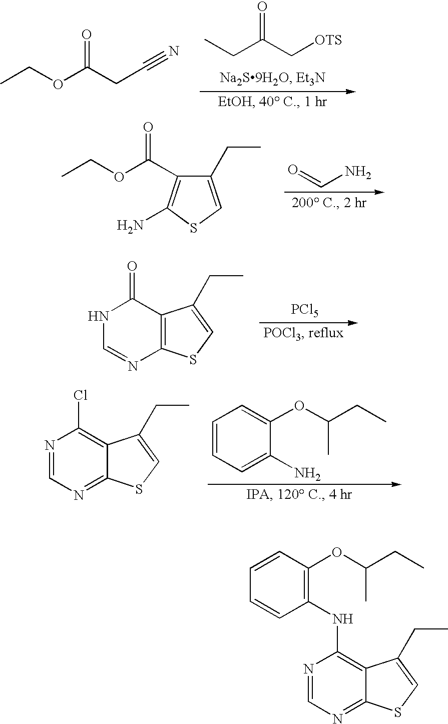

### Compound 151a. 2-Amino-5-ethyl-thiophene-3-carboxylic acid ethyl ester

A solution toluene-4-sulfonic acid 2-oxo-butyl ester (6.43 g, 26.52 mmol, 1.0 eq) in EtOH (5 ml) was added drop-wise to a solution of ethyl cyanoacetate (3.0 g, 26.52 mmol, 1.0 eq) and sodium sulphide nonhydrate (6.37 g, 26.52 mmol, 1.0 eq) in EtOH (30 ml) cooled to 0° C. Triethylamine (1.94 g, 26.52 mmol, 1.0 eq) was added drop-wise to the reaction at room temperature, the reaction stirred for an hour at room temperature before being heated at 40° C. for an additional hour. The reaction allowed to cool to room temperature before water (100 ml) was added. The mixture was then extracted with DCM (3×100 ml), the organics combined, washed with brine, dried over sodium sulphate, and the solvent removed in vacuo. The resultant residue was purified by column chromatography to give the title compound as a pink solid (1.34 g, 6.72 mmol, 25%). LCMS; [M+H]⁺=200, Rt=1.43 min, 89% purity.

### Compound 151b. 5-Ethyl-3H-thieno[2,3-d]pyrimidin-4-one

2-Amino-5-ethyl-thiophene-3-carboxylic acid ethyl ester (1.34 g, 6.72 mmol, 1.0 eq) was suspended in formamide (3 ml) and the reaction heated at 200° C. for 2 hours. The reaction was allowed to cool to room temperature, the resultant precipitate isolated by filtration, washed with cyclohexane and dried to give the title compound as a brown solid. On standing the filtrate gave further precipitate which was isolated by filtration, washed with cyclohexane and dried to give the title compound as a brown solid. The two solids were combined to give the title compound (0.44 g, 2.43 mmol, 36%). LCMS; [M+H]⁺=181, Rt=0.98 min, 98% purity.

### Compound 151c. 4-Chloro-5-ethyl-thieno[2,3-d]pyrimidine

5-Ethyl-3H-thieno[2,3-d]pyrimidin-4-one (0.44 g, 2.42 mmol, 1.0 eq) was added to a solution of phosphorous pentachloride (0.5 g, 2.42 mmol, 1.0 eq) in phosphorous oxychloride (3 ml) and the reaction heated at 130° C. for 1 hour. The reaction mixture was allowed to cool to room temperature and the solvent removed in vacuo. The resultant residue was purified by column chromatography to give the title compound as an off-white solid (0.19 g, 0.95 mmol, 39%). LCMS; [M+H]⁺=199, Rt=1.43 min, 97% purity.

### Compound 151d. (2-sec-Butoxy-phenyl)-(5-ethyl-thieno[2,3-d]pyrimidin-4-yl)-amine

2-sec-butoxy-phenylamine (39.3 mg, 0.238 mmol, 1.0 eq) and 4-chloro-5-ethyl-thieno[2,3-d]pyrimidine were suspended in IPA (3.0 ml) then heated at 120° C. for 16 hours. The reaction was allowed to cool to room temperature, ammonium hydroxide solution (1 ml) and water (4 ml) were added sequentially, the mixture extracted with DCM (2×3 ml). The organics were combined, dried over sodium sulphate, and the solvent removed in vacuo. The resultant residue was purified by column chromatography using 1% MeOH/DCM as eluent to give the title compound as yellow oil (32.0 mg, 0.1 mmol, 41%). LCMS; [M+H]⁺=328, Rt=2.30 min, 96% purity.

The compounds listed below were prepared via route 8, utilising anilines prepared as per routes 1 & 6;

### Compound 152a: (2-Cyclopentyloxy-phenyl)-(5-ethyl-thieno[2,3-d]pyrimidin-4-yl)-amine

Yield; 44.9 mg, 0.13 mmol, 56%

LCMS; [M+H]⁺=340, Rt=2.35 min, 97% purity

### Compound 150a: (5-Ethyl-thieno[2,3-d]pyrimidin-4-yl)-(2-isopropoxy-phenyl)-amine

Yield; 31.0 mg, 0.10 mmol, 42%

LCMS; [M+H]⁺=314, Rt=2.22 min, 100% purity

### Compound 157a: (5-Ethyl-thieno[2,3-d]pyrimidin-4-yl)-(2-isopropoxy-phenyl)-amine

Yield; 44.5 mg, 0.13 mmol, 55%

LCMS; [M+H]⁺=342, Rt=1.99 min, 100% purity

### Example 1j

### Synthesis Route 10

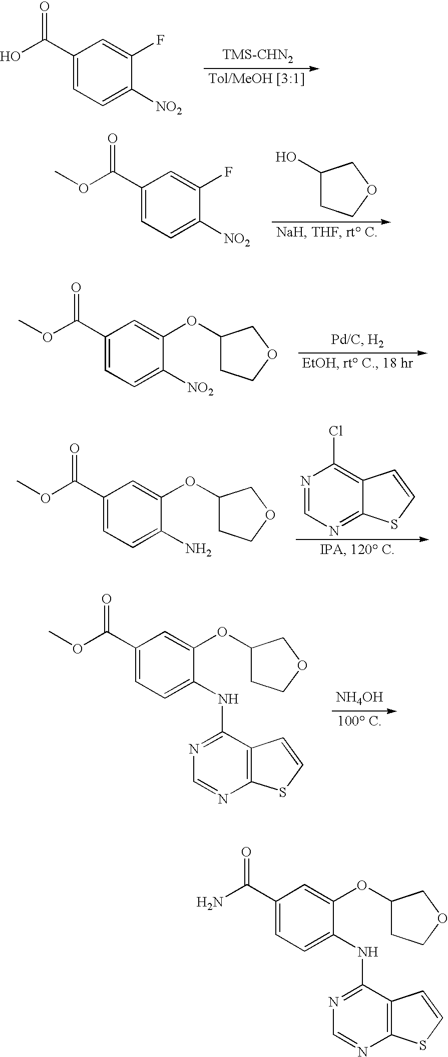

### Compound 153a: 3-Fluoro-4-nitro-benzoic acid methyl ester

A solution of 3-fluoro-4-nitrobenzoic acid (0.5 g, 2.7 mmol, 1.0 eq) in 3:1 toluene/methanol (8 ml) was cooled to 0° C. and 2.0M TMS-diazomethane/Et₂O (1.8 ml, 3.5 mmol, 1.3 eq) was added dropwise. The reaction was stirred for 1 hour and allowed to warm to room temperature. The solvent was removed in vacuo to give the title compound as a yellow solid (0.54 g, 2.7 mmol, 100%). ¹H NMR shows product in >95% purity.

### Compound 153b: 4-Nitro-3-(tetrahydro-furan-3-yloxy)-benzoic acid methyl ester

A 60% dispersion of sodium hydride in mineral oil (0.11 g, 2.76 mmol, 1.1 eq) was added to a solution of 3-hydroxytetrahydrofuran (0.2 ml, 2.51 mmol, 1.0 eq) in THF (4 ml) and the mixture stirred at room temperature for 10 minutes. A solution of 3-fluoro-4-nitro-benzoic acid methyl ester (0.5 g, 2.51 mmol, 1.0 eq) in THF (4 ml) was added to the mixture and the reaction stirred for 18 hours at room temperature. The solvent was removed in vacuo and the resultant residue was purified by column chromatography using 15% EtOAc/cyclohexane as eluent to give the title compound as a white solid. (0.45 g, 1.69 mmol, 67%). 1H NMR shows product in >95% purity.

### Compound 153c: 4-Amino-3-(tetrahydro-furan-3-yloxy)-benzoic acid methyl ester

A suspension of 4-nitro-3-(tetrahydro-furan-3-yloxy)-benzoic acid methyl ester (0.15 g, 0.56 mmol, 1.0 eq) and 10% w/w Palladium on carbon (15 mg, 10% w/w) in ethanol (5 ml) was stirred under a hydrogen atmosphere for 18 hours at room temperature. The mixture was filtered through celite and the solvent removed in vacuo. The resultant oil was triturated with diethylether and the solvent removed in vacuo to give the title compound as a white solid (0.13 g, 0.55 mmol, 97%). LCMS; [M+H]⁺=238, Rt=0.96 min, 95% purity.

### Compound 153d: 3-(Tetrahydro-furan-3-yloxy)-4-(thieno[2,3-d]pyrimidin-4-ylamino)-benzoic acid methyl ester

4-Amino-3-(tetrahydro-furan-3-yloxy)-benzoic acid methyl ester (50 mg, 0.293 mmol, 1.0 eq) and 4-chlorothieno[2,3d]pyrimidine (69 mg, 0.293 mmol, 1.0 eq) were dissolved in IPA (2 ml) and heated at 120° C. for 18 hours. The reaction was allowed to cool to room temperature, the resultant precipitate was isolated by filtration, washed with acetone, and dried on the sinter to give the title compound as a green solid (69 mg, 0.19 mmol, 63%). LCMS; [M+H]⁺=372, Rt=1.29 min, 100% purity.

### Compound 153d: 3-(Tetrahydro-furan-3-yloxy)-4-(thieno[2,3-d]pyrimidin-4-ylamino)-benzamide

A suspension of 3-(tetrahydro-furan-3-yloxy)-4-(thieno[2,3-d]pyrimidin-4-ylamino)-benzoic acid methyl ester (60 mg, 0.16 mmol, 1.0 eq) in 28% ammonium hydroxide solution (3 ml) was heated at 100° C. for 18 hours. The reaction was allowed to cool, the resultant precipitate isolated by filtration, washed with acetone, and dried in vacuo to give the title compound as a yellow solid (25.0 mg, 0.07 mmol, 43%). LCMS; [M+H]⁺=357, Rt=0.98 min, 88% purity.

The compounds listed below were prepared via route 10.

### Compound 154a: 4-(5-Methyl-thieno[2,3-d]pyrimidin-4-ylamino)-3-(tetrahydro-furan-3-yloxy)-benzoic acid methyl ester

Yield; 48 mg, 0.12 mmol, 46%

LCMS; [M+H]⁺=386, Rt=1.45 min, 94% purity

### Compound 155a: 4-(5-Methyl-thieno[2,3-d]pyrimidin-4-ylamino)-3-(tetrahydro-furan-3-yloxy)-benzoic acid methyl ester

Yield; 37 mg, 0.09 mmol, 34%

LCMS; [M+H]⁺=386, Rt=1.61 min, 100% purity

### Example 1k

### Synthesis Route 11

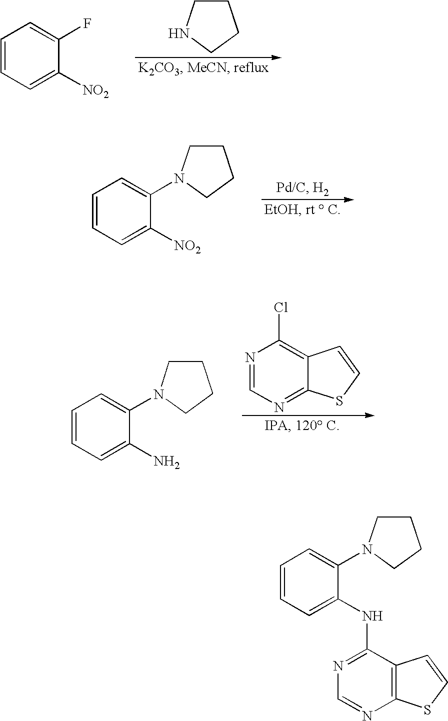

### Compound 158a. 1-(2-Nitro-phenyl)-pyrrolidine

A suspension of 2-fluoro-nitrobenzene (1.0 g, 7.09 mmol, 1.0 eq), pyrrolidine (0.5 g, 7.09 mmol, 1.0 eq), and potassium carbonate (1.18 g, 8.51 mmol, 1.2 eq) in acetonitrile was heated at reflux for 3 hours then allowed to cool with stirring for 18 hours. The reaction was diluted with water (10 ml) and ethyl acetate (20 ml) and the organic layer removed. The aqueous phase was then re-extracted twice more with ethyl acetate (2×20 ml), the organics combined, dried over sodium sulphate, and the solvent removed in vacuo to give the title compound (1.36 g, 7.09 mmol, 100%). ¹H NMR shows product in >95% purity.

### Compound 158b. 2-Pyrrolidin-1-yl-phenylamine

A suspension of 1-(2-nitro-phenyl)-pyrrolidine (1.36 g, 7.09 mmol, 1.0 eq) and 10% w/w palladium on carbon (0.14 g, 10% w/w) in ethanol (40 ml) was stirred at room temperature under a hydrogen atmosphere for 20 hours. The reaction was filtered through celite and the filtrate was concentrated to dryness in vacuo to give the title compound (1.24 g, 7.6 mmol, 100% corrected). LCMS; [M+H]⁺=163, Rt=0.71 min, 94% purity

### Compound 158c. (2-Pyrrolidin-1-yl-phenyl)-thieno[2,3-d]pyrimidin-4-yl-amine

A solution of 2-pyrrolidin-1-yl-phenylamine (0.1 g, 0.62 mmol, 1.0 eq) and 4-chloror-thieno[2,3-d]pyrimidine (0.106 g, 0.62 mmol, 1.0 eq) in IPA (4 ml) was heated at 120° C. for 20 hours in an ACE pressure tube. The reaction was allowed to cool to room temperature and ammonium hydroxide (1 ml) added followed by water (5 ml). The resultant precipitate was isolated by filtration, and purified by column chromatography using DCM as eluent to give the title compound (62.8 mg, 0.21 mmol, 34%). LCMS; [M+H]⁺=297, Rt=1.46 min, 100% purity

The compounds listed below were prepared via route 11;

### Compound 159a: (5-Methyl-thieno[2,3-d]pyrimidin-4-yl)-(2-pyrrolidin-1-yl-phenyl)-amine

Yield; 21 mg, 0.07 mmol, 11%

LCMS; [M+H]⁺=311, Rt=1.57 min, 100% purity

### Compound 160a: (5,6-Dimethyl-thieno[2,3-d]pyrimidin-4-yl)-(2-pyrrolidin-1-yl-phenyl)-amine

Yield; 35.1 mg, 0.11 mmol, 17%

LCMS; [M+H]⁺=324, Rt=1.64 min, 100% purity

### Example 1l

### Synthesis Route 12

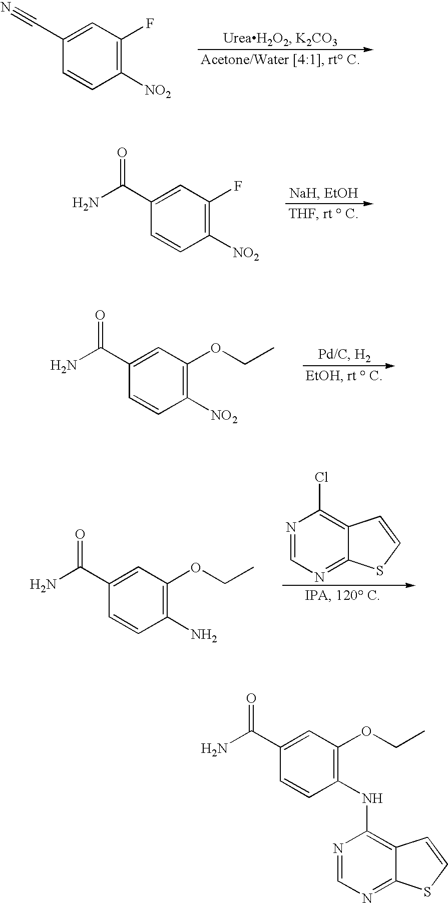

### Compound 161a: 3-Fluoro-4-nitro-benzamide

The urea/hydrogen peroxide complex (22.65 g, 240.8 mmol, 2.0 eq) was added to a solution of 3-fluoro-4-nitro-benzonitrile (20.0 g, 120.4 mmol, 1.0 eq) and potassium carbonate (33.28 g, 240.8 mmol, 2.0 eq) in 20% water/acetone (500 ml). The reaction was stirred at room temperature for 22 hours when urea/hydrogen peroxide complex (11.33 g, 120.4 mmol, 1.0 eq) and potassium carbonate (16.64 g, 120.4 mmol, 1.0 eq) were added. The reaction was stirred for a further 2 hours at room temperature then diluted with water (300 ml) and DCM (500 ml). The organic layer was removed and the aqueous extracted with DCM (2×500 ml). The organics were combined, washed with brine, dried over sodium sulphate, and the solvent removed in vacuo to give the title compound as an orange solid (14.065 g, 76.31 mmol, 63%). ¹H NMR shows product in >95% purity.

### Compound 161 b: 3-Ethoxy-4-nitro-benzamide

Ethanol (0.83 g, 16.29 mmol, 2.0 eq) was added drop-wise to a suspension of 60% sodium hydride as a dispersion in mineral oil (0.36 g, 8.96 mmol, 1.1 eq) in THF (25 ml) cooled to 0° C. The suspension was stirred for 30 minutes at 0° C. and the mixture added drop-wise to a solution of 3-fluoro-4-nitro-benzamide (1.5 g, 8.15 mmol, 1.0 eq) in THF (15 ml), the reaction was stirred at room temperature for 18 hours. The reaction was diluted with water (25 ml) and DCM (50 ml), the organic layer separated. The aqueous layer was extracted twice with DCM (2×50 ml), the organics combined, washed with brine, dried over sodium sulphate, and the solvent removed to give the title compound as an orange solid (1.14 g, 5.42 mmol, 67%). LCMS; [M+H]⁺=211, Rt=1.05 min, 100% purity

### Compound 161c: 3-Ethoxy-4-amino-benzamide

A suspension of 3-ethoxy-4-nitro-benzamide (1.14 g, 5.42 mmol, 1.0 eq) and 10% w/w palladium on carbon (0.14 g, 10% w/w) in ethanol (100 ml) was stirred under a hydrogen atmosphere for 18 hours at room temperature. The reaction was filtered through a celite pad and the filtrate concentrated to dryness in vacuo to give the title compound as a green solid (0.96 g, 0.533 mmol, 98%). LCMS; [M+H]⁺=181, Rt=0.55 min, 97% purity.

### Compound 161d: 3-Ethoxy-4-(thieno[2,3-d]pyrimidin-4-ylamino)-benzamide

A suspension of 3-ethoxy-4-amino-benzamide (55 mg, 0.303 mmol, 1.0 eq) and 4-chloro-thieno[2,3d]pyrimidine in IPA (2 ml) was heated at 120° C. for 4 hours. The reaction was allowed to cool to room temperature, water (4 ml) and ammonium hydroxide (1 ml) were then added. The resultant precipitate was isolated by filtration, washed with water and dried in vacuo to give the title compound (38 g, 0.12 mmol, 40%). LCMS; [M+H]⁺=315, Rt=1.54 min, 100% purity.

The compounds listed below were prepared via route 12;

### Compound 162a: 3-Isopropoxy-4-(thieno[2,3-d]pyrimidin-4-ylamino)-benzamide

Yield; 74 mg, 0.23 mmol, 74%

LCMS; [M+H]⁺=329, Rt=1.61 min, 100% purity

### Compound 163a: 3-sec-Butoxy-4-(thieno[2,3-d]pyrimidin-4-ylamino)-benzamide

Yield; 24 mg, 0.07 mmol, 23%

LCMS; [M+H]⁺=343, Rt=1.69 min, 100% purity

### Compound 164a: 3-Cyclopentyloxy-4-(thieno[2,3-d]pyrimidin-4-ylamino)-benzamide

Yield; 32 mg, 0.08 mmol, 28%

LCMS; [M+H]⁺=355, Rt=1.71 min, 100% purity

### Compound 165a: 3-Ethoxy-4-(5-methyl-thieno[2,3-d]pyrimidin-4-ylamino)-benzamide

Yield; 82.0 mg, 0.25 mmol, 77%

LCMS; [M+H]⁺=329, Rt=1.78 min, 100% purity

### Compound 166a: 3-Isopropoxy-4-(5-methyl-thieno[2,3-d]pyrimidin-4-ylamino)-benzamide

Yield; 84.8 mg, 0.25 mmol, 77%

LCMS; [M+H]⁺=343, Rt=1.84 min, 100% purity

### Compound 167a: 3-sec-Butoxy-4-(5-methyl-thieno[2,3-d]pyrimidin-4-ylamino)-benzamide

Yield; 91.7 mg, 0.26 mmol, 79%

LCMS; [M+H]⁺=357, Rt=1.91 min, 96% purity

### Compound 168a: 3-Cyclopentyloxy-4-(5-methyl-thieno[2,3-d]pyrimidin-4-ylamino)-benzamide

Yield; 99.2 mg, 0.27 mmol, 83%

LCMS; [M+H]⁺=369, Rt=1.94 min, 100% purity

### Compound 169a: 4-(5-Methyl-thieno[2,3-d]pyrimidin-4-ylamino)-3-(tetrahydro-furan-3-yloxy)-benzamide

Yield; 86.6 mg, 0.23 mmol, 72%

LCMS; [M+H]⁺=371, Rt=1.68 min, 100% purity

### Compound 170a: 4-(Thieno[2,3-d]pyrimidin-4-ylamino)-3-(3,3,3-trifluoro-propoxy)-benzamide

Yield; 58 mg, 0.15 mmol, 51%

LCMS; [M+H]⁺=383, Rt=1.70 min, 97% purity

### Compound 171a: 4-(5-Methyl-thieno[2,3-d]pyrimidin-4-ylamino)-3-(3,3,3-trifluoro-propoxy)-benzamide

Yield; 48 mg, 0.12 mmol, 38%

LCMS; [M+H]⁺=397 Rt=1.87 min, 96% purity

### Compound 172a: 4-(5,6-Dimethyl-thieno[2,3-d]pyrimidin-4-ylamino)-3-(3,3,3-trifluoro-propoxy)-benzamide

Yield; 79 mg, 0.19 mmol, 64%

LCMS; [M+H]⁺=411, Rt=1.93 min, 96% purity

¹H NMR shows title compound in >90%

### Compound 173a: 4-(5,6-Dimethyl-thieno[2,3-d]pyrimidin-4-ylamino)-3-ethoxy-benzamide

Yield; 71.4 mg, 0.21 mmol, 66%

LCMS; [M+H]⁺=434, Rt=1.31 min, 54% purity.

¹H NMR shows title compound in >90%

### Compound 174a: 4-(5,6-Dimethyl-thieno[2,3-d]pyrimidin-4-ylamino)-3-isopropoxy-benzamide

Yield; 78.4 mg, 0.22 mmol, 73%

LCMS; [M+H]⁺=357, Rt=1.36 min, 39% purity.

¹H NMR shows title compound in >90%

### Compound 175a: 3-Cyclopentyloxy-4-(5,6-dimethyl-thieno[2,3-d]pyrimidin-4-ylamino)-benzamide

Yield; 90.9 mg, 0.24 mmol, 77%

LCMS; [M+H]⁺=383, Rt=1.46 min, 53% purity

¹H NMR shows title compound in >90%

### Compound 176a: 4-(5,6-Dimethyl-thieno[2,3-d]pyrimidin-4-ylamino)-3-(tetrahydro-furan-3-yloxy) benzamide

Yield; 84.5 mg, 0.22 mmol, 71%

LCMS; [M+H]⁺=385, Rt=1.22 min, 97% purity

### Compound 187a: 3-sec-Butoxy-4-(5,6-dimethyl-thieno[2,3-d]pyrimidin-4-ylamino)-benzamide

Yield; 80.6 mg, 0.22 mmol, 72%

LCMS; [M+H]⁺=371, Rt=2.26 min, 95% purity

### Example 1m

### Synthesis Route 13

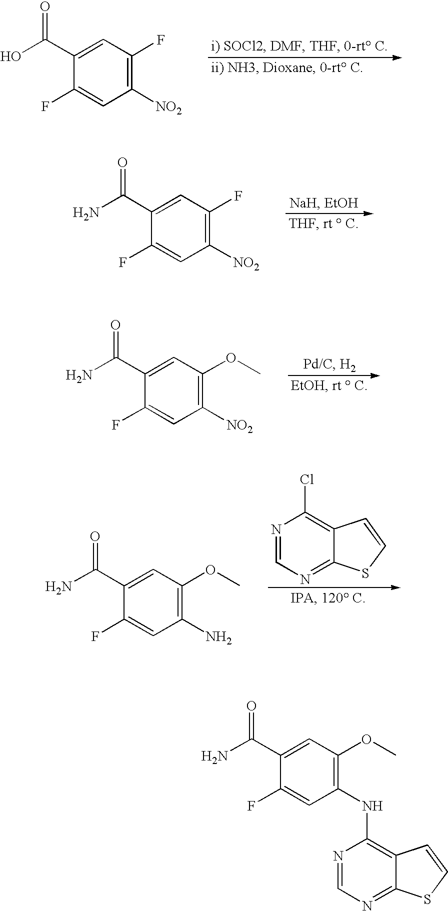

### Compound 179a: 2,5-Difluoro-4-nitro-benzamide

A solution of 2,5-difluoro-4-nitro-benzoic acid (4.82 g, 23.73 mmol, 1.0 eq) in THF (50 ml) was cooled to 0° C., then thionyl chloride (22.59 g, 189.86 mmol, 8.0 eq) and DMF (1 ml) were added and the reaction stirred at room temperature for 1.5 hours. DIPEA (24.54 g, 189.86 mmol, 8.0 eq) and 0.5M ammonia/dioxane (142.4 ml, 71.03 mmol, 3.0 eq) were sequentially added to the mixture, and the reaction heated to 50° C. for 17 hours. The reaction had not gone to completion so was stirred at room temperature for an additional 66 hours. The solvent was removed in vacuo and the resultant residue purified by column chromatography using cyclohexane/ethyl acetate [1:1] as eluent to give the title compound as a dark solid (0.94 g, 4.6 mmol, 12%). ¹H NMR shows product in >95% purity

### Compound 179b: 2-Fluoro-5-methoxy-4-nitro-benzamide

Methanol (109 mg, 3.4 mmol, 2.2 eq) was added drop-wise to a suspension of 60% sodium hydride as a dispersion in mineral oil (67.9 mg, 1.7 mmol, 1.1 eq) in THF (2 ml) cooled to 0° C. The suspension was stirred for 30 minutes at 0° C. and the mixture added drop-wise to a solution of 2,5-difluoro-4-nitro-benzamide (312 mg, 1.54 mmol, 1.0 eq) in THF (3 ml), the reaction was stirred at room temperature for 18 hours. The reaction was diluted with water (5 ml) and DCM (10 ml), the organic layer separated. The aqueous layer was extracted twice with DCM (2×10 ml), the organics combined, washed with brine, dried over sodium sulphate, and the solvent removed in vacuo. The resultant residue was purified by column chromatography to give the title compound as an orange solid (228 mg, 1.06 mmol, 69%). ¹H NMR shows product in >95% purity.

### Compound 179c: 4-Amino-2-fluoro-5-methoxy-benzamide

A suspension of 2-fluoro-5-methoxy-4-nitro-benzamide (228 mg, 1.06 mmol, 1.0 eq) and 10% w/w palladium on carbon (23 mg, 10% w/w) in ethanol (20 ml) was stirred under a hydrogen atmosphere for 18 hours at room temperature. The reaction was filtered through a celite pad and the filtrate concentrated to dryness in vacuo to give the title compound as an off-white solid (198 mg, 1.06 mmol, 100%). LCMS; [M+H]⁺=185, Rt=1.19 min, 90% purity.

### Compound 179d: 2-Fluoro-5-methoxy-4-(thieno[2,3-d]pyrimidin-4-ylamino)-benzamide

A suspension of 4-amino-2-fluoro-5-methoxy-benzamide (66 mg, 0.358 mmol, 1.0 eq) and 4-chloro-thieno[2,3d]pyrimidine (61 mg, 0.358 mmol, 1.0 eq) in IPA (2 ml) was heated at 120° for 5 hours. The reaction allowed to cool to room temperature, water (4 ml) and ammonium hydroxide (1 ml) were then added. The resultant precipitate was isolated by filtration, washed with water and dried in vacuo to give the title compound as a green solid (97.0 mg, 0.30 mmol, 85%). LCMS; [M+H]⁺=319, Rt=1.80 min, 100% purity.

The compounds listed below were prepared via route 13;

### Compound 180a: 2-Fluoro-5-isopropoxy-4-(thieno[2,3-d]pyrimidin-4-ylamino)-benzamide

Yield; 47.7 mg, 0.14 mmol, 52%

LCMS; [M+H]⁺=347, Rt=2.03 min, 100% purity

### Compound 181a: 2-Fluoro-5-(tetrahydro-furan-3-yloxy)-4-(thieno[2,3-d]pyrimidin-4-ylamino)-benzamide

Yield; 30.2 mg, 0.08 mmol, 36%

LCMS; [M+H]⁺=375, Rt=1.79 min, 100% purity

### Compound 182a: 2-Fluoro-5-methoxy-4-(5-methyl-thieno[2,3-d]pyrimidin-4-ylamino)-benzamide

Yield; 56.3 mg, 0.18 mmol, 49%

LCMS; [M+H]⁺=333, Rt=2.00 min, 89% purity

### Compound 183a: 2-Fluoro-4-(5-methyl-thieno[2,3-d]pyrimidin-4-ylamino)-5-(tetrahydro-furan-3-yloxy)-benzamide

Yield; 15.9 mg, 0.04 mmol, 19%

LCMS; [M+H]⁺=389, Rt=1.96 min, 89% purity

### Compound 184a: 4-(5,6-Dimethyl-thieno[2,3-d]pyrimidin-4-ylamino)-2-fluoro-5-methoxy-benzamide

Yield; 40.0 mg, 0.12 mmol, 32%

LCMS; [M+H]⁺=347, Rt=2.12 min, 83% purity

### Compound 185a: 4-(5,6-Dimethyl-thieno[2,3-d]pyrimidin-4-ylamino)-2-fluoro-5-isopropoxy-benzamide

Yield; 35.4 mg, 0.09mmol, 36%

LCMS; [M+H]⁺=375, Rt=2.34 min, 98% purity

### Compound 186a: 4-(5,6-Dimethyl-thieno[2,3-d]pyrimidin-4-ylamino)-2-fluoro-5-(tetrahydro-furan-3-yloxy)-benzamide

Yield; 18.5 mg, 0.05mmol, 21%

LCMS; [M+H]⁺=403, Rt=2.08 min, 96% purity

### Example 1n

### Synthesis Route 14

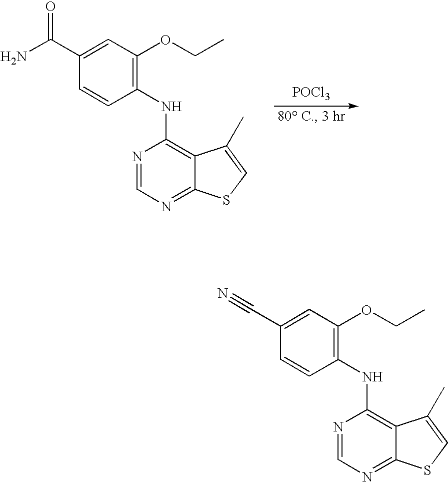

### Compound 188a: 3-Ethoxy-4-(5-methyl-thieno[2,3-d]pyrimidin-4-ylamino)-benzonitrile

A solution of 3-ethoxy-4-(5-methyl-thieno[2,3-d]pyrimidin-4-ylamino)-benzamide (97.8 mg, 0.30 mmol) in phosphorous oxychloride (2 ml) was heated at 80° C. for 3 hours. The mixture was diluted with toluene (10 ml) and the solvent was removed in vacuo. 880 Ammonia solution (2 ml) and water (2 ml) were added to the resultant residue, the precipitate isolated. The precipitate was washed with water, cyclohexane, and dried in vacuo to give the title compound (63.1 mg, 0.20 mmol, 68%). LCMS; [M+H]⁺=371, Rt=2.26 min, 95% purity.

### Compound 189a: 3-lsopropoxy-4-(5-methyl-thieno[2,3-d]pyrimidin-4-ylamino)-benzonitrile

Yield; 72.8 mg, 0.24 mmol, 86%

LCMS; [M+H]⁺=311, Rt=2.52 min, 100% purity

### Compound 190a: 3-sec-Butoxy-4-(5,6-dimethyl-thieno[2,3-d]pyrimidin-4-ylamino)-benzonitrile

Yield; 5.8 mg, 0.02 mmol, 17%

LCMS; [M+H]⁺=325, Rt=2.59 min, 100% purity

### Example 1o

### Synthesis Route 15

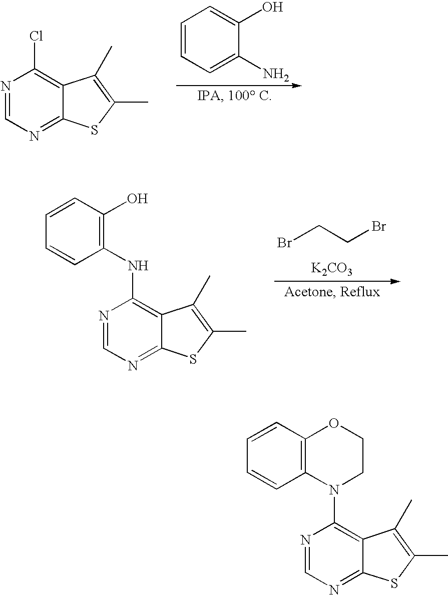

### Compound 191a: 2-(5,6-Dimethyl-thieno[2,3-d]pyrimidin-4-ylamino)-phenol

A solution of 4-chloro-5,6-dimethyl-thieno[2,3-d]pyrimidine (364 mg, 1.83 mmol, 1.0 eq) and 2-hydroxyaniline (200 mg, 1.83 mmol, 1.0 eq) in IPA (5 ml) was heated at 100° C. for 2 hours. The reaction mixture was allowed to cool to room temperature and the resultant precipitate was isolated by filtration. The solid was washed with water and dried in vacuo to give the title compound (270 mg, 0.99 mmol, 54%). LCMS; [M+H]⁺=271, Rt=1.07 min, 97% purity

### Compound 191b: 4-(5,6-Dimethyl-thieno[2,3-d]pyrimidin-4-yl)-3,4-dihydro-2H-benzo[1,4]oxazine

A suspension of 2-(5,6-dimethyl-thieno[2,3-d]pyrimidin-4-ylamino)-phenol (100 mg, 0.37 mmol, 1.0 eq), 1,2-dibromoethane (103 mg, 0.55 mmol, 1.5 eq), and potassium carbonate (128 mg, 0.93 mmol, 2.5 mmol) in acetone (5 ml) was heated at reflux for 6 hours. The reaction was allowed to cool to room temperature, diluted with water (10 ml), extracted with ethyl acetate (2×10 ml), the organics combined, dried over sodium sulphate, and the solvent was removed in vacuo. The resultant residue was purified by column chromatography using DCM as eluent to give the title compound (27.3 mg, 0.10 mmol, 26%). LCMS; [M+H]⁺=298, Rt=1.57 min, 98% purity

### Example 1p

### Synthesis Route 16

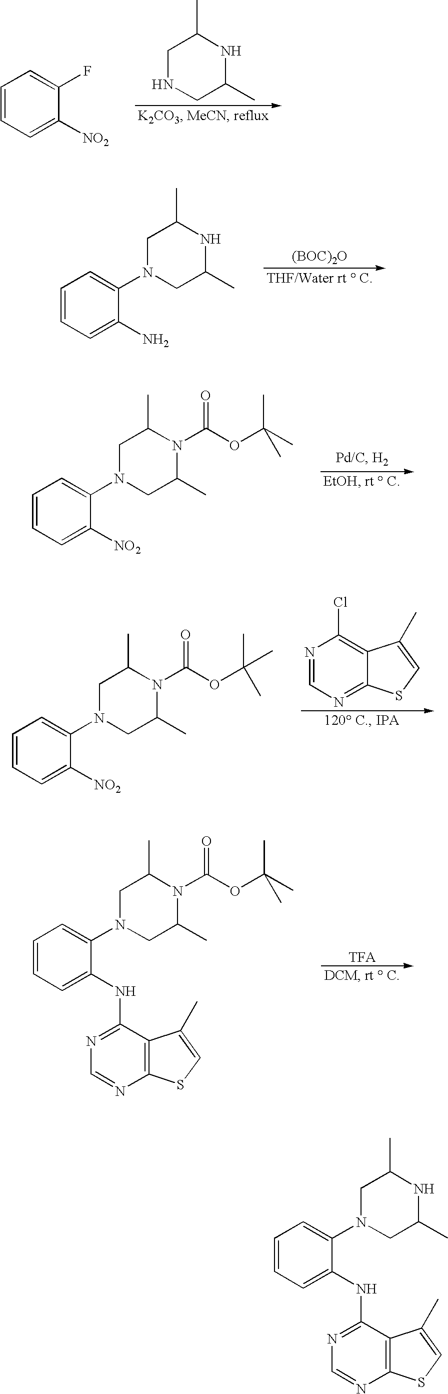

### Compound 192b: 4-(2-Nitro-phenyl)-2,6-dimethyl-piperazine-1-carboxylic acid tert-butyl ester

BOC Anhydride (3.4 g, 15.58 mmol, 1.0 eq) was added to a solution of 3,5-dimethyl-1-(2-nitro-phenyl)-piperazine (3.6 g, 15.58 mmol, 1.0 eq) in THF (40 ml) and water (40 ml). The reaction was stirred at room temperature for 4 days. The reaction mixture was extracted with ethyl acetate, the organics dried over sodium sulphate, and the solvent removed in vacuo. The resultant residue was purified by column chromatography using DCM as the eluent to give the title compound (5.04 g, 15.03 mmol, 96%). ¹H NMR shows product in >95% purity.

### Compound 192c: 4-(2-Amino-phenyl)-2,6-dimethyl-piperazine-1-carboxylic acid tert-butyl ester

A suspension of 4-(2-nitro-phenyl)-2,6-dimethyl-piperazine-1-carboxylic acid tert-butyl ester (5.0 g, 14.9 mmol, 1.0 eq) and 10% w/w palladium on carbon (500 mg, 10% w/w) in ethanol (100 ml) was stirred under a hydrogen atmosphere for 18 hours at room temperature. The reaction was filtered through a celite pad and the filtrate concentrated to dryness in vacuo to give the title compound (3.95 g, 12.9 mmol, 87%). LCMS; [M+H]⁺=2.06, Rt=0.55 min, 90% purity.

### Compound 192d: 2,6-Dimethyl-4-[2-(thieno[2,3-cl]pyrimidin-4-ylamino)-phenyl]-poperazine-1-carboxylic acid tert-butyl ester

A suspension of 4-(2-amino-phenyl)-2,6-dimethyl-piperazine-1-carboxylic acid tert-butyl ester (100 mg, 0.33 mmol, 1.0 eq) and 4-chloro-thieno[2,3d]pyrimidine (56 mg, 0.33 mmol, 1.0 eq) in IPA (4 ml) was heated at 120° C. for 3 days. The reaction was allowed to cool to room temperature, the solvent was removed in vacuo and the resultant residue was purified by column chromatography using DCM as eluent to give the title compound (41.0 mg, 0.09 mmol, 11%). LCMS; [M+H]⁺=440, Rt=1.44 min, 97% purity.

### Compound 192e: [2-(3,5-Dimethyl-piperazin-1-yl)-phenyl]-thieno[2,3-d]pyrimidin-4-yl-amine

A solution of 2,6-dimethyl-4-[2-(thieno[2,3-d]pyrmidin-4-ylamino)-phenyl]-piperazine-1-carboxylic acid tert-butyl ester (0.3 g, 0.85 mmol, 1.0 eq) and trifluoroacetic acid (0.5 ml) in DCM (2 ml) was stirred at room temperature for 24 hours, the solvent was removed in vacuo. The residue was portioned between DCM (6 ml) and 1M sodium hydroxide solution (6 ml), the organic layer removed and the aqueous extracted with DCM (3 ml). The organics were combined, dried over sodium sulphate, and the solvent removed in vacuo. The resultant residue was then purified by column chromatography using 10% MeOH/DCM as eluent to give the title compound (146 mg, 0.43 mmol, 65%). LCMS; [M+H]⁺=340, Rt=1.01 min, 100% purity.

The compounds listed below were prepared via route 16:

### Compound 196a: [2-(3,5-Dimethyl-piperazin-1-yl)-phenyl]-thieno[2,3-d]pyrimidin-4-yl-amine

Yield; 19 mg, 0.04 mmol, 13%

LCMS; [M+H]⁺=454, Rt=1.54 min, 91% purity

### Example 1q

### Synthesis Route 17

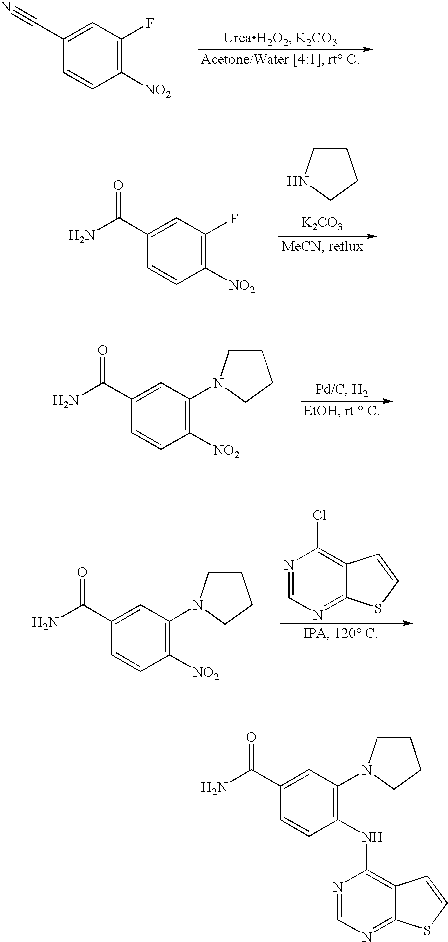

### Compound 193a: 3-Fluoro-4-nitro-benzamide

As per route 12, compound 161a.

### Compound 193b: 4-Nitro-3-pyrrolidin-1-yl-benzamide

Pyrrolidine (0.58 g, 8.15 mmol, 1.0 eq) was added to a suspension of 3-fluoro-4-nitro-benzamide (1.5 g, 8.15 mmol, 1.0 eq) and potassium carbonate (2.25 g, 9.78 mmol, 1.2 eq) in acetonitrile (25 ml). The suspension was heated at reflux for 2.5 hours. The reaction was quenched with water (10 ml), extracted with DCM (3×50 ml), organics combined, dried over sodium sulphate and the solvent removed in vacuo to give the title compound as an orange solid (1.56 g, 6.64 mmol, 81%).¹H NMR shows product in ca. 95% purity

### Compound 193c: 4-Amino-3-pyrrolidin-1-yl-benzamide

A suspension of 4-nitro-3-pyrrolidin-1-yl-benzamide (1.56 g, 6.64 mmol, 1.0 eq) and 10% w/w palladium on carbon (200 mg, 13% w/w) in ethanol (100 ml) was stirred under a hydrogen atmosphere for 18 hours at room temperature. The reaction was filtered through a celite pad and the filtrate concentrated to dryness in vacuo to give the title compound as dark solid (1.35 g, 6.58 mmol, 99%). LCMS; [M+H]⁺=2.06, Rt=0.55 min, 90% purity.

### Compound 193d: 3-Pyrrolidin-1-y1-4-(thieno[2,3-d]pyrimidin-4-ylamino)-benzamide

A suspension of 4-amino-3-pyrrolidin-1-yl-benzamide (75 mg, 0.365 mmol, 1.0 eq) and 4-chloro-thieno[2,3d]pyrimidine (62 mg, 0.3658 mmol, 1.0 eq) in IPA (2 ml) was heated at 120° C. for 40 hours. The reaction was allowed to cool to room temperature, water (4 ml) and ammonium hydroxide (1 ml) were then added. The resultant precipitate was isolated by filtration, washed with water and dried in vacuo to give the title compound as a green solid (41.0 mg, 0.12 mmol, 33%). LCMS; [M+H]⁺=40, Rt=1.47 min, 100% purity.

The compounds listed below were prepared via route 17;

### Compound 194a: 4-(5-Methyl-thieno[2,3-d]pyrimidin-4-ylamino)-3-pyrrolidin-1-yl-benzamide

Yield; 45 mg, 0.13 mmol, 35%

LCMS; [M+H]⁺=354, Rt=1.60 min, 94% purity

### Compound 195a: 4-(5,6-Dimethyl-thieno[2,3-d]pyrimidin-4-ylamino)-3-pyrrolidin-1-yl-benzamide

Yield; 43 mg, 0.11 mmol, 32%

LCMS; [M+H]⁺=368, Rt=1.68 min, 92% purity

### Example 1r

### Synthesis Route 18

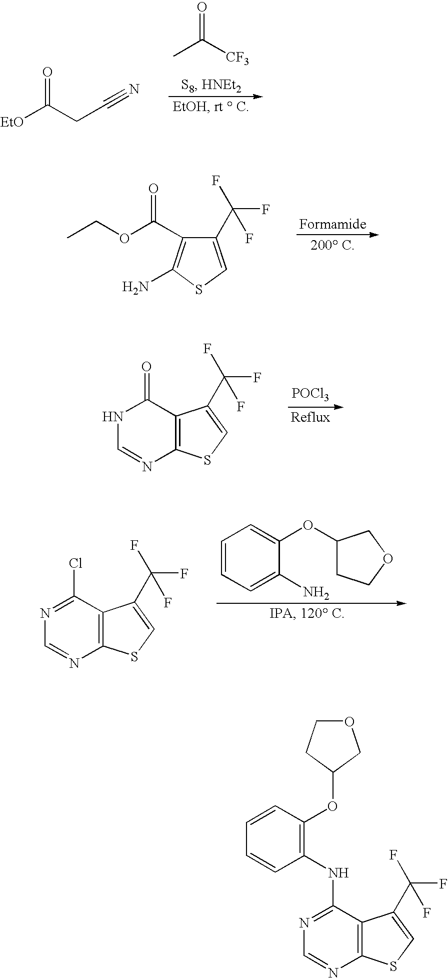

### Compound 197a: 2-Amino-4-trifluoromethyl-thiophene-3-carboxylic acid ethyl ester

A suspension of ethyl cyanoacetate (5.05 g, 44.6 mmol, 1.0 eq), trifluoroacetone (5.0 g, 44.6 mmol 1.0 eq), sulphur (1.43 g, 44.6 mmol 1.0 eq), and diethylamine (3.26 g, 44.6 mmol 1.0 eq) in ethanol (15 ml) was stirred for 1 hour at room temperature. The solvent was removed in vacuo and the resultant residue was purified by column chromatography using 1% MeOH/DCM as eluent to give the title compound (0.25 g, 1.0 mmol, 2%). ¹H NMR shows product in ca. 95% purity.

### Compound 197b: 5-Trifluoromethyl-3H-thieno[2,3-d]pyrimidin-4-one

A suspension of 2-amino-4-trifluoromethyl-thiophene-3-carboxylic acid ethyl ester (0.25 g, 1.05 mmol, 1.0 eq) in formamide (2 ml) was heated at 200° C. for 2 hours. The reaction was allowed to cool to room temperature, diluted with water (10 ml), extracted with ethyl acetate (3×10 ml), the organics combined and the solvent removed in vacuo. The resultant residue was purified by column chromatography using ethyl acetate as eluent to give the title compound (90 mg, 0.41 mmol, 39%).

### Compound 197c: 4-Chloro-5-trifluoromethyl-thieno[2,3-d]pyrimidine

A suspension of 5-trifluoromethyl-3H-thieno[2,3-d]pyrimidin-4-one (90 mg, 0.41 mmol, 1.0 eq) in phosphorous oxychloride (2 ml) was heated at reflux for 2 hours and the phosphorous oxychloride was removed in vacuo to give the title compound (0.1 g, 0.41 mmol, 100%).

### Compound 197d: [2-(Tetrahydro-furan-3-yloxy)-phenyl]-(5-trifluoromethyl-thieno[2,3-d]pyrimidin-4-yl)-amine

A suspension of 4-chloro-5-trifluoromethyl-thieno[2,3-d]pyrimidine (45 mg, 0.19 mmol, 1.0 eq) and 2-(tetrahydro-furan-3-yloxy)-phenylamine (34 mg, 0.19 mmol, 1.0 eq) in IPA (1 ml) was heated to 120° C. for 18 hours. The reaction was allowed to cool to room temperature, diluted with water (2 ml), and ammonium hydroxide solution was added (1 ml). The reaction mixture was extracted with ethyl acetate (2×10 ml), the organics combined and the solvent removed in vacuo. The resultant residue was purified by column chromatography using 40% cyclohexane/ethyl acetate as eluent to give the title compound (17 mg, 0.04 mmol, 23%). LCMS; [M+H]⁺=382, Rt=1.66 min, 97% purity

The compounds listed below were prepared via route 17, utilising anilines prepared as per routes 1 & 6;

### Compound 198a: (2-Cyclopentyloxy-phenyl)-(5-trifluoromethyl-thieno[2,3-d]pyrimidin-4-yl)-amine

Yield; 18.6 mg, 0.05 mmol, 26%

LCMS; [M+H]⁺=380 Rt=2.01 min, 100% purity

### Compound 199a: (2-Isopropoxy-phenyl)-(5-trifluoromethyl-thieno[2,3-d]pyrimidin-4-yl)-amine

Yield; 2.0 mg, 0.006 mmol, 9%

LCMS; [M+H]⁺=354, Rt=2.46 min, 100% purity

### Compound 200a: (2-sec-Butoxy-phenyl)-(5-trifluoromethyl-thieno[2,3-d]pyrimidin-4-yl)-amine

Yield; 2.9 mg, 0.008 mmol, 13%

LCMS; [M+H]⁺=368, Rt=2.55 min, 100% purity

### Compound 201a: 3-(Tetrahydro-furan-3-yloxy)-4-(5-trifluoromethyl-thieno[2,3-d]pyrimidin-4-ylamino)-benzamide

Yield; 6.0 mg, 0.014 mmol, 11%

LCMS; [M+H]⁺=425, Rt=1.82 min, 100% purity

### Compound 202a: 3-Methoxy-4-(5-trifluoromethyl-thieno[2,3-d]pyrimidin-4-ylamino)-benzamide

Yield; 6.0 mg, 0.02 mmol, 8%

LCMS; [M+H]⁺=369, Rt=1.98 min, 100% purity

### Compound 203a: 3-Ethoxy-4-(5-trifluoromethyl-thieno[2,3-d]pyrimidin-4-ylamino)-benzamide

Yield; 5.9 mg, 0.02 mmol, 7%

LCMS; [M+H]⁺=383, Rt=2.09 min, 100% purity

### Compound 204a: 3-Isopropoxy-4-(5-trifluoromethyl-thieno[2,3-d]pyrimidin-4-ylamino)-benzamide

Yield; 7.2 mg, 0.02 mmol, 9%

LCMS; [M+H]⁺=400, Rt=2.06 min, 100% purity

### Compound 205a: (4-Fluoro-2-methoxy-phenyl)-(5-trifluoromethyl-thieno[2,3-d]pyrimidin-4-yl)-amine

Yield; 10.4 mg, 0.03 mmol, 14%

LCMS; [M+H]⁺=344, Rt=2.54 min, 100% purity

### Compound 206a: (4-Fluoro-2-isopropoxy-phenyl)-(5-trifluoromethyl-thieno[2,3-d]pyrimidin-4-yl)-amine

Yield; 11.6 mg, 0.03 mmol, 15%

LCMS; [M+H]⁺=372, Rt=2.74 min, 100% purity

### Compound 207a: [4-Fluoro-2-(tetrahydro-furan-3-yloxy)-phenyl]-(5-trifluoromethyl-thieno[2,3-]pyrimidin-4-yl)-amine

Yield; 8.1 mg, 0.02 mmol, 10%

LCMS; [M+H]⁺=400, Rt=2.46 min, 100% purity

### Example 1s

### Synthesis Route 19

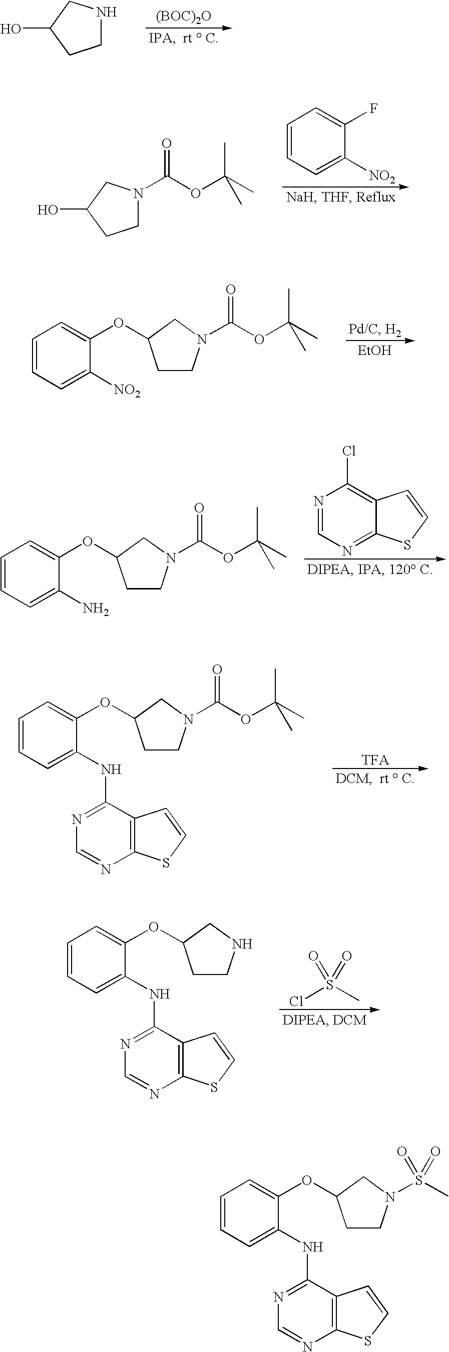

### Compound 208a: 3-Hydroxy-pyrrolidine-1-carboxylic acid tert-butyl ester

A solution of 3-hydroxypyrrolidine (1.5 g, 17.2 mmol, 1.0 eq) and BOC anhydride (3.76 g, 17.2 mmol, 1.0 eq) in IPA (20 ml) was stirred at room temperature for 2 hours and the solvent removed °to give the title compound as a tan solid (3.73 g, 17.2 mmol, 100% corrected). ¹H NMR shows product in ca. 90% purity.

### Compound 208b: 3-(2-Nitro-phenoxy)-pyrrolidine-1-carboxylic acid tert-butyl ester

Anhydrous tetrahydrofuran (30 ml) was added to sodium hydride as a 60% dispersion in mineral oil (0.77 g, 1.2 eq, 19.2 mmol.) in a flask fitted with a condenser, a nitrogen inlet and a bubbler. While stirring, 3-hydroxy-pyrrolidine-1-carboxylic acid tert-butyl ester (3.0 g, 16.0 mmol, 1.0 eq) was added slowly and the mixture was left to stir at room temperature for 10-15 minutes. To the solution of sodium alkoxide in THF was added 2-fluoronitrobenzene (2.49 g, 17.6 mmol, 1.1 eq). The reaction mixture was heated at reflux with stirring for 5 hours. The reaction was then allowed to cool down to room temperature, then water (15 ml) was added to the reaction mixture. The resulting mixture was extracted three times with ethyl acetate (30 ml), the organics dried over sodium sulphate, filtered and the filtrate evaporated to dryness in vacuo. The resultant residue was purified by column chromatography using 40% ethyl acetate/heptane to give the title compound as a yellow solid (3.57 g, 11.58 mmol, 72%). ¹H NMR indicates desired compound in ca. 95% purity.

### Compound 208c: 3-(2-Amino-phenoxy)-pyrrolidine-1-carboxylic acid tert-butyl ester

A suspension of 3-(2-nitro-phenoxy)-pyrrolidine-1-carboxylic acid tert-butyl ester (3.5 g, 11.4 mmol, 1.0 eq) and 10% w/w palladium on carbon (0.35 g, 10% w/w) in ethanol (70 ml) was stirred under a hydrogen atmosphere for 18 hours at room temperature. The mixture was filtered through celite and the solvent removed in vacuo to give the title compound (3.0 g, 10.78 mmol, 95%). ¹H NMR indicates desired compound in ca. 95% purity.

### Compound 208d: 3-[2-(Thieno[2,3-d]pyrimidin-4-ylamino)-phenoxy]-pyrrolidine-1-carboxylic acid tert-butyl ester

A suspension of 3-(2-nitro-phenoxy)-pyrrolidine-1-carboxylic acid tert-butyl ester (1.0 g, 3.6 mmol, 1.0 eq), 4-chloro-thieno[2,3d]pyrimidine (0.61 g, 3.6 mmol, 1.0 eq) and DIPEA (0.74 g, 5.76 mmol, 1.6 eq) in IPA (8 ml) was heated at 120° C. for 5 days. The reaction was allowed to cool to room temperature and the solvent removed in vacuo. The resultant residue was purified by column chromatography using ethyl acetate/cyclohexane [1:1] as eluent to give the title compound (0.64 g, 1.56 mmol, 43%). ¹H NMR indicates desired compound in ca. 95% purity.

### Compound 208e: [2-(Pyrrolidin-3-yloxy)-phenyl]-thieno[2,3-d]pyrimidin-4-yl-amine TFA salt

A solution of 3-[2-(thieno[2,3-d]pyrimidin-4-ylamino)-phenoxy]-pyrrolidine-1-carboxylic acid tert-butyl ester (0.64 g, 1.56 mmol, 1.0 eq) and trifluoroacetic acid (2 ml) in DCM (10 ml) was stirred at room temperature for 18 hours. The solvent was removed in vacuo to give the title compounds as green oil (1.37 g, 1.56 mmol, 100% corrected). LCMS; [M+H]⁺=313, Rt=0.81 min, 100% purity

### Compound 208f: [2-(Pyrrolidin-3-yloxy)-phenyl]-thieno[2,3-d]pyrimidin-4-yl-amine

A solution [2-(Pyrrolidin-3-yloxy)-phenyl]-thieno[2,3-d]pyrimidin-4-yl-amine TFA salt (65 mg, 0.15 mmol, 1.0 eq) in 1M NaOH (2 ml) was extracted with DCM (3×2 ml), the organics combined and the solvent removed in vacuo to give the title compound as yellow oil (21 mg, 0.07 mmol, 45%). LCMS; [M+H]⁺=313, Rt=1.10 min, 100% purity

### Compound 208g: [2-(1-Methanesulfonyl-pyrrolidin-3-yloxy)-phenyl]-thieno[2,3-d]pyrimidin-4-yl-amine

A solution of [2-(pyrrolidin-3-yloxy)-phenyl]-thieno[2,3-d]pyrimidin-4-yl-amine TFA salt (60 mg, 0.14 mmol, 1.0 eq) and DIPEA (73 mg, 0.56 mmol, 4.0 eq) in DCM (2 ml) was stirred at room temperature, methanesulphonyl chloride was added and the reaction stirred for 18 hours at room temperature. The reaction was diluted with 1M NaOH solution (2 ml), the organic layer separated, dried over sodium sulphate, and the solvent removed in vacuo. The resultant residue was purified by semi-preparative HPLC to give the title compound as yellow oil (14.3 mg, 0.04 mmol, 26%). LCMS; [M+H]⁺=391, Rt=1.42 min, 93% purity

The compounds listed below were prepared via route 19;

### Compound 209a: 1-{3-[2-(Thieno[2,3-d]pyrimidin-4-ylamino)-phenoxy]-pyrrolidin-1-yl}-ethanone

Yield; 12.3 mg, 0.03 mmol, 25%

LCMS; [M+H]⁺=355, Rt=1.33 min, 94% purity

### Compound 210a: 3-[2-(Thieno[2,3-d]pyrimidin-4-ylamino)-phenoxy]-pyrrolidine-1-carboxylic acid dimethylamide

Yield; 16 mg, 0.04 mmol, 30%

LCMS; [M+H]⁺=384, Rt=1.43 min, 98% purity

### Compound 211a: {2-[1-(Propane-2-sulfonyl)-pyrrolidin-3-yloxy]-phenyl}-thieno[2,3-d]pyrimidin-4-yl-amine

Yield; 20 mg, 0.05 mmol, 34%

LCMS; [M+H]⁺=419, Rt=1.54 min, 97% purity

### Compound 212a: 3-[2-(Thieno[2,3-d]pyrimidin-4-ylamino)-phenoxy]-pyrrolidine-1-sulfonic acid dimethylamide

Yield; 14 mg, 0.03 mmol, 28%

LCMS; [M+H]⁺=420, Rt=1.54 min, 97% purity

### Compound 213a: 2-Methyl-1-{3-[2-(thieno[2,3-d]pyrimidin-4-ylamino)-phenoxy]-pyrrolidin-1-yl}-propan-1-one

Yield; 10.5 mg, 0.03 mmol, 23%

LCMS; [M+H]⁺=383, Rt=1.05 min, 100% purity

### Compound 214a: Pyridin-3-yl-{3-[2-(thieno[2,3-d]pyrimidin-4-ylamino)-phenoxy]-pyrrolidin-1-yl}-methanone

Yield; 27 mg, 0.07 mmol, 47%

LCMS; [M+H]⁺=418, Rt=1.35 min, 97% purity

### Compound 215a: Pyridin-4-yl-{3-[2-(thieno[2,3-d]pyrimidin-4-ylamino)-phenoxy]-pyrrolidin-1-yl}-methanone

Yield; 24 mg, 0.06 mmol, 49%

LCMS; [M+H]⁺=418, Rt=1.32 min, 98% purity

### Compound 216a: [2-(1-Cyclopropanesulfonyl-pyrrolidin-3-yloxy)-phenyl]-thieno[2,3-d]pyrimidin-4-yl-amine

Yield; 21 mg, 0.05 mmol, 41%

LCMS; [M+H]⁺=417, Rt=1.52 min, 98% purity

### Compound 217a: Cyclopropyl-{3-[2-(thieno[2,3-d]pyrimidin-4-ylamino)-phenoxy]-pyrrolidin-1-yl}-methanone

Yield; 7 mg, 0.02 mmol, 15%

LCMS; [M+H]⁺=381, Rt=1.41 min, 97% purity

### Compound 218a: 3-[2-(Thieno[2,3-d]pyrimidin-4-ylamino)-phenoxy]-pyrrolidine-1-carboxylic acid 4-methoxy-benzylamide

Yield; 116 mg, 0.24 mmol, 52%

LCMS; [M+H]⁺=476, Rt=1.58 min, 98% purity

### Compound 219a: 3-[2-(5-Methyl-thieno[2,3-d]pyrimidin-4-ylamino)-phenoxy]-pyrrolidine-1-carboxylic acid tert-butyl ester

Yield; 28 mg, 0.07 mmol, 9%

LCMS; [M+H]⁺=427, Rt=2.12 min, 97% purity

### Compound 220a: 3-[2-(5,6-Dimethyl-thieno[2,3-d]pyrimidin-4-ylamino)-phenoxy]-pyrrolidine-1-carboxylic acid tert-butyl ester

Yield; 16 mg, 0.04 mmol, 5%

LCMS; [M+H]⁺=441, Rt=2.15 min, 95% purity

### Example 1t

### Synthesis Route 20

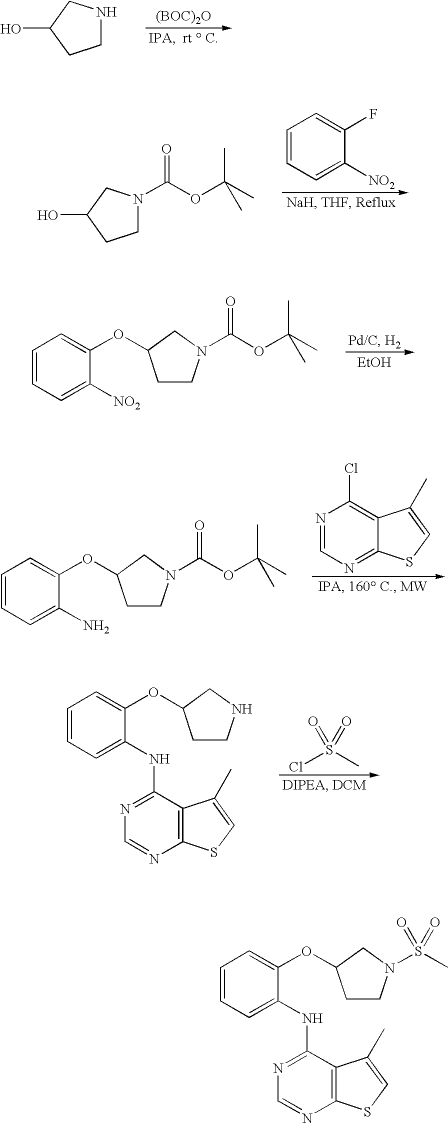

### Compound 221a: 3-(2-Amino-phenoxy)-pyrrolidine-1-carboxylic acid tert-butyl ester

Prepared as per route 19.

### Compound 221 b: (5-Methyl-thieno[2,3-d]pyrimidin-4-yl)-[2-(pyrrolidin-3-yloxy)-phenyl]-amine

A solution of 3-(2-amino-phenoxy)-pyrrolidine-1-carboxylic acid tert-butyl ester (1.51 g, 5.42 mmol, 1.0 eq) and 4-chloro-5-methylthieno[2,3-d]pyrimidine (1.0 g, 5.42 mmol, 1.0 eq) in IPA (20 ml) was heated in a microwave at 160° C. for 45 minutes. The reaction was allowed to cool to room temperature, diluted with water (40 ml), and ammonium hydroxide solution (20 ml) added. The resultant precipitate was isolated by filtration, washed with cyclohexane (2×50 ml), washed with diethyl ether (2×50 ml). The solid was then purified by column chromatography using 10% MeOH/DCM as eluent to give the title compound (0.78 g, 2.4 mmol, 44%). LCMS; [M+H]⁺=327, Rt=1.53 min, 100% purity

### Compound 221c: [2-(1-Methanesulfonyl-pyrrolidin-3-yloxy)-phenyl]-(5-methyl-thieno[2,3-d]pyrimidin-4-yl)-amine

A solution of (5-methyl-thieno[2,3-d]pyrimidin-4-yl)-[2-(pyrrolidin-3-yloxy)-phenyl]-amine (60 mg, 0.18 mmol, 1.0 eq) DIPEA (95 mg, 7.4 mmol, 4.0 eq) in a 1:1 mixture of DCM/DMF (2 ml) was cooled to 0° C. and methanesulphonyl chloride was added. The reaction was stirred at room temperature for 18 hours, diluted with 1M NaOH (2 ml) and extracted with DCM (3×2 ml). The organics were combined, dried over sodium sulphate, and the solvent removed in vacuo. The resultant residue was purified by mass directed preparative HPLC to give the title compound (32 mg, 0.08 mmol, 44%). LCMS; [M+H]⁺=405, Rt=2.12 min, 98% purity

The compounds listed below were prepared via route 20;

### Compound 222a: 1-{3-[2-(5-Methyl-thieno[2,3-d]pyrimidin-4-ylamino)-phenoxy]-pyrrolidin-1-yl}-ethanone

Yield; 41 mg, 0.11 mmol, 61%

LCMS; [M+H]⁺=369, Rt=1.93 min, 93% purity

### Compound 223a: 3-[2-(5-Methyl-thieno[2,3-d]pyrimidin-4-ylamino)-phenoxy]-pyrrolidine-1-carboxylic acid dimethylamide

Yield; 40 mg, 0.10 mmol, 56%

LCMS; [M+H]⁺=398, Rt=2.09 min, 100% purity

### Compound 224a: 2-Methyl-1-{3-[2-(5-methyl-thieno[2,3-d]pyrimidin-4-ylamino)-phenoxy]-pyrrolidin-1-yl}-propan-1-one

Yield; 41 mg, 0.10 mmol, 57%

LCMS; [M+H]⁺=397, Rt=2.15 min, 100% purity

### Compound 225a: Cyclopropyl{3-[2-(5-methyl-thieno[2,3-d]pyrimidin-4-ylamino)-phenoxy]pyrrolidin-1-yl}-methanone

Yield; 45 mg, 0.11 mmol, 63%

LCMS; [M+H]⁺=395, Rt=2.115 min, 100% purity

### Compound 226a: Cyclopentyl-{3-[2-(5-methyl-thieno[2,3-d]pyrimidin-4-ylamino)-phenoxy]-pyrrolidin-1-yl}-methanone

Yield; 36 mg, 0.08 mmol, 47%

LCMS; [M+H]⁺=423, Rt=2.32 min, 100% purity

### Compound 227a: 3-[2-(5-Methyl-thieno[2,3-d]pyrimidin-4-ylamino)-phenoxy]-pyrrolidine-1-sulfonic acid dimethylamide

Yield; 44 mg, 0.10 mmol, 56%

LCMS; [M+H]⁺=434, Rt=2.27 min, 100% purity

### Compound 228a: (5-Methyl-thieno[2,3-d]pyrimidin-4-yl)-{2-[1-(propane-2-sulfonyl)-pyrrolidin-3-yloxy]-phenyl}-amine

Yield; 43 mg, 0.10 mmol, 55%

LCMS; [M+H]⁺=433, Rt=2.28 min, 98% purity

### Compound 229a: {3-[2-(5-Methyl-thieno[2,3-d]pyrimidin-4-ylamino)-phenoxy]-pyrrolidin-1-yl}-pyridin-3-yl-methanone

Yield; 55 mg, 0.13 mmol, 71%

LCMS; [M+H]⁺=432, Rt=1.85 min, 97% purity

### Compound 230a: {3-[2-(5-Methyl-thieno[2,3-d]pyrimidin-4-ylamino)-phenoxy]-pyrrolidin-1-yl}-pyridin-4-yl-methanone

Yield; 29 mg, 0.07 mmol, 37%

LCMS; [M+H]⁺=432, Rt=1.90 min, 99% purity

### Compound 231a: (5,6-Dimethyl-thieno[2,3-d]pyrimidin-4-yl)-[2-(1-methanesulfonyl-pyrrolidin-3-yloxy)-phenyl]-amine

Yield; 34 mg, 0.08 mmol, 28%

LCMS; [M+H]⁺=419, Rt=2.22 min, 94% purity

### Example 1u

### Synthesis Route 21

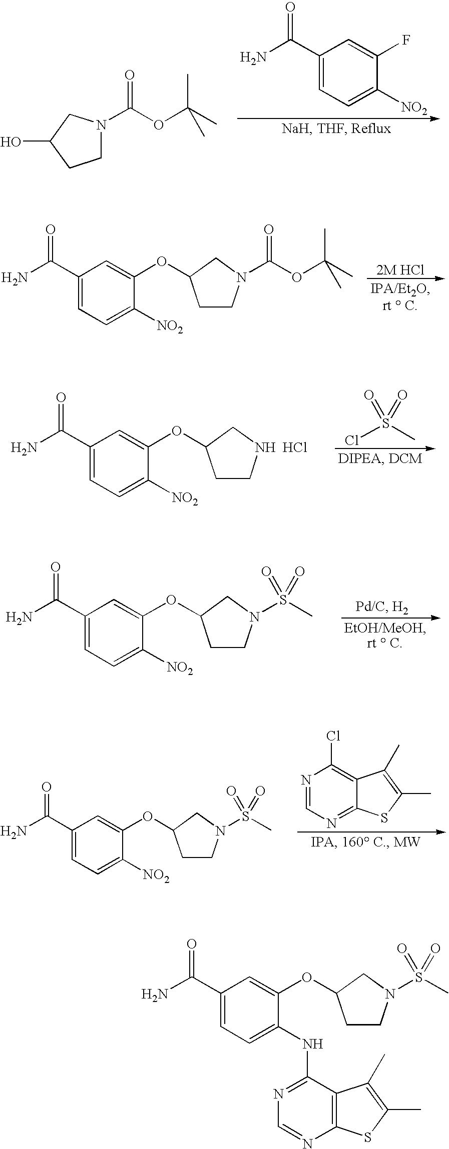

### Compound 232a: 3-(5-Carbamoyl-2-nitro-phenoxy)-pyrrolidine-1-carboxylic acid tert-butyl ester

3-(2-Amino-phenoxy)-pyrrolidine-1-carboxylic acid tert-butyl ester was prepared as per route 19. 3-Fluoro-4-nitro-benzamide was prepared as per route 12.

A solution of 3-(2-amino-phenoxy)-pyrrolidine-1-carboxylic acid tert-butyl ester (2.46 g, 13.14 mmol, 1.2 eq) in THF (10 ml) was cooled to 0° C. and sodium hydride as a 60% dispersion in mineral oil (0.48 g, 11.95 mmol, 1.1 eq) was added, the reaction was stirred at 0° C. for 30 minutes. This was then added drop-wise to a solution of 3-fluoro-4-nitro-benzamide (2.0 g, 10.86 mmol, 1.0 eq) in THF (20 ml) at 0° C. The reaction was stirred at room temperature for 2 hours, diluted with water (20 ml) and extracted with DCM (3×30 ml). The organics were combined, washed with brine, dried over sodium sulphate and the solvent removed in vacuo to give the title compound as a yellow solid (4.25 g, 12.10 mmol, 100% corrected). LCMS; [M+H]⁺=NA, Rt=1.47 min, 100% purity

### Compound 232b: 4-Nitro-3-(pyrrolidin-3-yloxy)-benzamide HCl salt

A 2M solution of HCl in diethyl ether (60 ml, 120.0 mmol, 9.9 eq) was added to a solution of 3-(5-carbamoyl-2-nitro-phenoxy)-pyrrolidine-1-carboxylic acid tert-butyl ester (4.25 g, 12.1 mmol, 1.0 eq) in IPA (60 ml) and the reaction stirred at room temperature for 6 hours. The solvent was removed in vacuo to give the title compound as a yellow solid (3.47 g, 12.1 mmol, 100%). LCMS; [M+H]⁺=252, Rt=1.16 min, 91% purity

### Compound 232c: 3-(1-Methanesulfonyl-pyrrolidin-3-yloxy)-4-nitro-benzamide

A solution of 4-nitro-3-(pyrrolidin-3-yloxy)-benzamide HCl salt (2.54 g, 8.84 mmol, 1.0 eq) and DIPEA (4.57 g, 35.34 mmol, 1.0 eq) in DCM (50 ml) was prepared and methanesulphonyl chloride added (1.01 g, 8.84 mmol, 1.0 eq). The reaction was stirred at room temperature for 18 hours, solvent removed and the resultant residue purified by column chromatography using 5% MeOH/DCM to give the title compound (3.01 g, 9.14 mmol, 88% corrected). LCMS; [M+H]⁺=NA, Rt=1.46 min, 100% purity.

### Compound 232d: 4-Amino-3-(1-methanesulfonyl-pyrrolidin-3-yloxy)-benzamide

A suspension of 3-(1-methanesulfonyl-pyrrolidin-3-yloxy)-4-nitro-benzamide (2.9 g, 8.82 mmol, 1.0 eq) and palladium on carbon (0.30 g, 10% w/w) in 1:1 methanol/ethanol mixture (160 ml) was stirred under a hydrogen atmosphere at room temperature for 18 hours. The reaction was filtered through a celite pad and the solvent removed in vacuo to give the title compound as yellow oil (2.47 g, 8.2 mmol, 93%). LCMS; [M+H]⁺=300, Rt=1.31 min, 100% purity.

### Compound 232e: 3-(1-Methanesulfonyl-pyrrolidin-3-yloxy)-4-(5-methyl-thieno[2,3-d]pyrimidin-4-ylamino)-benzamide

A solution of 4-amino-3-(1-methanesulfonyl-pyrrolidin-3-yloxy)-benzamide (120 mg, 0.40 mmol, 1.0 eq) and 4-chloro-5-methylthieno[2,3-d]pyrimidine (74 mg, 0.40 mmol, 1.0 eq) in IPA (2 ml) was heated at 120° C. for 18 hours. The reaction was allowed to cool to room temperature, diluted with water (4 ml), and ammonium hydroxide solution (4 ml) added. The resultant precipitate was isolated by filtration, washed with water (3×2 ml), washed with cyclohexane (3×2 ml) and dried in vacuo to give the title compound as a brown solid (40 mg, 0.09 mmol, 22%). LCMS; [M+H]⁺=448, Rt=1.83 min, 95% purity.

The compounds listed below were prepared via route 20;

### Compound 233e: 3-(1-Methanesulfonyl-pyrrolidin-3-yloxy)-4-(5,6-dimethyl-thieno[2,3-d]pyrimidin-4-ylamino)-benzamide

Yield; 50 mg, 0.11 mmol, 27%

LCMS; [M+H]⁺=462, Rt=1.91 min, 100% purity

### Example 1v

### Synthesis Route 22

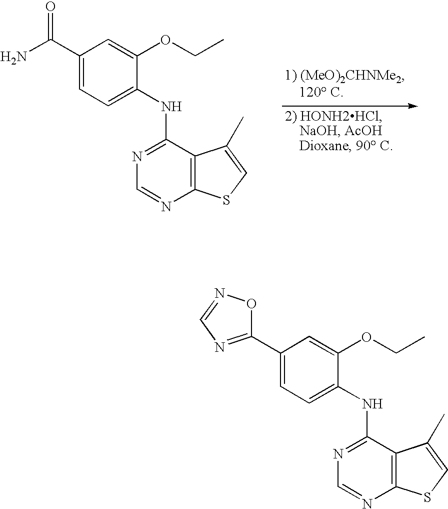

3-Ethoxy-4-(5-methyl-thieno[2,3-d]pyrimidin-4-ylamino)-benzamide prepared as per route 12.

### Compound 234a: (2-Ethoxy-4-[1,2,4]oxadiazol-5-yl-phenyl)-(5-methyl-thieno[2,3-d]pyrimidin-4-yl)-amine

A solution of 3-ethoxy-4-(5-methyl-thieno[2,3-d]pyrimidin-4-ylamino)-benzamide (0.2 g, 0.61 mmol, 1.0 eq) in N,N-dimethylformamide diemethylacetal (1 ml) was heated at 120° C. for 2 hours, allowed to cool to room temperature, the solvent was removed in vacuo. The resultant residue was dissolved in dioxane (2 ml) and the solution was added to a solution of hydroxylamine hydrochloride (51 mg, 0.73 mmol, 1.2 eq), 5M sodium hydroxide solution (0.15 ml, 0.73mmol, 1.2 eq) and acetic acid. The reaction was heated at 90° C. for 1 hour. The reaction mixture was allowed to cool to room temperature and the resultant precipitate was isolated by filtration, washed with cyclohexane, and dried in vacuo. The resultant solid was purified by semi-preparative HPLC, followed by column chromatography using 1% MeOH/DCM to give the title compound as a white solid (32 mg, 0.9 mmol, 15%). LCMS; [M+H]⁺=354, Rt=2.58 min, 89% purity.

### Example 1w

### Synthesis Route 23

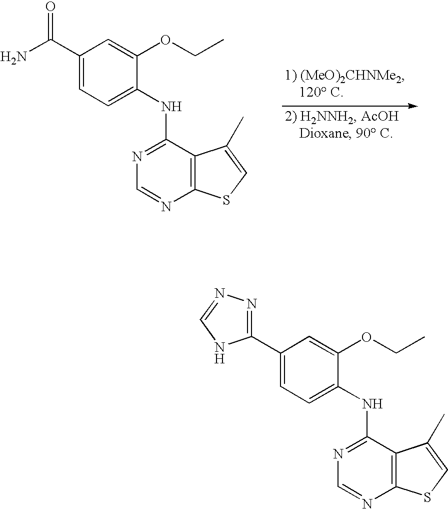

3-Ethoxy-4-(5-methyl-thieno[2,3-d]pyrimidin-4-ylamino)-benzamide prepared as per route 12.

### Compound 235a: [2-Ethoxy-4-(4H-[1,2,4]triazol-3-yl)-phenyl]-(5-methyl-thieno[2,3-d]pyrimidin-4-yl)-amine

A solution of 3-ethoxy-4-(5-methyl-thieno[2,3-d]pyrimidin-4-ylamino)-benzamide (0.2 g, 0.61 mmol, 1.0 eq) in N,N-dimethylformamide diemethylacetal (2 ml) was heated at 120° C. for 2 hours, allowed to cool to room temperature, the solvent was removed in vacuo. The resultant residue was added to a solution of hydrazine monohydrate (34 mg, 0.67 mmol, 1.1 eq) in acetic acid (2 ml) and heated at 90° C. for 1.5 hours. The reaction was allowed to cool to room temperature and the solvent was removed in vacuo. The resultant solid was triturated in a 1:1 mixture of IPA and diethyl ether (20 ml), the precipitate isolated by filtration, washed with diethyl ether (2×15 ml) and dried in vacuo to give the title compound as a grey solid (159 mg, 0.45 mmol, 74%). LCMS; [M+H]⁺=353, Rt=1.93 min, 100% purity.

### Example 1x

### Synthesis Route 24

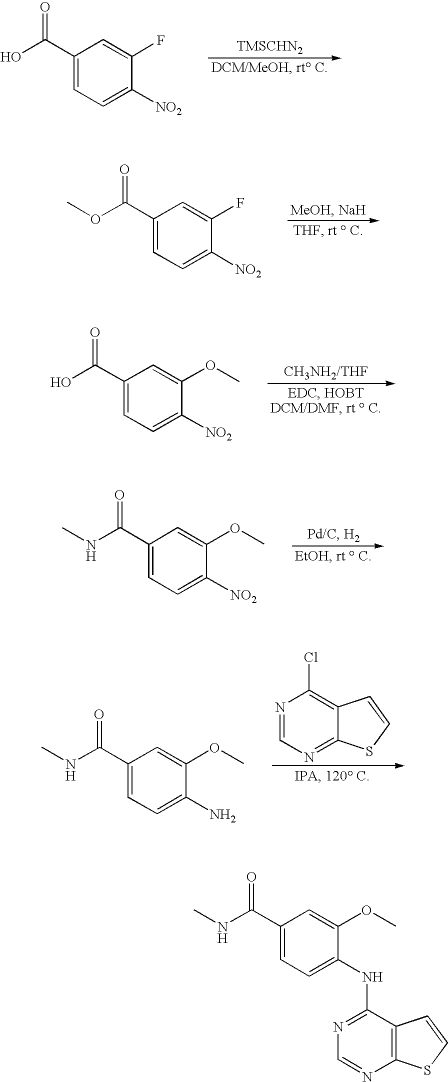

### Compound 236a: 3-Fluoro-4-nitro-benzoic acid methyl ester

A solution of 3-fluoro-4-nitro-benzoic acid (3.0 g, 16.12 mmol, 1.0 eq) in 4:1 DCM/MeOH (50 ml) was stirred at room temperature for 5 minutes and a 2.0M solution of TMS-diazomethane in hexanes (8.1 ml, 16.12 mmol, 1.0 eq) was added drop-wise over 10 minutes, the reaction then stirred at room temperature for 30 minutes. The reaction was quenched with a few drops of acetic acid and the solvent removed in vacuo to give the title compound (3.4 g, 17.09 mmol, 100% corrected). ¹H NMR shows the desired product in ca. 90% purity.

### Compound 236b: 3-Methoxy-4-nitro-benzoic acid

A solution of methanol (0.18g, 5.5 mmol, 1.1 eq) in THF (10 ml) was added drop-wise to sodium hydride as a 60% dispersion in mineral oil (0.22 g, 9.2 mmol, 1.8 eq) whilst being cooled to 0° C. The reaction stirred for 15 minutes, a solution of 3-fluoro-4-nitro-benzoic acid methyl ester (1.0 g, 5.0 mmol, 1.0 eq) in THF (10 ml) was added and the reaction stirred at room temperature for 1 hour. The reaction had not gone to completion so a solution of methanol (0.18 g, 5.5 mmol, 1.1 eq) and sodium hydride as a 60% dispersion in mineral oil (0.22 g, 9.2 mmol, 1.8 eq) in THF (10 ml) was prepared and added to the reaction mixture. The reaction was stirred for at room temperature for an additional hour. The reaction was diluted with water (20 ml), extracted with ethyl acetate (2×20 ml); extracted with DCM (20 ml), the organics combined, dried over sodium sulphate, and the solvent removed in vacuo. The aqueous layer was separated, the solvent removed and the resultant residue purified by column chromatography using 20% ethyl acetate/cyclohexane as eluent to give the title compound (0.89 g, 4.5 mmol, 82%). ¹H NMR shows product in ca. 95% purity.

### Compound 236c: 3-Methoxy-N-methyl-4-nitro-benzamide

A solution of 3-methoxy-4-nitro-benzoic acid (0.24 g, 1.2 mmol, 1.0 eq), EDC (0.37 g, 2.4 mmol, 2.0 eq) and HOBT (0.32 g, 2.4 mmol, 2.0 eq) in DMF (5 ml) was stirred at room temperature for 15 minutes, methylamine as a 2.0M solution in THF (1.2 ml, 2.4 mmol, 2.0 eq) was added. The reaction was stirred at room temperature for 18 hours, the solvent was removed in vacuo, and the resultant residue was purified by column chromatography using 10% ethyl acetate/heptane as eluent to give the title compound (0.21 g, 1.0 mmol, 83%). ¹H NMR indicates desired product in ca. 95% purity.

### Compound 236d: 4-Amino-3-methoxy-N-methyl-benzamide

A suspension of 3-methoxy-N-methyl-4-nitro-benzamide (0.21 g, 1.0 mmol, 1.0 eq) and 10% palladium on carbon (21 mg, 10% w/w) in ethanol (10 ml) was stirred under a hydrogen atmosphere at room temperature for 18 hours. The reaction mixture was filtered through a celite pad, the solvent removed in vacuo to give the title compound (174 mg, 0.97 mmol, 97%). ¹H NMR shows desired product in ca. 95% purity.

### Compound 236e: 3-Methoxy-N-methyl-4-(thieno[2,3-d]pyrimidin-4-ylamino)-benzamide

A solution of 4-amino-3-methoxy-N-methyl-benzamide (35 mg, 0.19 mmol, 1.0 eq) and 4-chlorothieno[3,2-d]pyrimidine (33 mg, 0.19 mmol, 1.0 eq) in IPA (2 ml) was heated at 120° C. for 16 hours. The reaction was allowed to cool to room temperature, diluted with water (4 ml), ammonium hydroxide solution (1 ml) added, and the resultant precipitate isolated by filtration, washed with cyclohexane (2×5 ml), washed with diethyl ether (2×5 ml), then dried in vacuo. The solid was purified by column chromatography to using 5% MeOH/DCM to give the title compound (34 mg, 0.11 mmol, 57%). LCMS; [M+H]⁺=315, Rt=1.69 min, 100% purity

The compounds listed below were prepared via route 24;

### Compound 237a: 3-Methoxy-N-methyl-4-(5-methyl-thieno[2,3-d]pyrimidin-4-ylamino)-benzamide

Yield; 38 mg, 0.11 mmol, 59%

LCMS; [M+H]⁺=329, Rt=1.95 min, 100% purity

### Compound 238a: 4-(5,6-Dimethyl-thieno[2,3-d]pyrimidin-4-ylamino)-3-methoxy-N-methyl-benzamide

Yield; 33 mg, 0.09 mmol, 49%

LCMS; [M+H]⁺=343, Rt=2.06 min, 100% purity

### Compound 239a: 3-Methoxy-N,N-dimethyl-4-(thieno[2,3-d]pyrimidin-4-ylamino)-benzamide

Yield; 24 mg, 0.07 mmol, 32%

LCMS; [M+H]⁺=329, Rt=1.69 min, 100% purity

### Compound 240a: 3-Methoxy-N,N-dimethyl-4-(5-methyl-thieno[2,3-d]pyrimidin-4-ylamino)-benzamide Yield; 5.9 mg, 0.02 mmol, 8%

LCMS; [M+H]⁺=343, Rt=1.98 min, 100% purity

### Example 1y

### Synthesis Route 25

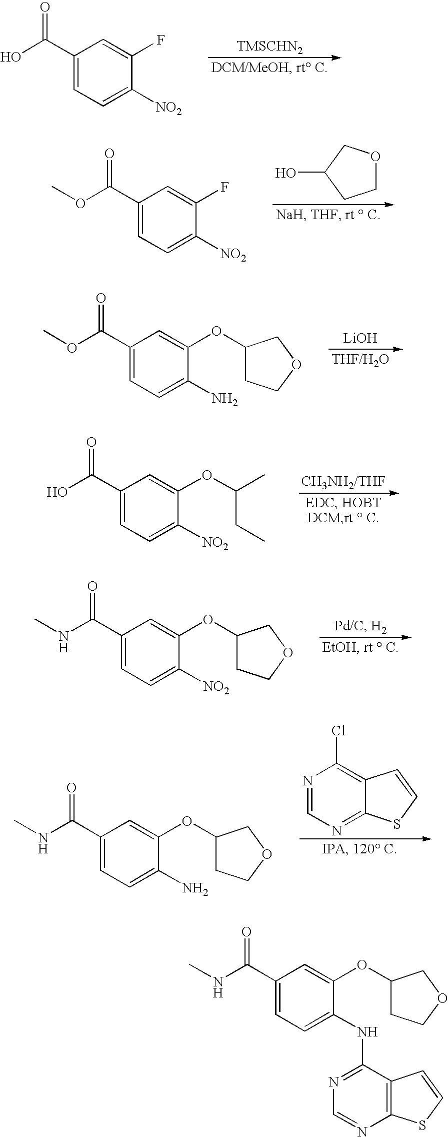

### Compound 241a: 3-Fluoro-4-nitro-benzoic acid methyl ester (Prepared as per route 24)

### Compound 241 b: 4-Nitro-3-(tetrahydro-furan-3-yloxy)-benzoic acid methyl ester

A solution of 3-hydroxytetrahydrofuran (0.23 g, 2.59 mmol, 1.1 eq) in THF (5 ml) was added drop-wise to sodium hydride as a 60% dispersion in mineral oil (0.10 g, 4.33 mmol, 1.8 eq) whilst being cooled to 0° C. The reaction stirred for 15 minutes, a solution of 3-fluoro-4-nitro-benzoic acid methyl ester (0.47 g, 2.36 mmol, 1.0 eq) in THF (5 ml) was added and the reaction stirred at room temperature for 1 hour. The reaction was diluted with water (15 ml), extracted with ethyl acetate (3×25 ml), the organics combined, dried over sodium sulphate, and the solvent removed in vacuo. The resultant residue purified by column chromatography using 20% ethyl acetate/cyclohexane as eluent to give the title compound (0.11 g, 0.4 mmol, 18%). ¹H NMR shows product in ca. 95% purity.

### Compound 241c: 4-Nitro-3-(tetrahydro-furan-3-yloxy)-benzoic acid

A solution of 4-nitro-3-(tetrahydro-furan-3-yloxy)-benzoic acid methyl ester (100 mg, 0.37 mmol, 1.0 eq) and lithium hydroxide (18 mg, 0.75 mmol, 2.0 eq) in 2:1 THF/water (3 ml) was stirred at room temperature for 3 hours. The solvent was removed in vacuo to give the title compound (82 mg, 0.32 mmol, 88%). ¹H NMR shows product in ca. 95% purity.

### Compound 241d: 4-Nitro-3-(tetrahydro-furan-3-yloxy)-N-methyl-benzamide

A solution of 4-Nitro-3-(tetrahydro-furan-3-yloxy)-benzoic acid (82 mg, 0.32 mmol, 1.0 eq), EDC (47 mg, 0.64 mmol, 2.0 eq) and HOBT (43 mg, 0.64 mmol, 2.0 eq) in DCM (5 ml) was stirred at room temperature for 15 minutes, methylamine as a 2.0M solution in THF (0.32 ml, 0.64 mmol, 2.0 eq) was added. The reaction was stirred at room temperature for 18 hours, the solvent was removed in vacuo, and the resultant residue was purified by column chromatography using 7% MeOH/DCM as eluent to give the title compound (84 mg, 0.32 mmol, 98%). ¹H NMR indicates desired product in ca. 95% purity.

### Compound 241e: 4-Amino-3-(tetrahydro-furan-3-yloxy)-N-methyl-benzamide

A suspension of 4-nitro-3-(tetrahydro-furan-3-yloxy)-N-methyl-benzamide (84 mg, 0.32 mmol, 1.0 eq) and 10% palladium on carbon (8.4 mg, 10% w/w) in ethanol (10 ml) was stirred under a hydrogen atmosphere at room temperature for 18 hours. The reaction mixture was filtered through a celite pad, the solvent removed in vacuo to give the title compound (68 mg, 0.29 mmol, 90%). ¹H NMR shows desired product in ca. 95% purity.

### Compound 241f: N-Methyl-3-(tetrahydro-furan-3-yloxy)-4-(thieno[2,3-d]pyrimidin-4-ylamino)-benzamide

A solution of 4-amino-3-(tetrahydro-furan-3-yloxy)-N-methyl-benzamide (20 mg, 0.08 mmol, 1.0 eq) and 4-chlorothieno[3,2-d]pyrimidine (14 mg, 0.08 mmol, 1.0 eq) in IPA (2 ml) was heated at 120° C. for 3 hours. The reaction was allowed to cool to room temperature, diluted with water (2 ml), ammonium hydroxide solution (0.5 ml) added, the mixture extracted with ethyl acetate (2×5 ml), extracted with DCM (2×5 ml), the organics combined, dried over sodium sulphate and the solvent removed in vacuo. The resultant residue was purified by column chromatography to using 5% MeOH/DCM to give the title compound (4.1 mg, 0.01 mmol, 14%). LCMS; [M+H]⁺=371, Rt=1.67 min, 93% purity

The compounds listed below were prepared via route 25;

### Compound 242a: 4-(5,6-Dimethyl-thieno[2,3-d]pyrimidin-4-ylamino)-N-methyl-3-(tetrahydro-furan-3-yloxy)-benzamide

Yield; 0.8 mg, 0.002 mmol, 2%

LCMS; [M+H]⁺=399, Rt=2.01 min, 98% purity

### Example 2

### Kinase Fluorescence Polarization Assays

Assay principle: Inhibitory potency of compounds against Mnk1, Mnk2a and other kinases was assessed with assays based on a format known to those skilled in the art as the indirect (competitive) fluorescence polarization. The assay detection system comprises a small fluorophore-labeled phospho-peptide (termed ligand) bound to a phospho-specific antibody. The product generated by the kinase reaction competes with the ligand for antibody binding. Based on the larger molecular volume of the bound ligand, which results in a lower rotation rate in solution, its emitted light has a higher degree of polarization than the one from the free ligand.

Description of the Specific Homogenous Kinase Assay

### Example 2a

### Mnk1 and Mnk2a in vitro Kinase Assay

As a source of enzyme, human Mnk1 and human Mnk2a were expressed as GST fusion proteins in E. coli, purified to >80% homogeneity by glutathione affinity chromatography and activated in vitro with pre-activated ERK2. In brief, the open reading frames of human Mnk1 and Mnk2a were amplified from cDNA using the forward/reverse primer pairs

| | |
|---|---|
| SEQ ID NO: 1 | 5′TTTAGGATCCGTATCTTCTCAAAAGTTGG/ |
| SEQ ID NO: 2 | 5′ CTGGGTCGACTCAGAGTGCTGTGGGCGG |
| and | |
| SEQ ID NO: 3 | 5′ACAGGGATCCGTGCAGAAGAAACCAGCC/ |
| SEQ ID NO: 4 | 5′GATGGTCGACTCAGGCGTGGTCTCCCACC |

(utilized restriction sites underlined), respectively, and cloned into the BamHI and SalI sites of the vector pGEX-4T1 (Amersham, Sweden, cat. no. 27-4580-01). These constructs allow prokaryotic expression of Mnk1 or Mnk2a as fusion protein with a N-terminal glutathione S-transferase (GST) tag, referred to as GST-Mnk1 or GST-Mnk2a. The following expression and purification procedure was identical for GST-Mnk1 and GST-Mnk2a, referring in general to GST-Mnk, when not distinguishing between the two isoforms. Expression of GST-Mnk was in E. coli BL21 (Merck Biosciences, Germany, cat. no. 69449). Cells were grown in LB-Bouillon (Merck, Germany, cat. no. 1.10285) supplemented with 100 μg/ml ampicillin (Sigma, Germany, cat. no. A9518) at 37° C. When the culture had reached a density corresponding to an A₆₀₀ of 0.8, an equal volume of ice cold LB/ampicillin was added, the culture transferred to 25° C. and induced for 4 h with 1 mM isopropyl thiogalactoside (IPTG, Roth, Germany, cat. no. 2316.4). Cells harvested by centrifugation were resuspended in 10 ml lysis buffer (50 mM tris(hydroxymethyl)aminomethane hydrochloride (Tris/HCl, Sigma, Germany, cat. no. T5941) pH 7.5, 300 mM sodium chloride (NaCl, Sigma, Germany, cat. no. S7653), 5% (w/v) glycerol (Sigma, Germany, cat. no. G5516), 3 mM DTT dithiotreitol (DTT, Sigma, Germany, cat. no. D9779)) per gram wet weight cell pellet. Lysates were prepared by disruption of cells with a sonifier and subsequent clearing by centrifugation at 38000 g for 45 min at 4° C.

The lysate was applied to a GSTPrep FF 16/10 column (Amersham, Sweden, cat. no. 17-5234-01) equilibrated with lysis buffer. Removal of unbound material was with 3 column volumes (CV) lysis buffer. Elution was with 2 CV of elution buffer (50 mM Tris/HCl pH 7.5, 300 mM NaCl, 5% (w/v) glycerol, 20 mM glutathione (Sigma, Germany, cat. no. G4251)). Peak fractions were pooled and the protein transferred into storage buffer (50 mM Tris/HCl pH 7.5, 200 mM NaCl, 0.1 mM ethylene glycol-bis(2-aminoethylether)-N,N,N′,N′-tetraacetic acid (EGTA, Aldrich, Germany, cat. no. 23,453-2), 1 mM DTT, 10% (w/v) glycerol, 0.5 M sucrose (Sigma, Germany, cat. no. S0389) by gel filtration on a PD10 desalting column (Amersham, Sweden, cat. no. 17-0851-01). Aliquots were shock frozen in liquid nitrogen and stored at −80° C.

Activation of Mnk1 and Mnk2a was at a concentration of 2.5 μM of either purified GST-Mnk1 or GST-Mnk2a by incubation with 150 nM pre-activated NHis-ERK2 (see ERK2 assay for preparation) and 50 μM adenosine triphosphate (ATP, Sigma, cat. no. A2699) in a buffer comprising 20 mM N-(2-hydroxyethyl)piperazine-N′-(2-ethanesulfonic acid) (HEPES, Fluka, Germany, cat. no 54459)/potassium hydroxide (KOH, Roth, Germany, cat. no 6751.1) pH 7.4, 10 mM magnesium chloride (MgCl₂, Sigma, Germany, cat. no. M2670), 0.25 mM DTT, 0.05% (w/v) polyoxyethylene 20 stearylether (Brij 78, Sigma, Germany, cat. no. P4019) (HMDB buffer) for 45 min at 30° C. After the incubation, the preparation was aliquoted into single-use samples, shock frozen in liquid nitrogen, stored at −80° C. and utilized for Mnk1 or Mnk2a kinase assays as detailed below. The presence of activating kinase has been tested to not interfere with the Mnk activity assay.

SUBSTRATE: A carboxy-terminal amidated 12 mer peptide with the sequence

| | |
|---|---|
| SEQ ID NO: 5 | TATKSGSTTKNR, |

derived from the amino acid sequence around serine 209 of the eukaryotic translation initiation factor 4E (eIF4E) has been synthesized and purified by high performance liquid chromatography (HPLC) to >95% (Thermo, Germany). The serine residue phosphorylated by Mnk kinases is underlined.

LIGAND: The peptide TATKSG-pS-TTKNR, containing an amidated carboxy-terminus and conjugated at the amino-terminus with the oxazine derived fluorophore depicted below was synthesized and used as ligand.

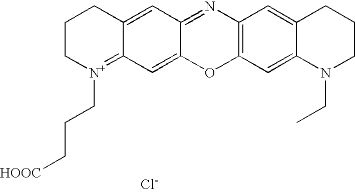

ANTIBODY: SPF New Zealand White Rabbits have been immunized according to standard protocols with the peptide NH2-CTATKSG-pS-TTKNR-CONH2, coupled to keyhole limpet hemocyanin (KLH). The immune globulin G (IgG) fraction was purified from serum of boosted animals by techniques known in the art. In brief, serum was subjected to protein A affinity chromatography. Eluted material was precipitated at 50% cold saturated ammonium sulfate, pellets dissolved and desalted. The resulting material was appropriate for use in below described assay without further antigen-specific purification.

ASSAY SETUP: Inhibition of kinase activity of Mnk1 and Mnk2a was assessed with the same assay system, using pre-activated GST-Mnk1 or GST-Mnk2a, respectively. The kinase reaction contains 30 μM substrate peptide, 20 μM ATP, 60 nM ligand and one of either 25 nM pre-activated Mnk1 or 2.5 nM pre-activated Mnk2a. The reaction buffer conditions are 16 mM HEPES/KOH pH 7.4, 8 mM MgCl₂, 0.4 mM DTT, 0.08% (w/v) bovine serum albumin (BSA, Sigma, Germany, cat. no. A3059), 0.008% (w/v) Pluronic F127 (Sigma, Germany, cat. no. P2443), 3% (v/v) DMSO (Applichem, Germany, cat. no. A3006). The kinase reaction is at 30° C. for 40 min. The kinase reaction is terminated by addition of 0.67 reaction volumes of 1 μM antibody in 20 mM HEPES/KOH pH 7.4, 50 mM ethylenediaminetetraacetic acid, disodium salt (EDTA, Sigma, Germany, cat. no. E5134), 0.5 mM DTT, 0.05% (w/v) polyoxyethylene-sorbitan monolaureate (Tween 20, Sigma, Germany, cat. no. P7949). After 1 h equilibration time at room temperature, samples are subjected to fluorescence polarization measurement. The fluorescence polarization readout was generated on an Analyst AD multimode reader (Molecular Devices, Sunnyvale, Calif., USA) equipped with a DLRP650 dichroic mirror (Omega Opticals, Brattleboro, Vt., USA, cat. no. XF2035), a 630AF50 band pass filter (Omega Opticals, Brattleboro, Vt., USA, cat. no. XF1069) on the excitation and a 695AF55 band pass filter on the emission side (Omega Opticals, Brattleboro, Vt., USA, cat. no. XF3076).

### Example 2b

### ERK2 in vitro Kinase Assay

KINASE: As a source of enzyme, human ERK2 was expressed as N-terminal hexa-histidin fusion protein in E. coil, purified to >80% homogeneity by immobilized metal ion affinity chromatography (IMAC) and activated in vitro with a constitutively active mutant of MEK1.

In brief, the open reading frame of human ERK2 was amplified from cDNA using the forward/reverse primer pair

| | |
|---|---|
| SEQ ID NO: 6 | 5′AGCCGTCGACGCGGCGGCGGCGGCGGCGGGC/ |
| SEQ ID NO: 7 | 5′TGACAAGCTTAAGATCTGTATCCTGGCTGG |

(utilized restriction sites underlined) and cloned into the SalI and HindIII sites of the vector pQE81L (Qiagen, Germany, cat. no. 32923). This construct allows prokaryotic expression of ERK2 as fusion protein with a N-terminal hexa-histidin tag, referred to as NHis-ERK2. Expression of NHis-ERK2 was in E. coli BL21. Cells were grown in LB-Bouillon supplemented with 100 μg/ml ampicillin at 37° C. When the culture had reached a density corresponding to an A₆₀₀ of 0.8, an equal volume of ice cold LB/ampicillin was added, the culture transferred to 25° C. and induced for 4 h with 1 mM IPTG. Cells harvested by centrifugation were resuspended in 10 ml lysis buffer (50 mM Tris/HCl pH 7.5, 300 mM NaCl, 5% (w/v) glycerol, 10 mM β-mercapto ethanol (Sigma, Germany, cat. no. M3148) per gram wet weight cell pellet. Lysates were prepared by disruption of cells with a sonifier and subsequent clearing by centrifugation at 38000 g for 45 min at 4° C.

The lysate was applied to a column containing 25 ml Ni-NTA Superflow matrix (Qiagen, Germany, cat. no. 1018611) equilibrated with lysis buffer. Removal of unbound material was with 3 column volumes (CV) wash buffer (50 mM Tris/HCl pH 7.5, 300 mM NaCl, 5% (w/v) glycerol, 10 mM β-mercapto ethanol, 20 mM imidazol (Sigma, Germany, cat. no. I2399)/HCl pH 7.5). Elution was with 2 CV of elution buffer (50 mM Tris/HCl pH 7.5, 300 mM NaCl, 5% (w/v) glycerol, 300 mM imidazol). Peak fractions were pooled and the protein transferred into storage buffer (50 mM Tris/HCl pH 7.5, 200 mM NaCl, 0.1 mM EGTA, 1 mM DTT, 10% (w/v) glycerol, 0.5 M sucrose) by gel filtration on a PD10 desalting column. Aliquots were shock frozen in liquid nitrogen and stored at −80° C.

The open reading frame of human MEK1 was amplified from cDNA using the forward/reverse primer pair

| | |
|---|---|
| SEQ ID NO: 8 | 5′GTCCGGATCCCCCAAGAAGAAGCCGACGCCC |
| SEQ ID NO: 9 | 5′ TCCCGTCGACTTAGACGCCAGCAGCATGGG |

(utilized restriction sites underlined) and cloned into the BamHI and SalI sites of the vector pQE80L (Qiagen, Germany, cat. no. 32923). By techniques known in the art, the serine codons 212 and 214 were mutagenized to encode aspartate and glutamate. The resulting expression construct is referred to as NHis-MEK1 SSDE. This construct allows prokaryotic expression of MEK1 as a constitutively active mutant. NHis-MEK1 SSDE was expressed and purified under the conditions described for NHis-ERK2.

Activation of NHis-ERK2 was at a concentration of 11.3 μM of purified enzyme by incubation with 1 μM NHis-MEK1 SSDE and 100 μM ATP in a buffer comprising 20 mM HEPES/KOH pH 7.4, 10 mM MgCl₂, 0.25 mM DTT, 0.05% (w/v) Brij 78 (HMDB buffer) for 20 min at 30° C. After the incubation, the preparation was aliquoted into single-use samples, shock frozen in liquid nitrogen, stored at −80° C. and utilized for ERK2 kinase assay as detailed below and for activation of Mnk1 and Mnk2a as described above. The presence of MEK1 SSDE has been tested to not interfere with the ERK2 activity assay.

SUBSTRATE: A carboxy-terminal amidated 17 mer peptide with the sequence

| | |
|---|---|
| SEQ ID NO: 10 | FFKNIVTPRTPPPSQGK |

(synthesis by Thermo, Germany), derived from the amino acid sequence around threonine 98 of the myelin basic protein (MBP) has been synthesized and purified by HPLC to >95%. The relevant residue phosphorylated by ERK2 is underlined.

LIGAND: The peptide KNIVTPR-pT-PPPS, containing an amidated carboxy-terminus and conjugated at the amino-terminus with the fluorophore 5-carboxytetramethylrhodamine (5-TAMRA) was purchased from Thermo (Germany) and used as ligand.

ANTIBODY: Anti-phospho-MBP antibody (clone P12) was purchased from Upstate, Waltham, Mass., USA (cat. no. 05-429).

ASSAY SETUP: The kinase reaction contains 60 μM substrate peptide, 10 μM ATP and 30 nM pre-activated NHis-ERK2. The reaction buffer conditions are 16 mM HEPES/KOH pH 7.4, 8 mM MgCl₂, 0.4 mM DTT, 0.08% (w/v) BSA, 0.008% (w/v) Pluronic F127, 3% (v/v) DMSO.

The kinase reaction is at 30° C. for 40 min. The kinase reaction is terminated by addition of 0.67 reaction volumes of 5 nM ligand and 50 nM antibody in 20 mM HEPES/KOH pH 7.4, 50 mM EDTA, 0.5 mM DTT, 0.05% (w/v) Tween 20. After 30 min equilibration time at room temperature, samples are subjected to fluorescence polarization measurement. The fluorescence polarization readout was generated on an Analyst AD multimode reader (Molecular Devices, Sunnyvale, Calif., USA) equipped with a 561 nm dichroic mirror (Molecular Devices, Sunnyvale, Calif., USA, cat. no. 42-000-0048), a 550/10 nm band pass filter (Molecular Devices, Sunnyvale, Calif., USA, cat. no. 42-000-0130) on the excitation and a 580/10 nm band pass filter (Molecular Devices, Sunnyvale, Calif., USA , cat. no. 42-000-0034) on the emission side.

### Example 2c

### MAPKAP-K2 in vitro Kinase Assay

KINASE: Human, pre-activated MAPKAP-K2 has been purchased from Upstate, Waltham, Mass., USA (cat. no. 14-337).

SUBSTRATE: A carboxy-terminal amidated 17 mer peptide with the sequence

| | |
|---|---|
| SEQ ID NO: 11 | APAYSRALSRQLSSGVS, |

derived from the amino acid sequence around serine 78 of the heat-shock protein 27 (HSP27) has been synthesized and purified by HPLC to >95% (Thermo, Germany). The residue phosphorylated by MAPKAP-K2 is underlined.

LIGAND: The peptide YSRAL-pS-RQLSS, containing an amidated carboxy-terminus and conjugated at the amino-terminus with the fluorophore 5-carboxytetramethylrhodamine (5-TAMRA) was purchased from Thermo (Germany) and used as ligand.

ANTIBODY: Anti-phospho-HSP27 antibody (clone JBW502) was purchased from Upstate, Waltham, Mass., USA (cat. no. 05-645).

ASSAY SETUP: The kinase reaction contains 3 μM substrate peptide, 10 μM ATP and 0.5 nM MAPKAP-K2. The reaction buffer conditions are 16 mM HEPES/KOH pH 7.4, 8 mM MgCl₂, 0.4 mM DTT, 0.08% (w/v) BSA, 0.008% (w/v) Pluronic F127, 3% (v/v) DMSO. The kinase reaction is at 30° C. for 30 min. The kinase reaction is terminated by addition of 0.67 reaction volumes of 12.5 nM ligand and 25 nM antibody in 20 mM HEPES/KOH pH 7.4, 50 mM EDTA, 0.5 mM DTT, 0.05% (w/v) Tween 20. After 30 min equilibration time at room temperature, samples are subjected to fluorescence polarization measurement. The fluorescence polarization readout was generated on an Analyst AD multimode reader (Molecular Devices) with a filter setup as described for the ERK2 assay.

### Example 2d

### EGFR in vitro Kinase Assay

KINASE: Human EGFR has been purchased from Sigma, Germany (cat. no. E3614).

SUBSTRATE: Poly(Glu, Tyr) purchased from Sigma, Germany (cat. no. P0275) has been employed as kinase substrate.

LIGAND: Ligand was from the Tyrosine Kinase Assay Kit, Green (Invitrogen, Germany, cat. no. P2837), supplied as 10fold concentrate.

ANTIBODY: Phospho-tyrosine specific antibody was from the Tyrosine Kinase Assay Kit, Green (Invitrogen, Germany, cat. no. P2837), supplied as 10fold concentrate.

ASSAY SETUP: The kinase reaction contains 3 μg/ml poly(Glu, Tyr), 3 μM ATP and 10 nM EGFR. The reaction buffer conditions are 20 mM HEPES/KOH pH 7.4, 5 mM MgCl₂, 2 mM manganese chloride (MnCl₂, Roth, Germany, cat. no. T881.1), 0.25 mM DTT, 0.03% Tween 20, 50 μM sodium orthovanadate (Na₃VO₄, Sigma, Germany, cat. no. S6508), 3% (v/v) DMSO. The kinase reaction is at 22° C. for 30 min. The kinase reaction is terminated by addition of 0.67 reaction volumes of 2.5 fold concentrated ligand and 2.5 fold concentrated antibody in 25 mM HEPES/KOH pH 7.4, 100 mM EDTA, 0.3 mM DTT, 0.05% (w/v) Tween 20. After 30 min equilibration time at room temperature, samples are subjected to fluorescence polarization measurement. The fluorescence polarization, readout was generated on an Analyst AD multimode reader (Molecular Devices, Sunnyvale, Calif., USA) equipped with a 505 nm dichroic mirror (Molecular Devices, Sunnyvale, Calif., USA, cat. no. 42-000-0033), a 485/20 nm band pass filter (Molecular Devices, Sunnyvale, Calif., USA, cat. no. 42-000-0031) on the excitation and a 530/10 nm band pass filter (Molecular Devices, Sunnyvale, Calif., USA , cat. no. 42-000-0140) on the emission side.

### Example 2e

### CDK2 in vitro Kinase Assay

KINASE: Active human CDK2/cyclinE has been purchased from Upstate, Waltham, Mass., USA (cat. no. 14-475).

SUBSTRATE: RB^{ING} peptide purchased from Invitrogen, Germany (cat. no. P2939) has been employed as kinase substrate.

LIGAND: Ligand was from the CDK RB^{ING} Kinase Assay Kit (Invitrogen, Germany, cat. no. P2929), supplied as 10 fold concentrate.

ANTIBODY: Phospho-specific antibody was from the CDK RB^{ING} Kinase Assay Kit (Invitrogen, Germany, cat. no. P2929), supplied as 4 fold concentrate.

ASSAY SETUP: The kinase reaction contains 2 μM RB^{ING} peptide, 1.66 fold concentrated tracer, 20 μM ATP and 0.36 μg/ml CDK2. The reaction buffer conditions are 16 mM HEPES/KOH pH 7.4, 8 mM MgCl₂, 0.4 mM DTT, 0.08% (w/v) BSA, 0.008% (w/v) Pluronic F127, 3% (v/v) DMSO. The kinase reaction is at 30° C. for 40 min. The kinase reaction is terminated by addition of 0.67 reaction volumes of 2.5 fold conc. antibody in 20 mM HEPES/KOH pH 7.4, 50 mM EDTA, 0.5 mM DTT, 0.05% (w/v) Tween 20. After 30 min equilibration time at room temperature, samples are subjected to fluorescence polarization measurement. The fluorescence polarization readout was generated on an Analyst AD multimode reader (Molecular Devices) with a filter setup as described for the EGFR assay.

1. A compound of the general formula (1) 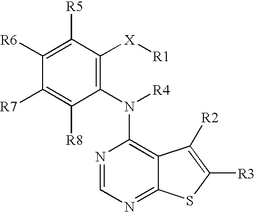
wherein X is O, S, SO₂, CH₂, CHR₁ₐ, CR₁ₐR_{1b}, CH(halogen), C(halogen)₂, C═O, C(O)NR₁ₐ, NH or NR₁ₐ, wherein R₁ₐ and R_{1b} are C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, wherein R₁ₐ and R_{1b} are optionally substituted with one or more R₉;
R₁ is hydrogen, C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, C₆₋₁₀ aryl, C₁₋₆ alkyl C₆₋₁₀ aryl, C₅₋₁₀ heteroaryl comprising at least one heteroatom selected from N, S and O, C₁₋₆ alkyl C₅₋₁₀ heteroaryl comprising at least one heteroatom selected from N, S and O, wherein R₁ is optionally substituted with one or more R₉;
or if X is NR₁ₐ, CHR₁ₐ, C(O)NR₁ₐ or CR₁ₐR_{1b}, R₁ may form a carbocyclic or heterocyclic ring with R₁ₐ and the N or C atom to which they are attached, which may contain one or more additional heteroatoms selected from N, S and O, which may be substituted with one or more R₉;
R₂ and R₃ are the same or different and are independently selected from hydrogen, C₁₋₆ alkyl, C₁₋₆ alkyl C₃₋₁₀ cycloalkyl, C₃₋₁₀ cycloalkyl, C₆₋₁₀ aryl, C₁₋₆ alkyl C₆₋₁₀ aryl, C₅₋₁₀ heteroaryl comprising at least one heteroatom selected from N, S and O, C₁₋₆ alkyl C₅₋₁₀ heteroaryl comprising at least one heteroatom selected from N, S and O, C₁₋₆ alkyl 3 to 10 membered heterocycloalkyl comprising at least one heteroatom selected from N, S and O, 3 to 10 membered heterocycloalkyl comprising at least one heteroatom selected from N, S and O, or together with the C atoms that they are attached to form a C₃₋₇ cycloalkyl or a 3 to 10 membered heterocycloalkyl group, wherein R₂ and R₃ are optionally substituted with one or more R₉, R₂ may also be R₉ and R₃ may also be R₁₀;
R₄ is hydrogen, C₁₋₄ alkyl, urea, thiourea or acetyl optionally substituted with one or more R₉;
or R₄ may form a 5 or 6 membered heterocyclic ring with R₁:
R₅, R₆, R₇ and R₈ are the same or different and are independently selected from H or R₉;
R₉ is independently halogen; CN; COOR₁₁; OR₁₁; C(O)N(R₁₁R₁₁ₐ); S(O)₂N(R₁₁R₁₁ₐ); S(O)N(R₁₁R₁₁ₐ); S(O)₂R₁₁; N(R₁₁)S(O)₂N(R₁₁ₐR_{11b}); SR₁₁; N(R₁₁R₁₁ₐ); OC(O)R₁₁; N(R₁₁)C(O)R₁₁ₐ; N(R₁₁)S(O)₂R₁₁ₐ; N(R₁₁)S(O)R₁₁ₐ; N(R₁₁)C(O)N(R₁₁ₐR_{11b}); N(R₁₁)C(O)OR₁₁ₐ; OC(O)N(R₁₁R₁₁ₐ); oxo (═O), where the ring is at least partially saturated; C(O)R₁₁; C₁₋₆ alkyl; phenyl; C₃₋₇ cycloalkyl; or heterocyclyl, wherein C₁₋₆ alkyl; phenyl; C₃₋₇ cycloalkyl; and heterocyclyl are optionally substituted with one or more R₁₀;
R₁₀ is independently halogen; CN; OR₁₁; S(O)₂N(R₁₁R₁₁ₐ); S(O)N(R₁₁R₁₁ₐ); S(O)₂R₁₁; N(R₁₁)S(O)₂N(R₁₁ₐR_{11b}); SR₁₁; N(R₁₁R₁₁ₐ); OC(O)R₁₁; N(R₁₁)C(O)R₁₁ₐ; N(R₁₁)S(O)₂R₁₁ₐ; N(R₁₁)S(O)R₁₁ₐ; N(R₁₁)C(O)N(R₁₁ₐR_{11b}); N(R₁₁)C(O)OR₁₁ₐ; OC(O)N(R₁₁R₁₁ₐ); oxo (═O), where the ring is at least partially saturated; C(O)R₁₁; C₁₋₆ alkyl; phenyl; C₃₋₇ cycloalkyl; or heterocyclyl, wherein C₁₋₆ alkyl; phenyl; C₃₋₇ cycloalkyl; and heterocyclyl are optionally substituted with one or more R₉;
R₁₁, R₁₁ₐ, R_{11b} are independently selected from the group consisting of hydrogen, C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, C₆₋₁₀ aryl, 5 to 10 membered heteroaryl comprising at least one heteroatom selected from N, S and O, wherein R₁₁, R₁₁ₐ, R_{11b} are optionally substituted with one or more R₉;
or a metabolite, prodrug or a pharmaceutically acceptable salt thereof.

2. Compound according to claim 1, wherein X is O, S, SO₂, CH₂, CHR₁ₐ, CR₁ₐR_{1b}, CH(halogen), C(halogen)₂, C═O, C(O)NR₁ₐ, NH or NR₁ₐ, wherein R₁ₐ and R_{1b} are C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, wherein R₁ₐ and R_{1b} are optionally substituted with one or more R₉;
R₁ is hydrogen, C₁₋₆ alkyl, C₁₋₆ alkyl C₃₋₁₀ cycloalkyl, C₃₋₁₀ cycloalkyl, C₁₋₆ alkyl 3 to 10 membered heterocycloalkyl comprising at least one heteroatom selected from N, S and O, 3 to 10 membered heterocycloalkyl comprising at least one heteroatom selected from N, S and O, C₆₋₁₀ aryl, C₁₋₆ alkyl C₆₋₁₀ aryl, C₅₋₁₀ heteroaryl comprising at least one heteroatom selected from N, S and O, C₁₋₆ alkyl C₅₋₁₀ heteroaryl comprising at least one heteroatom selected from N, S and O, wherein R₁ is optionally substituted with one or more R₉;
or if X is NR₁ₐ, CHR₁ₐ, C(O)NR₁ₐ or CR₁ₐR_{1b}, R₁ may form a carbocyclic or heterocyclic ring with R₁ₐ and the N or C atom to which they are attached, which may contain one or more additional heteroatoms selected from N, S and O, which may be substituted with one or more R₉;
R₂ and R₃ are the same or different and are independently selected from hydrogen, methyl, phenyl, ethyl, propyl, perfluoromethyl, or form together with the C atoms to which they are attached a 5-membered carbocyclic ring;
R₄ is hydrogen or C₁ alkyl;
R₅, R₆, R₇ and R₈ are the same or different and are independently selected from hydrogen, CONH₂, CO₂H, CO₂CH₃, Cl and F;
R₉ is as defined in claim 1;
or a metabolite, prodrug or pharmaceutically acceptable salt thereof.

3. Compound according to claim 1 or 2, wherein X is O, S, SO₂, CH₂, CHR₁ₐ, CR₁ₐR_{1b}, CH(halogen), C(halogen)₂, C═O, C(O)NR₁ₐ, NH or NR₁ₐ, wherein R₁ₐ and R_{1b} are C₁₋₆ alkyl;
R₁ is hydrogen, methyl, ethyl, propyl, butyl, difluoromethyl, bromoethyl, 1,1,2,2-tetrafluoroethyl, 1,1,1-trifluoropropyl, perfluoromethyl, cyclopropylmethyl, cyclopentyl, cyclohexyl, adamantyl, norbonanyl, tetrahydrofuranyl, tetrahydropyranyl, phenyl or pyrrolidin-3-yl substituted at the nitrogen with R₉;
or if X is NR₁ₐ, R₁ forms a morpholino group, a pyrrolidino group or a piperidino group together with R₁ₐ and the N atom to which they are attached, which may be substituted with —CH₃ or —C(O)OC₄H₉;
R₂ and R₃ are the same or different and are independently selected from hydrogen, methyl, phenyl, ethyl, propyl, perfluoromethyl, or form together with the C atoms to which they are attached a 5-membered carbocyclic ring;
R₄ is hydrogen or C₁₋₄ alkyl;
R₅, R₆, R₇ and R₈ are the same or different and are independently selected from hydrogen, CONH₂, CO₂H, CO₂CH₃, Cl and F;
R₉ is as defined in claim 1;
or a metabolite, prodrug or pharmaceutically acceptable salt thereof.

4. Compound according to any one of claims 1 to 3, wherein R₂ and R₃ are the same or different and are selected from methyl, hydrogen and perfluoromethyl.

5. Compound according to claim 1, wherein X is O, S, SO₂, CH₂, CHR₁ₐ, CR₁ₐR_{1b}, CH(halogen), C(halogen)₂, C═O, C(O)NR₁ₐ, NH or NR₁ₐ, wherein R₁ₐ and R_{1b} are C₁₋₆ alkyl;
R₁ is hydrogen, C₁₋₆ alkyl, C₁₋₆ alkyl C₃₋₁₀ cycloalkyl, C₃₋₁₀ cycloalkyl, 5 to 10 membered heterocyclyl comprising at least one heteroatom selected from N, S and O, C₆₋₁₀ aryl, C₁₋₆ alkyl C₆₋₁₀ aryl, C₅₋₁₀ heteroaryl comprising at least one heteroatom selected from N, S and O, C₁₋₆ alkyl C₅₋₁₀ heteroaryl comprising at least one heteroatom selected from N, S and O, wherein R₁ is optionally substituted with one or more R₉;
or if X is NR₁ₐ, R₁ may form a heterocyclic ring together with R₁ₐ and the N atom to which they are attached, which may contain an additional heteroatom selected from N, S and O, which may be substituted with one
or more R₉;
R₂ and R₃ are the same or different and are independently selected from hydrogen, C₁₋₄ alkyl which may optionally be substituted with one or more halogen atoms, an acetyl group, a urea, a hydroxyl, a phenyl group and an amino group or form together with the C atoms to which they are attached a C₃₋₆ cycloalkyl group;
R₄ is hydrogen or C₁₋₄ alkyl;
R₅, R₆, R₇ and R₈ are the same or different and are independently selected from hydrogen, CO₂H, CO₂R_{1c}, CONH₂, CONHR_{1d} and halogen, whereby R_{1c} and R_{1d} are C₁₋₆ alkyl;
R₉ is as defined in claim 1;
with the proviso that if R₃ is H or C₁₋₄ alkyl, R₂ cannot be hydrogen;
or a metabolite, prodrug or pharmaceutically acceptable salt thereof.

6. Compound according to any one of claims 1 to 5, wherein R₄ is hydrogen.

7. Compound according to any one of claims 1 to 6, wherein X is O.

8. Compound according to any one of claims 1 to 7, wherein the cycloalkyl group is adamantyl or norbonanyl, cyclohexyl or cyclopentyl.

9. Compound according to any one of claims 1 to 8, wherein the halogen atom is selected from Cl, Br and F.

10. Compound according to any one of claims 1 to 9, wherein R₅, R₆, R₇ and R₈ are hydrogen.

11. Compound according to any one of claims 1 to 9, wherein at least one of R₅, R₆, R₇ and R₈ is F, CONH₂ or CO₂CH₃.

12. Compound according to any one of claims 5 to 11, wherein R₁ is hydrogen, methyl, ethyl, propyl, butyl, difluoromethyl, bromoethyl, 1,1,2,2-tertrafluoroethyl, 1,1,1-trifluoropropyl, perfluoromethyl, cyclopropylmethyl, cyclopentyl, cyclohexyl, adamantyl, norbonanyl, tetrahydrofuranyl, tetrahydropyranyl, phenyl or pyrrolidin-3-yl substituted at the nitrogen with R₉, wherein R₉ is as defined in claim 1.

13. Compound according to claim 1 selected from:
(5,6-Dimethyl-thieno[2,3-d]pyrimidin-4-yl)-[2-(tetrahydro-furan-3-yloxy)-phenyl]-amine,
(5,6-Dimethyl-thieno[2,3-d]pyrimidin-4-yl)-[2-((R)-tetrahydro-furan-3-yloxy)-phenyl]-amine,
(5,6-Dimethyl-thieno[2,3-d]pyrimidin-4-yl)-[2-((S)-tetrahydro-furan-3-yloxy)-phenyl]-amine,
(5-Methyl-thieno[2,3-d]pyrimidin-4-yl)-[2-(tetrahydro-furan-3-yloxy)-phenyl]-amine,
(2-Cyclopentyloxy-phenyl)-(5-methyl-thieno[2,3-d]pyrimidin-4-yl)-amine,
(5,6-Dimethyl-thieno[2,3-d]pyrimidin-4-yl)-[2-(tetrahydro-pyran-4-yloxy)-phenyl]-amine,
(2-sec-Butoxy-phenyl)-(5,6-dimethyl-thieno[2,3-d]pyrimidin-4-yl)-amine,
(2-Isopropoxy-phenyl)-(5-methyl-thieno[2,3-d]pyrimidin-4-yl)-amine,
(5,6-Dimethyl-thieno[2,3-d]pyrimidin-4-yl)-(2-methoxy-phenyl)-amine,
(5-Methyl-thieno[2,3-d]pyrimidin-4-yl)-[2-((R)-tetrahydro-furan-3-yloxy)-phenyl]-amine,
(5-Methyl-thieno[2,3-d]pyrimidin-4-yl)-[2-(tetrahydro-pyran-4-yloxy)-phenyl]-amine,
(2-sec-Butoxy-phenyl)-(5-methyl-thieno[2,3-d]pyrimidin-4-yl)-amine,
4-(5,6-Dimethyl-thieno[2,3-d]pyrimidin-4-ylamino)-3-methoxy-benzamide,
(2-Cyclopropylmethoxy-phenyl)-(5-methyl-thieno[2,3-d]pyrimidin-4-yl)-amine,
(5,6-Dimethyl-thieno[2,3-d]pyrimidin-4-yl)-(2-methoxy-phenyl)-amine,
(5,6-Dimethyl-thieno[2,3-d]pyrimidin-4-yl)-(2-isopropoxy-phenyl)-amine,
(2-Ethoxy-phenyl)-(5-methyl-thieno[2,3-d]pyrimidin-4-yl)-amine,
3-Methoxy-4-(5-methyl-thieno[2,3-d]pyrimidin-4-ylamino)-benzamide,
(5-Methyl-thieno[2,3-d]pyrimidin-4-yl)-[2-((S)-tetrahydro-furan-3-yloxy)-phenyl]-amine,
(2-Cyclohexyloxy-phenyl)-(5-methyl-thieno[2,3-d]pyrimidin-4-yl)-amine,
(2-tert-Butoxy-phenyl)-(5,6-dimethyl-thieno[2,3-d]pyrimidin-4-yl)-amine,
(5,6-Dimethyl-thieno[2,3-d]pyrimidin-4-yl)-(2-ethoxy-phenyl)-amine,
(2-Cyclohexyloxy-phenyl)-(5,6-dimethyl-thieno[2,3-d]pyrimidin-4-yl)-amine,
(5-Methyl-thieno[2,3-d]pyrimidin-4-yl)-(2-propoxy-phenyl)-amine,
(2,4-Dimethoxy-phenyl)-(6-phenyl-thieno[2,3-d]pyrimidin-4-yl)-amine,
(2-Methoxy-phenyl)-(5,6,7,8-tetrahydro-benzo[4,5]thieno[2,3-d]pyrimidin-4-yl)-amine,
(2-Cyclopentyloxy-phenyl)-(5,6-dimethyl-thieno[2,3-d]pyrimidin-4-yl)-amine,
(5,6-Dimethyl-thieno[2,3-d]pyrimidin-4-yl)-[2-(1-ethyl-pyrrolidin-3-yloxy)-phenyl]-amine,
(2-tert-Butoxy-phenyl)-(5-methyl-thieno[2,3-d]pyrimidin-4-yl)-amine,
(5,6-Dimethyl-thieno[2,3-d]pyrimidin-4-yl)-(2-methylsulfanyl-phenyl)-amine,
(2-Methylsulfanyl-phenyl)-(5-methyl-thieno[2,3-d]pyrimidin-4-yl)-amine,
(3-Chloro-2-methoxy-phenyl)-(5-methyl-thieno[2,3-d]pyrimidin-4-yl)-amine,
(2-Difluoromethoxy-phenyl)-(5-methyl-thieno[2,3-d]pyrimidin-4-yl)-amine,
[2-(1-Ethyl-pyrrolidin-3-yloxy)-phenyl]-(5-methyl-thieno[2,3-d]pyrimidin-4-yl)-amine,
(5-Methyl-thieno[2,3-d]pyrimidin-4-yl)-[2-(1,1,2,2-tetrafluoro-ethoxy)-phenyl]-amine,
(2-sec-Butoxy-phenyl)-thieno[2,3-d]pyrimidin-4-yl-amine,
(2-Ethoxy-phenyl)-(6-methyl-thieno[2,3-d]pyrimidin-4-yl)-amine,
(2-Cyclopentyloxy-phenyl)-thieno[2,3-d]pyrimidin-4-yl-amine,
(5,6-Dimethyl-thieno[2,3-d]pyrimidin-4-yl)-(2-isobutoxy-phenyl)-amine,
(2-Isopropoxy-phenyl)-thieno[2,3-d]pyrimidin-4-yl-amine,
(2-Difluoromethoxy-phenyl)-(5,6-dimethyl-thieno[2,3-d]pyrimidin-4-yl)-amine,
(2-Cyclohexyloxy-phenyl)-thieno[2,3-d]pyrimidin-4-yl-amine,
(2-Isobutoxy-phenyl)-(5-methyl-thieno[2,3-d]pyrimidin-4-yl)-amine,
(5,6-Dimethyl-thieno[2,3-d]pyrimidin-4-yl)-[2-(1,1,2,2-tetrafluoro-ethoxy)-phenyl]-amine,
3-Methoxy-4-(thieno[2,3-d]pyrimidin-4-ylamino)-benzamide,
(6-Methyl-thieno[2,3-d]pyrimidin-4-yl)-[2-(tetrahydro-furan-3-yloxy)-phenyl]-amine,
[2-(Tetrahydro-furan-3-yloxy)-phenyl]-thieno[2,3-d]pyrimidin-4-yl-amine,
[2-(Adamantan-2-yloxy)-phenyl]-(5-methyl-thieno[2,3-d]pyrimidin-4-yl)-amine,
[2-((S)-Tetrahydro-furan-3-yloxy)-phenyl]-thieno[2,3-d]pyrimidin-4-yl-amine,
[2-(Adamantan-2-yloxy)-phenyl]-(5,6-dimethyl-thieno[2,3-c]pyrimidin-4-yl)-amine,
(5-Chloro-2-methoxy-phenyl)-thieno[2,3-d]pyrimidin-4-yl-amine,
(2-tert-Butoxy-phenyl)-thieno[2,3-d]pyrimidin-4-yl-amine,
(2-Morpholin-4-yl-phenyl)-thieno[2,3-d]pyrimidin-4-yl-amine,
[2-(Tetrahydro-pyran-4-yloxy)-phenyl]-thieno[2,3-d]pyrimidin-4-yl-amine,
(5-Methyl-thieno[2,3-d]pyrimidin-4-yl)-(2-phenoxy-phenyl)-amine,
(5,6-Dimethyl-thieno[2,3-d]pyrimidin-4-yl)-(2-isobutylsulfanyl-phenyl)-amine,
(5-Methyl-thieno[2,3-d]pyrimidin-4-yl)-(2-trifluoromethoxy-phenyl)-amine,
(2-Ethoxy-phenyl)thieno[2,3-d]pyrimidin-4-yl-amine,
(2-Methylsulfanyl-phenyl)-thieno[2,3-d]pyrimidin-4-yl-amine,
(5,6-Dimethyl-thieno[2,3-d]pyrimidin-4-yl)-(2-propyl-phenyl)-amine,
(5,6-Dimethyl-thieno[2,3-d]pyrimidin-4-yl)-(2-isopropyl-phenyl)-amine,
(2-Methoxy-phenyl)-(5-methyl-thieno[2,3-d]pyrimidin-4-yl)-amine,
(5,6-Dimethyl-thieno[2,3-d]pyrimidin-4-yl)-(2-ethyl-phenyl)-amine,
[2-(Bicyclo[2.2.1]hept-2-yloxy)-pheny]-thieno[2,3-d]pyrimidin-4-yl-amine,
[2-(Adamantan-2-yloxy)-phenyl]-thieno[2,3-d]pyrimidin-4-yl-amine,
(2-Methoxy-phenyl)-thieno[2,3-d]pyrimidin-4-yl-amine,
(2-Isobutoxy-phenyl)-thieno[2,3-d]pyrimidin-4-yl-amine,
(2-Methoxy-phenyl)-(6-methyl-thieno[2,3-d]pyrimidin-4-yl)-amine,
(2-sec-Butyl-phenyl)-(5,6-dimethyl-thieno[2,3-d]pyrimidin-4-yl)-amine,
(2-Piperidin-1-yl-phenyl)-thieno[2,3-d]pyrimidin-4-yl-amine,
[2-(Adamantan-1-yloxy)-phenyl]-(5-methyl-thieno[2,3-d]pyrimidin-4-yl)-amine,
(2-Isobutylsulfanyl-phenyl)-thieno[2,3-d]pyrimidin-4-yl-amine,
2-(5-Methyl-thieno[2,3-d]pyrimidin-4-ylamino)-phenol,
(3-Chloro-2-methoxy-phenyl)thieno[2,3-d]pyrimidin-4-yl-amine,
(2-sec-Butyl-phenyl)-(5-methyl-thieno[2,3-d]pyrimidin-4-yl)-amine,
(2-sec-Butyl-phenyl)-(5,6-dimethyl-thieno[2,3-d]pyrimidin-4-yl)-amine,
(6-Ethyl-thieno[2,3-d]pyrimidin-4-yl)-[2-(tetrahydro-furan-3-yloxy)-phenyl]-amine,
(2-Bromo-phenyl)-(5-methyl-thieno[2,3-d]pyrimidin-4-yl)-amine,
(2-Cyclohexylsulfanyl-phenyl)-thieno[2,3-d]pyrimidin-4-yl-amine,
(2-Phenoxy-phenyl)-thieno[2,3-d]pyrimidin-4-yl-amine,
2-(Thieno[2,3-d]pyrimidin-4-ylamino)-phenol,
(2-Isobutylsulfanyl-phenyl)-(5-methyl-thieno[2,3-d]pyrimidin-4-yl)-amine,
(2-Isopropoxy-phenyl)-(6-methyl-5-propyl-thieno[2,3-d]pyrimidin-4-yl)-amine,
[2-(Tetrahydro-furan-3-yloxy)-phenyl]-(5-trifluoromethyl-thieno[2,3-d]pyrimidin-4-yl)-amine,
(6-Isopropyl-thieno[2,3-d]pyrimidin-4-yl)[2-(tetrahydro-furan-3-yloxy)-phenyl]-amine,
(6-Isopropyl-thieno[2,3-d]pyrimidin-4-yl)-(2-methoxy-phenyl)-amine,
(6-Ethyl-thieno[2,3-d]pyrimidin-4-yl)-(2-isopropoxy-phenyl)-amine,
(2-Methanesulfonyl-phenyl)-(5-methyl-thieno[2,3-d]pyrimidin-4-yl)-amine,
(5,6-Dimethyl-thieno[2,3-d]pyrimidin-4-yl)-(2-phenoxy-phenyl)-amine,
(5-Methyl-thieno[2,3-d]pyrimidin-4-yl)-(2-piperidin-1-yl-phenyl)-amine,
(6-Isopropyl-thieno[2,3-d]pyrimidin-4-yl)-(2-methoxy-phenyl)-amine,
(2-sec-Butoxy-phenyl)-(6-isopropyl-thieno[2,3-d]pyrimidin-4-yl)-amine,
(2-Cyclopentylsulfanyl-phenyl)-thieno[2,3-d]pyrimidin-4-yl-amine,
[2-(endo-Bicyclo[2.2.1]hept-2-yloxy)-phenyl]-(5,6-dimethyl-thieno[2,3-d]pyrimidin-4-yl)-amine,
[2-(endo-Bicyclo[2.2.1]hept-2-yloxy)-phenyl]-thieno[2,3-d]pyrimidin-4-yl-amine,
[2-(endo-Bicyclo[2.2.1]hept-2-yloxy)-phenyl]-(5-methyl-thieno[2,3-d]pyrimidin-4-yl)-amine,
(5,6-Dimethyl-thieno[2,3-d]pyrimidin-4-yl)-[2-(tetrahydro-furan-3-ylmethoxy)-phenyl]-amine,
[2-(Tetrahydro-furan-3-ylmethoxy)-phenyl]-thieno[2,3-d]pyrimidin-4-yl-amine,
(5-Methyl-thieno[2,3-d]pyrimidin-4-yl)-[2-(tetrahydro-furan-3-ylmethoxy)-phenyl]-amine,
(2-Isopropoxy-phenyl)-(6-isopropyl-thieno[2,3-d]pyrimidin-4-yl)-amine,
[2-(1,2-Dimethyl-propoxy)-phenyl]-thieno[2,3-d]pyrimidin-4-yl-amine,
(2-Cyclopentyloxy-phenyl)-(6-isopropyl-thieno[2,3-d]pyrimidin-4-yl)-amine,
[2-(1,2-Dimethyl-propoxy)-phenyl]-(5-methyl-thieno[2,3-d]pyrimidin-4-yl)-amine,
[2-(1,2-Dimethyl-propoxy)-phenyl]-(5,6-dimethyl-thieno[2,3-d]pyrimidin-4-yl)-amine,
2,6-Dimethyl-4-[2-(thieno[2,3-d]pyrimidin-4-ylamino)-phenyl]-piperazine-1-carboxylic acid tert-butyl ester,
[5-Fluoro-2-(tetrahydro-furan-3-yloxy)-phenyl]-(5-methyl-thieno[2,3-d]pyrimidin-4-yl)-amine,
(5,6-Dimethyl-thieno[2,3-d]pyrimidin-4-yl)-[5-fluoro-2-(tetrahydro-furan-3-yloxy)-phenyl]-amine,
[5-Fluoro-2-(tetrahydro-furan-3-yloxy)-phenyl]-thieno[2,3-d]pyrimidin-4-yl-amine,
(2-Cyclopentyloxy-phenyl)-(6-ethyl-thieno[2,3-d]pyrimidin-4-yl)-amine,
(2-Methoxy-phenyl)-(6-methyl-5-propyl-thieno[2,3-d]pyrimidin-4-yl)-amine,
(2-Ethoxy-phenyl)-(6-methyl-5-propyl-thieno[2,3-d]pyrimidin-4-yl)-amine,
(2-Isopropoxy-phenyl)-(6-methyl-5-propyl-thieno[2,3-d]pyrimidin-4-yl)-amine,
(2-sec-Butoxy-phenyl)-(6-methyl-5-propyl-thieno[2,3-d]pyrimidin-4-yl)-amine,
3-(Tetrahydro-furan-3-yloxy)-4-(thieno[2,3-d]pyrimidin-4-ylamino)-benzoic acid methyl ester,
4-(5-Methyl-thieno[2,3-d]pyrimidin-4-ylamino)-3-(tetrahydro-furan-3-yloxy)-benzoic acid methyl ester,
4-(5,6-Dimethyl-thieno[2,3-d]pyrimidin-4-ylamino)-3-(tetrahydro-furan-3-yloxy)-benzoic acid methyl ester,
3-(Tetrahydro-furan-3-yloxy)-4-(thieno[2,3-d]pyrimidin-4-ylamino)-benzamide,
N-Isopropyl-N′-thieno[2,3-d]pyrimidin-4-yl-benzene-1,2-diamine,
(5,6-Dimethyl-thieno[2,3-d]pyrimidin-4-yl)-(2-methanesulfonyl-phenyl)-amine,
[2-(Tetrahydro-furan-3-yloxy)-phenyl]-(5-trifluoromethyl-thieno[2,3-d]pyrimidin-4-yl)-amine,
(2-Cyclopentyloxy-phenyl)-(5-trifluoromethyl-thieno[2,3-d]pyrimidin-4-yl)-amine,
2,6-Dimethyl-4-[2-(5-methyl-thieno[2,3-d]pyrimidin-4-ylamino)-phenyl]-piperazine-1-carboxylic acid tent-butyl ester,
(2-Ethoxy-5-fluoro-phenyl)thieno[2,3-d]pyrimidin-4-yl-amine,
(2-sec-Butoxy-phenyl)-(5-trifluoromethyl-thieno[2,3-d]pyrimidin-4-yl)-amine,
(5,6-Dimethyl-thieno[2,3-d]pyrimidin-4-yl)-[2-(1-ethyl-2-methyl-propoxy)-phenyl]-amine,
3-[2-(5-Methyl-thieno[2,3-d]pyrimidin-4-ylamino)-phenoxy]-pyrrolidine-1-carboxylic acid tert-butyl ester,
3-[2-(5,6-Dimethyl-thieno[2,3-d]pyrimidin-4-ylamino)-phenoxy]-pyrrolidine-1-carboxylic acid tert-butyl ester,
[2-(3,5-Dimethyl-piperazin-1-yl)-phenyl]-(5-methyl-thieno[2,3-d]pyrimidin-4-yl)-amine,
(2-Pyrrolidin-1-yl-phenyl)thieno[2,3-d]pyrimidin-4-yl-amine,
(5-Methyl-thieno[2,3-d]pyrimidin-4-yl)-(2-pyrrolidin-1-yl-phenyl)-amine,
(5,6-Dimethyl-thieno[2,3-d]pyrimidin-4-yl)-(2-pyrrolidin-1-yl-phenyl)-amine,
(2-Cyclopentyloxy-phenyl)-(6-methyl-5-propyl-thieno[2,3-d]pyrimidin-4-yl)-amine,
N-Isopropyl-N′-(5-methyl-thieno[2,3-d]pyrimidin-4-yl)-benzene-1,2-diamine,
N-Cyclopentyl-N′-(5-methyl-thieno[2,3-d]pyrimidin-4-yl)-benzene-1,2-diamine,
N-sec-Butyl-N′-(5-methyl-thieno[2,3-d]pyrimidin-4-yl)-benzene-1,2-diamine,
(6-Ethyl-thieno[2,3-d]pyrimidin-4-yl)-(2-methoxy-phenyl)-amine,
(2-Ethoxy-phenyl)-(6-ethyl-thieno[2,3-d]pyrimidin-4-yl)-amine,
(2-sec-Butoxy-phenyl)-(6-ethyl-thieno[2,3-d]pyrimidin-4-yl)-amine,
(2-Ethoxy-5-fluoro-phenyl)-(5-methyl-thieno[2,3-d]pyrimidin-4-yl)-amine,
(2-Ethoxy-phenyl)-(6-isopropyl-thieno[2,3-d]pyrimidin-4-yl)-amine,
[2-(1-Ethyl-2-methyl-propoxy)-phenyl]-(5-methyl-thieno[2,3-d]pyrimidin-4-yl)-amine,
(6-Methyl-5-propyl-thieno[2,3-d]pyrimidin-4-yl)-[2-(tetrahydro-furan-3-yloxy)-phenyl]-amine,
(2-Isopropoxy-phenyl)-(5-trifluoromethyl-thieno[2,3-d]pyrimidin-4-yl)-amine,
[2-(1-Ethyl-2-methyl-propoxy)-phenyl]-thieno[2,3-cl]pyrimidin-4-yl-amine,
Thieno[2,3-d]pyrimidin-4-yl-[2-(3,3,3-trifluoro-propoxy)-phenyl]-amine,
(5-Methyl-thieno[2,3-d]pyrimidin-4-yl)-[2-(3,3,3-trifluoro-propoxy)-phenyl]-amine,
(5,6-Dimethyl-thieno[2,3-cl]pyrimidin-4-yl)-[2-(3,3,3-trifluoro-propoxy)-phenyl]-amine,
(5-Ethyl-thieno[2,3-d]pyrimidin-4-yl)-(2-isopropoxy-phenyl)-amine,
(2-sec-Butoxy-phenyl)-(5-ethyl-thieno[2,3-d]pyrimidin-4-yl)-amine,
(2-Cyclopentyloxy-phenyl)-(5-ethyl-thieno[2,3-d]pyrimidin-4-yl)-amine,
[2-(3-Ethoxy-propoxy)-phenyl]-thieno[2,3-d]pyrimidin-4-yl-amine,
(5,6-Dimethyl-thieno[2,3-d]pyrimidin-4-yl)-[2-(3-ethoxy-propoxy)-phenyl]-amine,
(5-Ethyl-thieno[2,3-d]pyrimidin-4-yl)-[2-(tetrahydro-furan-3-yloxy)-phenyl]-amine,
3-Ethoxy-4-(5-methyl-thieno[2,3-d]pyrimidin-4-ylamino)-benzamide,
3-Isopropoxy-4-(5-methyl-thieno[2,3-d]pyrimidin-4-ylamino)-benzamide,
3-sec-Butoxy-4-(5-methyl-thieno[2,3-d]pyrimidin-4-ylamino)-benzamide,
3-Cyclopentyloxy-4-(5-methyl-thieno[2,3-d]pyrimidin-4-ylamino)-benzamide,
4-(5-Methyl-thieno[2,3-d]pyrimidin-4-ylamino)-3-(tetrahydro-furan-3-yloxy)-benzamide,
(5,6-Dimethyl-thieno[2,3-d]pyrimidin-4-yl)-[3-fluoro-2-(tetrahydro-furan-3-yloxy)-phenyl]-amine,
[2-(3-Ethoxy-propoxy)-phenyl]-(5-methyl-thieno[2,3-d]pyrimidin-4-yl)-amine,
[2-(2-Ethoxy-ethoxy)-phenyl]-(5-methyl-thieno[2,3-]pyrimidin-4-yl)-amine,
[2-(2-Ethoxy-ethoxy)-phenyl]-thieno[2,3-d]pyrimidin-4-yl-amine,
(5,6-Dimethyl-thieno[2,3-d]pyrimidin-4-yl)-[2-(2-ethoxy-ethoxy)-phenyl]-amine,
3-Ethoxy-4-(thieno[2,3-d]pyrimidin-4-ylamino)-benzamide,
3-Isopropoxy-4-(thieno[2,3-d]pyrimidin-4-ylamino)-benzamide,
3-sec-Butoxy-4-(thieno[2,3-d]pyrimidin-4-ylamino)-benzamide,
3-Cyclopentyloxy-4-(thieno[2,3-d]pyrimidin-4-ylamino)-benzamide,
3-(Tetrahydro-furan-3-yloxy)-4-(5-trifluoromethyl-thieno[2,3-d]pyrimidin-4-ylamino)-benzamide,
(2,3-Dihydro-1H-8-thia-5,7-diaza-cyclopenta[a]inden-4-yl)-(2-methoxy-phenyl)-amine,
[2-(exo-Bicyclo[2.2.1]hept-2-yloxy)-phenyl]-(5,6-dimethyl-thieno[2,3-d]pyrimidin-4-yl)-amine,
(5-Methyl-thieno[2,3-d]pyrimidin-4-yl)-[2-((R)-tetrahydro-furan-3-yloxy)-phenyl]-amine,
(5,6-Dimethyl-thieno[2,3-d]pyrimidin-4-yl)-(2-morpholin-4-yl-phenyl)-amine,
(5-Methyl-thieno[2,3-d]pyrimidin-4-yl)-(2-phenoxy-phenyl)-amine,
(2-Ethyl-phenyl)-(5-methyl-thieno[2,3-d]pyrimidin-4-yl)-amine,
(2-Isopropyl-phenyl)-(5-methyl-thieno[2,3-d]pyrimidin-4-yl)-amine,
[2-(2-Bromo-ethoxy)phenyl]-(5-methyl-thieno[2,3-d]pyrimidin-4-yl)-amine, and
(5-Methyl-thieno[2,3-d]pyrimidin-4-yl)-(2-propyl-phenyl)-amine,
4-(5,6-Dimethyl-thieno[2,3-d]pyrimidin-4-yl)-3,4-dihydro-2H-benzo[1,4]oxazine,
[2-(Bicyclo[2.2.1]hept-2-yloxy)-phenyl]-(5-methyl-thieno[2,3-d]pyrimidin-4-yl)-amine,
2,6-Dimethyl-4-[2-(thieno[2,3-d]pyrimidin-4-ylamino)-phenyl]-piperazine-1-carboxylic acid tert-butyl ester,
N-Isopropyl-N′-thieno[2,3-d]pyrimidin-4-yl-benzene-1,2-diamine,
2,6-Dimethyl-4-[2-(5-methyl-thieno[2,3-d]pyrimidin-4-ylamino)-phenyl]-piperazine-1-carboxylic acid tert-butyl ester,
[2-(3,5-Dimethyl-piperazin-1-yl)-phenyl]-thieno[2,3-d]pyrimidin-4-yl-amine,
N-Cyclopentyl-N′-thieno[2,3-d]pyrimidin-4-yl-benzene-1,2-diamine,
N-Cyclohexyl-N′-thieno[2,3-d]pyrimidin-4-yl-benzene-1,2-diamine,
N-sec-Butyl-N′-thieno[2,3-d]pyrimidin-4-yl-benzene-1,2-diamine,
N-Isopropyl-N′-(5-methyl-thieno[2,3-d]pyrimidin-4-yl)-benzene-1,2-diamine,
[2-(3-Ethoxy-propoxy)-phenyl]thieno[2,3-d]pyrimidin-4-yl-amine,
(5-Ethyl-thieno[2,3-d]pyrimidin-4-yl)-[2-(tetrahydro-furan-3-yloxy)-phenyl]-amine,
[2-(3-Ethoxy-propoxy)-phenyl]-(5-methyl-thieno[2,3-d]pyrimidin4-yl)-amine,
[2-(2-Ethoxy-ethoxy)-phenyl]-thieno[2,3-d]pyrimidin-4-yl-amine,
[2-(2-Ethoxy-ethoxy)-phenyl]-(5-methyl-thieno[2,3-d]pyrimidin-4-yl)-amine,
3-Ethoxy-4-(thieno[2,3-d]pyrimidin-4-ylamino)-benzamide,
(2-Cyclopentyloxy-4-fluoro-phenyl)-(5,6-dimethyl-thieno[2,3-d]pyrimidin-4-yl)-amine,
(2,6-Dimethoxy-phenyl)-thieno[2,3-d]pyrimidin-4-yl-amine,
(2,6-Dimethoxy-phenyl)-(5-methyl-thieno[2,3-d]pyrimidin-4-yl)-amine,
(2,6-Dimethoxy-phenyl)-(5,6-dimethyl-thieno[2,3-d]pyrimidin-4-yl)-amine,
1-{3-[2-(Thieno[2,3-d]pyrimidin-4-ylamino)-phenoxy]-pyrrolidin-1-yl}-ethanone,
3-[2-(Thieno[2,3-d]pyrimidin-4-ylamino)-phenoxy]-pyrrolidine-1-carboxylic acid dimethylamide,
2-Methyl-1-{3-[2-(thieno[2,3-d]pyrimidin-4-ylamino)-phenoxy]-pyrrolidin-1-yl}-propan-1-one,
3-Methoxy-N-methyl-4-(thieno[2,3-d]pyrimidin-4-ylamino)-benzamide,
3-Methoxy-N-methyl-4-(5-methyl-thieno[2,3-d]pyrimidin-4-ylamino)-benzamide,
4-(5,6-Dimethyl-thieno[2,3-d]pyrimidin-4-ylamino)-3-methoxy-N-methyl-benzamide,
3-Methoxy-N,N-dimethyl-4-(thieno[2,3-d]pyrimidin-4-ylamino)-benzamide,
Pyridin-3-yl-{3-[2-(thieno[2,3-d]pyrimidin-4-ylamino)-phenoxy]-pyrrolidin-1-yl}-methanone,
Pyridin-4-yl-{3-[2-(thieno[2,3-d]pyrimidin-4-ylamino)-phenoxy]-pyrrolidin-1-y}-methanone,
3-Methoxy-N,N-dimethyl-4-(5-methyl-thieno[2,3-d]pyrimidin-4-ylamino)-benzamide,
N-Methyl-3-(tetrahydro-furan-3-yloxy)-4-(thieno[2,3-d]pyrimidin-4-ylamino)-benzamide,
Cyclopropyl-{3-[2-(thieno[2,3-d]pyrimidin-4-ylamino)-phenoxy]-pyrrolidin-1-yl}-methanone,
(2-Cyclopentyloxy-4-fluoro-phenyl)-thieno[2,3-d]pyrimidin-4-yl-amine,
(5-Methyl-thieno[2,3-d]pyrimidin-4-yl)-[2-(pyrrolidin-3-yloxy)-phenyl]amine,
2-Fluoro-5-(tetrahydro-furan-3-yloxy)-4-(thieno[2,3-d]pyrimidin-4-ylamino)-benzamide,
2-Ethoxy-4-[1,2,4]oxadiazol-5-yl-phenyl)-(5-methyl-thieno[2,3-d]pyrimidin-4-yl)-amine,
[2-(Bicyclo[2.2.1]hept-2-yloxy)-phenyl]-(5,6-dimethyl-thieno[2,3-d]pyrimidin-4-yl)-amine,
(6-Ethyl-thieno[2,3-d]pyrimidin-4-yl)-(2-methoxy-phenyl)-amine,
(2-Ethoxy-phenyl)-(6-isopropyl-thieno[2,3-d]pyrimidin-4-yl)-amine,
(6-Isopropyl-thieno[2,3-d]pyrimidin-4-yl)-(2-methoxy-phenyl)-amine,
(2-sec-Butoxy-phenyl)-(6-isopropyl-thieno[2,3-d]pyrimidin-4-yl)-amine,
(2-Cyclopentylsulfanyl-phenyl)-thieno[2,3-d]pyrimidin-4-yl-amine,
(2-Cyclohexylsulfanyl-phenyl)-thieno[2,3-d]pyrimidin-4-yl-amine
[2-(Bicyclo[2.2.1]hept-2-yloxy)-phenyl]-thieno[2,3-d]pyrimidin-4-yl-amine,
[2-(Tetrahydro-furan-3-ylmethoxy)-phenyl]-thieno[2,3-d]pyrimidin-4-yl-amine,
[5-Fluoro-2-(tetrahydro-furan-3-yloxy)-phenyl]-thieno[2,3-d]pyrimidin-4-yl-amine,
(2-sec-Butoxy-phenyl)-(6-methyl-5-propyl-thieno[2,3-d]pyrimidin-4-yl)-amine,
4-(5,6-Dimethyl-thieno[2,3-d]pyrimidin-4-ylamino)-3-(tetrahydro-furan-3-yloxy)-benzoic acid methyl ester,
(5,6-Dimethyl-thieno[2,3-d]pyrimidin-4-yl)-(2-methanesulfonyl-phenyl)-amine,
(2-Ethoxy-5-fluoro-phenyl)thieno[2,3-d]pyrimidin-4-yl-amine,
(2-Ethoxy-5-fluoro-phenyl)-(5-methyl-thieno[2,3-d] pyrimidin-4-yl)-amine
[2-(3,5-Dimethyl-piperazin-1-yl)-phenyl]-(5-methyl-thieno[2,3-d]pyrimidin-4-yl)-amine,
(2-Cyclopentyloxy-phenyl)-(6-methyl-5-propyl-thieno[2,3-d]pyrimidin-4-yl)-amine,
(2-Pyrrolidin-1-yl-phenyl)thieno[2,3-d]pyrimidin-4-yl-amine,
3-[2-(5-Methyl-thieno[2,3-d]pyrimidin-4-ylamino)-phenoxy]-pyrrolidine-1-carboxylic acid tert-butyl ester,
N-sec-Butyl-N′-(5-methyl-thieno[2,3-d]pyrimidin-4-yl)-benzene-1,2-diamine,
N-Cyclopentyl-N′-(5-methyl-thieno[2,3-d]pyrimidin-4-yl)-benzene-1,2-diamine,
[3-Fluoro-2-(tetrahydro-furan-3-yloxy)-phenyl]thieno[2,3-d]pyrimidin-4-yl-amine,
(4-Fluoro-2-methoxy-phenyl)thieno[2,3-d]pyrimidin-4-yl-amine,
3-[2-(Thieno[2,3-d]pyrimidin-4-ylamino)-phenoxy]-pyrrolidine-1-carboxylic acid tert-butyl ester,
[2-(Pyrrolidin-3-yloxy)-phenyl]-thieno[2,3-d]pyrimidin-4-yl-amine,
[2-(1-Methanesulfonyl-pyrrolidin-3-yloxy)-phenyl]-thieno[2,3-d]pyrimidin-4-yl-amine,
{2-[1-(Propane-2-sulfonyl)-pyrrolidin-3-yloxy]-phenyl}-thieno[2,3-d]pyrimidin-4-yl-amine,
3-[2-(Thieno[2,3-d]pyrimidin-4-ylamino)-phenoxy]-pyrrolidine-1-sulfonic acid dimethylamide,
[2-(1-Cyclopropanesulfonyl-pyrrolidin-3-yloxy)-phenyl]-thieno[2,3-d]pyrimidin-4-yl-amine,
3-[2-(Thieno[2,3-d]pyrimidin-4ylamino)-phenoxy]-pyrrolidine-1-carboxylic acid 4-methoxy-benzylamide,
{3-[2-(5-Methyl-thieno[2,3-d]pyrimidin-4-ylamino)-phenoxy]-pyrrolidin-1-yl}-pyridin-3-yl-methanone,
[2-Ethoxy-4-(4H-[1,2,4]triazol-3-yl)-phenyl]-(5-methyl-thieno[2,3-d]pyrimidin-4-yl)-amine,
[2-(Bicyclo[2.2.1]hept-2-yloxy)-phenyl]-thieno[2,3-d]pyrimidin-4-yl-amine,
(2-Ethoxy-phenyl)-(6-ethyl-thieno[2,3-d]pyrimidin-4-yl)-amine,
(2-sec-Butoxy-phenyl)-(6-ethyl-thieno[2,3-d]pyrimidin-4-yl)-amine,
(6-Ethyl-thieno[2,3-d]pyrimidin-4-yl)-[2-(tetrahydro-furan-3-yloxy)-phenyl]-amine,
[2-(Bicyclo[2.2.1]hept-2-yloxy)-phenyl]-(5-methyl-thieno[2,3-d]pyrimidin-4-yl)-amine,
[2-(Bicyclo[2.2.1]hept-2-yloxy)-phenyl]-(5,6-dimethyl-thieno[2,3-d]pyrimidin-4-yl)-amine,
(5-Methyl-thieno[2,3-d]pyrimidin-4-yl)-[2-(tetrahydro-furan-3-ylmethoxy)-phenyl]-amine,
(5,6-Dimethyl-thieno[2,3-d]pyrimidin-4-yl)-[2-(tetrahydro-furan-3-ylmethoxy)-phenyl]-amine,
(2-Isopropoxy-phenyl)-(6-isopropyl-thieno[2,3-d]pyrimidin-4-yl)-amine,
(2-Cyclopentyloxy-phenyl)-(6-isopropyl-thieno[2,3-d]pyrimidin-4-yl)-amine,
(6-Isopropyl-thieno[2,3-d]pyrimidin-4-yl)-[2-(tetrahydro-furan-3-yloxy)-phenyl]-amine,
[2-(1,2-Dimethyl-propoxy)-phenyl]thieno[2,3-d]pyrimidin-4-yl-amine,
[2-(1,2-Dimethyl-propoxy)-phenyl]-(5-methyl-thieno[2,3-d]pyrimidin-4-yl)-amine,
[2-(1,2-Dimethyl-propoxy)-phenyl]-(5,6-dimethyl-thieno[2,3-d]pyrimidin-4-yl)-amine,
[5-Fluoro-2-(tetrahydro-furan-3-yloxy)-phenyl]-(5-methyl-thieno[2,3-d]pyrimidin-4-yl)-amine,
(5,6-Dimethyl-thieno[2,3-d]pyrimidin-4-yl)-[5-fluoro-2-(tetrahydro-furan-3-yloxy)-phenyl]-amine,
(6-Ethyl-thieno[2,3-d]pyrimidin-4-yl)-(2-isopropoxy-phenyl)-amine,
(2-Cyclopentyloxy-phenyl)-(6-ethyl-thieno[2,3-d]pyrimidin-4-yl)-amine,
(2-Methoxy-phenyl)-(6-methyl-5-propyl-thieno[2,3-d]pyrimidin-4-yl)-amine,
(2-Ethoxy-phenyl)-(6-methyl-5-propyl-thieno[2,3-d]pyrimidin-4-yl)-amine,
(2-Isopropoxy-phenyl)-(6-methyl-5-propyl-thieno[2,3-d]pyrimidin-4-yl)-amine,
3-(Tetrahydro-furan-3-yloxy)-4-(thieno[2,3-d]pyrimidin-4-ylamino)-benzoic acid methyl ester,
4-(5-Methyl-thieno[2,3-d]pyrimidin-4-ylamino)-3-(tetrahydro-furan-3-yloxy)-benzoic acid methyl ester,
3-(Tetrahydro-furan-3-yloxy)-4-(thieno[2,3-d]pyrimidin-4-ylamino)-benzamide,
[2-(Tetrahydro-furan-3-yloxy)-phenyl]-(5-trifluoromethyl-thieno[2,3-d]pyrimidin-4-yl)-amine,
(2-Cyclopentyloxy-phenyl)-(5-trifluoromethyl-thieno[2,3-d]pyrimidin-4-yl)-amine,
[2-(1-Ethyl-2-methyl-propoxy)-phenyl]-thieno[2,3-d]pyrimidin-4-yl-amine,
[2-(1-Ethyl-2-methyl-propoxy)-phenyl]-(5-methyl-thieno[2,3-d]pyrimidin-4-yl)-amine,
(6-Methyl-5-propyl-thieno[2,3-d]pyrimidin-4-yl)-[2-(tetrahydro-furan-3-yloxy)-phenyl]-amine,
(5,6-Dimethyl-thieno[2,3-d]pyrimidin-4-yl)-[2-(1-ethyl-2-methyl-propoxy)-phenyl]-amine,
(2-Isopropoxy-phenyl)-(5-trifluoromethyl-thieno[2,3-d]pyrimidin-4-yl)-amine,
(2-sec-Butoxy-phenyl)-(5-trifluoromethyl-thieno[2,3-d]pyrimidin-4-yl)-amine,
(5-Methyl-thieno[2,3-d]pyrimidin-4-yl)-(2-pyrrolidin-1-yl-phenyl)-amine,
(5,6-Dimethyl-thieno[2,3-d]pyrimidin-4-yl)-(2-pyrrolidin-1-yl-phenyl)-amine,
3-[2-(5,6-Dimethyl-thieno[2,3-d]pyrimidin-4-ylamino)-phenoxy]-pyrrolidine-1-carboxylic acid tert-butyl ester,
Thieno[2,3-d]pyrimidin-4-yl-[2-(3,3,3-trifluoro-propoxy)-phenyl]-amine,
(5-Methyl-thieno[2,3-d]pyrimidin-4-yl)-[2-(3,3,3-trifluoro-propoxy)-phenyl]-amine
(5,6-Dimethyl-thieno[2,3-d]pyrimidin-4-yl)-[2-(3,3,3-trifluoro-propoxy)-phenyl]-amine,
(5-Ethyl-thieno[2,3-d]pyrimidin-4-yl)-(2-isopropoxy-phenyl)-amine,
(2-sec-Butoxy-phenyl)-(5-ethyl-thieno[2,3-d]pyrimidin-4-yl)-amine,
(2-Cyclopentyloxy-phenyl)-(5-ethyl-thieno[2,3-d]pyrimidin-4-yl)-amine,
(5,6-Dimethyl-thieno[2,3-d]pyrimidin-4-yl)-[2-(3-ethoxy-propoxy)-phenyl]-amine,
[3-Fluoro-2-(tetrahydro-furan-3-yloxy)-phenyl]-(5-methyl-thieno[2,3-d]pyrimidin-4-yl)-amine,
(5,6-Dimethyl-thieno[2,3-d]pyrimidin-4-yl)-[3-fluoro-2-(tetrahydro-furan-3-yloxy)-phenyl]-amine,
(5,6-Dimethyl-thieno[2,3-d]pyrimidin-4-yl)-[2-(2-ethoxy-ethoxy)-phenyl]-amine,
3-Isopropoxy-4-(thieno[2,3-d]pyrimidin-4-ylamino)-benzamide,
3-sec-Butoxy-4-(thieno[2,3-d]pyrimidin-4-ylamino)-benzamide,
3-Cyclopentyloxy-4-(thieno[2, 3-d]pyrimidin-4-ylamino)-benzamide,
3-Ethoxy-4-(5-methyl-thieno[2,3-d]pyrimidin-4-ylamino)-benzamide,
3-Isopropoxy-4-(5-methyl-thieno[2,3-d]pyrimidin-4-ylamino)-benzamide,
3-sec-Butoxy-4-(5-methyl-thieno[2,3-d]pyrimidin-4-ylamino)-benzamide,
3-Cyclopentyloxy-4-(5-methyl-thieno[2,3-d]pyrimidin-4-ylamino)-benzamide,
4-(5-Methyl-thieno[2,3-d]pyrimidin-4-ylamino)-3-(tetrahydro-furan-3-yloxy)-benzamide,
3-(Tetrahydro-furan-3-yloxy)-4-(5-trifluoromethyl-thieno[2,3-d]pyrimidin-4-ylamino)-benzamide,
4-(Thieno[2,3-d]pyrimidin-4-ylamino)-3-(3,3,3-trifluoro-propoxy)-benzamide,
4-(5-Methyl-thieno[2,3-d]pyrimidin-4-ylamino)-3-(3,3,3-trifluoro-propoxy)-benzamide,
4-(5,6-Dimethyl-thieno[2,3-d]pyrimidin-4-ylamino)-3-(3,3,3-trifluoro-propoxy)-benzamide,
3-Pyrrolidin-1-yl-4-(thieno[2,3-d]pyrimidin-4-ylamino)-benzamide,
4-(5-Methyl-thieno[2,3-d]pyrimidin-4-ylamino)-3-pyrrolidin-1-yl-benzamide,
4-(5,6-Dimethyl-thieno[2,3-d]pyrimidin-4-ylamino)-3-pyrrolidin-1-yl-benzamide,
4-(5,6-Dimethyl-thieno[2,3-d]pyrimidin-4-ylamino)-3-ethoxy-benzamide,
4-(5,6-Dimethyl-thieno[2,3-d]pyrimidin-4-ylamino)-3-isopropoxy-benzamide,
3-Cyclopentyloxy-4-(5,6-dimethyl-thieno[2,3-d]pyrimidin-4-ylamino)-benzamide,
4-(5,6-Dimethyl-thieno[2,3-d]pyrimidin-4-ylamino)-3-(tetrahydro-furan-3-yloxy)-benzamide,
(2-Ethoxy-4-fluoro-phenyl)-thieno[2,3-d]pyrimidin-4-yl-amine,
[4-Fluoro-2-(tetrahydro-furan-3-yloxy)-phenyl]-thieno[2,3-d]pyrimidin-4-yl-amine,
(4-Fluoro-2-methoxy-phenyl)-(5-methyl-thieno[2,3-d]pyrimidin-4-yl)-amine,
(2-Ethoxy-4-fluoro-phenyl)-(5-methyl-thieno[2,3-d]pyrimidin-4-yl)-amine,
(2-Cyclopentyloxy-4-fluoro-phenyl)-(5-methyl-thieno[2,3-d]pyrimidin-4-yl)-amine,
[4-Fluoro-2-(tetrahydro-furan-3-yloxy)-phenyl]-(5-methyl-thieno[2,3-d]pyrimidin-4-yl)-amine,
(5,6-Dimethyl-thieno[2,3-d]pyrimidin-4-yl)-(4-fluoro-2-methoxy-phenyl)-amine,
(5,6-Dimethyl-thieno[2,3-d]pyrimidin-4-yl)-(2-ethoxy-4-fluoro-phenyl)-amine,
(5,6-Dimethyl-thieno[2,3-d]pyrimidin-4-yl)-(4-fluoro-2-isopropoxy-phenyl)-amine,
(5,6-Dimethyl-thieno[2,3-d]pyrimidin-4-yl)-[4-fluoro-2-(tetrahydro-furan-3-yloxy)-phenyl]-amine,
3-Methoxy-4-(5-trifluoromethyl-thieno[2,3-d]pyrimidin-4-ylamino)-benzamide,
3-Ethoxy-4-(5-trifluoromethyl-thieno[2,3-d]pyrimidin-4-ylamino)-benzamide,
3-Isopropoxy-4-(5-trifluoromethyl-thieno[2,3-d]pyrimidin-4-ylamino)-benzamide,
(4-Fluoro-2-isopropoxy-phenyl)-thieno[2,3-d]pyrimidin-4-yl-amine,
(2-sec-Butoxy-4-fluoro-phenyl)thieno[2,3-d]pyrimidin-4-yl-amine,
(4-Fluoro-2-isopropoxy-phenyl)-(5-methyl-thieno[2,3-d]pyrimidin-4-yl)-amine,
(2-sec-Butoxy-4-fluoro-phenyl)-(5-methyl-thieno[2,3-d]pyrimidin-4-yl)-amine,
(2-sec-Butoxy-4-fluoro-phenyl)-(5,6-dimethyl-thieno[2,3-d]pyrimidin-4-yl)-amine,
(4-Fluoro-2-methoxy-phenyl)-(5-trifluoromethyl-thieno[2,3-d]pyrimidin-4-yl)-amine,
(4-Fluoro-2-isopropoxy-phenyl)-(5-trifluoromethyl-thieno[2,3-d]pyrimidin-4-yl)-amine,
[4-Fluoro-2-(tetrahydro-furan-3-yloxy)-phenyl]-(5-trifluoromethyl-thieno[2,3-d]pyrimidin-4-yl)-amine,
[4-Fluoro-2-(3,3,3-trifluoro-propoxy)-phenyl]-thieno[2,3-d]pyrimidin-4-yl-amine,
[4-Fluoro-2-(3,3,3-trifluoro-propoxy)-phenyl]-(5-methyl-thieno[2,3-d]pyrimidin-4-yl)-amine,
(5,6-Dimethyl-thieno[2,3-d]pyrimidin-4-yl)-[4-fluoro-2-(3,3,3-trifluoro-propoxy)-phenyl]-amine,
4-(5,6-Dimethyl-thieno[2,3-d]pyrimidin-4-ylamino)-N-methyl-3-(tetrahydro-furan-3-yloxy)-benzamide,
2-Fluoro-5-methoxy-4-(thieno[2,3-d]pyrimidin-4-ylamino)-benzamide,
2-Fluoro-5-isopropoxy-4-(thieno[2,3-d]pyrimidin-4-ylamino)-benzamide,
2-Fluoro-5-methoxy-4-(5-methyl-thieno[2,3-d]pyrimidin-4-ylamino)-benzamide,
2-Fluoro-4-(5-methyl-thieno[2,3-d]pyrimidin-4-ylamino)-5-(tetrahydro-furan-3-yloxy)-benzamide,
4-(5,6-Dimethyl-thieno[2,3-d]pyrimidin-4-ylamino)-2-fluoro-5-methoxy-benzamide,
4-(5,6-Dimethyl-thieno[2,3-d]pyrimidin-4-ylamino)-2-fluoro-5-isopropoxy-benzamide,
4-(5,6-Dimethyl-thieno[2,3-d]pyrimidin-4-ylamino)-2-fluoro-5-(tetrahydro-furan-3-yloxy)-benzamide,
[2-(1-Methanesulfonyl-pyrrolidin-3-yloxy)-phenyl]-(5-methyl-thieno[2,3-d]pyrimidin-4-yl)-amine,
1-{3-[2-(5-Methyl-thieno[2,3-d]pyrimidin-4-ylamino)-phenoxy]-pyrrolidin-1-yl}-ethanone,
3-[2-(5-Methyl-thieno[2,3-d]pyrimidin-4-ylamino)-phenoxy]-pyrrolidine-1-carboxylic acid dimethylamide,
2-Methyl-1-{3-[2-(5-methyl-thieno[2,3-d]pyrimidin-4-ylamino)-phenoxy]-pyrrolidin-1-yl}-propan-1-one,
Cyclopropyl-{3-[2-(5-methyl-thieno[2,3-d]pyrimidin-4-ylamino)-phenoxy]-pyrrolidin-1-yl}-methanone,
Cyclopentyl-{3-[2-(5-methyl-thieno[2,3-d]pyrimidin-4-ylamino)-phenoxy]-pyrrolidin-1-yl}-methanone,
3-[2-(5-Methyl-thieno[2,3-d]pyrimidin-4-ylamino)-phenoxy]-pyrrolidine-1-sulfonic acid dimethylamide,
(5-Methyl-thieno[2,3-d]pyrimidin-4-yl)-{2-[1-(propane-2-sulfonyl)-pyrrolidin-3-yloxy]-phenyl}-amine,
{3-[2-(5-Methyl-thieno[2,3-d]pyrimidin-4-ylamino)-phenoxy]-pyrrolidin-1-yl}-pyridin-4-yl-methanone,
3-sec-Butoxy-4-(5,6-dimethyl-thieno[2,3-d]pyrimidin-4-ylamino)-benzamide,
3-Ethoxy-4-(5-methyl-thieno[2,3-d]pyrimidin-4-ylamino)-benzonitrile,
3-Isopropoxy-4-(5-methyl-thieno[2,3-d]pyrimidin-4-ylamino)-benzonitrile,
3-sec-Butoxy-4-(5,6-dimethyl-thieno[2,3-d]pyrimidin-4-ylamino)-benzonitrile,
(5,6-Dimethyl-thieno[2,3-d]pyrimidin-4-yl)-[2-(1-methanesulfonyl-pyrrolidin-3-yloxy)-phenyl]-amine,
3-(1-Methanesulfonyl-pyrrolidin-3-yloxy)-4-(5-methyl-thieno[2,3-d]pyrimidin-4-ylamino)-benzamide,
4-(5,6-Dimethyl-thieno[2,3-d]pyrimidin-4-ylamino)-3-(1-methanesulfonyl-pyrrolidin-3-yloxy)-benzamide,

14. A compound according to claim 13 selected from:
[2-(Bicyclo[2.2.1]hept-2-yloxy)-phenyl]-(5,6-dimethyl-thieno[2,3-d]pyrimidin-4-yl)-amine,
(6-Ethyl-thieno[2,3-d]pyrimidin-4-yl)-(2-methoxy-phenyl)-amine,
(2-Ethoxy-phenyl)-(6-isopropyl-thieno[2,3-d]pyrimidin-4-yl)-amine,
(6-Isopropyl-thieno[2,3-d]pyrimidin-4-yl)-(2-methoxy-phenyl)-amine,
(2-sec-Butoxy-phenyl)-(6-isopropyl-thieno[2,3-d]pyrimidin-4-yl)-amine,
(2-Cyclopentylsulfanyl-phenyl)-thieno[2,3-d]pyrimidin-4-yl-amine,
(2-Cyclohexylsulfanyl-phenyl)thieno[2,3-d]pyrimidin-4-yl-amine
[2-(Bicyclo[2.2.1]hept-2-yloxy)-phenyl]-thieno[2,3-d]pyrimidin-4-yl-amine,
[2-(Tetrahydro-furan-3-ylmethoxy)-phenyl]-thieno[2,3-d]pyrimidin-4-yl-amine,
[5-Fluoro-2-(tetrahydro-furan-3-yloxy)-phenyl]-thieno[2,3-d]pyrimidin-4-yl-amine,
(2-sec-Butoxy-phenyl)-(6-methyl-5-propyl-thieno[2,3-d]pyrimidin-4-yl)-amine,
4-(5,6-Dimethyl-thieno[2,3-d]pyrimidin-4-ylamino)-3-(tetrahydro-furan-3-yloxy)-benzoic acid methyl ester,
(5,6-Dimethyl-thieno[2,3-d]pyrimidin-4-yl)-(2-methanesulfonyl-phenyl)-amine,
(2-Ethoxy-5-fluoro-phenyl)thieno[2,3-d]pyrimidin-4-yl-amine,
(2-Ethoxy-5-fluoro-phenyl)-(5-methyl-thieno[2,3-d]pyrimidin-4-yl)-amine
[2-(3,5-Dimethyl-piperazin-1-yl)-phenyl]-(5-methyl-thieno[2,3-d]pyrimidin-4-yl)-amine,
(2-Cyclopentyloxy-phenyl)-(6-methyl-5-propyl-thieno[2,3-d]pyrimidin-4-yl)-amine,
(2-Pyrrolidin-1-yl-phenyl)thieno[2,3-d]pyrimidin-4-yl-amine,
3-[2-(5-Methyl-thieno[2,3-d]pyrimidin-4-ylamino)-phenoxy]-pyrrolidine-1-carboxylic acid tert-butyl ester,
N-sec-Butyl-N′-(5-methyl-thieno[2,3-d]pyrimidin-4-yl)-benzene-1,2-diamine,
N-Cyclopentyl-N′-(5-methyl-thieno[2,3-d]pyrimidin-4-yl)-benzene-1,2-diamine,
[3-Fluoro-2-(tetrahydro-furan-3-yloxy)-phenyl]-thieno[2,3-d]pyrimidin-4-yl-amine,
(4-Fluoro-2-methoxy-phenyl)-thieno[2,3-d]pyrimidin-4-yl-amine,
3-[2-(Thieno[2,3-d]pyrimidin-4-ylamino)-phenoxy]-pyrrolidine-1-carboxylic acid tert-butyl ester,
[2-(Pyrrolidin-3-yloxy)-phenyl]-thieno[2,3-d]pyrimidin-4-yl-amine,
[2-(1-Methanesulfonyl-pyrrolidin-3-yloxy)-phenyl]-thieno[2,3-d]pyrimidin-4-yl-amine,
{2-[1-(Propane-2-sulfonyl)-pyrrolidin-3-yloxy]-phenyl}-thieno[2,3-d]pyrimidin-4-yl-amine,
3-[2-(Thieno[2,3-d]pyrimidin-4-ylamino)-phenoxy]-pyrrolidine-1-sulfonic acid dimethylamide,
[2-(1-Cyclopropanesulfonyl-pyrrolidin-3-yloxy)-phenyl]-thieno[2,3-d]pyrimidin-4-yl-amine,
3-[2-(Thieno[2,3-d]pyrimidin-4-ylamino)-phenoxy]-pyrrolidine-1-carboxylic acid 4-methoxy-benzylamide,
{3-[2-(5-Methyl-thieno[2,3-d]pyrimidin-4-ylamino)-phenoxy]-pyrrolidin-1-yl}-pyridin-3-yl-methanone,
[2-Ethoxy-4-(4H-[1,2,4]triazol-3-yl)-phenyl]-(5-methyl-thieno[2,3-d]pyrimidin-4-yl)-amine,
[2-(Bicyclo[2.2.1]hept-2-yloxy)-phenyl]-thieno[2,3-d]pyrimidin-4-yl-amine,
(2-Ethoxy-phenyl)-(6-ethyl-thieno[2,3-d]pyrimidin-4-yl)-amine,
(2-sec-Butoxy-phenyl)-(6-ethyl-thieno[2,3-d]pyrimidin-4-yl)-amine,
(6-Ethyl-thieno[2,3-d]pyrimidin-4-yl)-[2-(tetrahydro-furan-3-yloxy)-phenyl]-amine,
[2-(Bicyclo[2.2.1]hept-2-yloxy)-phenyl]-(5-methyl-thieno[2,3-d]pyrimidin-4-yl)-amine,
[2-(Bicyclo[2.2.1]hept-2-yloxy)-phenyl]-(5,6-dimethyl-thieno[2,3-d]pyrimidin-4-yl)-amine,
(5-Methyl-thieno[2,3-d]pyrimidin-4-yl)-[2-(tetrahydro-furan-3-ylmethoxy)-phenyl]-amine,
(5,6-Dimethyl-thieno[2,3-d]pyrimidin-4-yl)-[2-(tetrahydro-furan-3-ylmethoxy)-phenyl]-amine,
(2-Isopropoxy-phenyl)-(6-isopropyl-thieno[2,3-d]pyrimidin-4-yl)-amine,
(2-Cyclopentyloxy-phenyl)-(6-isopropyl-thieno[2,3-d]pyrimidin-4-yl)-amine,
(6-Isopropyl-thieno[2,3-d]pyrimidin-4-yl)-[2-(tetrahydro-furan-3-yloxy)-phenyl]-amine,
[2-(1,2-Dimethyl-propoxy)-phenyl]-thieno[2,3-d]pyrimidin-4-yl-amine,
[2-(1,2-Dimethyl-propoxy)-phenyl]-(5-methyl-thieno[2,3-d]pyrimidin-4-yl)-amine,
[2-(1,2-Dimethyl-propoxy)-phenyl]-(5,6-dimethyl-thieno[2,3-d]pyrimidin-4-yl)-amine,
[5-Fluoro-2-(tetrahydro-furan-3-yloxy)-phenyl]-(5-methyl-thieno[2,3-d]pyrimidin-4-yl)amine,
(5,6-Dimethyl-thieno[2,3-d]pyrimidin-4-yl)-[5-fluoro-2-(tetrahydro-furan-3-yloxy)-phenyl]-amine,
(6-Ethyl-thieno[2,3-d]pyrimidin-4-yl)-(2-isopropoxy-phenyl)-amine,
(2-Cyclopentyloxy-phenyl)-(6-ethyl-thieno[2,3-d]pyrimidin-4-yl)-amine,
(2-Methoxy-phenyl)-(6-methyl-5-propyl-thieno[2,3-d]pyrimidin-4-yl)-amine,
(2-Ethoxy-phenyl)-(6-methyl-5-propyl-thieno[2,3-d]pyrimidin-4-yl)-amine,
(2-Isopropoxy-phenyl)-(6-methyl-5-propyl-thieno[2,3-d]pyrimidin-4-yl)-amine,
3-(Tetrahydro-furan-3-yloxy)-4-(thieno[2,3-d]pyrimidin-4-ylamino)-benzoic acid methyl ester,
4-(5-Methyl-thieno[2,3-d]pyrimidin-4-ylamino)-3-(tetrahydro-furan-3-yloxy)-benzoic acid methyl ester,
3-(Tetrahydro-furan-3-yloxy)-4-(thieno[2,3-d]pyrimidin-4-ylamino)-benzamide,
[2-(Tetrahydro-furan-3-yloxy)-phenyl]-(5-trifluoromethyl-thieno[2,3-d]pyrimidin-4-yl)-amine,
(2-Cyclopentyloxy-phenyl)-(5-trifluoromethyl-thieno[2,3-d]pyrimidin-4-yl)-amine,
[2-(1-Ethyl-2-methyl-propoxy)-phenyl]-thieno[2,3-d]pyrimidin-4-yl-amine,
[2-(1-Ethyl-2-methyl-propoxy)-phenyl]-(5-methyl-thieno[2,3-d]pyrimidin-4-yl)-amine,
(6-Methyl-5-propyl-thieno[2,3-d]pyrimidin-4-yl)-[2-(tetrahydro-furan-3-yloxy)-phenyl]-amine,
(5,6-Dimethyl-thieno[2,3-d]pyrimidin-4-yl)-[2-(1-ethyl-2-methyl-propoxy)-phenyl]-amine,
(2-Isopropoxy-phenyl)-(5-trifluoromethyl-thieno[2,3-d]pyrimidin-4-yl)-amine,
(2-sec-Butoxy-phenyl)-(5-trifluoromethyl-thieno[2,3-d]pyrimidin-4-yl)-amine,
(5-Methyl-thieno[2,3-d]pyrimidin-4-yl)-(2-pyrrolidin-1-yl-phenyl)-amine,
(5,6-Dimethyl-thieno[2,3-d]pyrimidin-4-yl)-(2-pyrrolidin-1-yl-phenyl)-amine,
3-[2-(5,6-Dimethyl-thieno[2,3-d]pyrimidin-4-ylamino)-phenoxy]-pyrrolidine-1-carboxylic acid tert-butyl ester,
Thieno[2,3-d]pyrimidin-4-yl-[2-(3,3,3-trifluoro-propoxy)-phenyl]-amine,
(5-Methyl-thieno[2,3-d]pyrimidin-4-yl)-[2-(3,3,3-trifluoro-propoxy)-phenyl]-amine
(5,6-Dimethyl-thieno[2,3-d]pyrimidin-4-yl)-[2-(3,3,3-trifluoro-propoxy)-phenyl]-amine,
(5-Ethyl-thieno[2,3-d]pyrimidin-4-yl)-(2-isopropoxy-phenyl)-amine,
(2-sec-Butoxy-phenyl)-(5-ethyl-thieno[2,3-d]pyrimidin-4-yl)-amine,
(2-Cyclopentyloxy-phenyl)-(5-ethyl-thieno[2,3-d]pyrimidin-4-yl)-amine,
(5,6-Dimethyl-thieno[2,3-d]pyrimidin-4-yl)-[2-(3-ethoxy-propoxy)-phenyl]-amine,
[3-Fluoro-2-(tetrahydro-furan-3-yloxy)-phenyl]-(5-methyl-thieno[2,3-d]pyrimidin-4-yl)amine,
(5,6-Dimethyl-thieno[2,3-d]pyrimidin-4-yl)-[3-fluoro-2-(tetrahydro-furan-3-yloxy)-phenyl]-amine,
(5,6-Dimethyl-thieno[2,3-d]pyrimidin-4-yl)-[2-(2-ethoxy-ethoxy)-phenyl]-amine,
3-Isopropoxy-4-(thieno[2,3-d]pyrimidin-4-ylamino)-benzamide,
3-sec-Butoxy-4-(thieno[2,3-d]pyrimidin-4-ylamino)-benzamide,
3-Cyclopentyloxy-4-(thieno[2,3-d]pyrimidin-4-ylamino)-benzamide,
3-Ethoxy-4-(5-methyl-thieno[2,3-d]pyrimidin-4-ylamino)-benzamide,
3-Isopropoxy-4-(5-methyl-thieno[2,3-d]pyrimidin-4-ylamino)-benzamide,
3-sec-Butoxy-4-(5-methyl-thieno[2,3-d]pyrimidin-4-ylamino)-benzamide,
3-Cyclopentyloxy-4-(5-methyl-thieno[2,3-d]pyrimidin-4-ylamino)-benzamide,
4-(5-Methyl-thieno[2,3-d]pyrimidin-4-ylamino)-3-(tetrahydro-furan-3-yloxy)-benzamide,
3-(Tetrahydro-furan-3-yloxy)-4-(5-trifluoromethyl-thieno[2,3-d]pyrimidin-4-ylamino)-benzamide,
4-(Thieno[2,3-d]pyrimidin-4-ylamino)-3-(3,3,3-trifluoro-propoxy)-benzamide,
4-(5-Methyl-thieno[2,3-d]pyrimidin-4-ylamino)-3-(3,3,3-trifluoro-propoxy)-benzamide,
4-(5,6-Dimethyl-thieno[2,3-d]pyrimidin-4-ylamino)-3-(3,3,3-trifluoro-propoxy)-benzamide,
3-Pyrrolidin-1-yl-4-(thieno[2,3-d]pyrimidin-4-ylamino)-benzamide,
4-(5-Methyl-thieno[2,3-d]pyrimidin-4-ylamino)-3-pyrrolidin-1-yl-benzamide,
4-(5,6-Dimethyl-thieno[2,3-d]pyrimidin-4-ylamino)-3-pyrrolidin-1-yl-benzamide,
4-(5,6-Dimethyl-thieno[2,3-d]pyrimidin-4-ylamino)-3-ethoxy-benzamide,
4-(5,6-Dimethyl-thieno[2,3-d]pyrimidin-4-ylamino)-3-isopropoxy-benzamide,
3-Cyclopentyloxy-4-(5,6-dimethyl-thieno[2,3-d]pyrimidin-4-ylamino)-benzamide,
4-(5,6-Dimethyl-thieno[2,3-d]pyrimidin-4-ylamino)-3-(tetrahydro-furan-3-yloxy)-benzamide,
(2-Ethoxy-4-fluoro-phenyl)-thieno[2,3-d]pyrimidin-4-yl-amine,
[4-Fluoro-2-(tetrahydro-furan-3-yloxy)-phenyl]-thieno[2,3-d]pyrimidin-4-yl-amine,
(4-Fluoro-2-methoxy-phenyl)-(5-methyl-thieno[2,3-d]pyrimidin-4-yl)-amine,
(2-Ethoxy-4-fluoro-phenyl)-(5-methyl-thieno[2,3-d]pyrimidin-4-yl)-amine,
(2-Cyclopentyloxy-4-fluoro-phenyl)-(5-methyl-thieno[2,3-d]pyrimidin-4-yl)-amine,
[4-Fluoro-2-(tetrahydro-furan-3-yloxy)-phenyl]-(5-methyl-thieno[2,3-d]pyrimidin-4-yl)-amine,
(5,6-Dimethyl-thieno[2,3-d]pyrimidin-4-yl)-(4-fluoro-2-methoxy-phenyl)-amine,
(5,6-Dimethyl-thieno[2,3-d]pyrimidin-4-yl)-(2-ethoxy-4-fluoro-phenyl)-amine,
(5,6-Dimethyl-thieno[2,3-d]pyrimidin-4-yl)-(4-fluoro-2-isopropoxy-phenyl)-amine,
(5,6-Dimethyl-thieno[2,3-d]pyrimidin-4-yl)-[4-fluoro-2-(tetrahydro-furan-3-yloxy)-phenyl]-amine,
3-Methoxy-4-(5-trifluoromethyl-thieno[2,3-d]pyrimidin-4-ylamino)-benzamide,
3-Ethoxy-4-(5-trifluoromethyl-thieno[2,3-d]pyrimidin-4-ylamino)-benzamide,
3-Isopropoxy-4-(5-trifluoromethyl-thieno[2,3-d]pyrimidin-4-ylamino)-benzamide,
(4-Fluoro-2-isopropoxy-phenyl)-thieno[2,3-d]pyrimidin-4-yl-amine,
(2-sec-Butoxy-4-fluoro-phenyl)-thieno[2,3-d]pyrimidin-4-yl-amine,
(4-Fluoro-2-isopropoxy-phenyl)-(5-methyl-thieno[2,3-d]pyrimidin-4-yl)-amine,
(2-sec-Butoxy-4-fluoro-phenyl)-(5-methyl-thieno[2,3-d]pyrimidin-4-yl)-amine,
(2-sec-Butoxy-4-fluoro-phenyl)-(5,6-dimethyl-thieno[2,3-d]pyrimidin-4-yl)-amine,
(4-Fluoro-2-methoxy-phenyl)-(5-trifluoromethyl-thieno[2,3-d]pyrimidin-4-yl)-amine,
(4-Fluoro-2-isopropoxy-phenyl)-(5-trifluoromethyl-thieno[2,3-d]pyrimidin-4-yl)-amine,
[4-Fluoro-2-(tetrahydro-furan-3-yloxy)-phenyl]-(5-trifluoromethyl-thieno[2,3-d]pyrimidin-4-yl)-amine,
[4-Fluoro-2-(3,3,3-trifluoro-propoxy)-phenyl]-thieno[2,3-d]pyrimidin-4-yl-amine,
[4-Fluoro-2-(3,3,3-trifluoro-propoxy)-phenyl]-(5-methyl-thieno[2,3-d]pyrimidin-4-yl)-amine,
(5,6-Dimethyl-thieno[2,3-d]pyrimidin-4-yl)-[4-fluoro-2-(3,3,3-trifluoro-propoxy)-phenyl]-amine,
4-(5,6-Dimethyl-thieno[2,3-d]pyrimidin-4-ylamino)-N-methyl-3-(tetrahydro-furan-3-yloxy)-benzamide,
2-Fluoro-5-methoxy-4-(thieno[2,3-d]pyrimidin-4-ylamino)-benzamide,
2-Fluoro-5-isopropoxy-4-(thieno[2,3-d]pyrimidin-4-ylamino)-benzamide,
2-Fluoro-5-methoxy-4-(5-methyl-thieno[2,3-d]pyrimidin-4-ylamino)-benzamide,
2-Fluoro-4-(5-methyl-thieno[2,3-d]pyrimidin-4-ylamino)-5-(tetrahydro-furan-3-yloxy)-benzamide,
4-(5,6-Dimethyl-thieno[2,3-d]pyrimidin-4-ylamino)-2-fluoro-5-methoxy-benzamide,
4-(5,6-Dimethyl-thieno[2,3-d]pyrimidin-4-ylamino)-2-fluoro-5-isopropoxy-benzamide,
4-(5,6-Dimethyl-thieno[2,3-d]pyrimidin-4-ylamino)-2-fluoro-5-(tetrahydro-furan-3-yloxy)-benzamide,
[2-(1-Methanesulfonyl-pyrrolidin-3-yloxy)-phenyl]-(5-methyl-thieno[2,3-d]pyrimidin-4-yl)-amine,
1-{3-[2-(5-Methyl-thieno[2,3-d]pyrimidin-4-ylamino)-phenoxy]pyrrolidin-1-yl}-ethanone,
3-[2-(5-Methyl-thieno[2,3-d]pyrimidin-4-ylamino)-phenoxy]-pyrrolidine-1-carboxylic acid dimethylamide,
2-Methyl-1-{3-[2-(5-methyl-thieno[2,3-d]pyrimidin-4-ylamino)-phenoxy]-pyrrolidin-1-yl}-propan-1-one,
Cyclopropyl-{3-[2-(5-methyl-thieno[2,3-d]pyrimidin-4-ylamino)-phenoxy]-pyrrolidin-1-yl}-methanone,
Cyclopentyl-{3-[2-(5-methyl-thieno[2,3-d]pyrimidin-4-ylamino)-phenoxy]-pyrrolidin-1-yl}-methanone,
3-[2-(5-Methyl-thieno[2,3-d]pyrimidin-4-ylamino)-phenoxy]-pyrrolidine-1-sulfonic acid dimethylamide,
(5-Methyl-thieno[2,3-d]pyrimidin-4-yl)-{2-[1-(propane-2-sulfonyl)-pyrrolidin-3-yloxy]-phenyl}-amine,
{3-[2-(5-Methyl-thieno[2,3-d]pyrimidin-4-ylamino)-phenoxy]-pyrrolidin-1-yl}-pyridin-4-yl-methanone,
3-sec-Butoxy-4-(5,6-dimethyl-thieno[2,3-d]pyrimidin-4-ylamino)-benzamide,
3-Ethoxy-4-(5-methyl-thieno[2,3-d]pyrimidin-4-ylamino)-benzonitrile,
3-Isopropoxy-4-(5-methyl-thieno[2,3-d]pyrimidin-4-ylamino)-benzonitrile,
3-sec-Butoxy-4-(5,6-dimethyl-thieno[2,3-d]pyrimidin-4-ylamino)-benzonitrile,
(5,6-Dimethyl-thieno[2,3-d]pyrimidin-4-yl)-[2-(1-methanesulfonyl-pyrrolidin-3-yloxy)-phenyl]-amine,
3-(1-Methanesulfonyl-pyrrolidin-3-yloxy)-4-(5-methyl-thieno[2,3-d]pyrimidin-4-ylamino)-benzamide,
4-(5,6-Dimethyl-thieno[2,3-d]pyrimidin-4-ylamino)-3-(1-methanesulfonyl-pyrrolidin-3-yloxy)-benzamide,
[2-(Bicyclo[2.2.1]hept-2-yloxy)-phenyl]-(5-methyl-thieno[2,3-d]pyrimidin-4-yl)-amine,
(5-Ethyl-thieno[2,3-d]pyrimidin-4-yl)-[2-(tetrahydro-furan-3-yloxy)-phenyl]-amine,
[2-(3-Ethoxy-propoxy)-phenyl]-(5-methyl-thieno[2,3-d]pyrimidin-4-yl)-amine,
[2-(2-Ethoxy-ethoxy)-phenyl]-(5-methyl-thieno[2,3-d]pyrimidin-4-yl)-amine,
3-Ethoxy-4-(thieno[2,3-d]pyrimidin-4-ylamino)-benzamide,
(2-Cyclopentyloxy-4-fluoro-phenyl)-thieno[2,3-d]pyrimidin-4-yl-amine,
2-Fluoro-5-(tetrahydro-furan-3-yloxy)-4-(thieno[2,3-d]pyrimidin-4-ylamino)benzamide.

15. A compound according to claim 14 selected from:
[2-(Bicyclo[2.2.1]hept-2-yloxy)-phenyl]-thieno[2,3-d]pyrimidin-4-yl-amine,
(2-Ethoxy-phenyl)-(6-ethyl-thieno[2,3-d]pyrimidin-4-yl)-amine,
(2-sec-Butoxy-phenyl)-(6-ethyl-thieno[2,3-d]pyrimidin-4-yl)-amine,
(6-Ethyl-thieno[2,3-d]pyrimidin-4-yl)-[2-(tetrahydro-furan-3-yloxy)-phenyl]-amine,
[2-(Bicyclo[2.2.1]hept-2-yloxy)-phenyl]-(5-methyl-thieno[2,3-d]pyrimidin-4-yl)-amine,
[2-(Bicyclo[2.2.1]hept-2-yloxy)-phenyl]-(5,6-dimethyl-thieno[2,3-d]pyrimidin-4-yl)-amine,
(5-Methyl-thieno[2,3-d]pyrimidin-4-yl)-[2-(tetrahydro-furan-3-ylmethoxy)-phenyl]-amine,
(5,6-Dimethyl-thieno[2,3-d]pyrimidin-4-yl)-[2-(tetrahydro-furan-3-ylmethoxy)-phenyl]-amine,
(2-Isopropoxy-phenyl)-(6-isopropyl-thieno[2,3-d]pyrimidin-4-yl)-amine,
(2-Cyclopentyloxy-phenyl)-(6-isopropyl-thieno[2,3-d]pyrimidin-4-yl)-amine,
(6-Isopropyl-thieno[2,3-d]pyrimidin-4-yl)-[2-(tetrahydro-furan-3-yloxy)-phenyl]-amine,
[2-(1,2-Dimethyl-propoxy)-phenyl]-thieno[2,3-d]pyrimidin-4-yl-amine,
[2-(1,2-Dimethyl-propoxy)-phenyl]-(5-methyl-thieno[2,3-d]pyrimidin-4-yl)-amine,
[2-(1,2-Dimethyl-propoxy)-phenyl]-(5,6-dimethyl-thieno[2,3-d]pyrimidin-4-yl)-amine,
[5-Fluoro-2-(tetrahydro-furan-3-yloxy)-phenyl]-(5-methyl-thieno[2,3-d]pyrimidin-4-yl)-amine,
(5,6-Dimethyl-thieno[2,3-d]pyrimidin-4-yl)-[5-fluoro-2-(tetrahydro-furan-3-yloxy)-phenyl]-amine,
(6-Ethyl-thieno[2,3-d]pyrimidin-4-yl)-(2-isopropoxy-phenyl)-amine,
(2-Cyclopentyloxy-phenyl)-(6-ethyl-thieno[2,3-d]pyrimidin-4-yl)-amine,
(2-Methoxy-phenyl)-(6-methyl-5-propyl-thieno[2,3-d]pyrimidin-4-yl)-amine,
(2-Ethoxy-phenyl)-(6-methyl-5-propyl-thieno[2,3-d]pyrimidin-4-yl)-amine,
(2-Isopropoxy-phenyl)-(6-methyl-5-propyl-thieno[2,3-d]pyrimidin-4-yl)-amine,
3-(Tetrahydro-furan-3-yloxy)-4-(thieno[2,3-d]pyrimidin-4-ylamino)-benzoic acid methyl ester,
4-(5-Methyl-thieno[2,3-d]pyrimidin-4-ylamino)-3-(tetrahydro-furan-3-yloxy)-benzoic acid methyl ester,
3-(Tetrahydro-furan-3-yloxy)-4-(thieno[2,3-d]pyrimidin-4-ylamino)-benzamide,
[2-(Tetrahydro-furan-3-yloxy)-phenyl]-(5-trifluoromethyl-thieno[2,3-d]pyrimidin-4-yl)-amine,
(2-Cyclopentyloxy-phenyl)-(5-trifluoromethyl-thieno[2,3-d]pyrimidin-4-yl)-amine,
[2-(1-Ethyl-2-methyl-propoxy)-phenyl]-thieno[2,3-d]pyrimidin-4-yl-amine,
[2-(1-Ethyl-2-methyl-propoxy)-phenyl]-(5-methyl-thieno[2,3-d]pyrimidin-4-yl)-amine,
(6-Methyl-5-propyl-thieno[2,3-d]pyrimidin-4-yl)-[2-(tetrahydro-furan-3-yloxy)-phenyl]-amine,
(5,6-Dimethyl-thieno[2,3-d]pyrimidin-4-yl)-[2-(1-ethyl-2-methyl-propoxy)-phenyl]-amine,
(2-Isopropoxy-phenyl)-(5-trifluoromethyl-thieno[2,3-d]pyrimidin-4-yl)-amine,
(2-sec-Butoxy-phenyl)-(5-trifluoromethyl-thieno[2,3-d]pyrimidin-4-yl)-amine,
(5-Methyl-thieno[2,3-d]pyrimidin-4-yl)-(2-pyrrolidin-1-yl-phenyl)-amine,
(5,6-Dimethyl-thieno[2,3-d]pyrimidin-4-yl)-(2-pyrrolidin-1-yl-phenyl)-amine,
3-[2-(5,6-Dimethyl-thieno[2,3-d]pyrimidin-4-ylamino)-phenoxy]pyrrolidine-1-carboxylic acid tert-butyl ester,
Thieno[2,3-d]pyrimidin-4-yl-[2-(3,3,3-trifluoro-propoxy)-phenyl]-amine,
(5-Methyl-thieno[2,3-d]pyrimidin-4-yl)-[2-(3,3,3-trifluoro-propoxy)-phenyl]-amine
(5,6-Dimethyl-thieno[2,3-d]pyrimidin-4-yl)-[2-(3,3,3-trifluoro-propoxy)-phenyl]-amine,
(5-Ethyl-thieno[2,3-d]pyrimidin-4-yl)-(2-isopropoxy-phenyl)-amine,
(2-sec-Butoxy-phenyl)-(5-ethyl-thieno[2,3-d]pyrimidin-4-yl)-amine,
(2-Cyclopentyloxy-phenyl)-(5-ethyl-thieno[2,3-d]pyrimidin-4-yl)-amine,
(5,6-Dimethyl-thieno[2,3-d]pyrimidin-4-yl)-[2-(3-ethoxy-propoxy)-phenyl]-amine,
[3-Fluoro-2-(tetrahydro-furan-3-yloxy)-phenyl]-(5-methyl-thieno[2,3-d]pyrimidin-4-yl)-amine,
(5,6-Dimethyl-thieno[2,3-d]pyrimidin-4-yl)-[3-fluoro-2-(tetrahydro-furan-3-yloxy)-phenyl]-amine,
(5,6-Dimethyl-thieno[2,3-d]pyrimidin-4-yl)-[2-(2-ethoxy-ethoxy)-phenyl]-amine,
3-Isopropoxy-4-(thieno[2,3-d]pyrimidin-4-ylamino)-benzamide,
3-sec-Butoxy-4-(thieno[2,3-d]pyrimidin-4-ylamino)-benzamide,
3-Cyclopentyloxy-4-(thieno[2,3-d]pyrimidin-4-ylamino)-benzamide,
3-Ethoxy-4-(5-methyl-thieno[2,3-d]pyrimidin-4-ylamino)-benzamide,
3-Isopropoxy-4-(5-methyl-thieno[2,3-d]pyrimidin-4-ylamino)-benzamide,
3-sec-Butoxy-4-(5-methyl-thieno[2,3-d]pyrimidin-4-ylamino)-benzamide,
3-Cyclopentyloxy-4-(5-methyl-thieno[2,3-d]pyrimidin-4-ylamino)-benzamide,
4-(5-Methyl-thieno[2,3-d]pyrimidin-4-ylamino)-3-(tetrahydro-furan-3-yloxy)-benzamide,
3-(Tetrahydro-furan-3-yloxy)-4-(5-trifluoromethyl-thieno[2,3-d]pyrimidin-4-ylamino)-benzamide,
4-(Thieno[2,3-d]pyrimidin-4-ylamino)-3-(3,3,3-trifluoro-propoxy)-benzamide,
4-(5-Methyl-thieno[2,3-d]pyrimidin-4-ylamino)-3-(3,3,3-trifluoro-propoxy)-benzamide,
4-(5,6-Dimethyl-thieno[2,3-d]pyrimidin-4-ylamino)-3-(3,3,3-trifluoro-propoxy)-benzamide,
3-Pyrrolidin-1-yl-4-(thieno[2,3-d]pyrimidin-4-ylamino)-benzamide,
4-(5-Methyl-thieno[2,3-d]pyrimidin-4-ylamino)-3-pyrrolidin-1-yl-benzamide,
4-(5,6-Dimethyl-thieno[2,3-d]pyrimidin-4-ylamino)-3-pyrrolidin-1-yl-benzamide,
4-(5,6-Dimethyl-thieno[2,3-d]pyrimidin-4-ylamino)-3-ethoxy-benzamide,
4-(5,6-Dimethyl-thieno[2,3-d]pyrimidin-4-ylamino)-3-isopropoxy-benzamide,
3-Cyclopentyloxy-4-(5,6-dimethyl-thieno[2, 3-d]pyrimidin-4-ylamino)-benzamide,
4-(5,6-Dimethyl-thieno[2,3-d]pyrimidin-4-ylamino)-3-(tetrahydro-furan-3-yloxy)-benzamide,
(2-Ethoxy-4-fluoro-phenyl)thieno[2,3-d]pyrimidin-4-yl-amine,
[4-Fluoro-2-(tetrahydro-furan-3-yloxy)-phenyl]-thieno[2,3-d]pyrimidin-4-yl-amine,
(4-Fluoro-2-methoxy-phenyl)-(5-methyl-thieno[2,3-d]pyrimidin-4-yl)-amine,
(2-Ethoxy-4-fluoro-phenyl)-(5-methyl-thieno[2,3-d]pyrimidin-4-yl)-amine,
(2-Cyclopentyloxy-4-fluoro-phenyl)-(5-methyl-thieno[2,3-d]pyrimidin-4-yl)-amine,
[4-Fluoro-2-(tetrahydro-furan-3-yloxy)-phenyl]-(5-methyl-thieno[2,3-d]pyrimidin-4-yl)-amine,
(5,6-Dimethyl-thieno[2,3-d]pyrimidin-4-yl)-(4-fluoro-2-methoxy-phenyl)-amine,
(5,6-Dimethyl-thieno[2,3-d]pyrimidin-4-yl)-(2-ethoxy-4-fluoro-phenyl)-amine,
(5,6-Dimethyl-thieno[2,3-d]pyrimidin-4-yl)-(4-fluoro-2-isopropoxy-phenyl)-amine,
(5,6-Dimethyl-thieno[2,3-d]pyrimidin-4-yl)-[4-fluoro-2-(tetrahydro-furan-3-yloxy)-phenyl]-amine,
3-Methoxy-4-(5-trifluoromethyl-thieno[2,3-d]pyrimidin-4-ylamino)-benzamide,
3-Ethoxy-4-(5-trifluoromethyl-thieno[2,3-d]pyrimidin-4-ylamino)-benzamide,
3-Isopropoxy-4-(5-trifluoromethyl-thieno[2,3-d]pyrimidin-4-ylamino)-benzamide,
(4-Fluoro-2-isopropoxy-phenyl)-thieno[2,3-d]pyrimidin-4-yl-amine,
(2-sec-Butoxy-4-fluoro-phenyl)-thieno[2,3-d]pyrimidin-4-yl-amine,
(4-Fluoro-2-isopropoxy-phenyl)-(5-methyl-thieno[2,3-d]pyrimidin-4-yl)-amine,
(2-sec-Butoxy-4-fluoro-phenyl)-(5-methyl-thieno[2,3-d]pyrimidin-4-yl)-amine,
(2-sec-Butoxy-4-fluoro-phenyl)-(5,6-dimethyl-thieno[2,3-d]pyrimidin-4-yl)-amine,
(4-Fluoro-2-methoxy-phenyl)-(5-trifluoromethyl-thieno[2,3-d]pyrimidin-4-yl)-amine,
(4-Fluoro-2-isopropoxy-phenyl)-(5-trifluoromethyl-thieno[2,3-d]pyrimidin-4-yl)-amine,
[4-Fluoro-2-(tetrahydro-furan-3-yloxy)-phenyl]-(5-trifluoromethyl-thieno[2,3-d]pyrimidin-4-yl)-amine,
[4-Fluoro-2-(3,3,3-trifluoro-propoxy)-phenyl]-thieno[2,3-d]pyrimidin-4-yl-amine,
[4-Fluoro-2-(3,3,3-trifluoro-propoxy)-phenyl]-(5-methyl-thieno[2,3-d]pyrimidin-4-yl)-amine,
(5,6-Dimethyl-thieno[2,3-d]pyrimidin-4-yl)-[4-fluoro-2-(3,3,3-trifluoro-propoxy)-phenyl]-amine,
4-(5,6-Dimethyl-thieno[2,3-d]pyrimidin-4-ylamino)-N-methyl-3-(tetrahydro-furan-3-yloxy)-benzamide,
2-Fluoro-5-methoxy-4-(thieno[2,3-d]pyrimidin-4-ylamino)-benzamide,
2-Fluoro-5-isopropoxy-4-(thieno[2,3-d]pyrimidin-4-ylamino)-benzamide,
2-Fluoro-5-methoxy-4-(5-methyl-thieno[2,3-d]pyrimidin-4-ylamino)-benzamide,
2-Fluoro-4-(5-methyl-thieno[2,3-d]pyrimidin-4-ylamino)-5-(tetrahydro-furan-3-yloxy)-benzamide,
4-(5,6-Dimethyl-thieno[2,3-d]pyrimidin-4-ylamino)-2-fluoro-5-methoxy-benzamide,
4-(5,6-Dimethyl-thieno[2,3-d]pyrimidin-4-ylamino)-2-fluoro-5-isopropoxy-benzamide,
4-(5,6-Dimethyl-thieno[2,3-d]pyrimidin-4-ylamino)-2-fluoro-5-(tetrahydro-furan-3-yloxy)-benzamide,
[2-(1-Methanesulfonyl-pyrrolidin-3-yloxy)-phenyl]-(5-methyl-thieno[2,3-d]pyrimidin-4-yl)-amine,
1-{3-[2-(5-Methyl-thieno[2,3-d]pyrimidin-4-ylamino)-phenoxy]-pyrrolidin-1-yl}-ethanone,
3-[2-(5-Methyl-thieno[2,3-d]pyrimidin-4-ylamino)-phenoxy]-pyrrolidine-1-carboxylic acid dimethylamide,
2-Methyl-1-{3-[2-(5-methyl-thieno[2,3-d]pyrimidin-4-ylamino)-phenoxy]-pyrrolidin-1-yl}-propan-1-one,
Cyclopropyl-{3-[2-(5-methyl-thieno[2,3-d]pyrimidin-4-ylamino)-phenoxy]-pyrrolidin-1-yl}-methanone,
Cyclopentyl-{3-[2-(5-methyl-thieno[2,3-d]pyrimidin-4-ylamino)-phenoxy]-pyrrolidin-1-yl}-methanone,
3-[2-(5-Methyl-thieno[2,3-d]pyrimidin-4-ylamino)-phenoxy]-pyrrolidine-1-sulfonic acid dimethylamide,
(5-Methyl-thieno[2,3-d]pyrimidin-4-yl)-{2-[1-(propane-2-sulfonyl)-pyrrolidin-3-yloxy]-phenyl}-amine,
{3-[2-(5-Methyl-thieno[2,3-d]pyrimidin-4-ylamino)-phenoxy]-pyrrolidin-1-yl}-pyridin-4-yl-methanone,
3-sec-Butoxy-4-(5,6-dimethyl-thieno[2,3-d]pyrimidin-4-ylamino)-benzamide,
3-Ethoxy-4-(5-methyl-thieno[2,3-d]pyrimidin-4-ylamino)-benzonitrile,
3-Isopropoxy-4-(5-methyl-thieno[2,3-d]pyrimidin-4-ylamino)-benzonitrile,
3-sec-Butoxy-4-(5,6-dimethyl-thieno[2,3-d]pyrimidin-4-ylamino)-benzonitrile,
(5,6-Dimethyl-thieno[2,3-d]pyrimidin-4-yl)-[2-(1 -methanesulfonyl-pyrrolidin-3-yloxy)-phenyl]-amine,
3-(1-Methanesulfonyl-pyrrolidin-3-yloxy)-4-(5-methyl-thieno[2,3-d]pyrimidin-4-ylamino)-benzamide,
4-(5,6-Dimethyl-thieno[2,3-d]pyrimidin-4-ylamino)-3-(1-methanesulfonyl-pyrrolidin-3-yloxy)-benzamide,
[2-(Bicyclo[2.2.1]hept-2-yloxy)-phenyl]-(5-methyl-thieno[2,3-d]pyrimidin-4-yl)-amine,
(5-Ethyl-thieno[2,3-d]pyrimidin-4-yl)-[2-(tetrahydro-furan-3-yloxy)-phenyl]-amine,
[2-(3-Ethoxy-propoxy)-phenyl]-(5-methyl-thieno[2,3-d]pyrimidin-4-yl)-amine,
(2-Cyclopentyloxy-4-fluoro-phenyl)-thieno[2,3-d]pyrimidin-4-yl-amine.

16. Pharmaceutical composition comprising a compound according to any one of claims 1 to 15 and optionally a pharmaceutically acceptable carrier.

17. Pharmaceutical composition according to claim 16 further comprising an additional therapeutic agent.

18. Pharmaceutical composition according to claim 17, wherein the additional therapeutic agent is selected from an antidiabetic agent, a lipid lowering agent, a cardiovascular agent, an antihypertensive agent, a diuretic agent, a thrombocyte aggregation inhibitor, an antineoplastic agent or an anti-obesity agent.

19. Pharmaceutical composition according to claim 17 or 18, wherein the additional therapeutic agent is selected from human NPH insulin, human lente or ultralente insulin, insulin Lispro, insulin Aspatart, or insulin Glargine, atenolol, bisoprolol, metoprolol, esmolol, celiprolol, talinolol, oxprenolol, pindolol, propanolol, bupropanolol, penbutolol, mepindolol, sotalol, certeolol, nadolol, carvedilol, nifedipin, nitrendipin, amlodipin, nicardipin, nisoldipin, diltiazem, enalapril, verapamil, gallopamil, quinapril, captopril, lisinopril, benazepril, ramipril, peridopril, fosinopril, trandolapril, irbesatan, losartan, valsartan, telmisartan, eprosartan, olmesartan, hydrochlorothiazide, piretanid, chlorotalidone, mefruside, furosemide, bendroflumethiazid, triamterene, dehydralazine, acetylsalicylic acid, tirofiban-HCl, dipyramidol, triclopidin, iloprost-trometanol, eptifibatide, clopidogrel, piratecam, abciximab, trapidil, simvastatine, bezafibrate, fenofibrate, gemfibrozil, etofyllin, clofibrate, etofibrate, fluvastatine, lovastatine, pravastatin, colestyramide, colestipol-HCl, xantinol nicotinat, inositol nicotinate, acipimox, nebivolol, glycerolnitrate, isosorbide mononitrate, isosorbide dinitrate, pentaerythrityl tetranitrate, indapamide, cilazepril, urapidil, eprosartan, nilvadipin, metoprolol, doxazosin, molsidormin, moxaverin, acebutolol, prazosine, trapidil, clonidine, vinbiastin, vincristin, vindesin, vinorelbin, podophyllotoxine derivatives, etoposid, teniposid, alkylating agents, nitroso ureas, N-lost analogues, cycloplonphamid, estamustin, melphalan, ifosfamid, mitoxantron, idarubicin, doxorubicin, bleomycin, mitomycin, dactinomycin, daptomycin, cytarabin, fluorouracil, fluoroarabin, gemcitabin, tioguanin, capecitabin, adriamydin/daunorubicin, cytosine arabinosid/cytarabine, 4-HC, or other phosphamides.

20. Pharmaceutical composition according to any one of claims 16 to 19, for oral, parenteral (e.g. bronchopulmonary), local, or topical administration.

21. Use of a compound as defined in any one of claims 1 to 15 for the production of a pharmaceutical composition for inhibiting the activity of the kinase activity of Mnk1 or Mnk2 (Mnk2a, Mnk2b) or variants thereof.

22. Use of a compound as defined in any one of claims 1 to 15 for the production of a pharmaceutical composition for the prophylaxis or therapy of metabolic diseases, hematopoietic disorders and cancer and their consecutive complications and diseases.

23. Use according to claim 21 or 22 for the prophylaxis or therapy of metabolic diseases of the carbohydrate and/or lipid metabolism and their consecutive complications and disorders.

24. Use according to claim 23 for the prophylaxis or therapy of diseases of the carbohydrate metabolism and their consecutive complications and disorders selected from impaired glucose tolerance, diabetes mellitus type II, LADA, diabetes mellitus type I, obesity, metabolic syndrome, eating disorders, chachexia, osteoarthritis, biliary stones, diabetic complications such as diabetic gangrene, diabetic arthropathy, diabetic osteopenia, diabetic glomerosclerosis, diabetic nephropathy, diabetic dermopathy, diabetic neuropathy, diabetic cataract and diabetic retinopathy, diabetic maculopathy, diabetic feet syndrome, diabetic coma with or without ketoacidosis, diabetic hyperosmolar coma, hypoglycaemic coma, hyperglycaemic coma, diabetic acidosis, diabetic ketoacidosis, intracapillary glomerulonephrosis, Kimmelstiel-Wilson syndrome, diabetic amyotrophy, diabetic autonomic neuropathy, diabetic mononeuropathy, diabetic polyneuropathy, diabetic autonomic neuropathy, diabetic angiopathies, diabetic peripheral angiopathy, diabetic ulcer, diabetic arthropathy, or obesity in diabetes.

25. Use according to claim 23 for the treatment and/or prophylaxis of metabolic diseases of the lipid metabolism (i.e. lipid disorders) and their consecutive complications and disorders selected from hypercholesterolemia, dislipidemia familial hypercholesterolemia, Fredrickson's hyperlipoproteinemia, hyperbetalipoproteinemia, hyperlipidaemia, low-density-lipoprotein-type [LDL] hyperlipoproteinemia, pure hyperglyceridemia, endogenous hyperglyceridemia, isolated hypercholesterolemia, isolated hypertroglyceridemia, cardiovascular diseases selected from hypertension, ischemia, varicose veins, retinal vein occlusion, coronary heart disease, angina pectoris, myocardial infarction, stenocardia, pulmonary hypertension, congestive heart failure, glomerulopaty, tubulointestitial disorders, renal failure, angiostenosis, cerebrovascular disorders, or cerebral apoplexy.

26. Use according to claim 25 for the prophylaxis or therapy of diabetes mellitus type I or diabetes mellitus type II or LADA and their consecutive complications and disorders.

27. Use according to claim 21 or 22 for the prophylaxis or therapy of hematopoietic disorders.

28. Use according to claim 24 or 25 for the prophylaxis or therapy of diabetes mellitus type II and its consecutive complications and disorders.

29. Use according to claim 21 or 22 for the prophylaxis or therapy of obesity.

30. Use according to any one of claims 21 to 29, wherein the pharmaceutical composition is to be administered to a patient concomitantly or sequentially in combination with an additional therapeutic agent.

31. Use according to claim 30, wherein the additional therapeutic agent is selected from an antidiabetic agent, a lipid lowering agent, a cardiovascular agent, an antihypertensive agent, a diuretic agent, a thrombocyte aggregation inhibitor, an antineoplastic agent or an anti-obesity agent.

32. Use according to claim 30 or 31, wherein the additional therapeutic agent is selected from human NPH insulin, human lente or ultralente insulin, insulin Lispro, insulin Aspatart, or insulin Glargine, atenolol, bisoprolol, metoprolol, esmolol, celiprolol, talinolol, oxprenolol, pindolol, propanolol, bupropanolol, penbutolol, mepindolol, sotalol, certeolol, nadolol, carvedilol, nifedipin, nitrendipin, amlodipin, nicardipin, nisoldipin, diltiazem, enalapril, verapamil, gallopamil, quinapril, captopril, lisinopril, benazepril, ramipril, peridopril, fosinopril, trandolapril, irbesatan, losartan, valsartan, telmisartan, eprosartan, olmesartan, hydrochlorothiazide, piretanid, chlorotalidone, mefruside, furosemide, bendroflumethiazid, triamterene, dehydralazine, acetylsalicylic acid, tirofiban-HCl, dipyramidol, triclopidin, iloprost-trometanol, eptifibatide, clopidogrel, piratecam, abciximab, trapidil, simvastatine, bezafibrate, fenofibrate, gemfibrozil, etofyllin, clofibrate, etofibrate, fluvastatine, lovastatine, pravastatin, colestyramide, colestipol-HCl, xantinol nicotinat, inositol nicotinate, acipimox, nebivolol, glycerolnitrate, isosorbide mononitrate, isosorbide dinitrate, pentaerythrityl tetranitrate, indapamide, cilazepril, urapidil, eprosartan, nilvadipin, metoprolol, doxazosin, molsidormin, moxaverin, acebutolol, prazosine, trapidil, clonidine, vinblastin, vincristin, vindesin, vinorelbin, podophyllotoxine derivatives, etoposid, teniposid, alkylating agents, nitroso ureas, N-lost analogues, cyclophosphamid, estramustin, melphalan, ifosfamid, mitoxantron, idarubicin, doxorubicin, bleomycin, mitomycin, dactinomycin, daptomycin, cytarabin, fluorouracil, fluoroarabin, gemcitabin, tioguanin, capecitabin, adriamydin/daunorubicin, cytosine arabinosid/cytarabine, 4-HC, or other phosphamides.

33. Use according to any one of claims 21 to 32, wherein the pharmaceutical composition is adapted to oral, parenteral (e.g. bronchopulmonary), local or topical application.
